(19) United States
(12) Patent Application Publication
(10) Pub. No.: US 2023/0118116 A1
(43) Pub. Date: Apr. 20, 2023

(54) ARYLAMINE COMPOUND AND ORGANIC ELECTROLUMINESCENT DEVICE

(71) Applicant: Shaanxi Lighte Optoelectronics Material Co., Ltd., Xi'an (CN)

(72) Inventors: Gaofei DENG, Xi'an (CN); Yu ZHAO, Xi'an (CN); Zhiwei CHEN, Xi'an (CN); Jinping WANG, Xi'an (CN)

(21) Appl. No.: 17/787,626

(22) PCT Filed: Dec. 24, 2020
(86) PCT No.: PCT/CN2020/138946

§ 371 Date: Jun. 21, 2022

(30) Foreign Application Priority Data
Dec. 27, 2019 (CN) 201911382479.9
Dec. 18, 2020 (CN) 202011507383.3

(51) Int. Cl. H01L 51/00; C07C 211/54; C07C 211/58; C07D 307/91; C07D 251/24; C07D 403/04; C07D 405/04; C07D 405/14; C07D 333/76; C07D 215/38; C07D 401/14; C07D 239/42; C07D 213/74

(52) U.S. Cl. CPC H01L 51/0059; C07C 211/54; C07C 211/58; H01L 51/006; C07D 307/91; H01L 51/0061; C07D 251/24; C07D 403/04; C07D 405/04; C07D 405/14; C07D 333/76; C07D 215/38; C07D 401/14; C07D 239/42; C07D 213/74; H01L 51/0073; H01L 51/0052; H01L 51/0072; H01L 51/0067; H01L 51/5056

(57) ABSTRACT

The present disclosure relates to an arylamine compound and an organic electroluminescent device. The arylamine compound has a structure represented by Formula (1), and contains N atoms rich in electrons and a number of large conjugated systems, so that the electron mobility and the transition rate can be improved. The device prepared from the arylamine compound has high electrical stability, and device efficiency and long device lifetime.

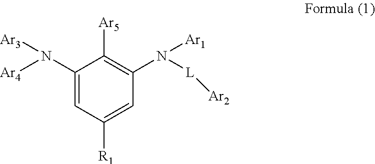

### CROSS REFERENCE TO RELATED APPLICATIONS

The present application claims the priority of the Chinese patent application No. CN201911382479.9 filed on Dec. 27, 2019 and the priority of the Chinese patent application No. CN202011507383.3 filed on Dec. 18, 2020, and the contents of the Chinese patent applications are hereby incorporated by reference in their entirety as a part of the present application.

### TECHNICAL FIELD

The present disclosure relates to the technical field of organic electroluminescent material, and specifically provides an arylamine compound and an organic electroluminescent device using the same.

### BACKGROUND

An organic light-emitting diodes, referred to as OLED, its the principle is that when an electric field is applied to a cathode and an anode, holes on an anode side and electrons on a cathode side move to a light-emitting layer where the holes and electrons are combined to form excitons which release energy outward in an excited state, and light is emitted outwards in the process of changing from energy release in an excited state to energy release in a ground state. Research and development are carried out all over the world since organic molecule electroluminescence reported by American Kodak Company in 1987 and polymer electroluminescence reported by British Cambridge University in 1990. This material has the advantages of simple structure, high yield, low cost, active luminescence, fast response speed, high fraction and the like, has the properties of low driving voltage, full solid state, non-vacuum, oscillation resistance, low temperature (−40° C.) resistance and the like, is considered as a new technology most possibly replacing a liquid crystal display in the future, and has attracted great attention.

In order to improve the brightness, the efficiency and the lifetime of the organic electroluminescent device, multi-layer structures are generally used in the organic electroluminescent device, and the multi-layer structures can include one or more of the following film layers: Hole injection layer (HIL), Hole transport layer (HTL), Electron-blocking layer (EBL), Light-emitting layer (EML), Hole-blocking layer (HBL), Electron transport layer (ETL), Electron injection layer (EIL) and the like. The film layers can improve the injection efficiency of carriers (holes and electrons) among interfaces of the layers and balance the transport capacity of the carriers between the layers, so that the brightness and efficiency of the organic electroluminescent device are improved.

At present, the existing light-emitting layer materials in the organic electroluminescent device are NPB, TPD and m-MTDATA, but these materials are generally lower in luminous efficiency and poorer in thermal stability, so that the organic electroluminescent device is shorter in lifetime and lower in luminous efficiency.

### SUMMARY

The purpose of the present disclosure is to improve the luminous efficiency and lifetime of an organic electroluminescent device.

In order to achieve the purpose, a first aspect of the present disclosure provides an arylamine compound having a structure represented by Formula (1):

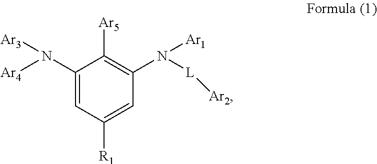

wherein R₁ and Ar₅ are the same or different, and are respectively independently selected from a substituted or unsubstituted aryl with 6 to 30 carbon atoms, a substituted or unsubstituted heteroaryl with 2 to 40 carbon atoms, a substituted or unsubstituted alkyl with 1 to 10 carbon atoms, hydrogen, deuterium, a halogen, a cyano, a trialkylsilyl with 3 to 10 carbon atoms, or a triphenylsilyl, and Ar₅ is not hydrogen;

Ar₁, Ar₂, Ar₃ and Ar₄ are the same or different, and are respectively independently selected from a substituted or unsubstituted aryl with 6 to 40 carbon atoms, or a substituted or unsubstituted heteroaryl with 2 to 40 carbon atoms;

L is selected from a single bond, a substituted or unsubstituted arylene with 6 to 30 carbon atoms, or a substituted or unsubstituted heteroarylene with 2 to 30 carbon atoms; and

the substituents of R₁, Ar₅, Ar₁, Ar₂, Ar₃ and Ar₄ and L are the same or different, and are respectively independently selected from deuterium, a halogen, a cyano, an aryl with 6 to 20 carbon atoms, a heteroaryl with 3 to 20 carbon atoms, an alkyl with 1 to 10 carbon atoms, a cycloalkyl with 3 to 6 carbon atoms, a trialkylsilyl with 1 to 10 carbon atoms, or a triarylsilyl with 6 to 48 carbon atoms.

A second aspect of the present disclosure provides an organic electroluminescent device, comprising an anode, a cathode and at least one functional layer between the anode layer and the cathode layer, the functional layer includes a hole injection layer, a hole transport layer, an organic electroluminescent layer, an electron transport layer, an electron injection layer and an electron-blocking layer. At least one of the organic electroluminescent layer, the hole transport layer and the electron transport layer contains the arylamine compound in the first aspect of the present disclosure.

According to the above technical solution, the molecular structure of the arylamine compound provided by the present disclosure contains an electron-donating aromatic amine group and a number of large conjugated systems, so that the electron mobility and the transition rate can be improved. The molecular structure of the arylamine compound has large steric hindrance, large distortion angle, and relatively high T1 value, and therefore the arylamine compound can be used as an organic electroluminescent device material, so that the electrical stability, device luminous efficiency and color purity of the organic electroluminescent device can be improved, and the device lifetime can be prolonged.

Other features and advantages of the present disclosure will be described in detail in the subsequent DETAILED DESCRIPTION OF THE EMBODIMENTS.

### BRIEF DESCRIPTION OF THE DRAWINGS

The FIGURES herein are incorporated into and constitute part of the description, illustrat examples conforming to the present application and are used together with the description to interpret the principles of the present application. Obviously, the FIGURES described below are only some examples of the present application and for those ordinary skilled in the art, other FIGURES can be obtained according to these FIGURES on the premise of not paying creative labor.

FIG. 1 is a structural schematic diagram of an organic electroluminescent device provided by one embodiment of the present application.

### DESCRIPTION OF REFERENCE SIGNS

100, Anode; 200, Cathode; 300, Functional layer; 310, Hole injection layer; 320, Hole transport layer; 321, First hole transport layer; 322, Second hole transport layer; 330, Organic electroluminescent layer; 340, Hole-blocking layer; 350, Electron transport layer; 360, Electron injection layer; 370, Electron-blocking layer.

### DETAILED DESCRIPTION OF THE EMBODIMENTS

The example embodiments are now described more thoroughly in combination with the FIGURES. However, the example embodiments can be implemented in multiple forms and shall not be construed as limitations to the embodiments set forth herein; on the contrary, these embodiments are provided to make the present disclosure more comprehensive and complete, and fully convey the concept of the example embodiments to those skilled in the art. The described characteristics, structures or features may be integrated into one or more embodiments in any suitable manner. In the following description, many specific details are provided to give a full understanding of the embodiments of the present disclosure.

The term “the” is used to indicate the existence of one or more elements/components/and the like; the terms “comprise” and “have” are used to express the meaning of open inclusion and mean that there may be other elements/components/and the like in addition to the listed elements/components/and the like.

A first aspect of the present disclosure provides an arylamine compound having a structure represented by Formula (1),

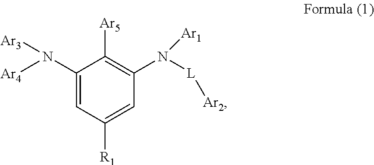

wherein R₁ and Ar₅ are the same or different, and are respectively independently selected from a substituted or unsubstituted aryl with 6 to 30 carbon atoms, a substituted or unsubstituted heteroaryl with 2 to 40 carbon atoms, a substituted or unsubstituted alkyl with 1 to 10 carbon atoms, hydrogen, deuterium, a halogen, a cyano, a trialkylsilyl with 3 to 10 carbon atoms, or a triphenylsilyl, and Ar₅ is not hydrogen;

Ar₁, Ar₂, Ar₃ and Ar₄ are the same or different, and are respectively independently selected from a substituted or unsubstituted aryl with 6 to 40 carbon atoms, or a substituted or unsubstituted heteroaryl with 2 to 40 carbon atoms;

L is selected from a single bond, a substituted or unsubstituted arylene with 6 to 30 carbon atoms, or a substituted or unsubstituted heteroarylene with 2 to 30 carbon atoms;

the substituents of R₁, Ar₅, Ar₁, Ar₂, Ar₃, Ar₄ and L are the same or different, and are respectively independently selected from deuterium, a halogen, a cyano, an aryl with 6 to 20 carbon atoms, a heteroaryl with 3 to 20 carbon atoms, an alkyl with 1 to 10 carbon atoms, a cycloalkyl with 3 to 6 carbon atoms, a trialkylsilyl with 1 to 10 carbon atoms, or a triarylsilyl with 6 to 48 carbon atoms.

The molecular structure of the arylamine compound provided by the present disclosure contains an electron-donating aromatic amine group and a number of large conjugated systems, so that the electron mobility and the transition rate can be improved, and the arylamine compound can be used as a light-emitting layer material of an organic electroluminescent device, so that the electrical stability, the color purity and the device luminous efficiency of the organic electroluminescent device can be improved, and the device lifetime can be prolonged.

In the present disclosure, the term of “with x to y carbon atoms” (x and y are positive integers satisfying x<y) means that the number of carbon atoms in a partial structure equivalent to a functional group name described behind the term is x to y. For example, “alkyl with 1 to 10 carbon atoms” refers to alkyl having 1 to 10 carbon atoms, and “aryl with 6 to 30 carbon atoms” refers to aryl having 6 to 30 carbon atoms.

In the present disclosure, a single bond means that no other atom is present in the moiety represented by L. For example, when L in Formula (1) is a single bond, N may be directly connected to Ar₂.

In the present disclosure, the term of “substituted or unsubstituted” means that a functional group described behind the term may have or do not have a substituent Ra. For example, “substituted or unsubstituted alkyl” refers to alkyl having the substituent Ra or unsubstituted alkyl. Ra may be deuterium; halogen; hydroxy; cyano; nitro; amino; alkyl; cycloalkyl; haloalkyl; heteroalkyl containing one or more of O, N, Si and S; heterocycloalkyl; alkoxy; alkylthio; dialkylamino; diarylamino; aryloxy; arylthio; silyl; trialkylsilyl; triarylsilyl; alkenyl; or cycloalkenyl. The groups may also have substituent selected from the above substituents.

In the specific embodiments of the present disclosure, the substituent Ra can be selected from one or more of deuterium, halogen, cyano, methyl, ethyl, isopropyl, tert-butyl, phenyl, biphenyl, naphthyl, phenanthryl, quinolinyl, isoquinolinyl, N-phenylcarbazolyl, pyridyl, pyrimidinyl, dibenzofuranyl, dibenzothienyl, 9,9-diphenylfluorenyl, anthracenyl, carbazolyl, 9,9-dimethylfluorenyl, spirobifluorenyl, chrysenyl, quinazolinyl, or quinoxalinyl.

In the present disclosure, the alkyl with 1 to 10 carbon atoms may be linear alkyl or branched alkyl. Specifically, the alkyl with 1 to 10 carbon atoms may be linear alkyl having 1 to 10 carbon atoms; or branched alkyl having 3 to 10 carbon atoms. More specifically, the alkyl with 1 to 10 carbon atoms may be methyl, ethyl, n-propyl, isopropyl, n-butyl, isobutyl, tert-butyl, n-amyl, isoamyl, neopentyl, hexyl, heptyl, octyl, 2-ethylhexyl, nonyl, decyl, 3,7-dimethyloctyl, and the like, and the cycloalkyl with 3 to 6 carbon atoms may be cyclopentyl or cyclohexyl, but is not limited thereto.

In the present disclosure, when no specific definition is additionally provided, ‘hetero’ means that one functional group includes at least one hetero atom such as B, O, N, P, Si or S, and the remaining atoms are carbon and hydrogen. The unsubstituted alkyl may be a “saturated alkyl group” without any double bond or triple bond.

In the present disclosure, aryl refers to an optional functional group or substituent derived from an aromatic hydrocarbon ring. The aryl may be monocyclic aryl or polycyclic aryl, in other words, the aryl can be monocyclic aryl, fused aryl, two or more monocyclic aryl conjugatedly connected through carbon-carbon bonds, monocyclic aryl and fused aryl conjugatedly connected through a carbon-carbon bond, and two or more fused aryl conjugatedly connected through carbon-carbon bonds. In other words, two or more aromatic groups conjugatedly connected by carbon-carbon bonds can also be regarded as the aryl of the present disclosure. The aryl does not contain heteroatoms such as B, O, N, P, Si or S. For example, in the present disclosure, biphenyl, terphenyl, etc. are aryl. Examples of the aryl may include phenyl, naphthyl, fluorenyl, anthracenyl, phenanthryl, biphenyl, terphenyl, quaterphenyl, quinquephenyl, sexiphenyl, benzo[9,10]phenanthryl, pyrenyl, benzofluoranthenyl, chrysenyl, fluorenyl, 9,9-dimethylfluorenyl, 9,9-diphenylfluorenyl, spirobifluorenyl, and the like, but is not limited thereto.

In the present disclosure, the unsubstituted aryl refers to aryl having 6 to 30 carbon atoms, such as phenyl, naphthyl, pyrenyl, dimethylfluorenyl, 9,9-diphenylfluorenyl, spirobifluorenyl, anthracenyl, phenanthryl, chrysenyl, azulenyl, acenaphthenyl, biphenyl, benzanthracenyl, spirobifluorenyl, perylenyl, indenyl and the like. The substituted aryl having 6 to 30 carbon atoms means that at least one hydrogen atom is substituted by deuterium, F, Cl, I, CN, hydroxy, nitro, amino and the like. The substituted aryl means that one or more hydrogen atoms in the aryl are substituted by other groups. For example, at least one hydrogen atom is substituted by deuterium, F, Cl, Br, I, CN, hydroxy, amino, branched alkyl, linear alkyl, cycloalkyl, alkoxy, alkylamino or other groups, such as 9,9-dimethylfluorenyl, 9,9-diphenylfluorenyl, spirobifluorenyl and the like. It can be understood that substituted aryl having 20 carbon atoms means that the total number of carbon atoms of the aryl and substituents on the aryl is 20. For example, the number of carbon atoms of 9,9-dimethylfluorenyl is 15.

In the present disclosure, the substituted aryl can be that one or two or more hydrogen atoms in the aryl are substituted by a group such as deuterium, halogen, —CN, aryl, heteroaryl, trialkylsilyl, alkyl, cycloalkyl, alkoxy, alkylthio and the like. Specific examples of heteroaryl-substituted aryl include, but are not limited to, dibenzofuranyl-substituted phenyl, dibenzothienyl-substituted phenyl, pyridyl-substituted phenyl, and the like. It should be understood that the number of carbon atoms in the substituted aryl refers to the total number of carbon atoms in the aryl and substituents on the aryl, for example, the substituted aryl having 18 carbon atoms means that the total number of carbon atoms in the aryl and substituents is 18.

In the present disclosure, the heteroaryl may be heteroaryl including at least one of B, O, N, P, Si, and S as a heteroatom. The heteroaryl may be monocyclic heteroaryl or polycyclic heteroaryl, in other words, the heteroaryl may be a single aromatic ring system or a plurality of aromatic ring systems conjugatedly connected through carbon-carbon bonds, and any one aromatic ring system is an aromatic monocyclic ring or an aromatic fused ring. Exemplarily, the heteroaryl may include thienyl, furanyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolinyl, quinazolinyl, quinoxalinyl, phenoxazinyl, phthalazinyl, pyridinopyrimidyl, pyridinopyrazinyl, pyrazinopyrazinyl, isoquinolinyl, indolyl, carbazolyl, N-arylcarbazolyl, N-heteroarylcarbazolyl, N-alkylcarbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, benzothiazolyl, phenothiazinyl, dibenzosilyl, dibenzofuranyl, phenyl-substituted dibenzofuranyl, dibenzofuranyl-substituted phenyl, and the like, but is not limited thereto. The thienyl, furanyl, phenanthrolinyl and the like are heteroaryl of the single aromatic ring system, and the N-arylcarbazolyl, N-heteroarylcarbazolyl, phenyl-substituted dibenzofuranyl, dibenzofuranyl-substituted phenyl and the like are heteroaryl of the plurality of aromatic ring systems conjugatedly connected through carbon-carbon bonds.

In the present disclosure, the substituted heteroaryl can be that one or more hydrogen atoms in the heteroaryl are substituted by a group such as deuterium atom, halogen, —CN, aryl, heteroaryl, trialkylsilyl, alkyl, cycloalkyl, alkoxy, alkylthio and the like. Specific examples of aryl-substituted heteroaryl include but are not limited to phenyl-substituted dibenzofuranyl, phenyl-substituted dibenzothienyl, phenyl-substituted pyridyl and the like. It should be understood that the number of carbon atoms of the substituted heteroaryl refers to the total number of carbon atoms of heteroaryl and substituents on the heteroaryl.

The description modes “each . . . are independently”, “ . . . are respectively independently” and “ . . . are independently selected from” adopted in the present disclosure can be interchanged, and should be understood in a broad sense, which means that in different groups, specific options expressed between the same symbols do not influence each other, or in a same group, specific options expressed between the same symbols do not influence each other.

For example, in the description of

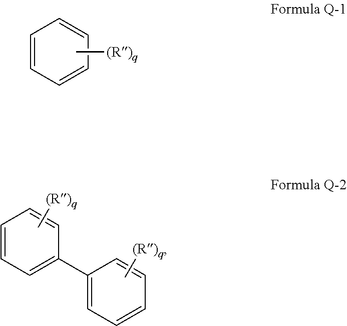

wherein each q is independently 0, 1, 2 or 3, and each R″ is independently selected from hydrogen, fluorine and chlorine”, its meaning is as follows: Formula Q-1 represents that q substituents R″ exist on a benzene ring, each R″ can be the same or different, and options of each R″ do not influence each other; Formula Q-2 represents that each benzene ring of biphenyl has q substituents R″, the number q of the substituents R″ on the two benzene rings can be the same or different, each R″ can be the same or different, and options of each R″ do not influence each other.

An unpositioned connecting bond in the present disclosure is a single bond

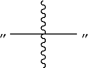

extending from a ring system, which means that one end of the connecting bond can be connected with any position in the ring system through which the bond penetrates, and the other end of the connecting bond is connected with the remaining part of a compound molecule. For example, as shown in the following Formula (f), naphthyl represented by the Formula (f) is connected to other positions of a molecule through two unpositioned connecting bonds penetrating a dicyclic ring, and its meaning includes any one possible connecting mode represented by Formulas (f-1)-(f-10).

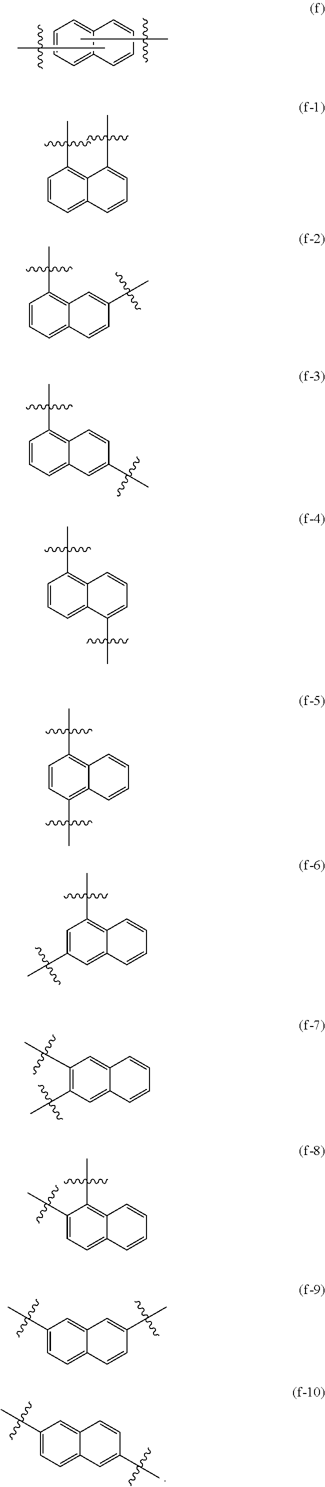

For example, as shown in the following Formula (X′), phenanthryl represented by Formula (X′) is connected with other positions of a molecule through one unpositioned connecting bond extending from the middle of a benzene ring on one side, and its meaning includes any possible connecting mode represented by Formulas (X′-1)-(X′-4):

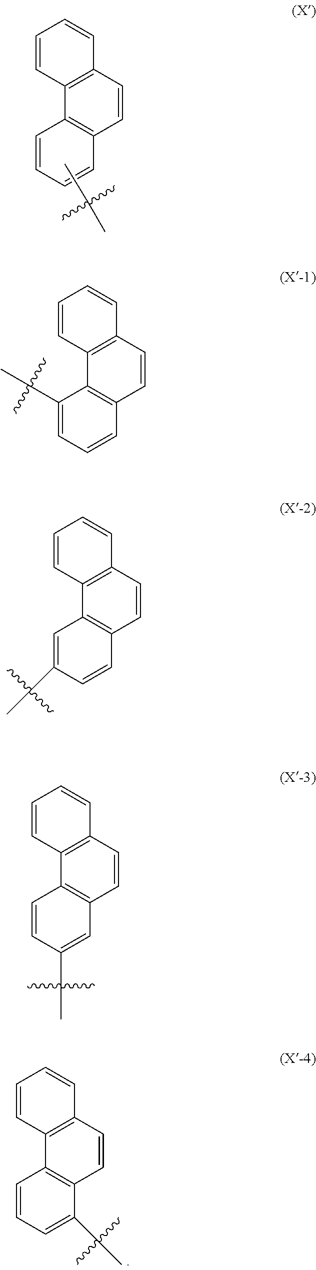

An unpositioned substituent in the present disclosure refers to a substituent connected through a single bond extending from the center of a ring system, which means that the substituent can be connected to any possible position in the ring system. For example, as shown in the following Formula (Y), a substituent R represented by Formula (Y) is connected with a quinoline ring through an unpositioned connecting bond, and its meaning includes any possible connecting mode represented by Formulas (Y-1)-(Y-7):

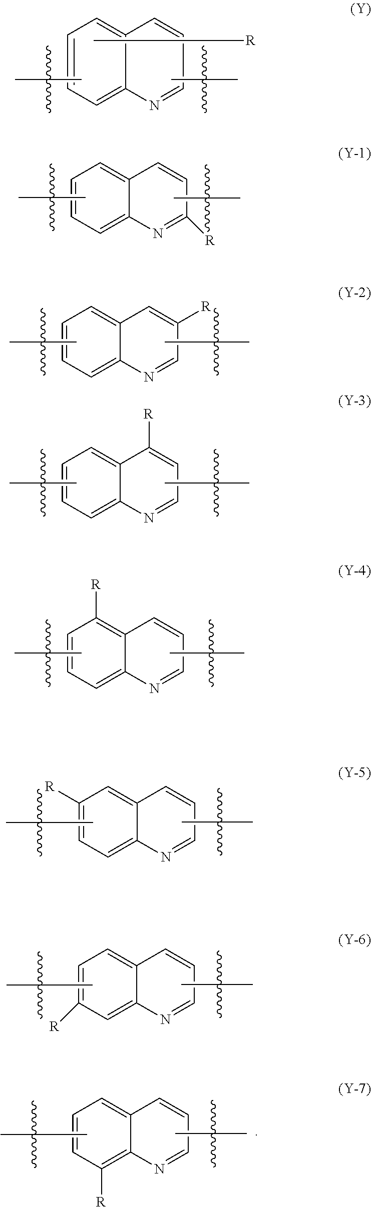

In the present disclosure, specific examples of trialkylsilyl include but are not limited to trimethylsilyl, triethylsilyl and the like.

In the present disclosure, specific examples of triarylsilyl include but are not limited to triphenylsilyl and the like.

In the present disclosure, the interpretation of aryl and heteroaryl is suitable for arylene and heteroarylene.

In one embodiment of the present disclosure, the arylamine compound has a structure represented by Formula (1):

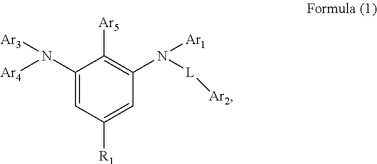

wherein R₁ is an alkyl with 1 to 10 carbon atoms;

Ar₁, Ar₂, Ar₃, Ar₄ and Ar₅ are the same or different, and are each independently selected from a substituted or unsubstituted aryl with 6 to 30 carbon atoms, or a substituted or unsubstituted heteroaryl with 2 to 40 carbon atoms;

L is selected from a single bond, a substituted or unsubstituted arylene with 6 to 30 carbon atoms, or a substituted or unsubstituted heteroarylene with 2 to 30 carbon atoms;

the substituents of Ar₁, Ar₂, Ar₃, Ar₄, Ar₅ and L are the same or different, and are respectively independently selected from deuterium, a halogen, a cyano, an alkyl with 1 to 10 carbon atoms, a cycloalkyl with 3 to 6 carbon atoms, a trialkylsilyl with 1 to 10 carbon atoms, or a triarylsilyl with 6 to 48 carbon atoms.

In one embodiment of the present disclosure, in the arylamine compound represented by Formula (1), L is selected from a single bond or a group represented by the following general formulas:

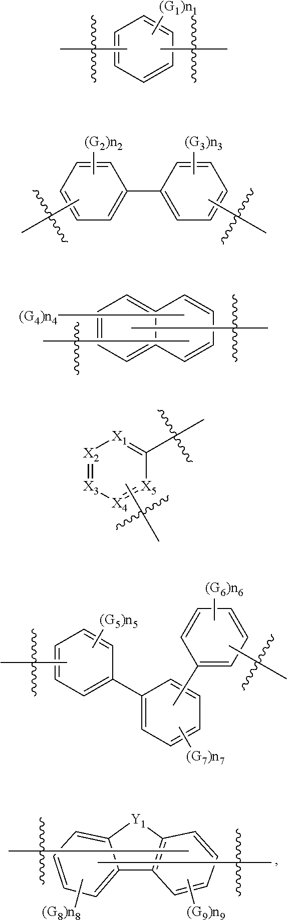

wherein, [symbol] represents a chemical bond;

n₁, n₂, n₃, n₅, n₆, n₇, n₈ and n₉ are each independently selected from 0, 1, 2, 3 or 4;

n₄ is selected from 0, 1, 2, 3, 4, 5 or 6;

Y₁ is selected from C(G₁₀G₁₁), O, S, Se, Si(G₁₂G₁₃) or N(G₁₄);

G₁ to G₁₄ are the same or different, and are respectively independently selected from hydrogen, deuterium, a halogen, a cyano, an alkyl with 1 to 10 carbon atoms, an aryl with 6 to 18 carbon atoms, a heteroaryl with 3 to 18 carbon atoms, or a cycloalkyl with 3 to 10 carbon atoms; or G₁₀ and G₁₁ are connected to form a ring, or G₁₂ and G₁₃ are connected to form a ring;

X₁ to X₅ are the same or different, and are respectively independently selected from C(R′) or N, at least one of X₁ to X₅ is N, and R′ in X₁ to X₅ are the same or different, and are respectively independently selected from hydrogen, an alkyl with 1 to 10 carbon atoms, an aryl with 6 to 18 carbon atoms, a heteroaryl with 3 to 18 carbon atoms or a cycloalkyl with 3 to 10 carbon atoms, or any two adjacent R′ are connected to form a ring.

In the present disclosure, when n₁ is greater than or equal to 2, G₁ are the same or different; when n₂ is greater than or equal to 2, G₂ are the same or different; when n₃ is greater than or equal to 2, G₃ are the same or different; when n₄ is greater than or equal to 2, G₄ are the same or different; when n₅ is greater than or equal to 2, G₅ are the same or different; when n₆ is greater than or equal to 2, G₆ are the same or different; when n₇ is greater than or equal to 2, G₇ are the same or different; when n₈ is greater than or equal to 2, G₈ are the same or different; and when n₉ is greater than or equal to 2, G₉ is the same or different.

In the present disclosure, when n₁ to n₁₁ are selected from 0, the benzene ring is not substituted.

In the present disclosure, “any two adjacent R′ are connected into a ring”, namely any two adjacent R′ are connected with each other so as to form a ring with atoms to which they are joined together. For example, a ring with 3 to 15 carbon atoms can be formed, and for example, a ring with 3 to 10 carbon atoms can be formed; the ring can be saturated (such as a five-membered ring or a six-membered ring), and can also be unsaturated, such as an aromatic ring.

In the present disclosure, the ring refers to a saturated or unsaturated ring, such as cyclohexane, cyclopentane, a 6- to 12-membered aromatic ring, a 5- to 12-membered heteroaromatic ring, and the like, but is not limited to thereto.

In the present disclosure, a ring system formed by n atoms is an n-membered ring. For example, phenyl is 6-membered aryl. A 6- to 10-membered aromatic ring refers to a benzene ring, an indene ring, a naphthalene ring or the like.

In the present disclosure, the ring refers to a saturated or unsaturated ring, and optionally, the number of carbon atoms of the ring may be 5, for example

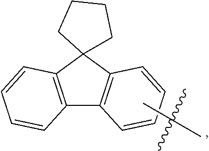

may also be 6, for example

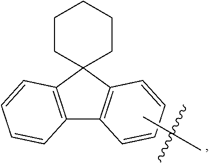

and may also be 13, for example

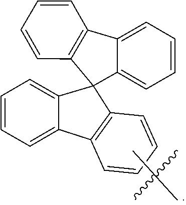

Certainly, the number of carbon atoms forming the ring can also be other numerical values which are not listed one by one, and the number of carbon atoms of the ring is not specially limited in the present disclosure.

In one embodiment of the present disclosure, in the arylamine compound represented by Formula (1), L is selected from a single bond, a substituted or unsubstituted phenylene, a substituted or unsubstituted naphthylene, a substituted or unsubstituted biphenylene, a substituted or unsubstituted dibenzofuranylene, or a substituted or unsubstituted diphenylfluorenylene.

In one embodiment of the present disclosure, in the arylamine compound represented by Formula (1), L is selected from a single bond, a substituted or unsubstituted arylene with 6 to 12 carbon atoms, or a substituted or unsubstituted heteroarylene with 4 to 12 carbon atoms;

preferably, L is selected from a substituted or unsubstituted pyridylene, a substituted or unsubstituted dibenzothienylene, or a substituted or unsubstituted pyrimidinylene.

In one embodiment of the present disclosure, in the arylamine compound represented by Formula (1), the substituents of L are the same or different, and are respectively independently selected from deuterium, a fluorine, a cyano, a methyl, an ethyl, an isopropyl, a tert-butyl, a phenyl, a naphthyl or a trimethylsilyl.

In one embodiment of the present disclosure, in the arylamine compound represented by Formula (1), L is selected from a single bond or a group consisting of groups represented by the following general formulas:

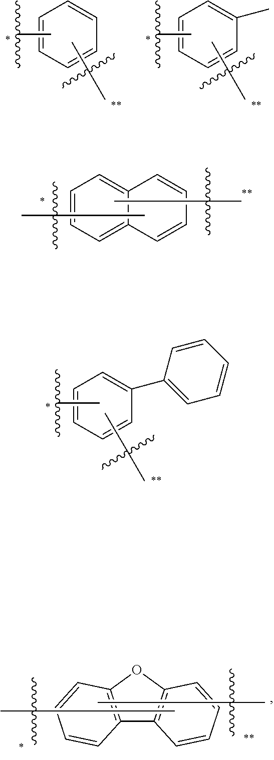

wherein, * is a linking point of L connected to

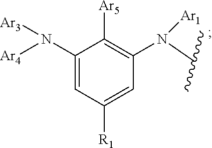

and ** is a linking point of L connected to

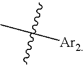

In one embodiment of the present disclosure, in the arylamine compound represented by Formula (1), L is selected from a single bond or a group consisting of groups represented by the following general formulas:

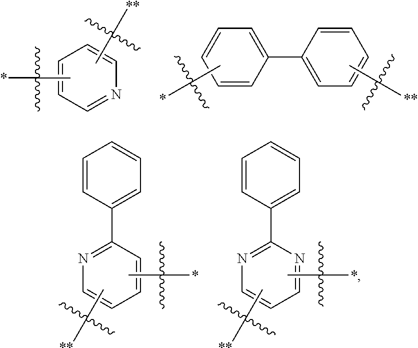

wherein, * is a linking point of L connected to

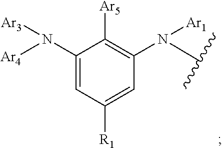

and ** is a linking point of L connected to

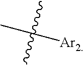

In one embodiment of the present disclosure, L is selected from a single bond, or a group consisting of the following groups;

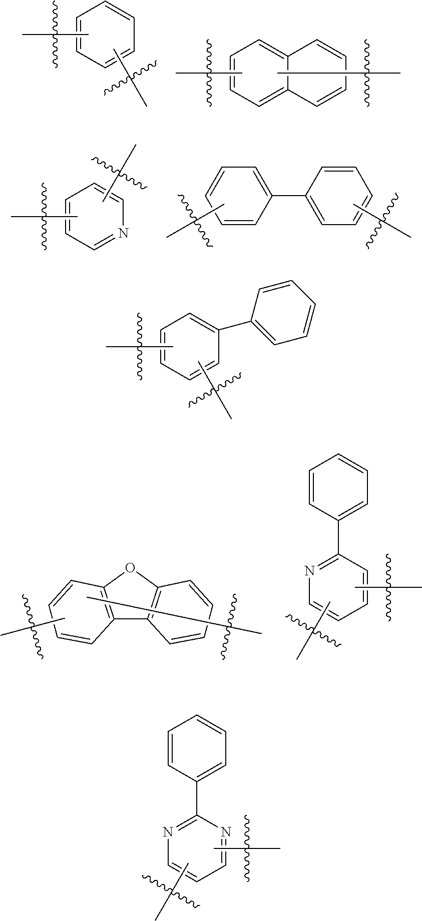

wherein, [symbol] represents a chemical bond.

In one embodiment of the present disclosure, L is selected from a single bond, or a group consisting of the following groups:

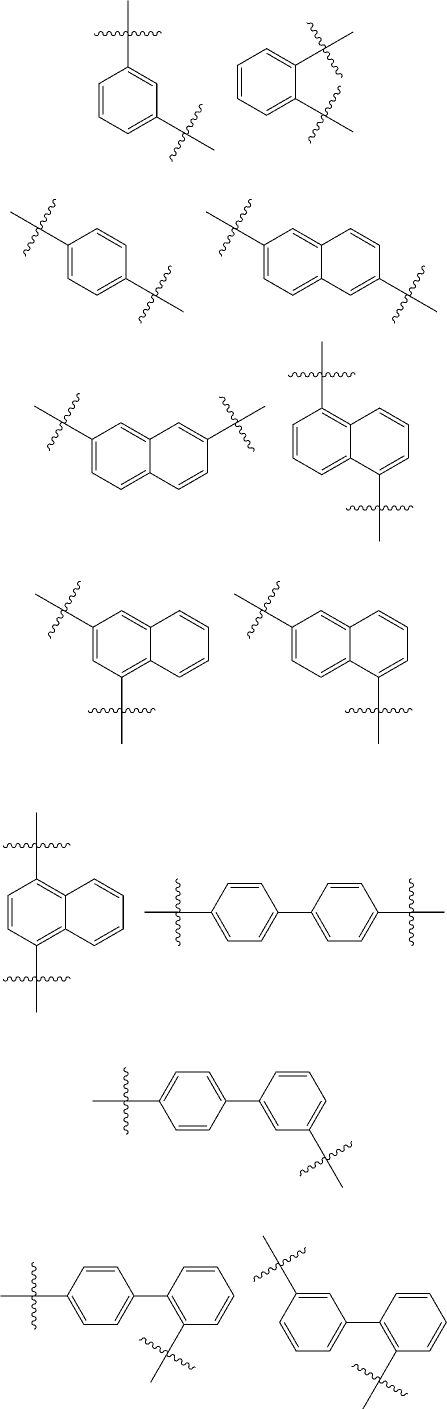

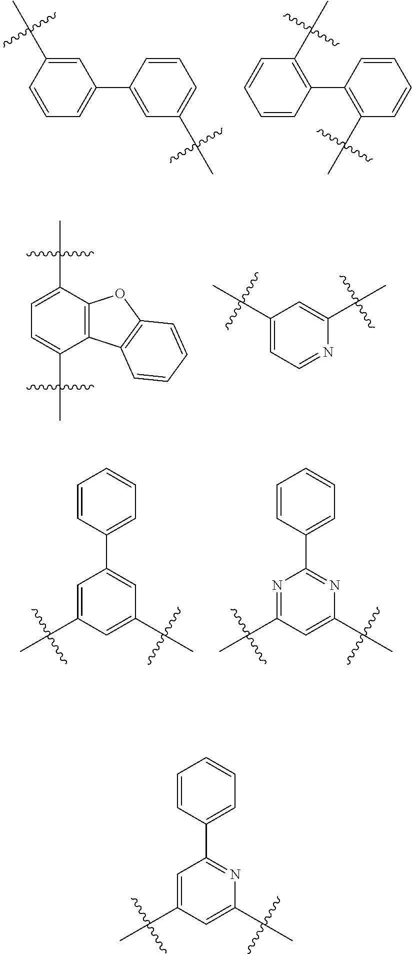

wherein, [symbol] represents a chemical bond.

In one specific embodiment of the present disclosure, Ar₁, Ar₂, Ar₃, Ar₄ and Ar₅ are the same or different, and are respectively independently selected from a substituted or unsubstituted aryl with 6 to 30 carbon atoms, or a substituted or unsubstituted heteroaryl with 2 to 30 carbon atoms.

Preferably, Ar₁, Ar₂, Ar₃, Ar₄ and Ar₅ are the same or different, are respectively independently selected from a substituted or unsubstituted aryl with 6 to 24 carbon atoms, or a substituted or unsubstituted heteroaryl with 3 to 25 carbon atoms, and are further independently selected from a substituted or unsubstituted aryl with 6 to 20 carbon atoms, or a substituted or unsubstituted heteroaryl with 5 to 20 carbon atoms.

In one embodiment of the present disclosure, Ar₁, Ar₂, Ar₃, Ar₄ and Ar₅ are the same or different, and are respectively independently selected from the following general formulas,

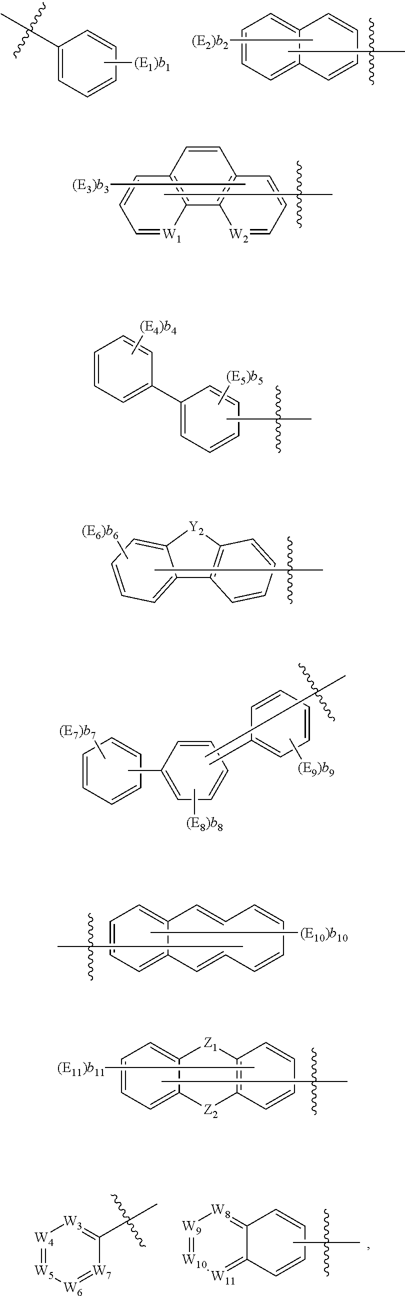

wherein,

b₁, b₄, b₇ and b₉ are the same or different, and are respectively independently selected from 0, 1, 2, 3, 4 or 5;

b₅, b₆ and b₈ are the same or different, and are respectively independently selected from 0, 1, 2, 3 or 4;

b₂, b₃ and b₁₁ are the same or different, and are respectively independently selected from 0, 1, 2, 3, 4, 5, 6 or 7;

b₁₀ is selected from 0, 1, 2, 3, 4, 5, 6, 7, 8 or 9;

W₁ and W₂ are the same or different, and are respectively independently selected from C or N;

Y₂ is selected from O, S, Si(E₁₂E₁₃), C(E₁₄E₁₅), N(E₁₆) or Se;

Z₁ and Z₂ are the same or different, and are respectively independently selected from O, S, C(E₁₇E₁₈) or N(E₁₉);

E₁ to E₁₉ are the same or different, and are respectively independently selected from hydrogen, deuterium, a halogen atom, a cyano, an alkyl with 1 to 10 carbon atoms, an aryl with 6 to 18 carbon atoms, a heteroaryl with 6 to 18 carbon atoms, or a cycloalkyl with 3 to 10 carbon atoms; or E₁₂ and E₁₃ are connected to form a ring, or E₁₄ and E₁₅ are connected to form a ring, or E₁₇ and E₁₈ are connected to form a ring;

W₃ to W₇ are the same or different, and are respectively independently selected from C(Q′) or N, at least one of W₃ to W₇ is N, Q in W₃ to W₇ are the same or different, and are respectively independently selected from hydrogen, an alkyl with 1 to 10 carbon atoms, an aryl with 6 to 18 carbon atoms, a heteroaryl with 3 to 18 carbon atoms, or a cycloalkyl with 3 to 10 carbon atoms, or any two adjacent Q are connected to form a ring;

W₈ to W₁₁ are the same or different, and are respectively independently selected from C(Q′) or N, at least one of W₈ to W₁₁ is N, Q′ in W₈ to W₁₁ are the same or different, and are respectively independently selected from hydrogen, an alkyl with 1 to 10 carbon atoms, an aryl with 6 to 18 carbon atoms, a heteroaryl with 3 to 18 carbon atoms, or a cycloalkyl with 3 to 10 carbon atoms, or any two adjacent Q′ are connected to form a ring.

In the present disclosure, when b₁ is greater than or equal to 2, E₁ are the same or different; when b₂ is greater than or equal to 2, E₂ are the same or different; when b₃ is greater than or equal to 2, E₃ are the same or different; when b₄ is greater than or equal to 2, E₄ are the same or different; when b₅ is greater than or equal to 2, E₅ are the same or different; when b₆ is greater than or equal to 2, E₆ are the same or different; when b₇ is greater than or equal to 2, E₇ are the same or different; when b₈ is greater than or equal to 2, E₈ are the same or different; when b₉ is greater than or equal to 2, E₉ are the same or different; when b₁₀ is greater than or equal to 2, E₁₀ are the same or different; and when b₁₁ is greater than or equal to 2, E₁₁ are the same or different.

In the present disclosure, when b₁ to b₁₁ are selected from 0, the benzene ring is not substituted.

The adjacent Q can be connected to form a ring, which means that W₃ and W₄ form a ring, or W₄ and W₅ form a ring, or W₅ and W₆ form a ring, or W₆ and W₇ form a ring, and certainly, also includes the condition that W₃ and W₄ form a ring, and W₅ and W₆ form a ring.

The adjacent Q′ can be connected to form a ring, which means that W₈ and W₉ form a ring, or W₉ and W₁₀ form a ring, or W₁₀ and W₁₁ form a ring, and certainly, also includes the condition that W₈ and W₉ form a ring, and W₁₀ and W₁₁ form a ring.

In one embodiment of the present disclosure, Ar₁, Ar₃ and Ar₄ are the same or different, and are respectively independently selected from a substituted or unsubstituted aryl with 6 to 20 carbon atoms, or a substituted or unsubstituted heteroaryl with 5 to 12 carbon atoms.

In one embodiment of the present disclosure, Ar₁, Ar₃, and Ar₄ are the same or different, and are respectively independently selected from a group consisting of the following groups:

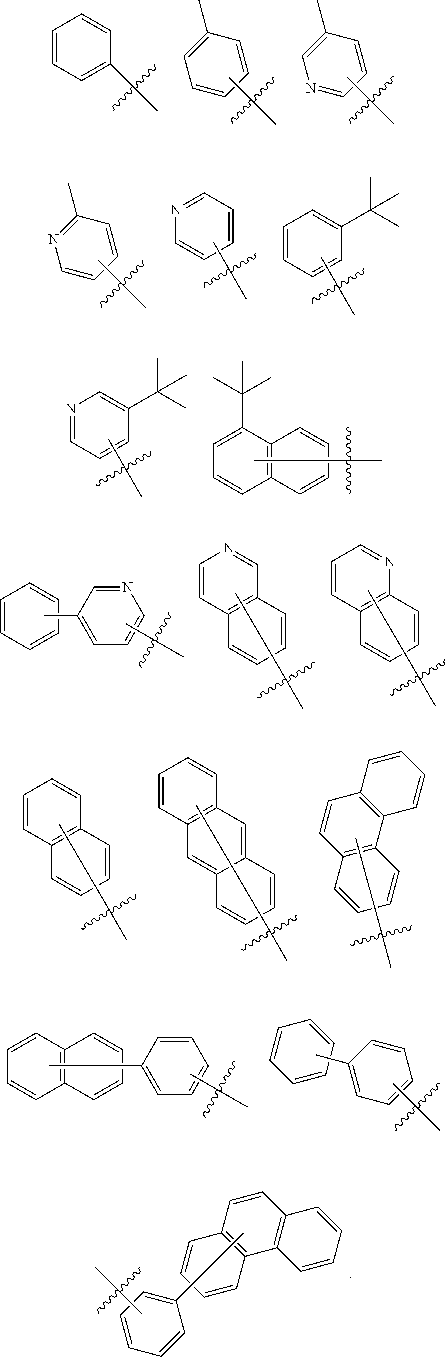

In one embodiment of the present disclosure, Ar₁, Ar₂, Ar₃ and Ar₄ are the same or different, and are respectively independently selected from a substituted or unsubstituted aryl with 6 to 25 carbon atoms, or a substituted or unsubstituted heteroaryl with 3 to 26 carbon atoms.

In one embodiment of the present disclosure, Ar₁, Ar₂, Ar₃ and Ar₄ are the same or different, and are respectively independently selected from a substituted or unsubstituted aryl with 6 to 20 carbon atoms, or a substituted or unsubstituted heteroaryl with 5 to 26 carbon atoms.

In one embodiment of the present disclosure, Ar₁, Ar₂, Ar₃, and Ar₄ are the same or different, and are respectively independently selected from a substituted or unsubstituted phenyl, a substituted or unsubstituted pyridyl, a substituted or unsubstituted pyrimidyl, a substituted or unsubstituted naphthyl, a substituted or unsubstituted biphenyl, a substituted or unsubstituted quinolinyl, a substituted or unsubstituted isoquinolinyl, a substituted or unsubstituted anthracenyl, a substituted or unsubstituted phenanthryl, a substituted or unsubstituted fluorenyl, a substituted or unsubstituted dibenzofuranyl, a substituted or unsubstituted dibenzothienyl, a substituted or unsubstituted carbazolyl, a substituted or unsubstituted N-phenylcarbazolyl, a substituted or unsubstituted triazinyl, a substituted or unsubstituted terphenyl, or a substituted or unsubstituted 9,9′-spirobifluorenyl.

In one embodiment of the present disclosure, the substituents of Ar₁, Ar₂, Ar₃ and Ar₄ are the same or different, and are respectively independently selected from deuterium, a halogen, a cyano, an alkyl with 1 to 5 carbon atoms, a trimethylsilyl, an aryl with 6 to 15 carbon atoms, or a heteroaryl with 3 to 20 carbon atoms.

In one embodiment of the present disclosure, Ar₁, Ar₂, Ar₃ and Ar₄ are the same or different, and are respectively independently selected from a substituted or unsubstituted group T, and the unsubstituted group T is selected from a group consisting of the following groups:

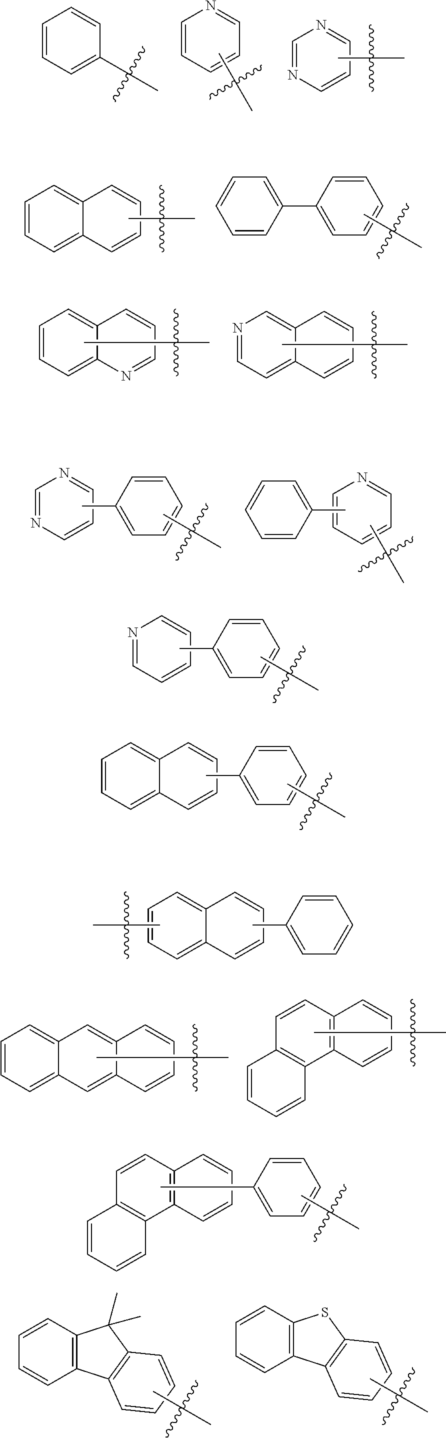

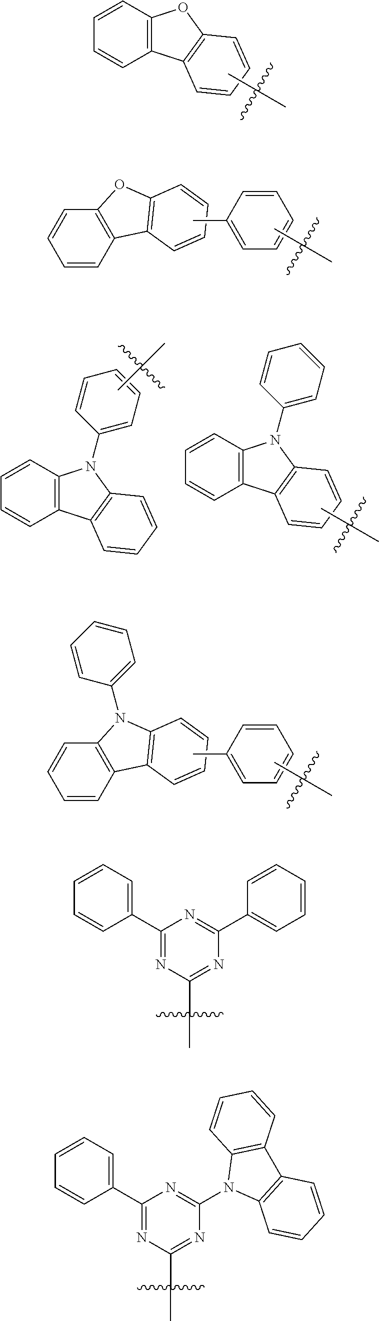

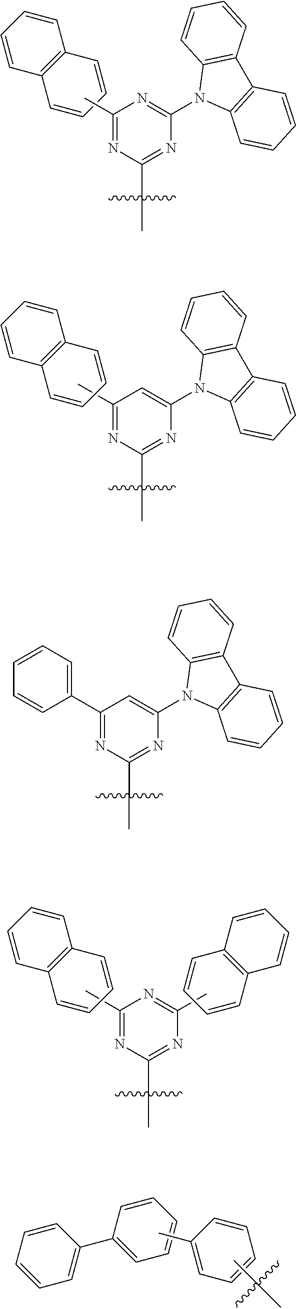

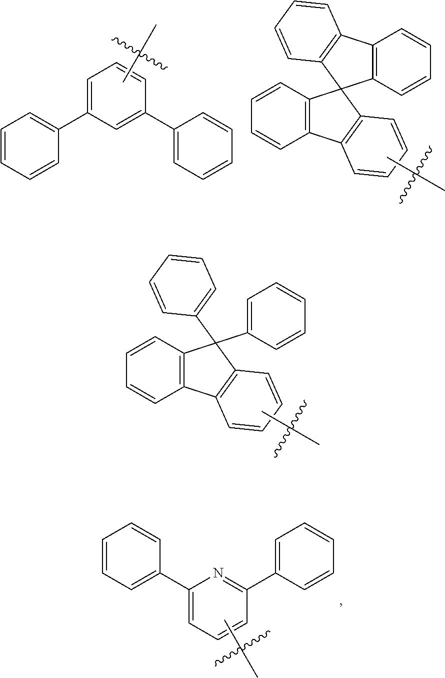

the substituted group T has one or more substituents, and the substituents of T are independently selected from deuterium, a fluorine, a cyano, a trimethylsilyl, a methyl, an ethyl, an isopropyl, a tert-butyl, a phenyl, a biphenyl, a pyridyl, a pyrimidyl, a N-phenylcarbazolyl, a phenanthryl, a naphthyl, a carbazolyl, a dibenzofuranyl, or a dibenzothienyl.

In one embodiment of the present disclosure, Ar₁, Ar₂, Ar₃, and Ar₄ are the same or different, and are respectively independently selected from a group consisting of the following groups:

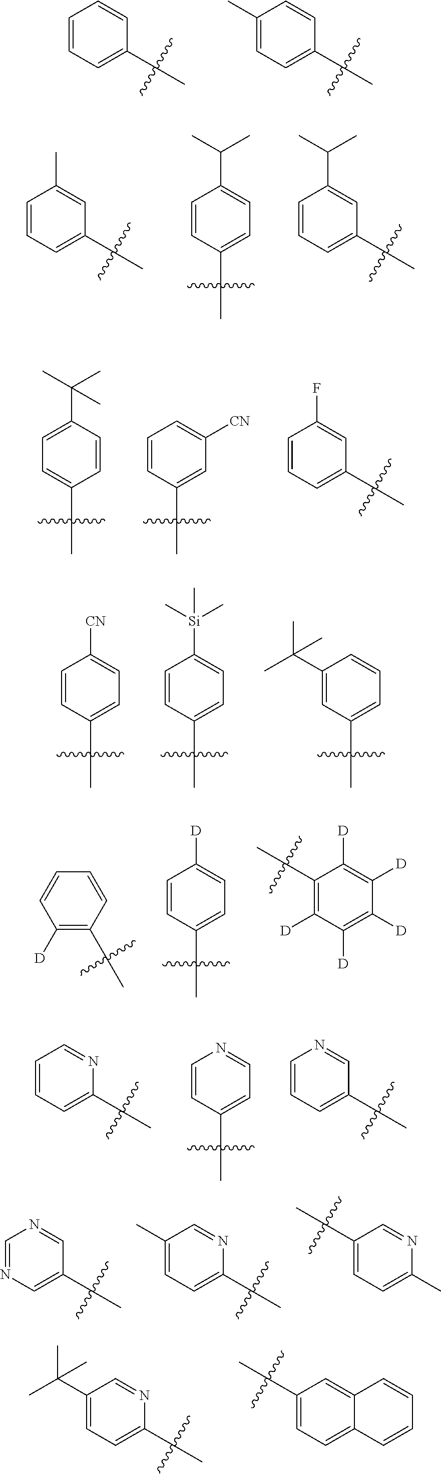

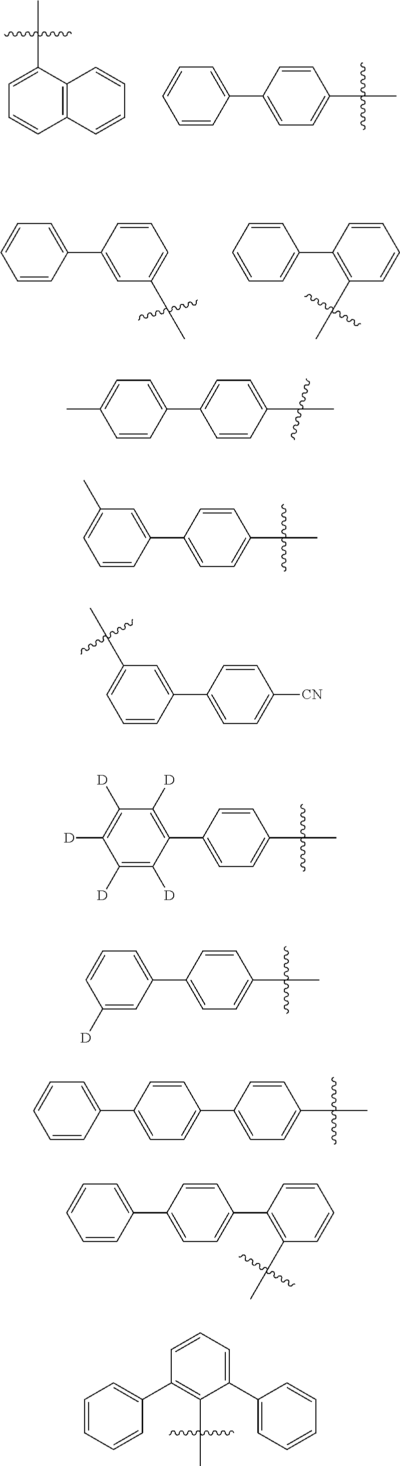

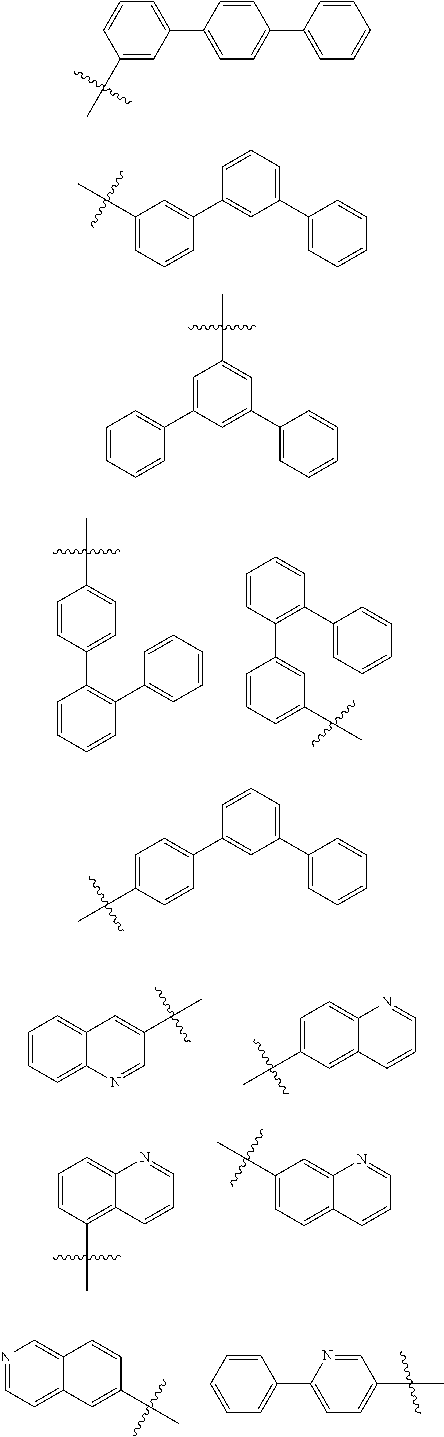

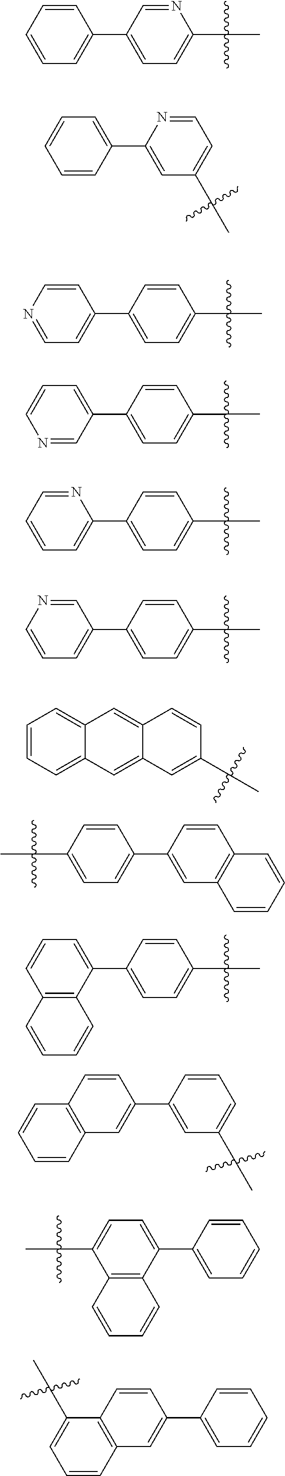

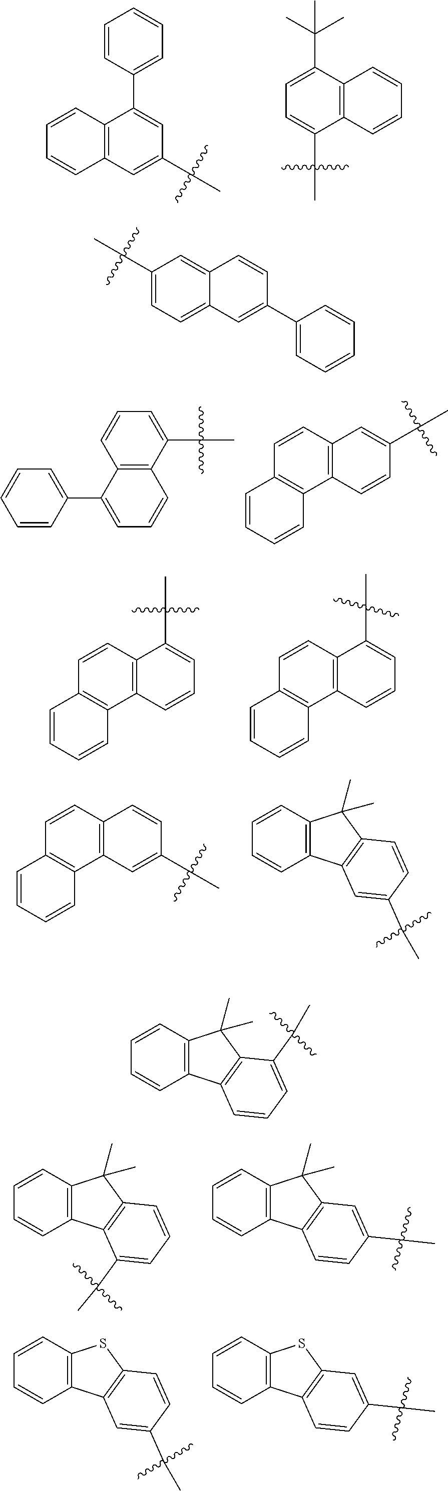

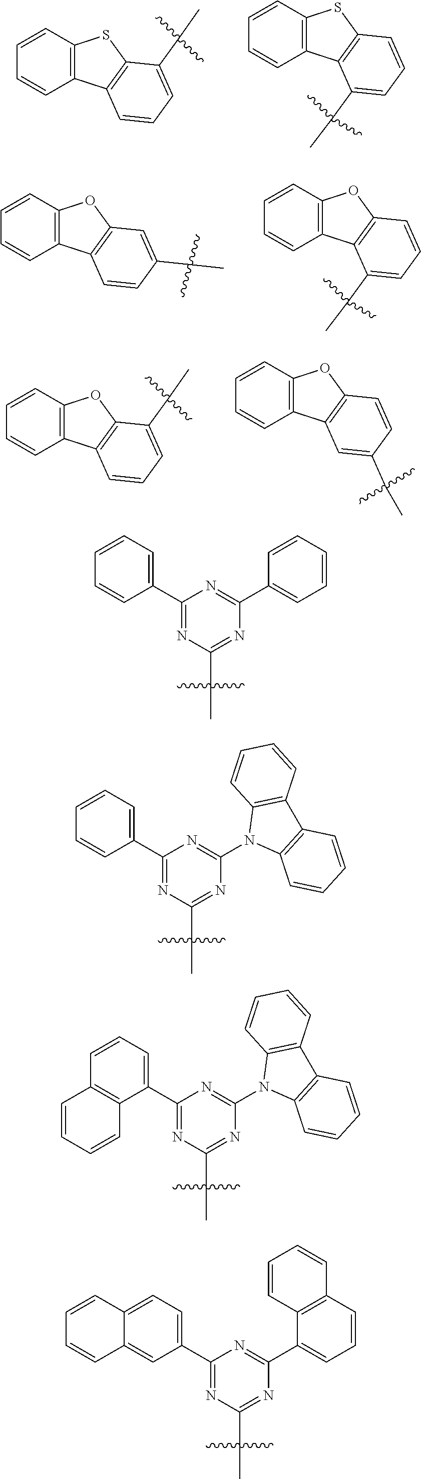

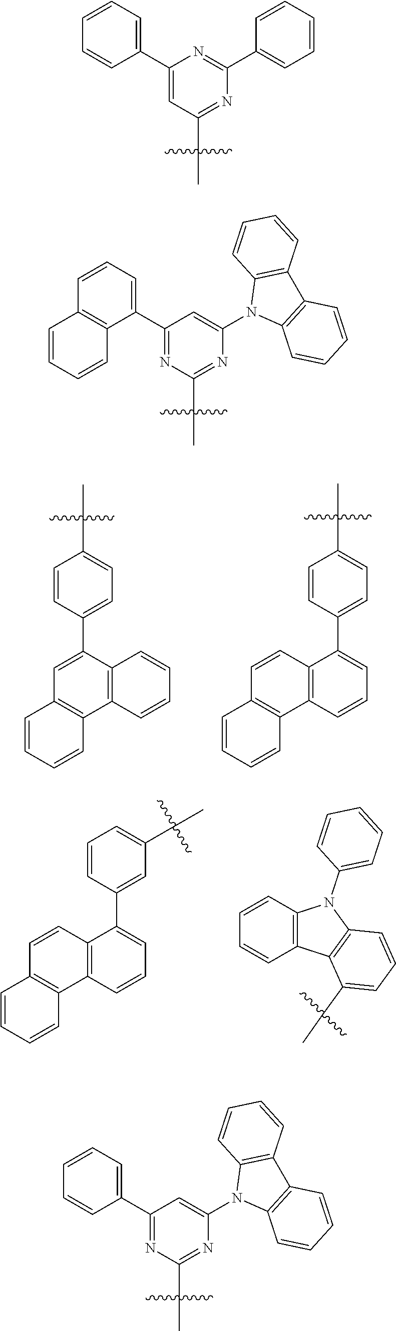

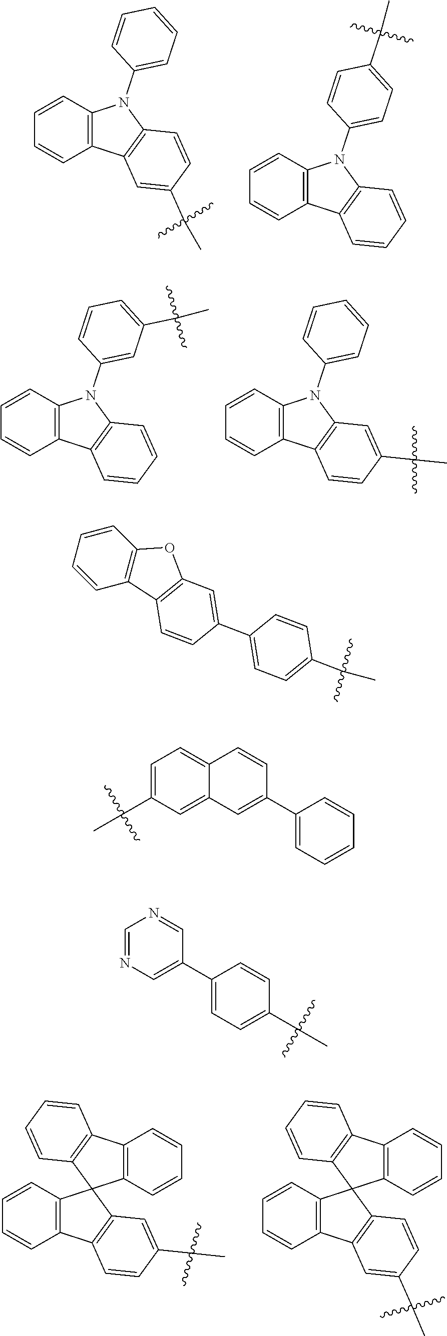

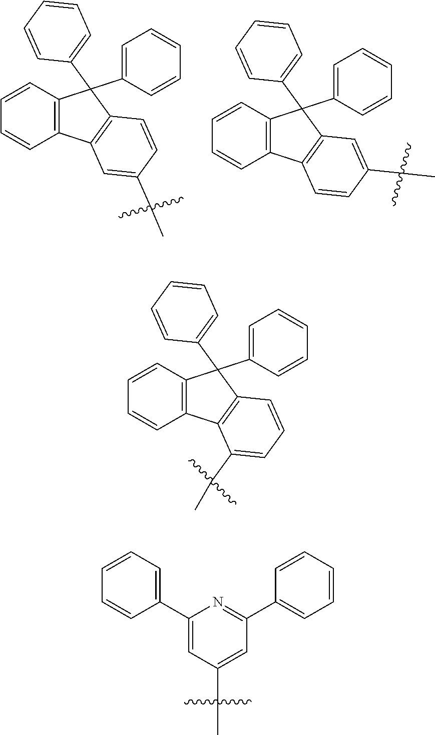 [SMILES: *c1ccc2c(c1)-c1ccccc1C2(c1ccccc1)c1ccccc1 ; *c1ccc2c(c1)C(c1ccccc1)(c1ccccc1)c1ccccc1-2 ; *c1cccc2c1-c1ccccc1C2(c1ccccc1)c1ccccc1 ; *c1cc(-c2ccccc2)nc(-c2ccccc2)c1]

In one embodiment of the present disclosure, the substituents of Ar₁, Ar₂, Ar₃ and Ar₄ are the same or different, and are respectively independently selected from deuterium, a fluorine, a cyano, a phenyl, a pyrimidyl, a pyridyl, a naphthyl, a phenanthryl, a methyl, an ethyl, an isopropyl, a tert-butyl, a dibenzofuranyl, a dibenzothienyl, a carbazolyl or a N-phenylcarbazolyl.

In one embodiment of the present disclosure, Ar₅ is selected from a substituted or unsubstituted aryl with 6 to 15 carbon atoms, or a substituted or unsubstituted heteroaryl with 4 to 10 carbon atoms.

In one embodiment of the present disclosure, Ar₅ is selected from a group consisting of the following groups:

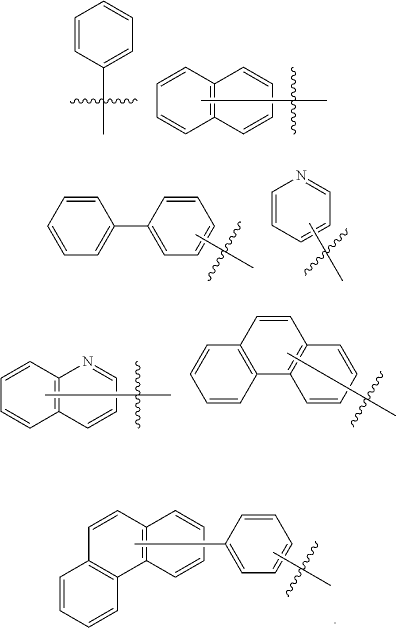

In one embodiment of the present disclosure, Ar₅ is selected from deuterium, a cyano, a fluorine, a trimethylsilyl, an alkyl with 1 to 5 with 1 to 5 carbon atoms, a substituted or unsubstituted aryl with 6 to 25 carbon atoms, or a substituted or unsubstituted heteroaryl with 4 to 18 carbon atoms.

In one embodiment of the present disclosure, Ar₅ is selected from deuterium, a cyano, a fluorine, a trimethylsilyl, a deuterated methyl, a methyl, an ethyl, an isopropyl, a tert-butyl, a substituted or unsubstituted phenyl, a substituted or unsubstituted pyridyl, a substituted or unsubstituted pyrimidyl, a substituted or unsubstituted naphthyl, a substituted or unsubstituted biphenyl, a substituted or unsubstituted quinolinyl, a substituted or unsubstituted isoquinoly, a substituted or unsubstituted phenanthryl, a substituted or unsubstituted dibenzofuranyl, a substituted or unsubstituted dibenzothienyl, a substituted or unsubstituted fluorenyl, a substituted or unsubstituted N-phenylcarbazolyl, or a substituted or unsubstituted carbazolyl.

In one embodiment of the present disclosure, Ar₅ is selected from deuterium, a cyano, a fluorine, a trimethylsilyl, a deuterated methyl, a methyl, an ethyl, an isopropyl, a tert-butyl or a group consisting of the following groups:

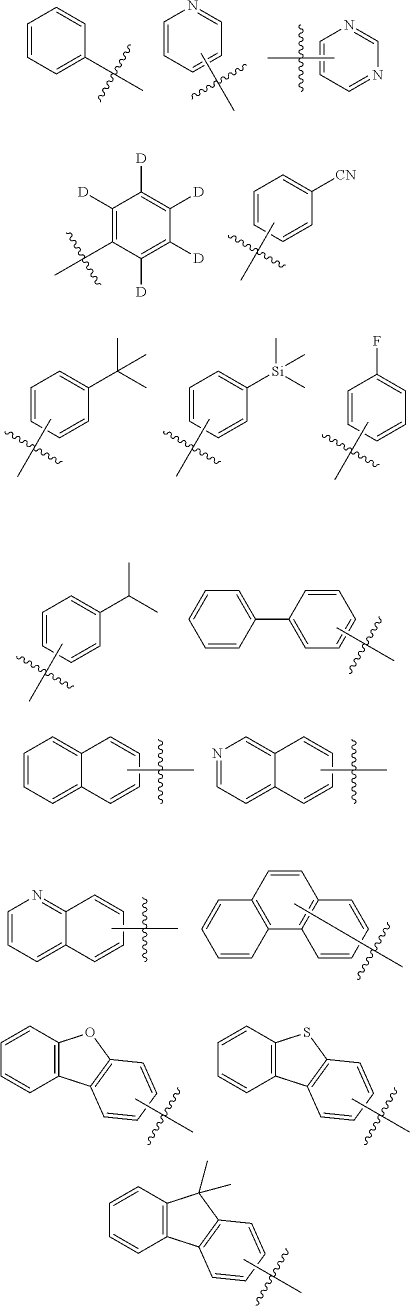

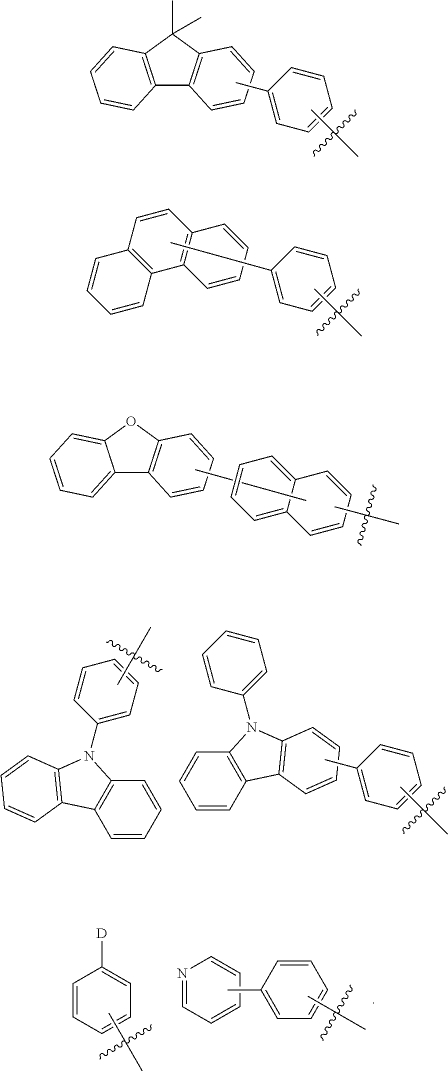

In one embodiment of the present disclosure, Ar₅ is selected from deuterium, a cyano, a fluorine, a trimethylsilyl, a deuterated methyl, a methyl, an ethyl, an isopropyl, a tert-butyl or a group consisting of the following groups:

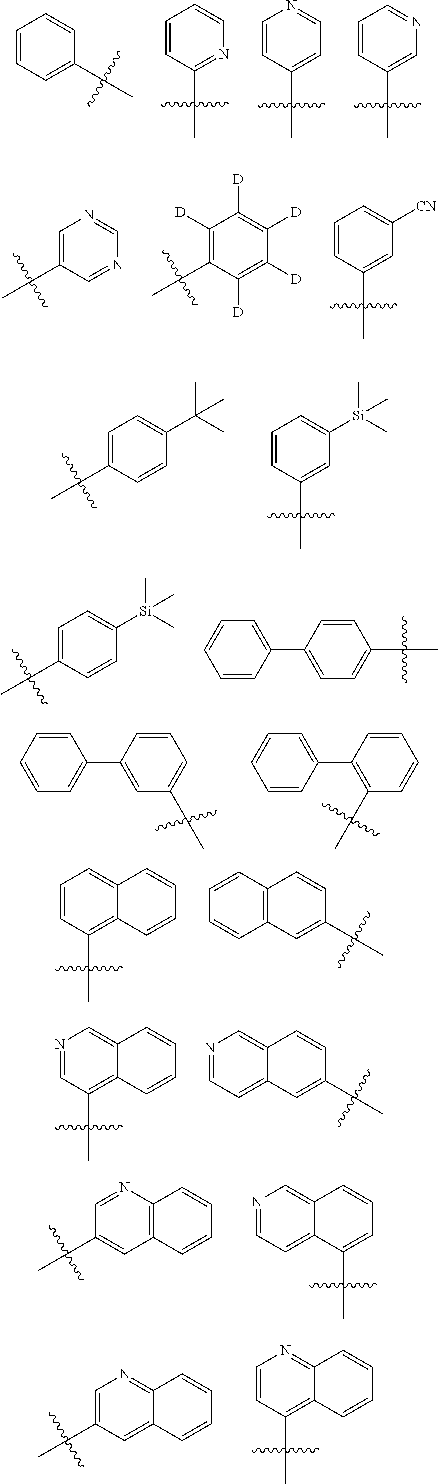

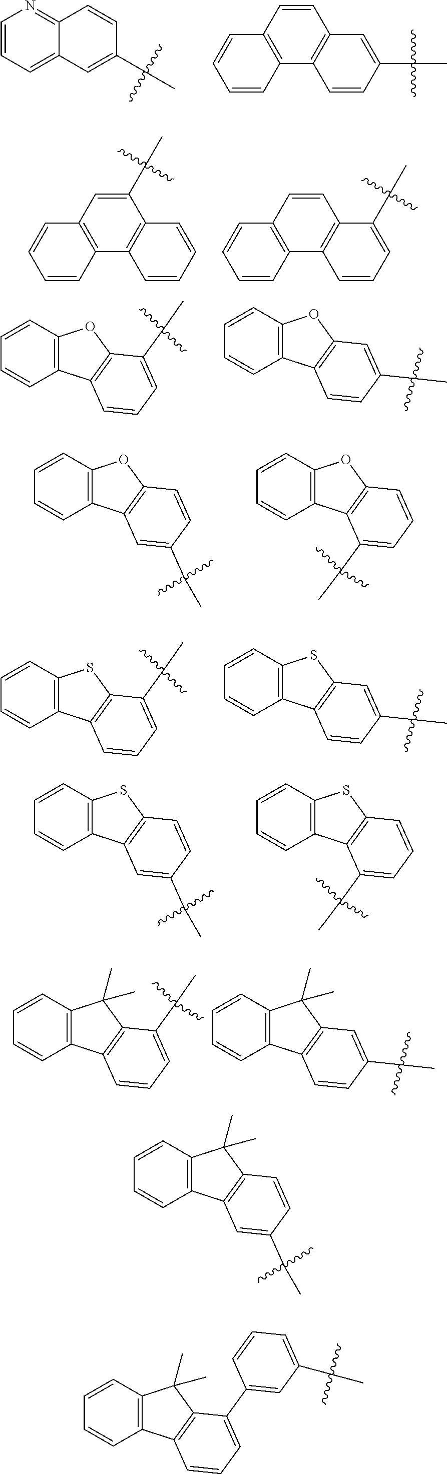

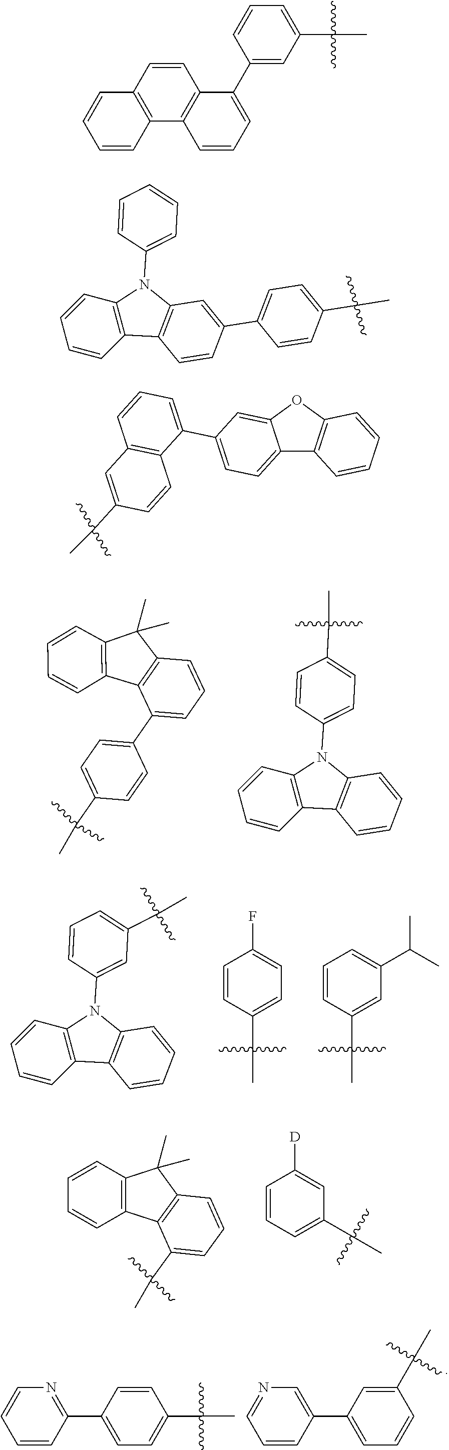

In one embodiment of the present disclosure, R₁ is selected from hydrogen, deuterium, a cyano, a fluorine, a trimethylsilyl, a triphenylsilyl, an alkyl with 1 to 10 carbon atoms, a substituted or unsubstituted aryl with 6 to 21 carbon atoms, or a substituted or unsubstituted heteroaryl with 4 to 12 carbon atoms.

In one embodiment of the present disclosure, R₁ is selected from hydrogen, deuterium, a cyano, a fluorine, a trimethylsilyl, a triphenylsilyl, an alkyl with 1 to 10 carbon atoms, a substituted or unsubstituted phenyl, a substituted or unsubstituted pyridyl, a substituted or unsubstituted pyrimidinyl, a substituted or unsubstituted naphthyl, a substituted or unsubstituted biphenyl, a substituted or unsubstituted quinolinyl, a substituted or unsubstituted isoquinolinyl, a substituted or unsubstituted phenanthryl, a substituted or unsubstituted dibenzofuranyl, a substituted or unsubstituted dibenzothienyl, or a substituted or unsubstituted fluorenyl.

In one embodiment of the present disclosure, R₁ is selected from hydrogen, deuterium, a cyano, a fluorine, a trimethylsilyl, a triphenylsilyl, an alkyl with 1 to 10 carbon atoms or a group consisting of the following groups:

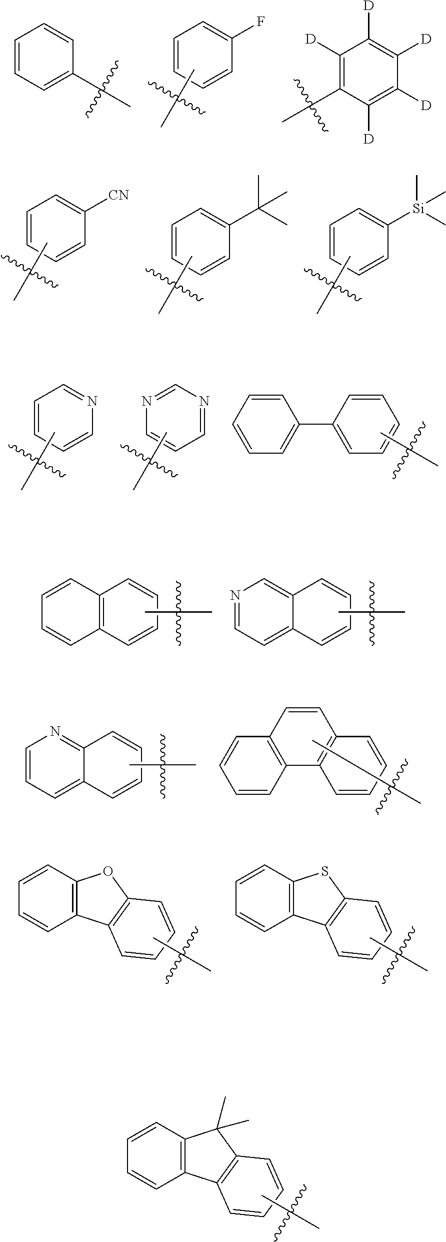

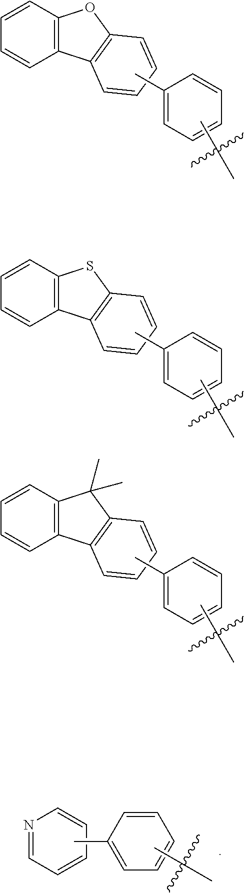

In one embodiment of the present disclosure, R₁ is selected from hydrogen, deuterium, a cyano, a fluorine, a trimethylsilyl, a triphenylsilyl, an alkyl with 1 to 10 carbon atoms or a group consisting of the following groups:

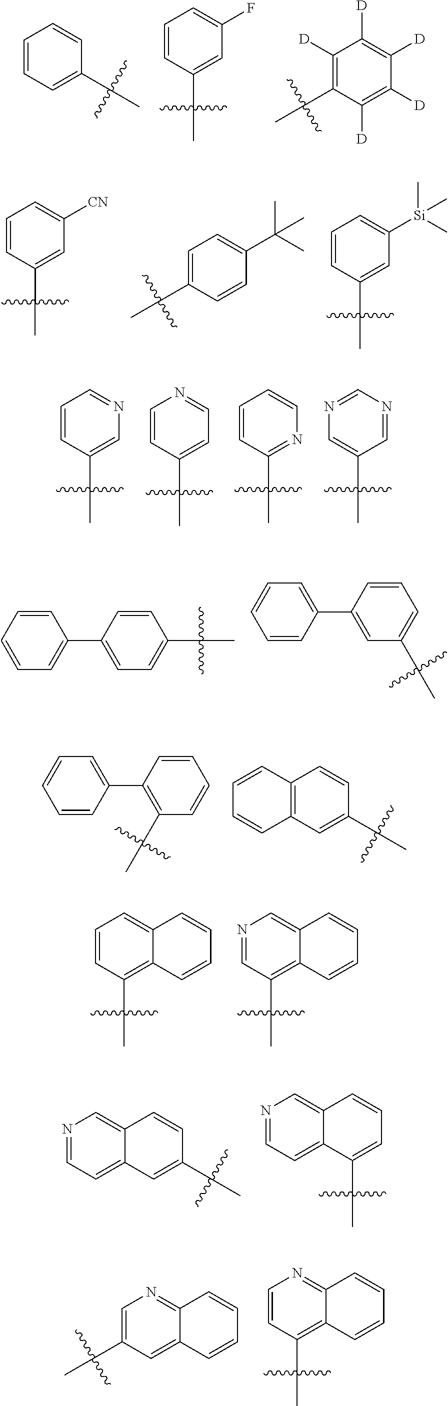

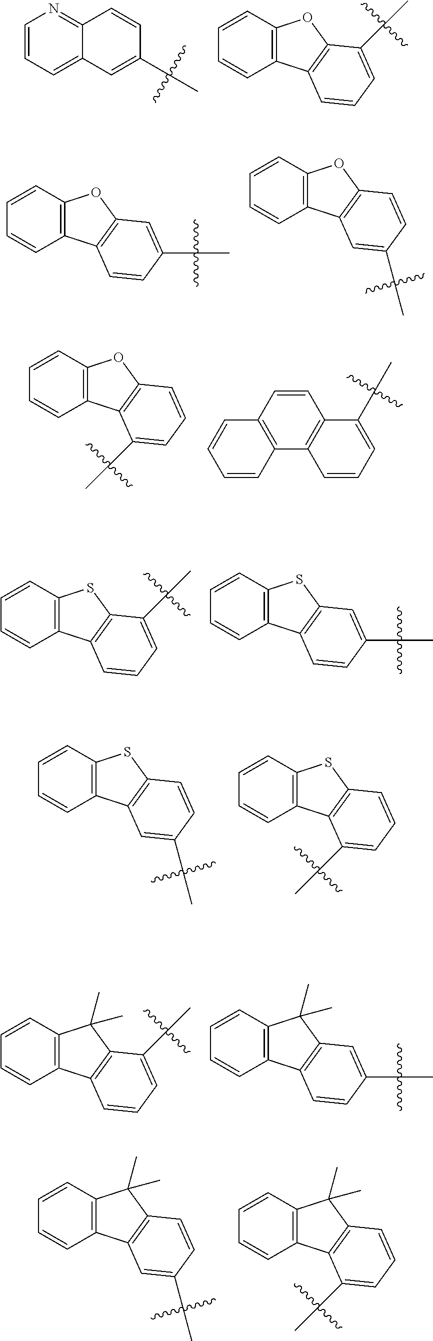

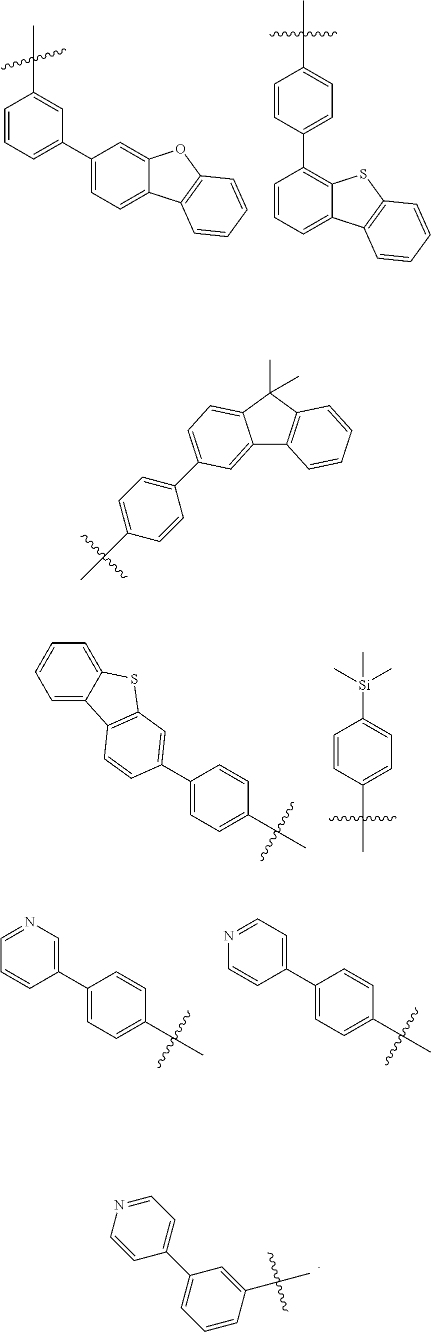

In one embodiment of the present disclosure, the substituents of R₁ and Ar₅ are the same or different, and are respectively independently selected from deuterium, a fluorine, a cyano, a methyl, an ethyl, an isopropyl, a tert-butyl, a trimethylsilyl, a phenyl, a dimethylfluorenyl, a phenanthryl, a N-phenylcarbazolyl, a dibenzofuranyl, a dibenzothienyl, a carbazolyl, a pyridyl, a pyrimidyl, a quinolinyl, or an isoquinolinyl.

In one embodiment of the present disclosure, the substituents of R₁ and Ar₅ are the same or different, and are respectively independently selected from deuterium, a fluorine, a cyano, an alkyl with 1 to 5 carbon atoms, a trimethylsilyl, an aryl with 6 to 15 carbon atoms, or a heteroaryl with 5 to 18 carbon atoms.

In one embodiment of the present disclosure, Ar₂ is selected from a substituted or unsubstituted aryl with 6 to 25 carbon atoms, or a substituted or unsubstituted heteroaryl with 3 to 33 carbon atoms.

In one embodiment of the present disclosure, Ar₂ is selected from a group consisting of the following groups:

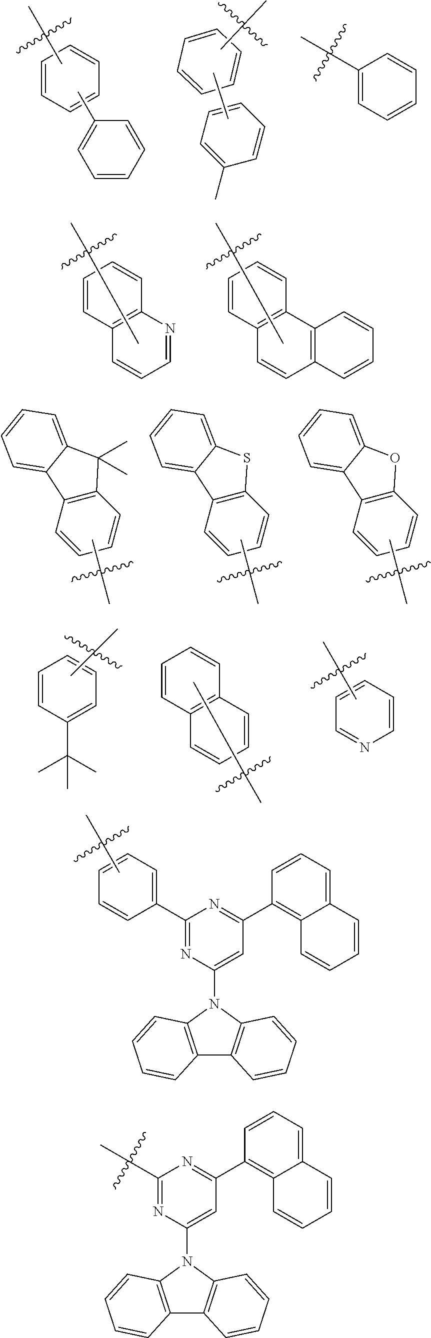

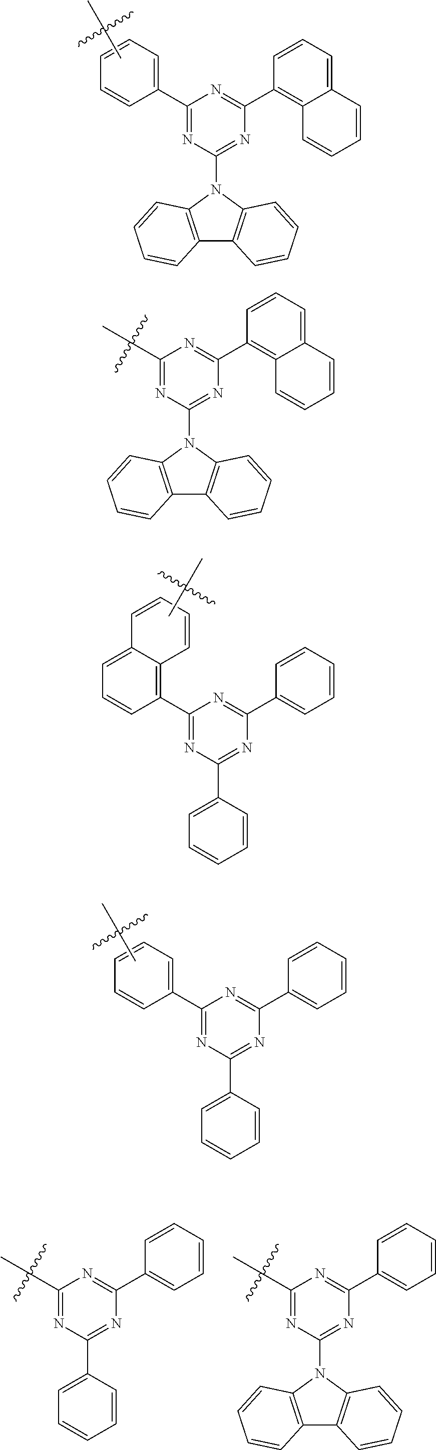

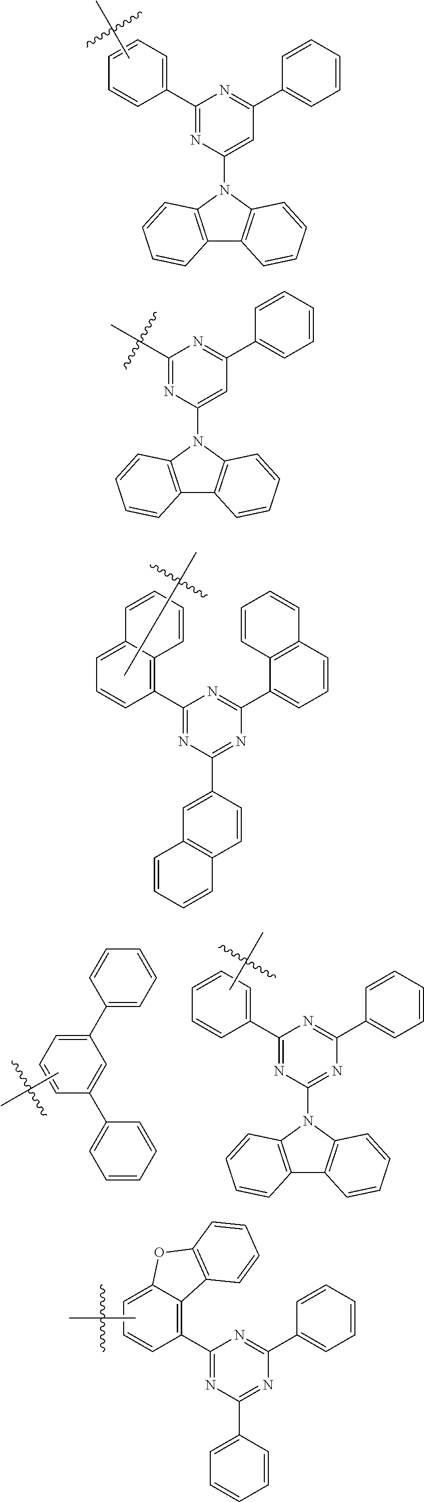

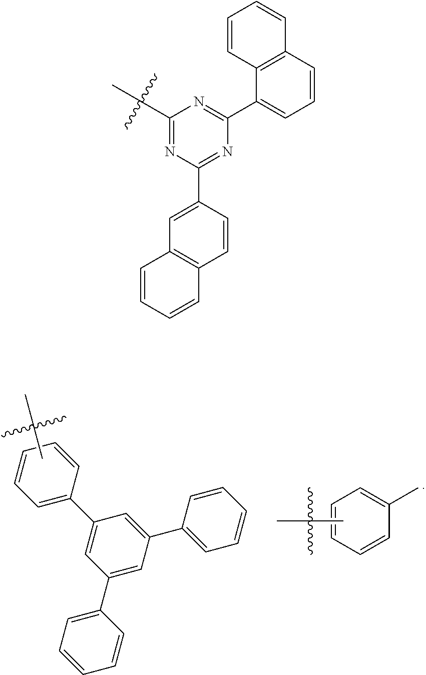

In one embodiment of the present disclosure, the arylamine compound is selected from one or more of the following compounds P1 to P243:

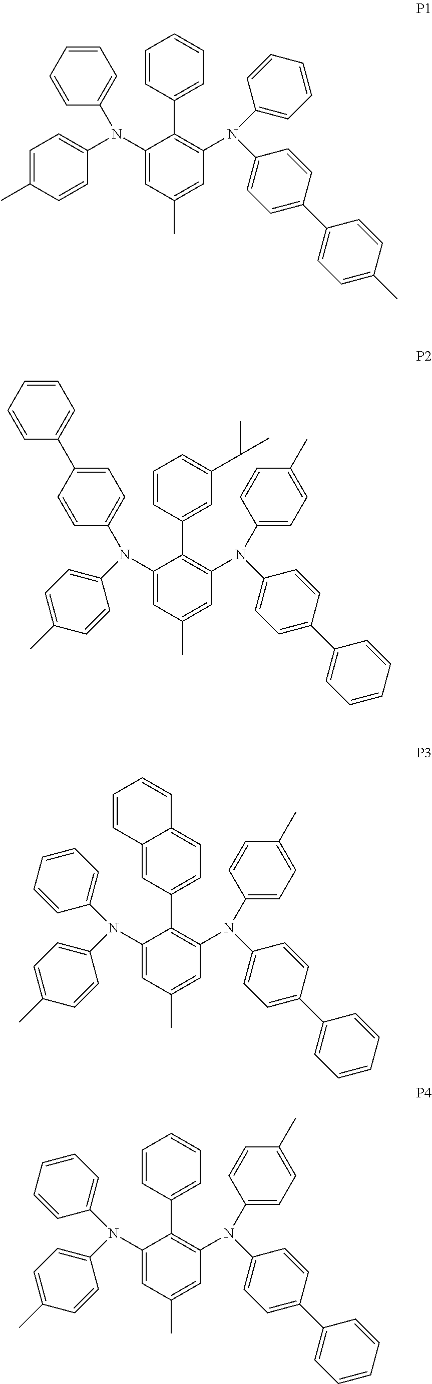

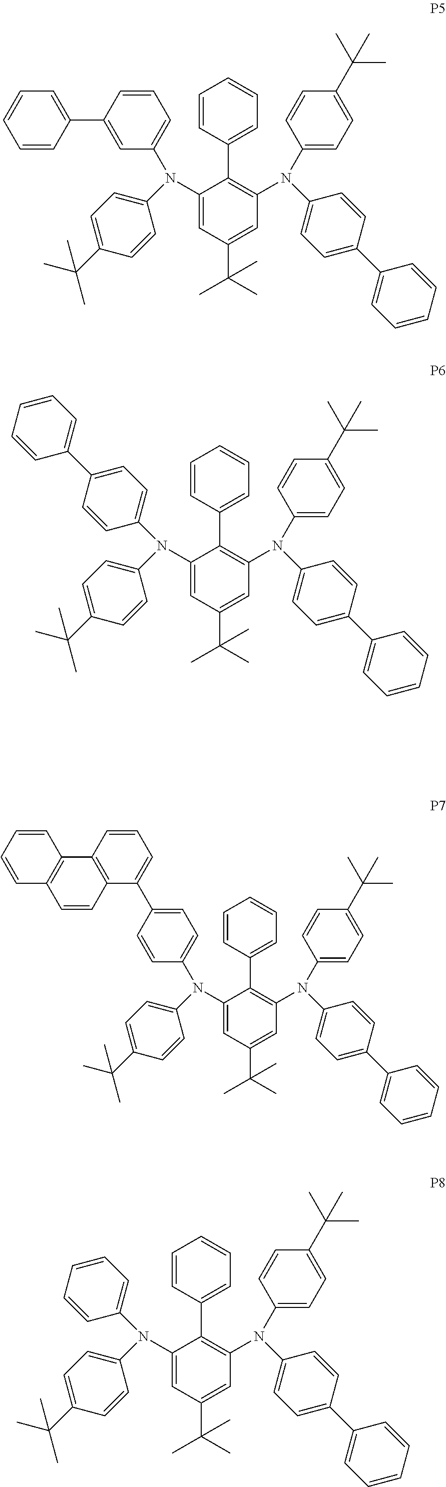 [SMILES: CC(C)(C)c1ccc(N(c2ccc(-c3ccccc3)cc2)c2cc(C(C)(C)C)cc(N(c3ccc(C(C)(C)C)cc3)c3cccc(-c4ccccc4)c3)c2-c2ccccc2)cc1 ; CC(C)(C)c1ccc(N(c2ccc(-c3ccccc3)cc2)c2cc(C(C)(C)C)cc(N(c3ccc(-c4ccccc4)cc3)c3ccc(C(C)(C)C)cc3)c2-c2ccccc2)cc1 ; CC(C)(C)c1ccc(N(c2ccc(-c3ccccc3)cc2)c2cc(C(C)(C)C)cc(N(c3ccc(-c4cccc5c4ccc4ccccc45)cc3)c3ccc(C(C)(C)C)cc3)c2-c2ccccc2)cc1 ; CC(C)(C)c1ccc(N(c2ccccc2)c2cc(C(C)(C)C)cc(N(c3ccc(-c4ccccc4)cc3)c3ccc(C(C)(C)C)cc3)c2-c2ccccc2)cc1]

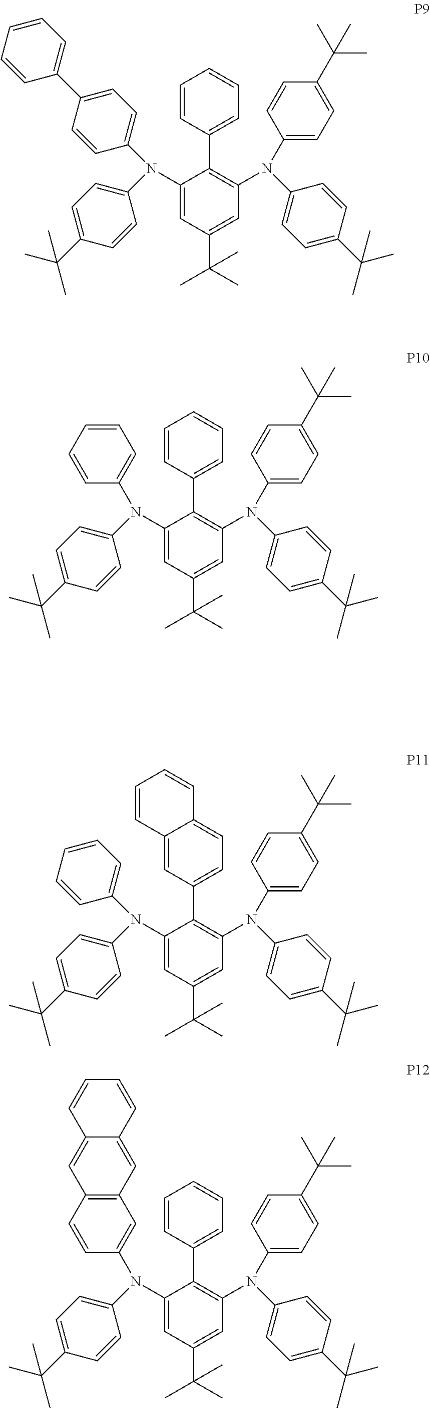

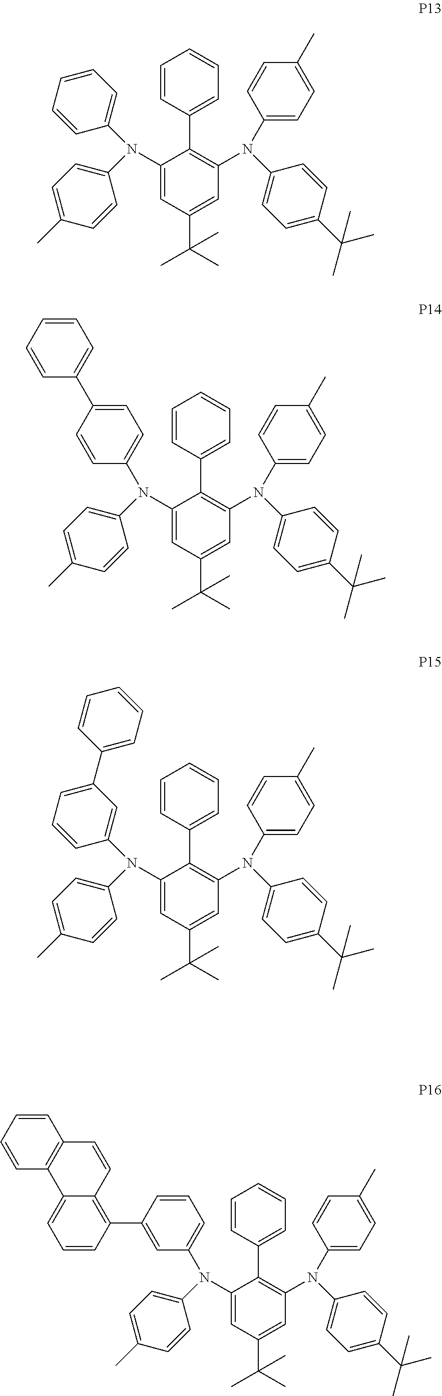

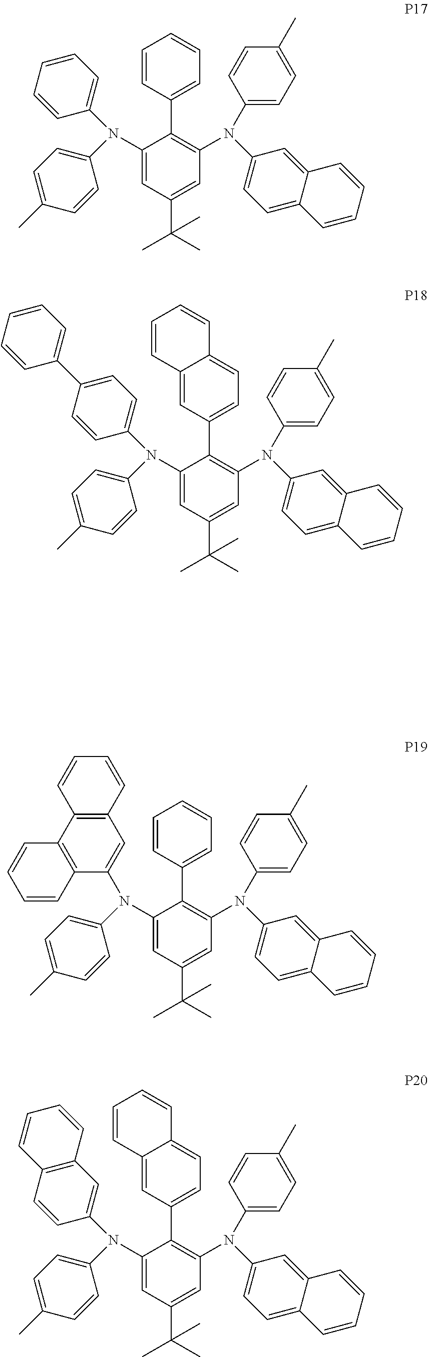 [SMILES: Cc1ccc(N(c2ccccc2)c2cc(C(C)(C)C)cc(N(c3ccc(C)cc3)c3ccc4ccccc4c3)c2-c2ccccc2)cc1 ; Cc1ccc(N(c2ccc(-c3ccccc3)cc2)c2cc(C(C)(C)C)cc(N(c3ccc(C)cc3)c3ccc4ccccc4c3)c2-c2ccc3ccccc3c2)cc1 ; Cc1ccc(N(c2ccc3ccccc3c2)c2cc(C(C)(C)C)cc(N(c3ccc(C)cc3)c3cc4ccccc4c4ccccc34)c2-c2ccccc2)cc1 ; Cc1ccc(N(c2ccc3ccccc3c2)c2cc(C(C)(C)C)cc(N(c3ccc(C)cc3)c3ccc4ccccc4c3)c2-c2ccc3ccccc3c2)cc1]

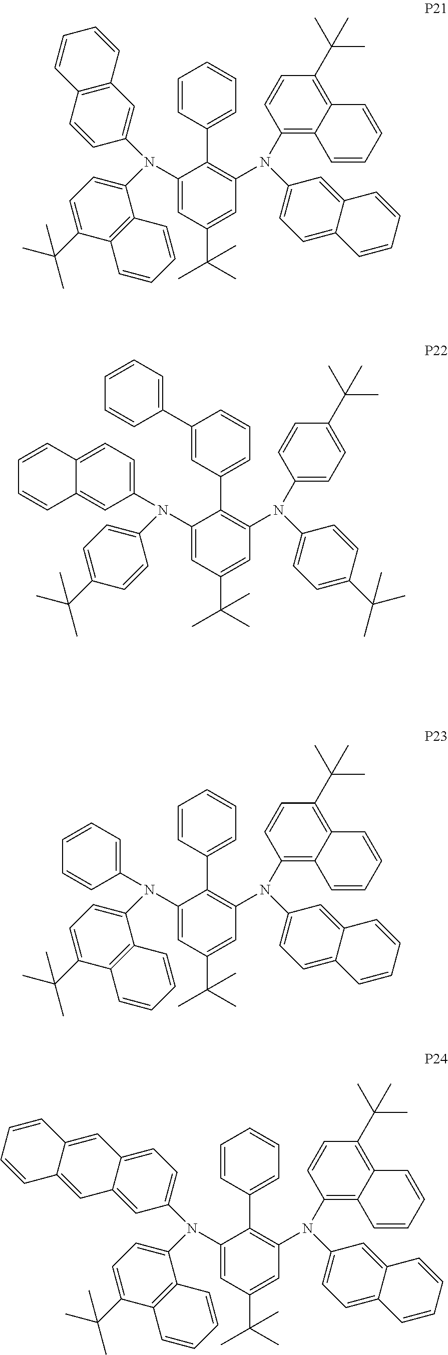

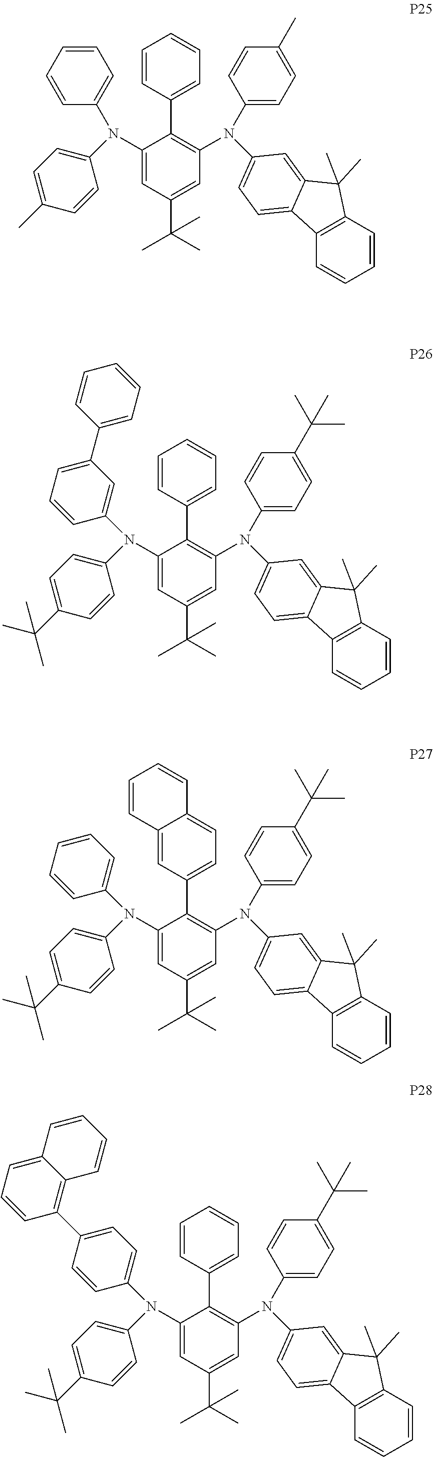 [SMILES: Cc1ccc(N(c2ccccc2)c2cc(C(C)(C)C)cc(N(c3ccc(C)cc3)c3ccc4c(c3)C(C)(C)c3ccccc3-4)c2-c2ccccc2)cc1 ; CC(C)(C)c1ccc(N(c2cccc(-c3ccccc3)c2)c2cc(C(C)(C)C)cc(N(c3ccc(C(C)(C)C)cc3)c3ccc4c(c3)C(C)(C)c3ccccc3-4)c2-c2ccccc2)cc1 ; CC(C)(C)c1ccc(N(c2ccccc2)c2cc(C(C)(C)C)cc(N(c3ccc(C(C)(C)C)cc3)c3ccc4c(c3)C(C)(C)c3ccccc3-4)c2-c2ccc3ccccc3c2)cc1 ; CC(C)(C)c1ccc(N(c2ccc(-c3cccc4ccccc34)cc2)c2cc(C(C)(C)C)cc(N(c3ccc(C(C)(C)C)cc3)c3ccc4c(c3)C(C)(C)c3ccccc3-4)c2-c2ccccc2)cc1]

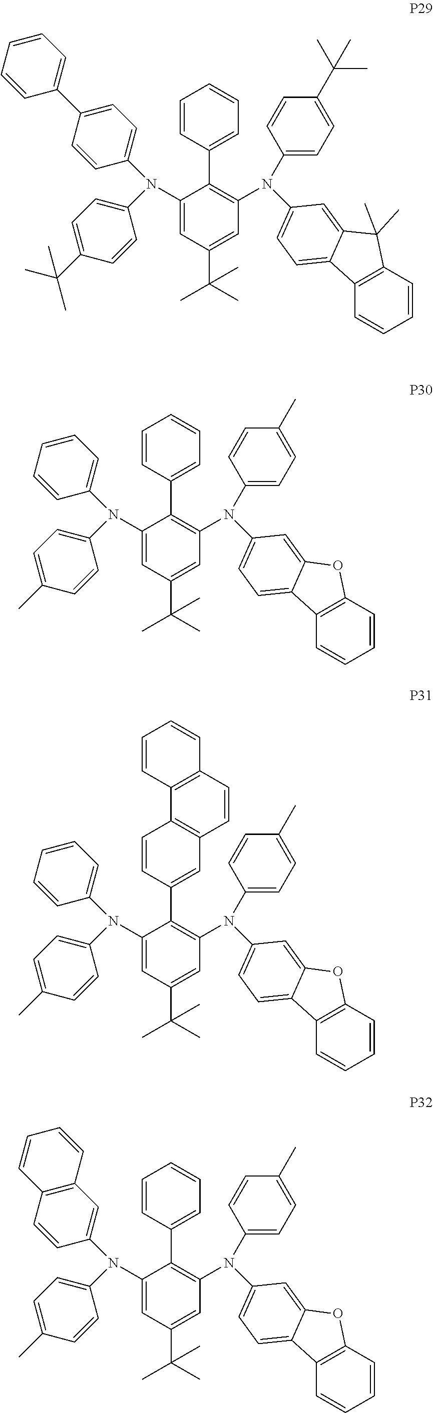 [SMILES: CC(C)(C)c1ccc(N(c2ccc(-c3ccccc3)cc2)c2cc(C(C)(C)C)cc(N(c3ccc(C(C)(C)C)cc3)c3ccc4c(c3)C(C)(C)c3ccccc3-4)c2-c2ccccc2)cc1 ; Cc1ccc(N(c2ccccc2)c2cc(C(C)(C)C)cc(N(c3ccc(C)cc3)c3ccc4c(c3)oc3ccccc34)c2-c2ccccc2)cc1 ; Cc1ccc(N(c2ccccc2)c2cc(C(C)(C)C)cc(N(c3ccc(C)cc3)c3ccc4c(c3)oc3ccccc34)c2-c2ccc3c(ccc4ccccc43)c2)cc1 ; Cc1ccc(N(c2ccc3ccccc3c2)c2cc(C(C)(C)C)cc(N(c3ccc(C)cc3)c3ccc4c(c3)oc3ccccc34)c2-c2ccccc2)cc1]

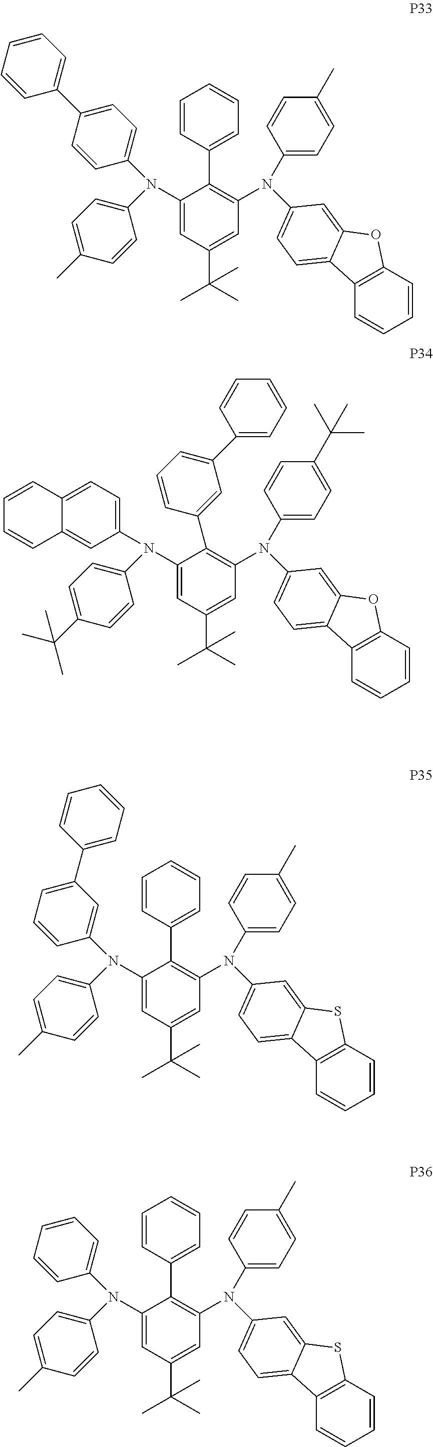 [SMILES: Cc1ccc(N(c2ccc(-c3ccccc3)cc2)c2cc(C(C)(C)C)cc(N(c3ccc(C)cc3)c3ccc4c(c3)oc3ccccc34)c2-c2ccccc2)cc1 ; CC(C)(C)c1ccc(N(c2ccc3ccccc3c2)c2cc(C(C)(C)C)cc(N(c3ccc(C(C)(C)C)cc3)c3ccc4c(c3)oc3ccccc34)c2-c2cccc(-c3ccccc3)c2)cc1 ; Cc1ccc(N(c2cccc(-c3ccccc3)c2)c2cc(C(C)(C)C)cc(N(c3ccc(C)cc3)c3ccc4c(c3)sc3ccccc34)c2-c2ccccc2)cc1 ; Cc1ccc(N(c2ccccc2)c2cc(C(C)(C)C)cc(N(c3ccc(C)cc3)c3ccc4c(c3)sc3ccccc34)c2-c2ccccc2)cc1]

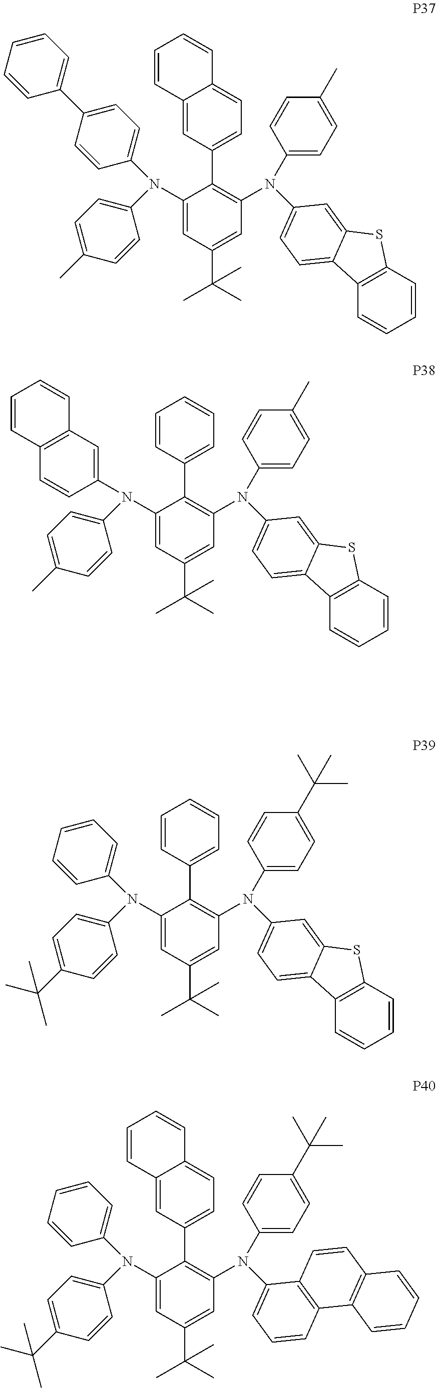 [SMILES: Cc1ccc(N(c2ccc(-c3ccccc3)cc2)c2cc(C(C)(C)C)cc(N(c3ccc(C)cc3)c3ccc4c(c3)sc3ccccc34)c2-c2ccc3ccccc3c2)cc1 ; Cc1ccc(N(c2ccc3ccccc3c2)c2cc(C(C)(C)C)cc(N(c3ccc(C)cc3)c3ccc4c(c3)sc3ccccc34)c2-c2ccccc2)cc1 ; CC(C)(C)c1ccc(N(c2ccccc2)c2cc(C(C)(C)C)cc(N(c3ccc(C(C)(C)C)cc3)c3ccc4c(c3)sc3ccccc34)c2-c2ccccc2)cc1 ; CC(C)(C)c1ccc(N(c2ccccc2)c2cc(C(C)(C)C)cc(N(c3ccc(C(C)(C)C)cc3)c3cccc4c3ccc3ccccc34)c2-c2ccc3ccccc3c2)cc1]

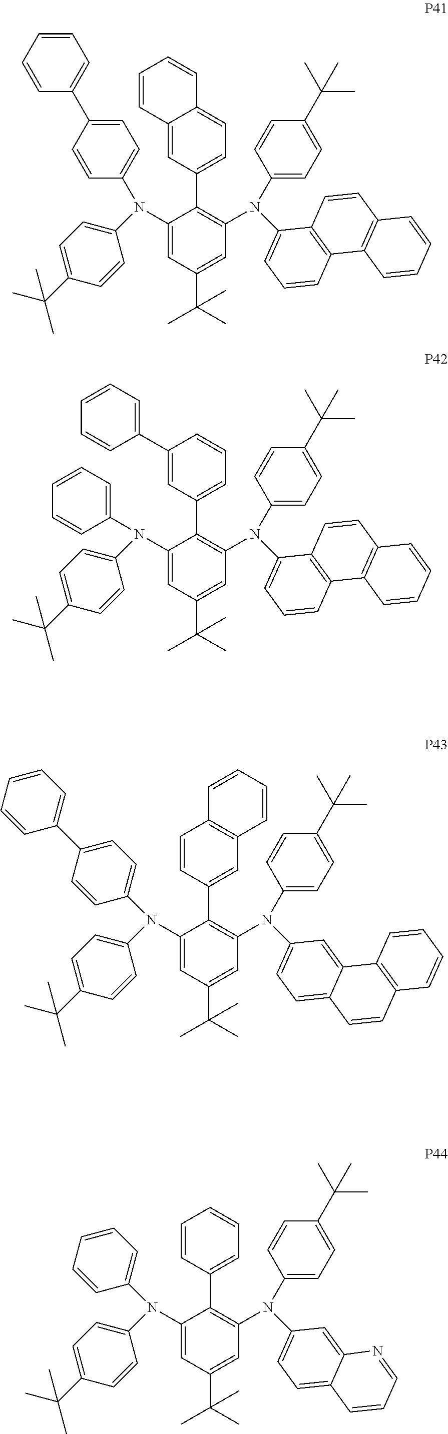 [SMILES: CC(C)(C)c1ccc(N(c2ccc(-c3ccccc3)cc2)c2cc(C(C)(C)C)cc(N(c3ccc(C(C)(C)C)cc3)c3cccc4c3ccc3ccccc34)c2-c2ccc3ccccc3c2)cc1 ; CC(C)(C)c1ccc(N(c2ccccc2)c2cc(C(C)(C)C)cc(N(c3ccc(C(C)(C)C)cc3)c3cccc4c3ccc3ccccc34)c2-c2cccc(-c3ccccc3)c2)cc1 ; CC(C)(C)c1ccc(N(c2ccc(-c3ccccc3)cc2)c2cc(C(C)(C)C)cc(N(c3ccc(C(C)(C)C)cc3)c3ccc4ccc5ccccc5c4c3)c2-c2ccc3ccccc3c2)cc1 ; CC(C)(C)c1ccc(N(c2ccccc2)c2cc(C(C)(C)C)cc(N(c3ccc(C(C)(C)C)cc3)c3ccc4cccnc4c3)c2-c2ccccc2)cc1]

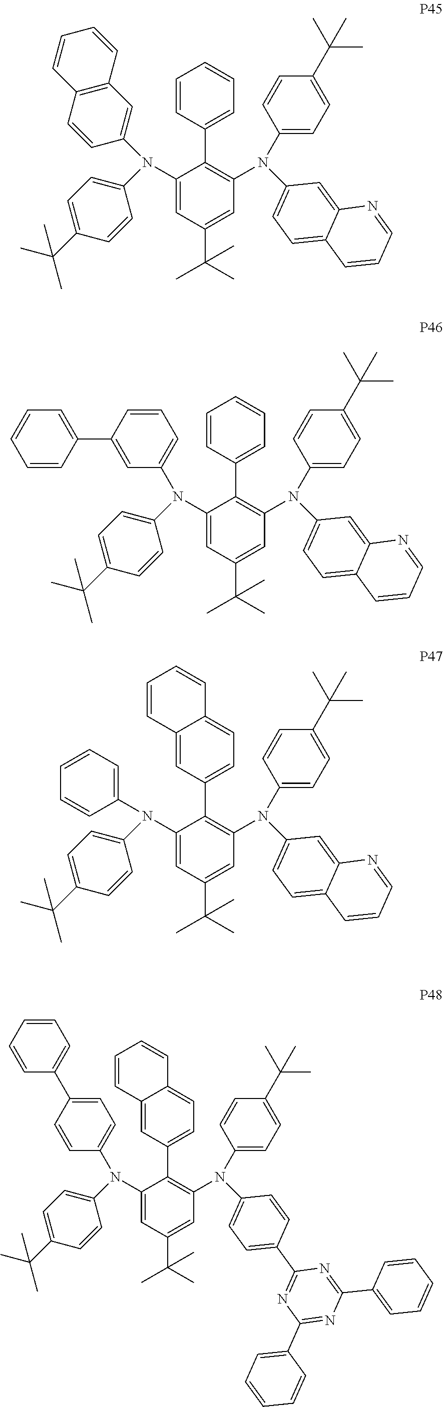 [SMILES: CC(C)(C)c1ccc(N(c2ccc3ccccc3c2)c2cc(C(C)(C)C)cc(N(c3ccc(C(C)(C)C)cc3)c3ccc4cccnc4c3)c2-c2ccccc2)cc1 ; CC(C)(C)c1ccc(N(c2cccc(-c3ccccc3)c2)c2cc(C(C)(C)C)cc(N(c3ccc(C(C)(C)C)cc3)c3ccc4cccnc4c3)c2-c2ccccc2)cc1 ; CC(C)(C)c1ccc(N(c2ccccc2)c2cc(C(C)(C)C)cc(N(c3ccc(C(C)(C)C)cc3)c3ccc4cccnc4c3)c2-c2ccc3ccccc3c2)cc1 ; CC(C)(C)c1ccc(N(c2ccc(-c3ccccc3)cc2)c2cc(C(C)(C)C)cc(N(c3ccc(-c4nc(-c5ccccc5)nc(-c5ccccc5)n4)cc3)c3ccc(C(C)(C)C)cc3)c2-c2ccc3ccccc3c2)cc1]

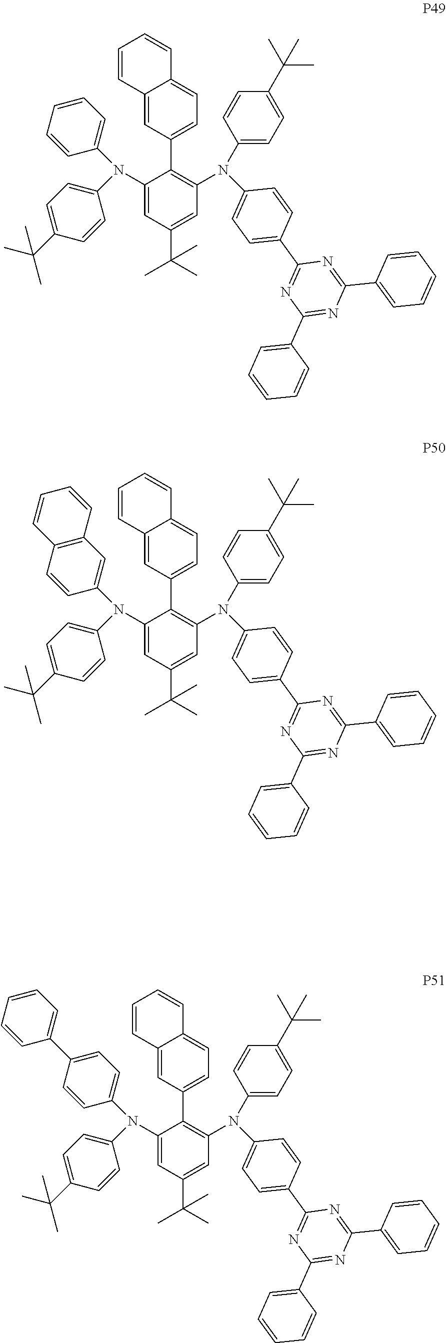 [SMILES: CC(C)(C)c1ccc(N(c2ccccc2)c2cc(C(C)(C)C)cc(N(c3ccc(-c4nc(-c5ccccc5)nc(-c5ccccc5)n4)cc3)c3ccc(C(C)(C)C)cc3)c2-c2ccc3ccccc3c2)cc1 ; CC(C)(C)c1ccc(N(c2ccc(-c3nc(-c4ccccc4)nc(-c4ccccc4)n3)cc2)c2cc(C(C)(C)C)cc(N(c3ccc(C(C)(C)C)cc3)c3ccc4ccccc4c3)c2-c2ccc3ccccc3c2)cc1 ; CC(C)(C)c1ccc(N(c2ccc(-c3ccccc3)cc2)c2cc(C(C)(C)C)cc(N(c3ccc(-c4nc(-c5ccccc5)nc(-c5ccccc5)n4)cc3)c3ccc(C(C)(C)C)cc3)c2-c2ccc3ccccc3c2)cc1]

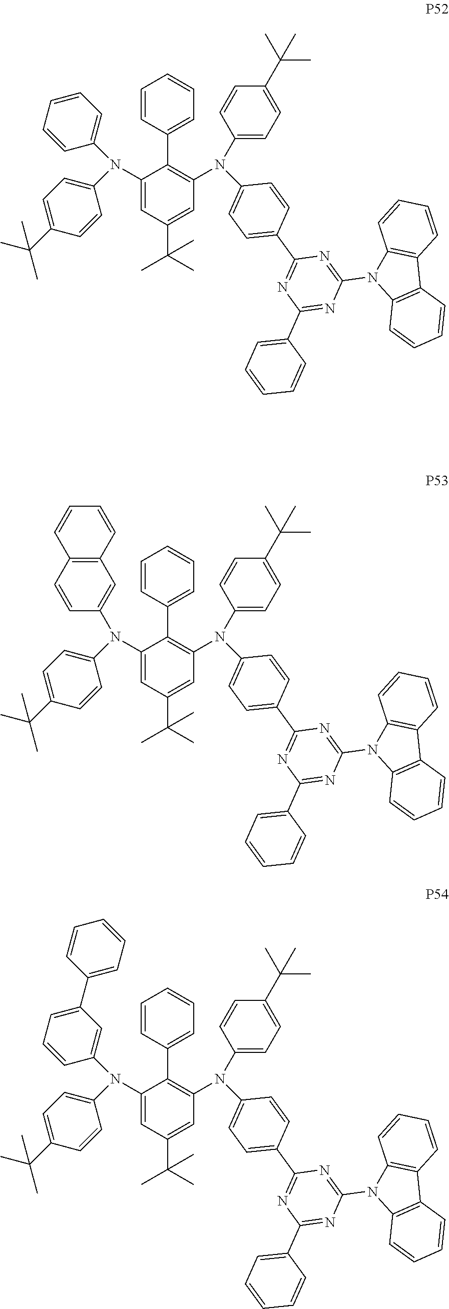 [SMILES: CC(C)(C)c1ccc(N(c2ccccc2)c2cc(C(C)(C)C)cc(N(c3ccc(-c4nc(-c5ccccc5)nc(-n5c6ccccc6c6ccccc65)n4)cc3)c3ccc(C(C)(C)C)cc3)c2-c2ccccc2)cc1 ; CC(C)(C)c1ccc(N(c2ccc(-c3nc(-c4ccccc4)nc(-n4c5ccccc5c5ccccc54)n3)cc2)c2cc(C(C)(C)C)cc(N(c3ccc(C(C)(C)C)cc3)c3ccc4ccccc4c3)c2-c2ccccc2)cc1 ; CC(C)(C)c1ccc(N(c2ccc(-c3nc(-c4ccccc4)nc(-n4c5ccccc5c5ccccc54)n3)cc2)c2cc(C(C)(C)C)cc(N(c3ccc(C(C)(C)C)cc3)c3cccc(-c4ccccc4)c3)c2-c2ccccc2)cc1]

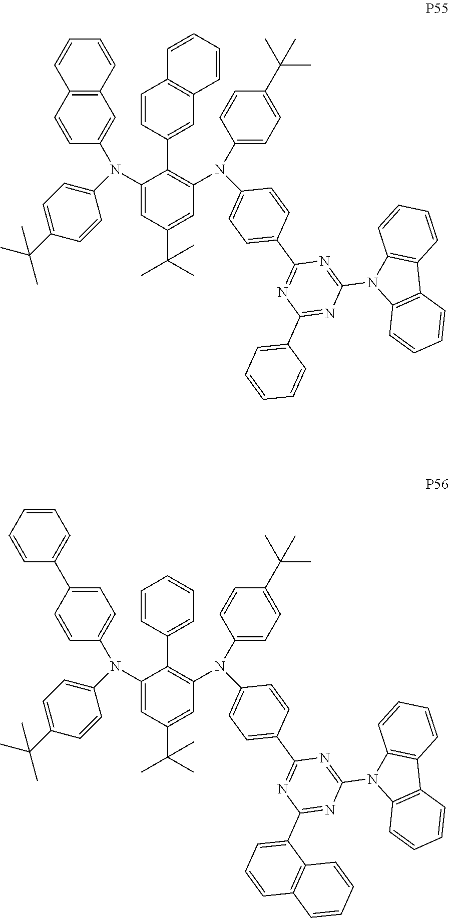 [SMILES: CC(C)(C)c1ccc(N(c2ccc(-c3nc(-c4ccccc4)nc(-n4c5ccccc5c5ccccc54)n3)cc2)c2cc(C(C)(C)C)cc(N(c3ccc(C(C)(C)C)cc3)c3ccc4ccccc4c3)c2-c2ccc3ccccc3c2)cc1 ; CC(C)(C)c1ccc(N(c2ccc(-c3ccccc3)cc2)c2cc(C(C)(C)C)cc(N(c3ccc(-c4nc(-c5cccc6ccccc56)nc(-n5c6ccccc6c6ccccc65)n4)cc3)c3ccc(C(C)(C)C)cc3)c2-c2ccccc2)cc1]

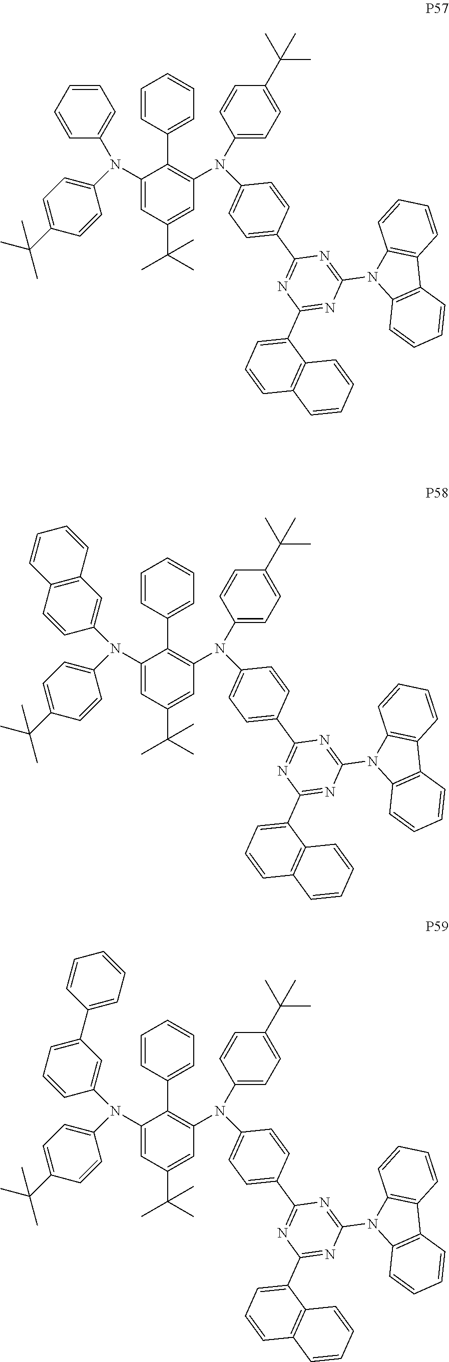 [SMILES: CC(C)(C)c1ccc(N(c2ccccc2)c2cc(C(C)(C)C)cc(N(c3ccc(-c4nc(-c5cccc6ccccc56)nc(-n5c6ccccc6c6ccccc65)n4)cc3)c3ccc(C(C)(C)C)cc3)c2-c2ccccc2)cc1 ; CC(C)(C)c1ccc(N(c2ccc(-c3nc(-c4cccc5ccccc45)nc(-n4c5ccccc5c5ccccc54)n3)cc2)c2cc(C(C)(C)C)cc(N(c3ccc(C(C)(C)C)cc3)c3ccc4ccccc4c3)c2-c2ccccc2)cc1 ; CC(C)(C)c1ccc(N(c2ccc(-c3nc(-c4cccc5ccccc45)nc(-n4c5ccccc5c5ccccc54)n3)cc2)c2cc(C(C)(C)C)cc(N(c3ccc(C(C)(C)C)cc3)c3cccc(-c4ccccc4)c3)c2-c2ccccc2)cc1]

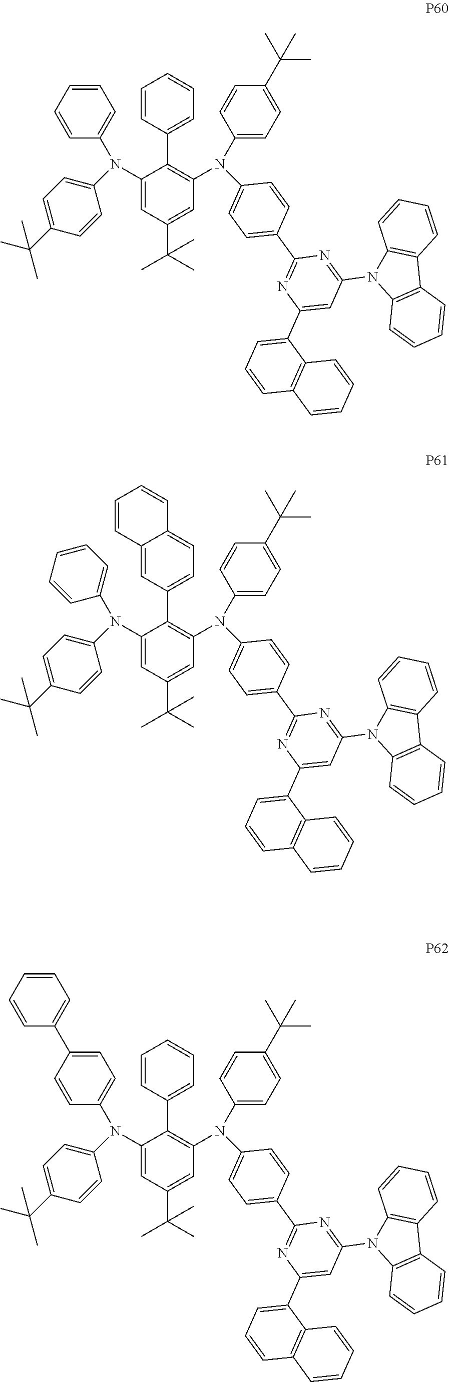 [SMILES: CC(C)(C)c1ccc(N(c2ccccc2)c2cc(C(C)(C)C)cc(N(c3ccc(-c4nc(-c5cccc6ccccc56)cc(-n5c6ccccc6c6ccccc65)n4)cc3)c3ccc(C(C)(C)C)cc3)c2-c2ccccc2)cc1 ; CC(C)(C)c1ccc(N(c2ccccc2)c2cc(C(C)(C)C)cc(N(c3ccc(-c4nc(-c5cccc6ccccc56)cc(-n5c6ccccc6c6ccccc65)n4)cc3)c3ccc(C(C)(C)C)cc3)c2-c2ccc3ccccc3c2)cc1 ; CC(C)(C)c1ccc(N(c2ccc(-c3ccccc3)cc2)c2cc(C(C)(C)C)cc(N(c3ccc(-c4nc(-c5cccc6ccccc56)cc(-n5c6ccccc6c6ccccc65)n4)cc3)c3ccc(C(C)(C)C)cc3)c2-c2ccccc2)cc1]

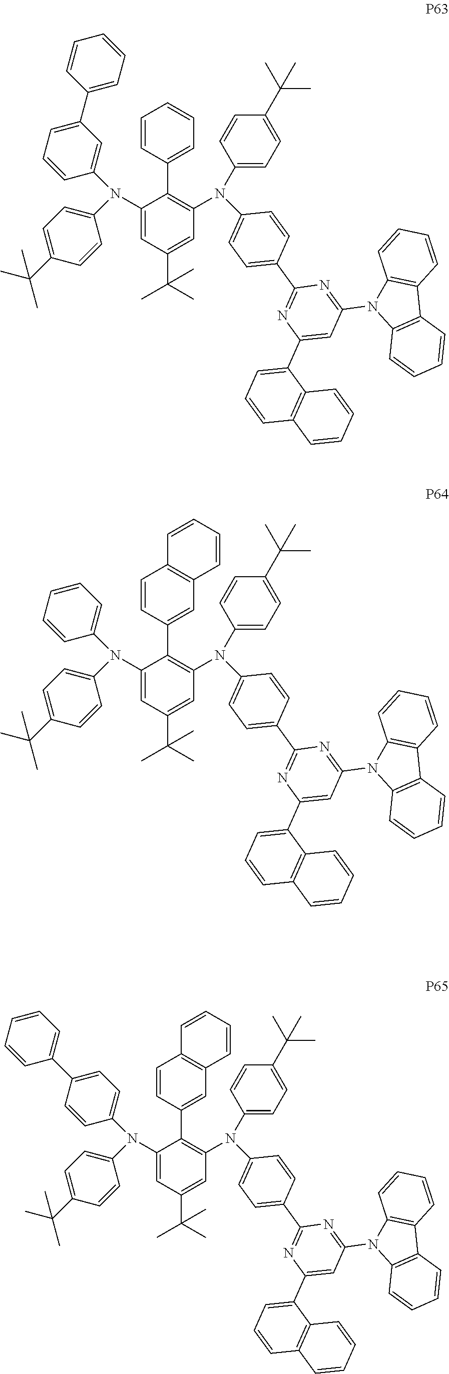 [SMILES: CC(C)(C)c1ccc(N(c2ccc(-c3nc(-c4cccc5ccccc45)cc(-n4c5ccccc5c5ccccc54)n3)cc2)c2cc(C(C)(C)C)cc(N(c3ccc(C(C)(C)C)cc3)c3cccc(-c4ccccc4)c3)c2-c2ccccc2)cc1 ; CC(C)(C)c1ccc(N(c2ccccc2)c2cc(C(C)(C)C)cc(N(c3ccc(-c4nc(-c5cccc6ccccc56)cc(-n5c6ccccc6c6ccccc65)n4)cc3)c3ccc(C(C)(C)C)cc3)c2-c2ccc3ccccc3c2)cc1 ; CC(C)(C)c1ccc(N(c2ccc(-c3ccccc3)cc2)c2cc(C(C)(C)C)cc(N(c3ccc(-c4nc(-c5cccc6ccccc56)cc(-n5c6ccccc6c6ccccc65)n4)cc3)c3ccc(C(C)(C)C)cc3)c2-c2ccc3ccccc3c2)cc1]

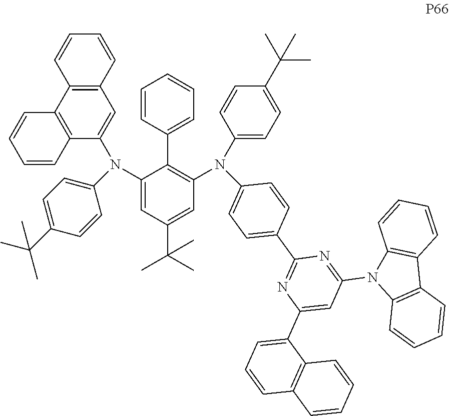 [SMILES: CC(C)(C)c1ccc(N(c2ccc(-c3nc(-c4cccc5ccccc45)cc(-n4c5ccccc5c5ccccc54)n3)cc2)c2cc(C(C)(C)C)cc(N(c3ccc(C(C)(C)C)cc3)c3cc4ccccc4c4ccccc34)c2-c2ccccc2)cc1]

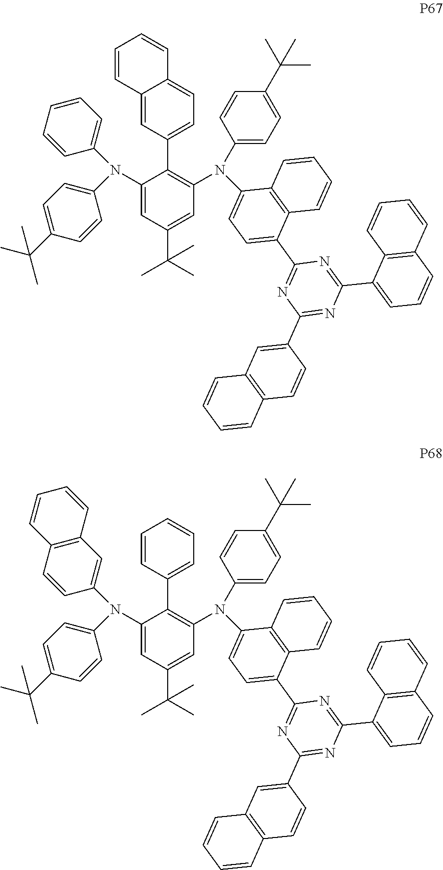 [SMILES: CC(C)(C)c1ccc(N(c2ccccc2)c2cc(C(C)(C)C)cc(N(c3ccc(C(C)(C)C)cc3)c3ccc(-c4nc(-c5ccc6ccccc6c5)nc(-c5cccc6ccccc56)n4)c4ccccc34)c2-c2ccc3ccccc3c2)cc1 ; CC(C)(C)c1ccc(N(c2ccc3ccccc3c2)c2cc(C(C)(C)C)cc(N(c3ccc(C(C)(C)C)cc3)c3ccc(-c4nc(-c5ccc6ccccc6c5)nc(-c5cccc6ccccc56)n4)c4ccccc34)c2-c2ccccc2)cc1]

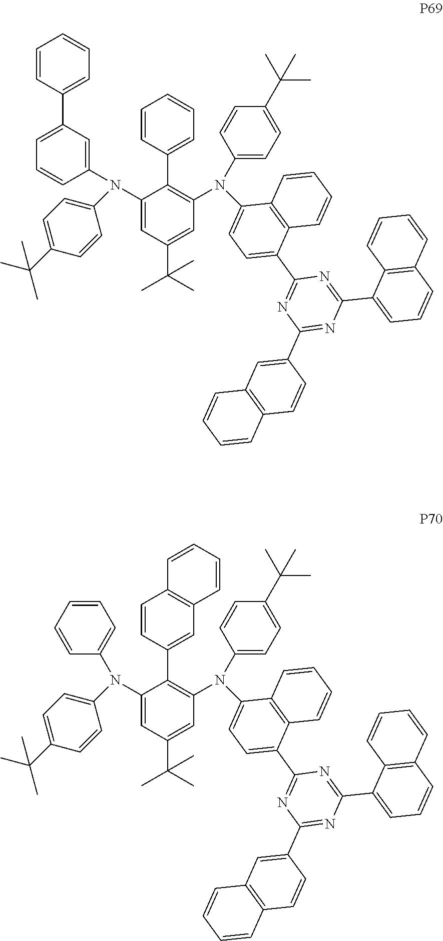 [SMILES: CC(C)(C)c1ccc(N(c2cccc(-c3ccccc3)c2)c2cc(C(C)(C)C)cc(N(c3ccc(C(C)(C)C)cc3)c3ccc(-c4nc(-c5ccc6ccccc6c5)nc(-c5cccc6ccccc56)n4)c4ccccc34)c2-c2ccccc2)cc1 ; CC(C)(C)c1ccc(N(c2ccccc2)c2cc(C(C)(C)C)cc(N(c3ccc(C(C)(C)C)cc3)c3ccc(-c4nc(-c5ccc6ccccc6c5)nc(-c5cccc6ccccc56)n4)c4ccccc34)c2-c2ccc3ccccc3c2)cc1]

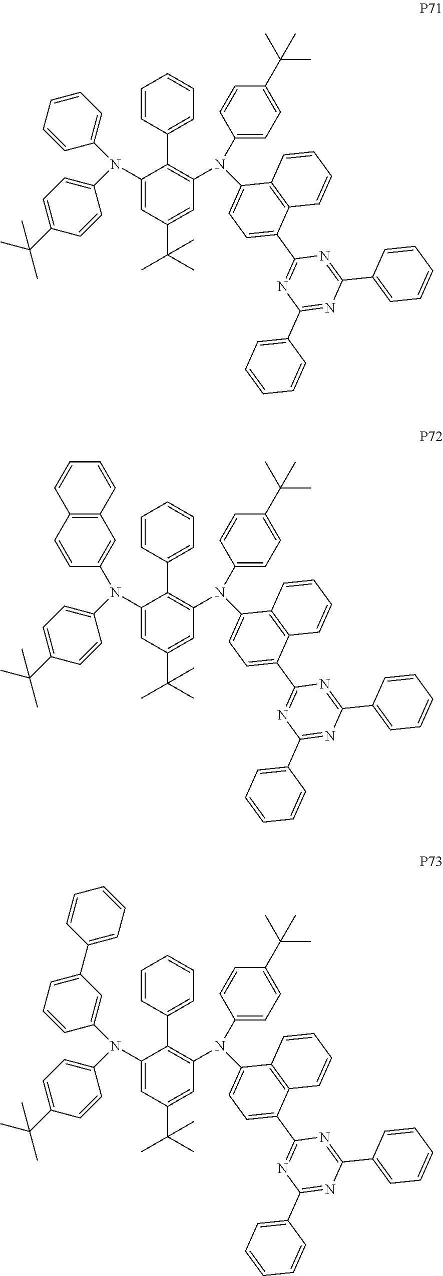 [SMILES: CC(C)(C)c1ccc(N(c2ccccc2)c2cc(C(C)(C)C)cc(N(c3ccc(C(C)(C)C)cc3)c3ccc(-c4nc(-c5ccccc5)nc(-c5ccccc5)n4)c4ccccc34)c2-c2ccccc2)cc1 ; CC(C)(C)c1ccc(N(c2ccc3ccccc3c2)c2cc(C(C)(C)C)cc(N(c3ccc(C(C)(C)C)cc3)c3ccc(-c4nc(-c5ccccc5)nc(-c5ccccc5)n4)c4ccccc34)c2-c2ccccc2)cc1 ; CC(C)(C)c1ccc(N(c2cccc(-c3ccccc3)c2)c2cc(C(C)(C)C)cc(N(c3ccc(C(C)(C)C)cc3)c3ccc(-c4nc(-c5ccccc5)nc(-c5ccccc5)n4)c4ccccc34)c2-c2ccccc2)cc1]

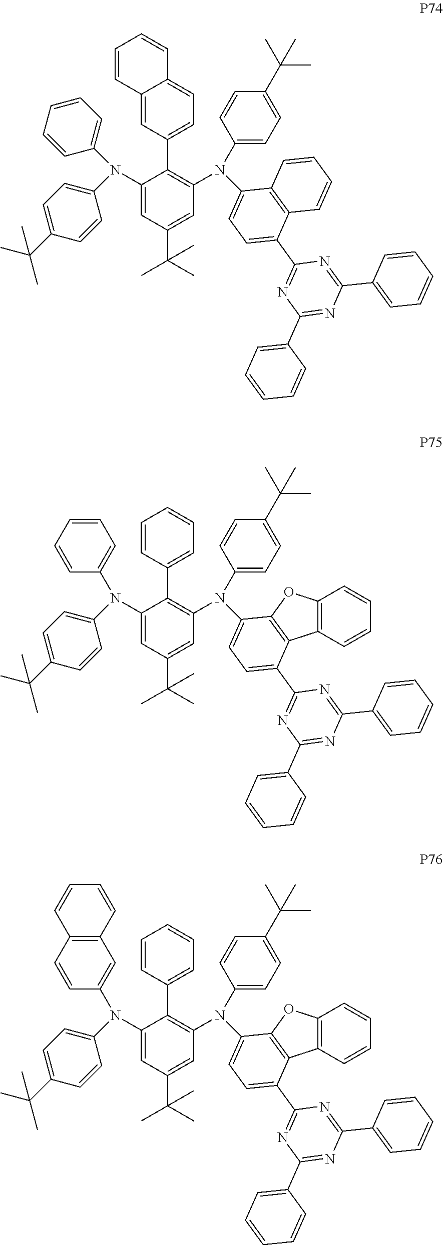 [SMILES: CC(C)(C)c1ccc(N(c2ccccc2)c2cc(C(C)(C)C)cc(N(c3ccc(C(C)(C)C)cc3)c3ccc(-c4nc(-c5ccccc5)nc(-c5ccccc5)n4)c4ccccc34)c2-c2ccc3ccccc3c2)cc1 ; CC(C)(C)c1ccc(N(c2ccccc2)c2cc(C(C)(C)C)cc(N(c3ccc(C(C)(C)C)cc3)c3ccc(-c4nc(-c5ccccc5)nc(-c5ccccc5)n4)c4c3oc3ccccc34)c2-c2ccccc2)cc1 ; CC(C)(C)c1ccc(N(c2ccc3ccccc3c2)c2cc(C(C)(C)C)cc(N(c3ccc(C(C)(C)C)cc3)c3ccc(-c4nc(-c5ccccc5)nc(-c5ccccc5)n4)c4c3oc3ccccc34)c2-c2ccccc2)cc1]

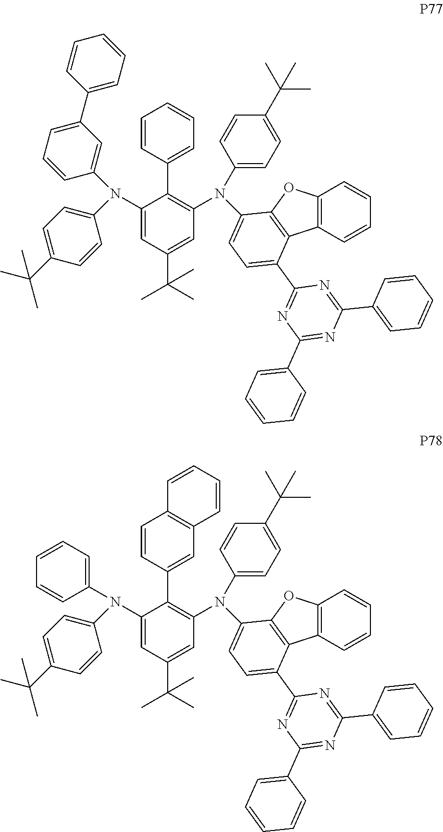 [SMILES: CC(C)(C)c1ccc(N(c2cccc(-c3ccccc3)c2)c2cc(C(C)(C)C)cc(N(c3ccc(C(C)(C)C)cc3)c3ccc(-c4nc(-c5ccccc5)nc(-c5ccccc5)n4)c4c3oc3ccccc34)c2-c2ccccc2)cc1 ; CC(C)(C)c1ccc(N(c2ccccc2)c2cc(C(C)(C)C)cc(N(c3ccc(C(C)(C)C)cc3)c3ccc(-c4nc(-c5ccccc5)nc(-c5ccccc5)n4)c4c3oc3ccccc34)c2-c2ccc3ccccc3c2)cc1]

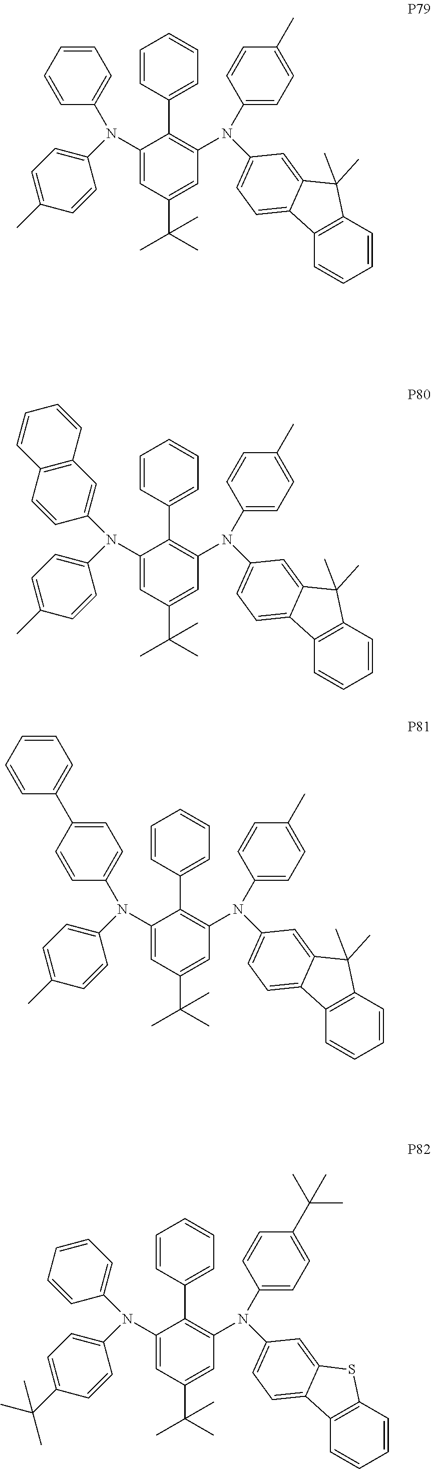 [SMILES: Cc1ccc(N(c2ccccc2)c2cc(C(C)(C)C)cc(N(c3ccc(C)cc3)c3ccc4c(c3)C(C)(C)c3ccccc3-4)c2-c2ccccc2)cc1 ; Cc1ccc(N(c2ccc3c(c2)C(C)(C)c2ccccc2-3)c2cc(C(C)(C)C)cc(N(c3ccc(C)cc3)c3ccc4ccccc4c3)c2-c2ccccc2)cc1 ; Cc1ccc(N(c2ccc(-c3ccccc3)cc2)c2cc(C(C)(C)C)cc(N(c3ccc(C)cc3)c3ccc4c(c3)C(C)(C)c3ccccc3-4)c2-c2ccccc2)cc1 ; CC(C)(C)c1ccc(N(c2ccccc2)c2cc(C(C)(C)C)cc(N(c3ccc(C(C)(C)C)cc3)c3ccc4c(c3)sc3ccccc34)c2-c2ccccc2)cc1]

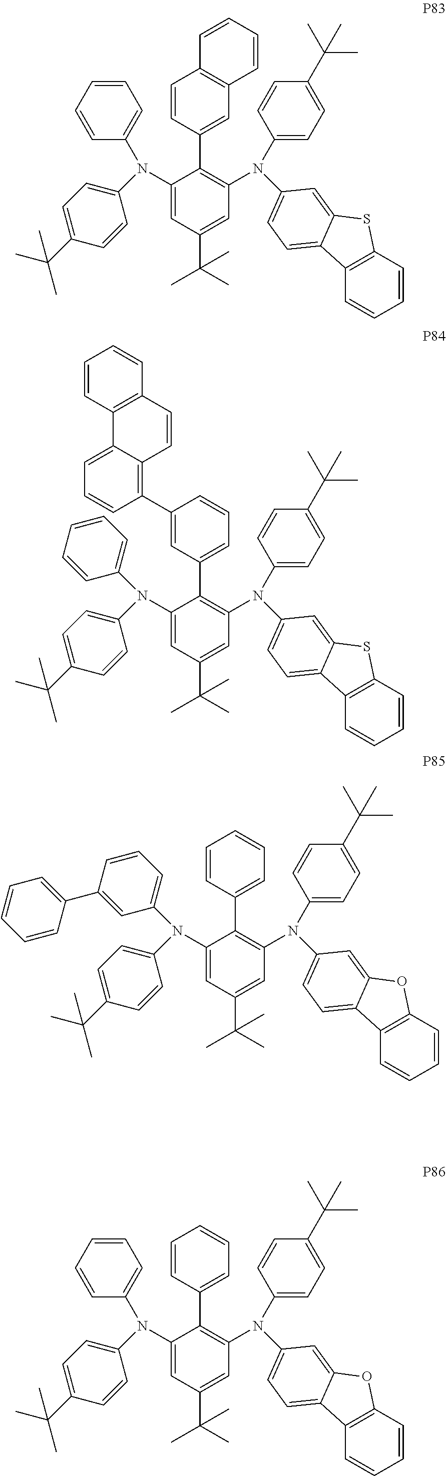

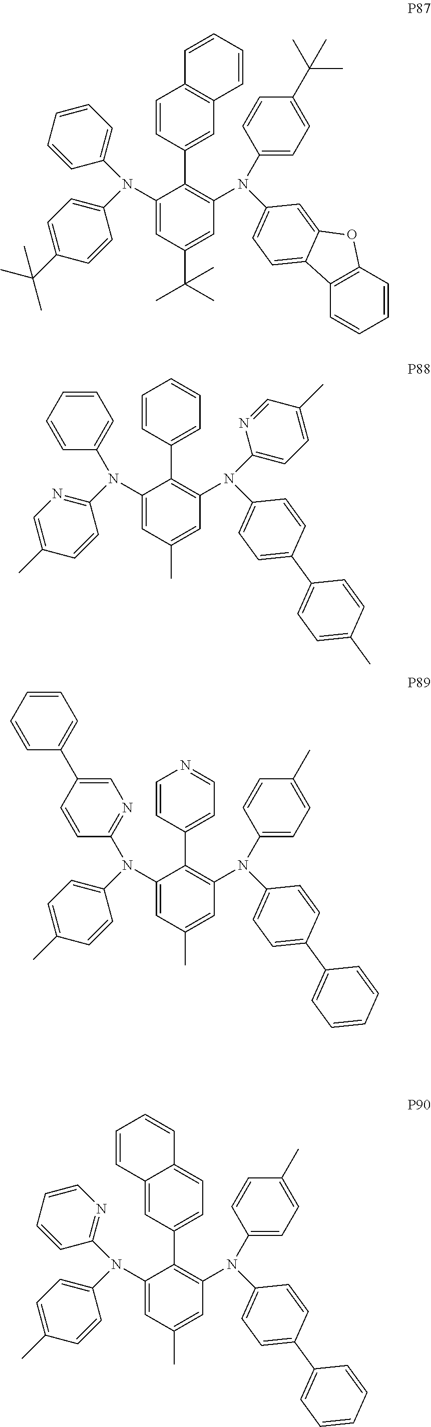

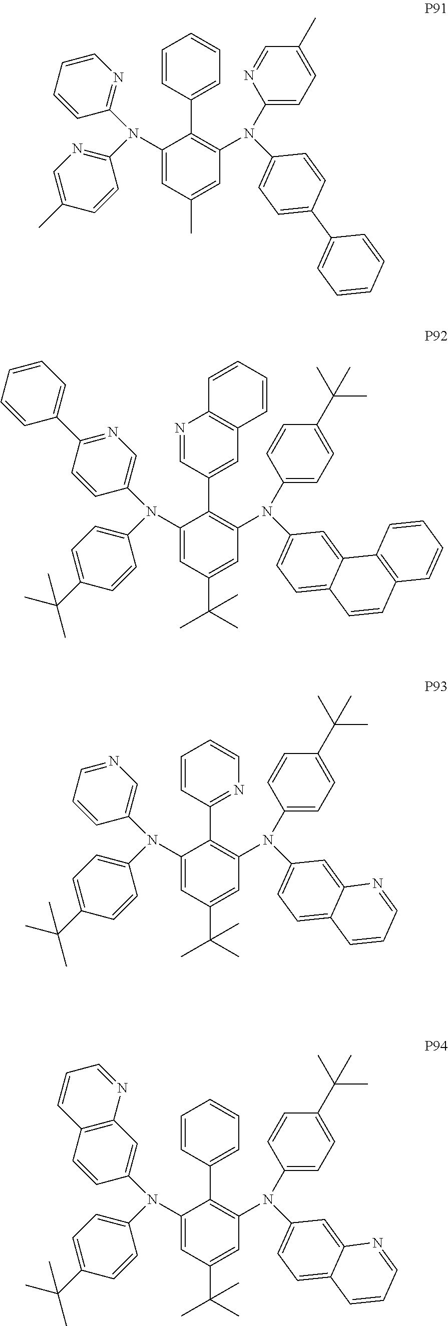 [SMILES: Cc1ccc(N(c2ccc(-c3ccccc3)cc2)c2cc(C)cc(N(c3ccccn3)c3ccc(C)cn3)c2-c2ccccc2)nc1 ; CC(C)(C)c1ccc(N(c2ccc(-c3ccccc3)nc2)c2cc(C(C)(C)C)cc(N(c3ccc(C(C)(C)C)cc3)c3ccc4ccc5ccccc5c4c3)c2-c2cnc3ccccc3c2)cc1 ; CC(C)(C)c1ccc(N(c2cccnc2)c2cc(C(C)(C)C)cc(N(c3ccc(C(C)(C)C)cc3)c3ccc4cccnc4c3)c2-c2ccccn2)cc1 ; CC(C)(C)c1ccc(N(c2ccc3cccnc3c2)c2cc(C(C)(C)C)cc(N(c3ccc(C(C)(C)C)cc3)c3ccc4cccnc4c3)c2-c2ccccc2)cc1]

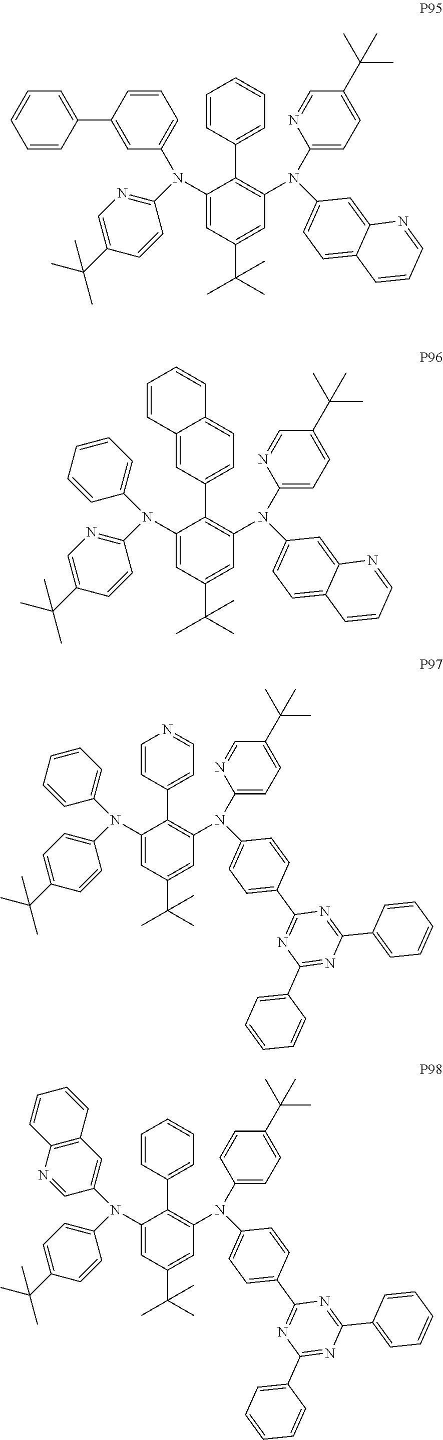

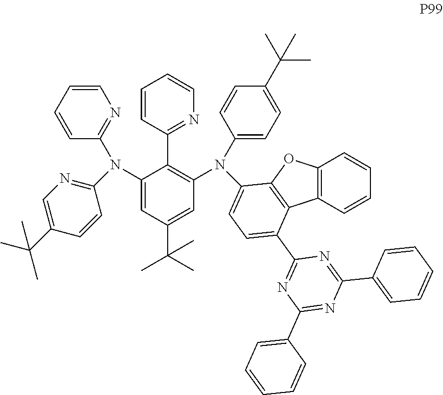 [SMILES: CC(C)(C)c1ccc(N(c2cc(C(C)(C)C)cc(N(c3ccccn3)c3ccc(C(C)(C)C)cn3)c2-c2ccccn2)c2ccc(-c3nc(-c4ccccc4)nc(-c4ccccc4)n3)c3c2oc2ccccc23)cc1]

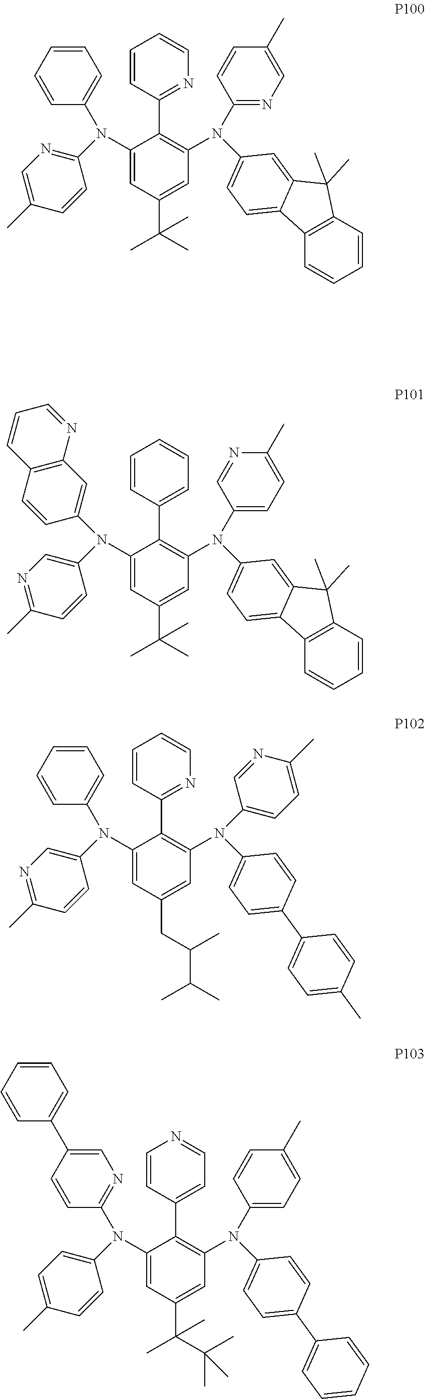

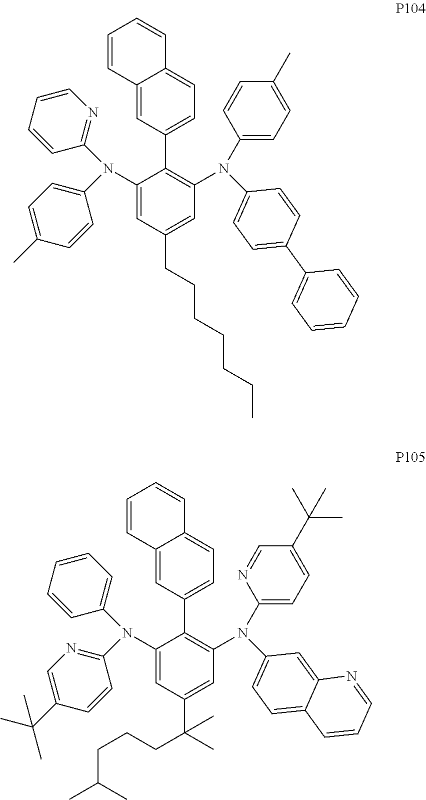 [SMILES: CCCCCCCc1cc(N(c2ccc(C)cc2)c2ccc(-c3ccccc3)cc2)c(-c2ccc3ccccc3c2)c(N(c2ccc(C)cc2)c2ccccn2)c1 ; CC(C)CCCC(C)(C)c1cc(N(c2ccccc2)c2ccc(C(C)(C)C)cn2)c(-c2ccc3ccccc3c2)c(N(c2ccc3cccnc3c2)c2ccc(C(C)(C)C)cn2)c1]

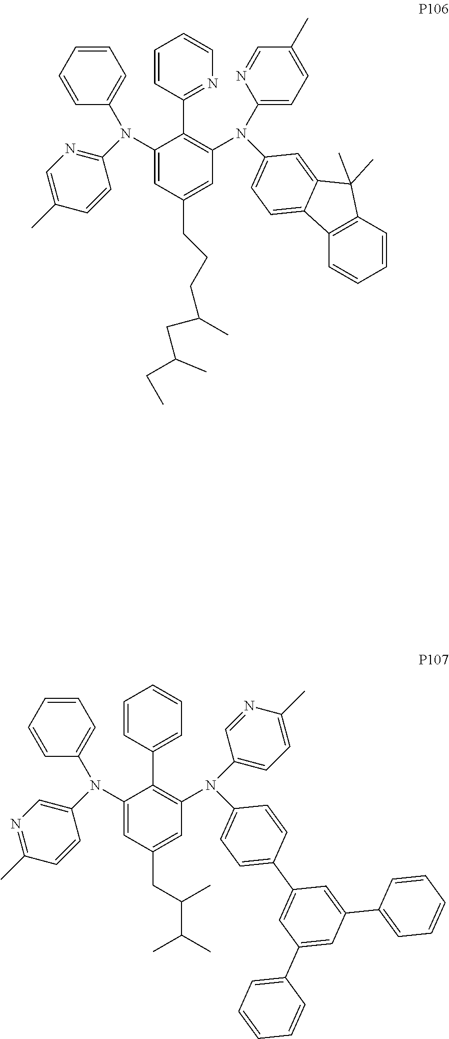 [SMILES: CCC(C)CC(C)CCCc1cc(N(c2ccccc2)c2ccc(C)cn2)c(-c2ccccn2)c(N(c2ccc3c(c2)C(C)(C)c2ccccc2-3)c2ccc(C)cn2)c1 ; Cc1ccc(N(c2ccccc2)c2cc(CC(C)C(C)C)cc(N(c3ccc(-c4cc(-c5ccccc5)cc(-c5ccccc5)c4)cc3)c3ccc(C)nc3)c2-c2ccccc2)cn1]

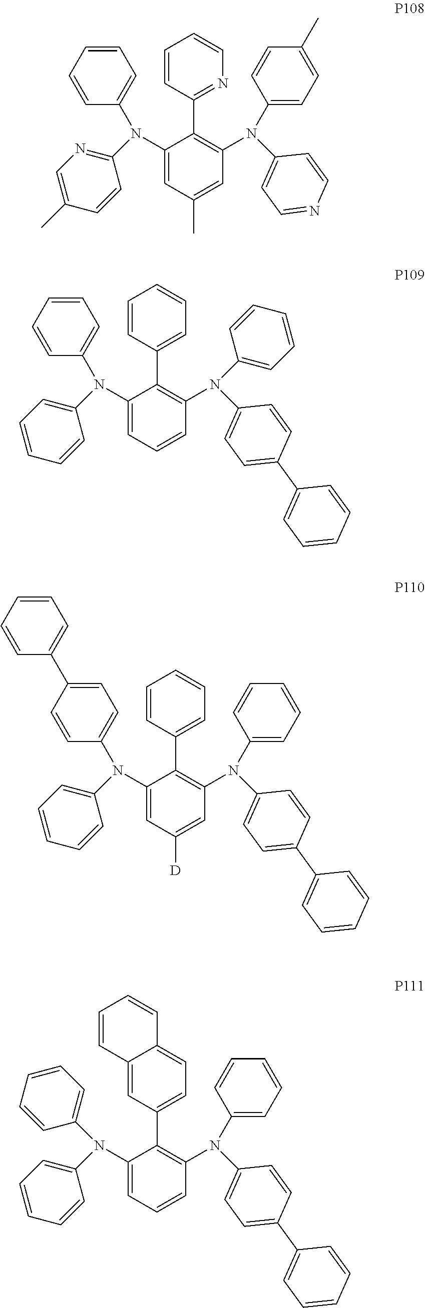 [SMILES: Cc1ccc(N(c2ccncc2)c2cc(C)cc(N(c3ccccc3)c3ccc(C)cn3)c2-c2ccccn2)cc1 ; c1ccc(-c2ccc(N(c3ccccc3)c3cccc(N(c4ccccc4)c4ccccc4)c3-c3ccccc3)cc2)cc1 ; [2H]c1cc(N(c2ccccc2)c2ccc(-c3ccccc3)cc2)c(-c2ccccc2)c(N(c2ccccc2)c2ccc(-c3ccccc3)cc2)c1 ; c1ccc(-c2ccc(N(c3ccccc3)c3cccc(N(c4ccccc4)c4ccccc4)c3-c3ccc4ccccc4c3)cc2)cc1]

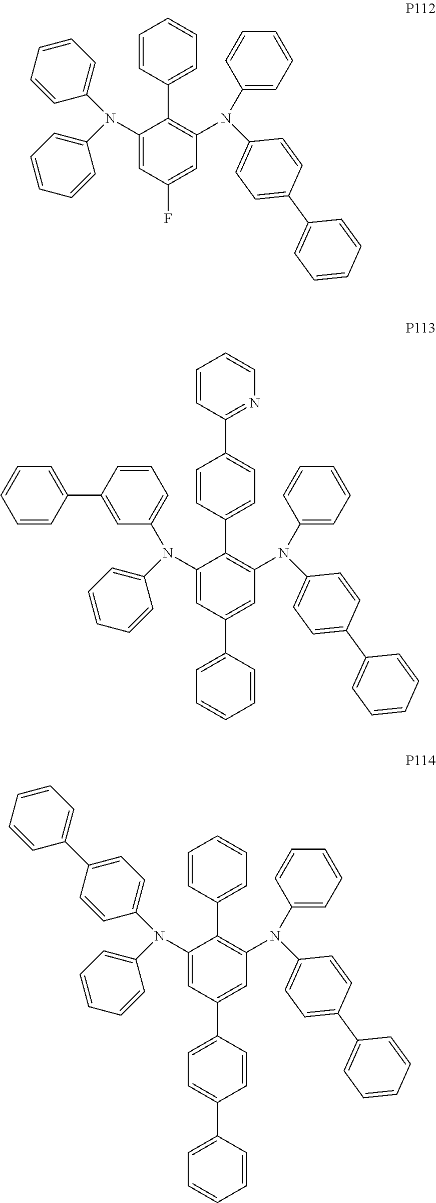 [SMILES: Fc1cc(N(c2ccccc2)c2ccccc2)c(-c2ccccc2)c(N(c2ccccc2)c2ccc(-c3ccccc3)cc2)c1 ; c1ccc(-c2ccc(N(c3ccccc3)c3cc(-c4ccccc4)cc(N(c4ccccc4)c4cccc(-c5ccccc5)c4)c3-c3ccc(-c4ccccn4)cc3)cc2)cc1 ; c1ccc(-c2ccc(-c3cc(N(c4ccccc4)c4ccc(-c5ccccc5)cc4)c(-c4ccccc4)c(N(c4ccccc4)c4ccc(-c5ccccc5)cc4)c3)cc2)cc1]

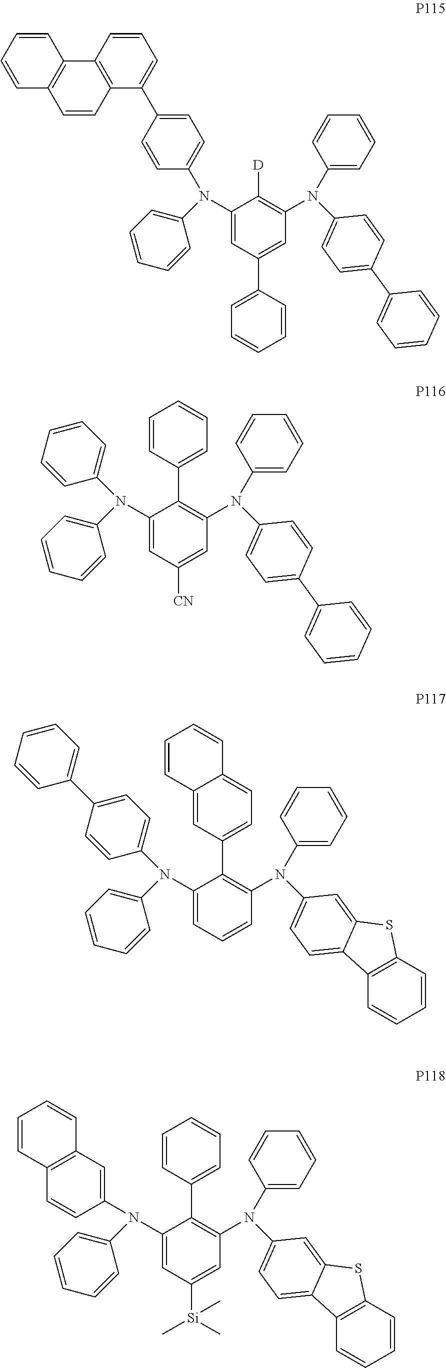 [SMILES: [2H]c1c(N(c2ccccc2)c2ccc(-c3ccccc3)cc2)cc(-c2ccccc2)cc1N(c1ccccc1)c1ccc(-c2cccc3c2ccc2ccccc23)cc1 ; N#Cc1cc(N(c2ccccc2)c2ccccc2)c(-c2ccccc2)c(N(c2ccccc2)c2ccc(-c3ccccc3)cc2)c1 ; c1ccc(-c2ccc(N(c3ccccc3)c3cccc(N(c4ccccc4)c4ccc5c(c4)sc4ccccc45)c3-c3ccc4ccccc4c3)cc2)cc1 ; C[Si](C)(C)c1cc(N(c2ccccc2)c2ccc3ccccc3c2)c(-c2ccccc2)c(N(c2ccccc2)c2ccc3c(c2)sc2ccccc23)c1]

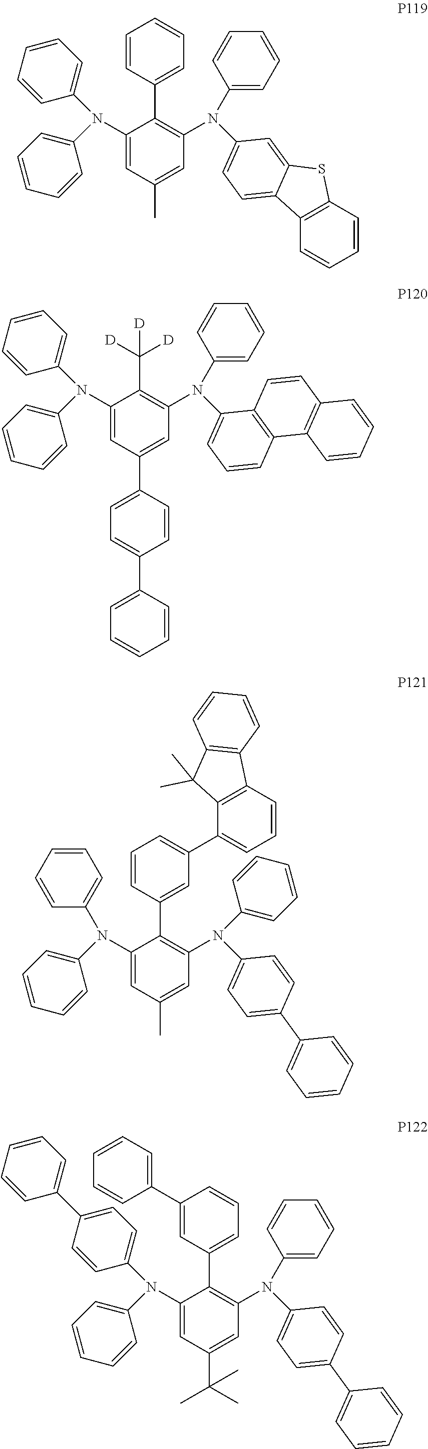 [SMILES: Cc1cc(N(c2ccccc2)c2ccccc2)c(-c2ccccc2)c(N(c2ccccc2)c2ccc3c(c2)sc2ccccc23)c1 ; [2H]C([2H])([2H])c1c(N(c2ccccc2)c2ccccc2)cc(-c2ccc(-c3ccccc3)cc2)cc1N(c1ccccc1)c1cccc2c1ccc1ccccc12 ; Cc1cc(N(c2ccccc2)c2ccccc2)c(-c2cccc(-c3cccc4c3C(C)(C)c3ccccc3-4)c2)c(N(c2ccccc2)c2ccc(-c3ccccc3)cc2)c1 ; CC(C)(C)c1cc(N(c2ccccc2)c2ccc(-c3ccccc3)cc2)c(-c2cccc(-c3ccccc3)c2)c(N(c2ccccc2)c2ccc(-c3ccccc3)cc2)c1]

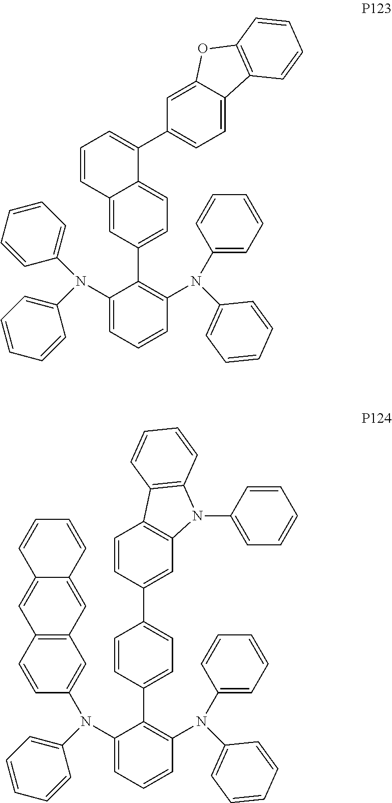 [SMILES: c1ccc(N(c2ccccc2)c2cccc(N(c3ccccc3)c3ccccc3)c2-c2ccc3c(-c4ccc5c(c4)oc4ccccc45)cccc3c2)cc1 ; c1ccc(N(c2ccccc2)c2cccc(N(c3ccccc3)c3ccc4cc5ccccc5cc4c3)c2-c2ccc(-c3ccc4c5ccccc5n(-c5ccccc5)c4c3)cc2)cc1]

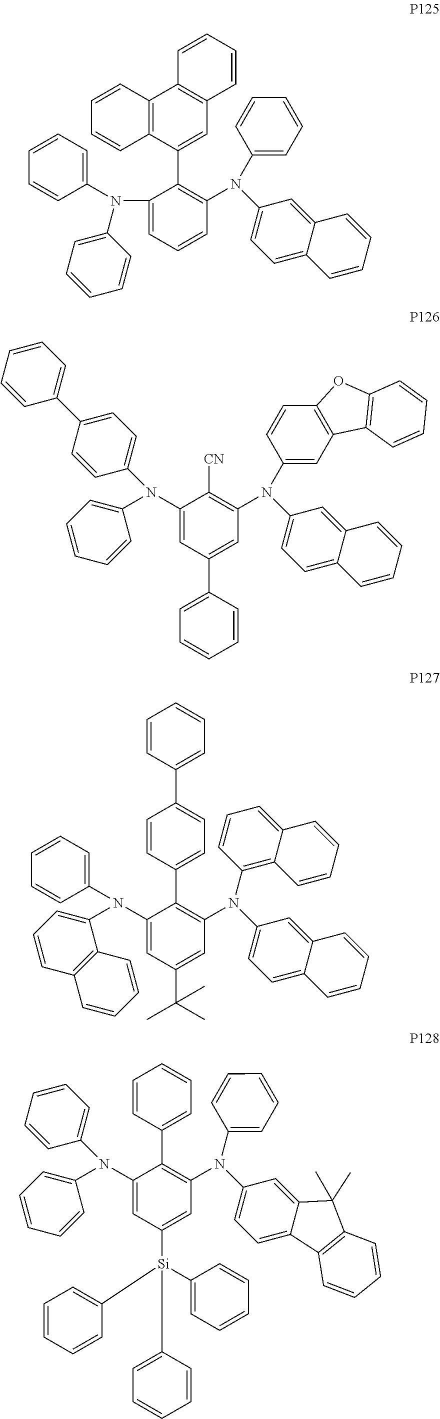

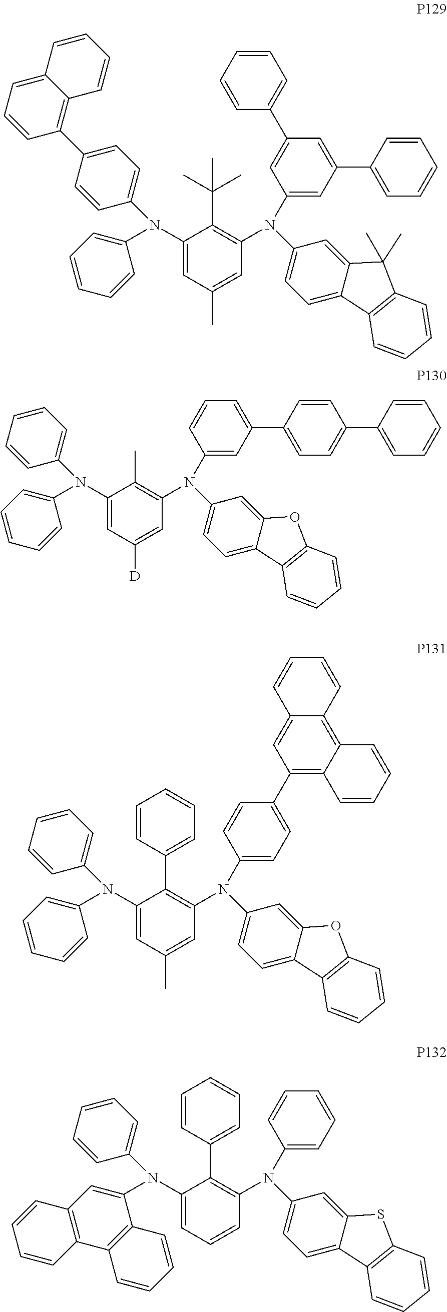

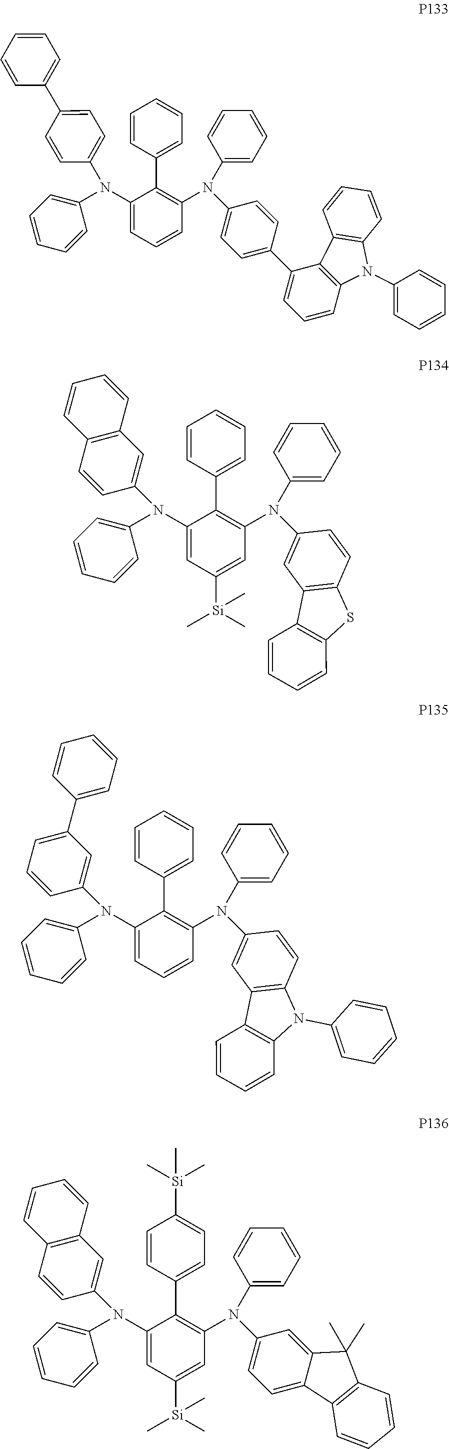 [SMILES: c1ccc(-c2ccc(N(c3ccccc3)c3cccc(N(c4ccccc4)c4ccc(-c5cccc6c5c5ccccc5n6-c5ccccc5)cc4)c3-c3ccccc3)cc2)cc1 ; C[Si](C)(C)c1cc(N(c2ccccc2)c2ccc3ccccc3c2)c(-c2ccccc2)c(N(c2ccccc2)c2ccc3sc4ccccc4c3c2)c1 ; c1ccc(-c2cccc(N(c3ccccc3)c3cccc(N(c4ccccc4)c4ccc5c(c4)c4ccccc4n5-c4ccccc4)c3-c3ccccc3)c2)cc1 ; CC1(C)c2ccccc2-c2ccc(N(c3ccccc3)c3cc([Si](C)(C)C)cc(N(c4ccccc4)c4ccc5ccccc5c4)c3-c3ccc([Si](C)(C)C)cc3)cc21]

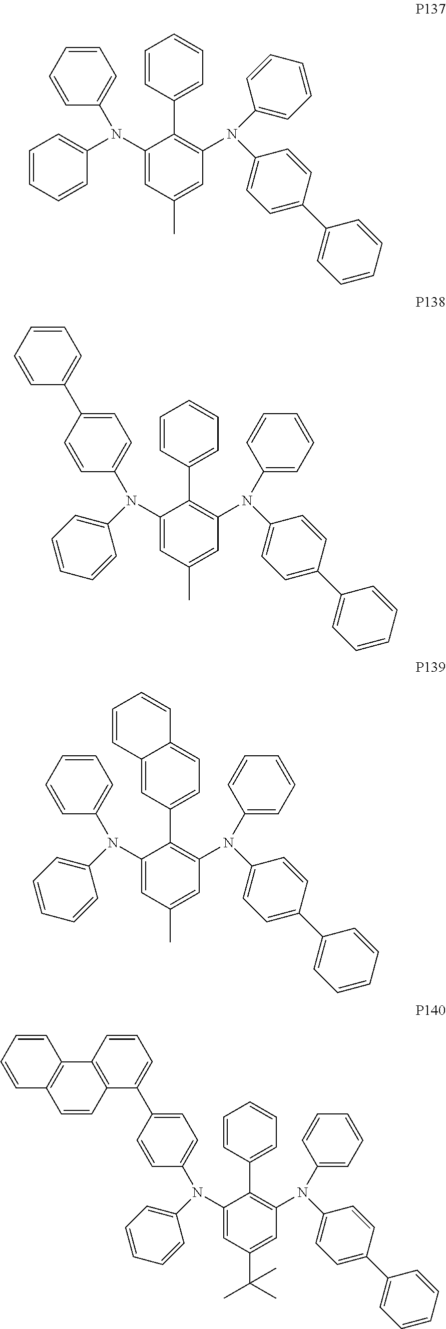 [SMILES: Cc1cc(N(c2ccccc2)c2ccccc2)c(-c2ccccc2)c(N(c2ccccc2)c2ccc(-c3ccccc3)cc2)c1 ; Cc1cc(N(c2ccccc2)c2ccc(-c3ccccc3)cc2)c(-c2ccccc2)c(N(c2ccccc2)c2ccc(-c3ccccc3)cc2)c1 ; Cc1cc(N(c2ccccc2)c2ccccc2)c(-c2ccc3ccccc3c2)c(N(c2ccccc2)c2ccc(-c3ccccc3)cc2)c1 ; CC(C)(C)c1cc(N(c2ccccc2)c2ccc(-c3ccccc3)cc2)c(-c2ccccc2)c(N(c2ccccc2)c2ccc(-c3cccc4c3ccc3ccccc34)cc2)c1]

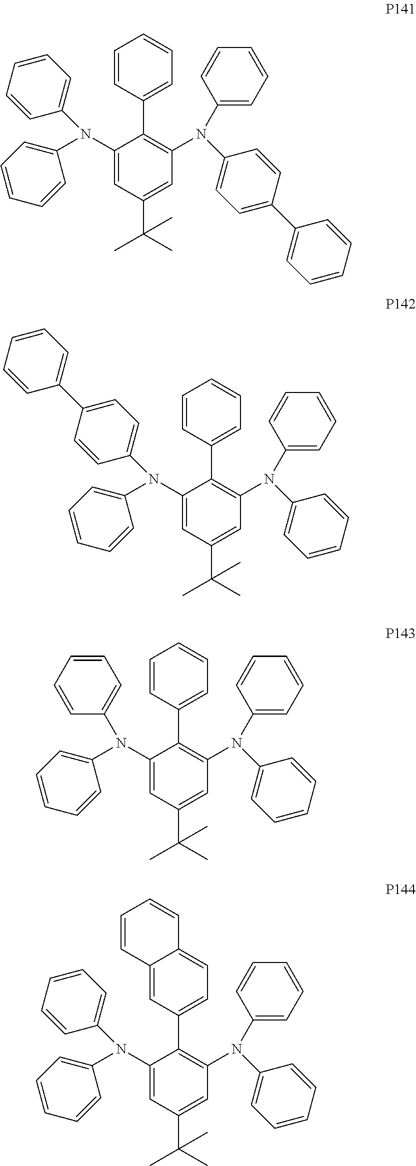

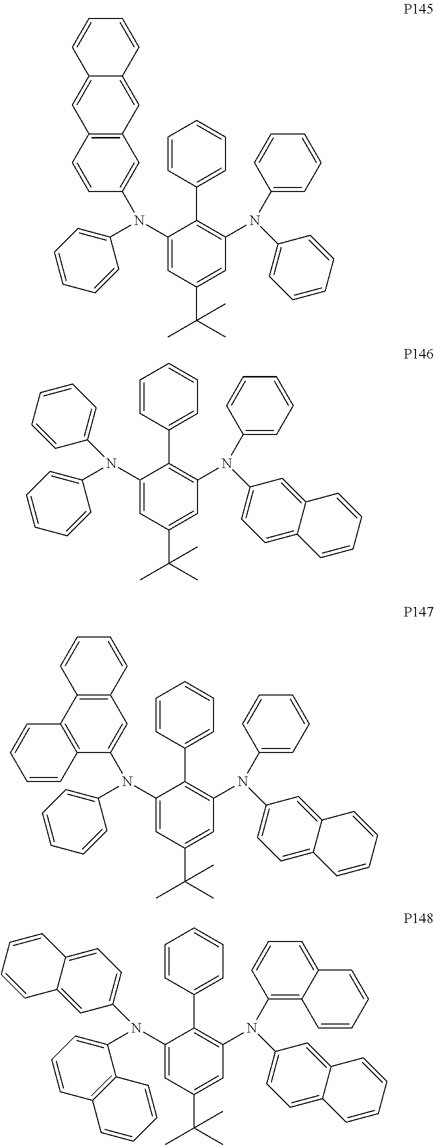

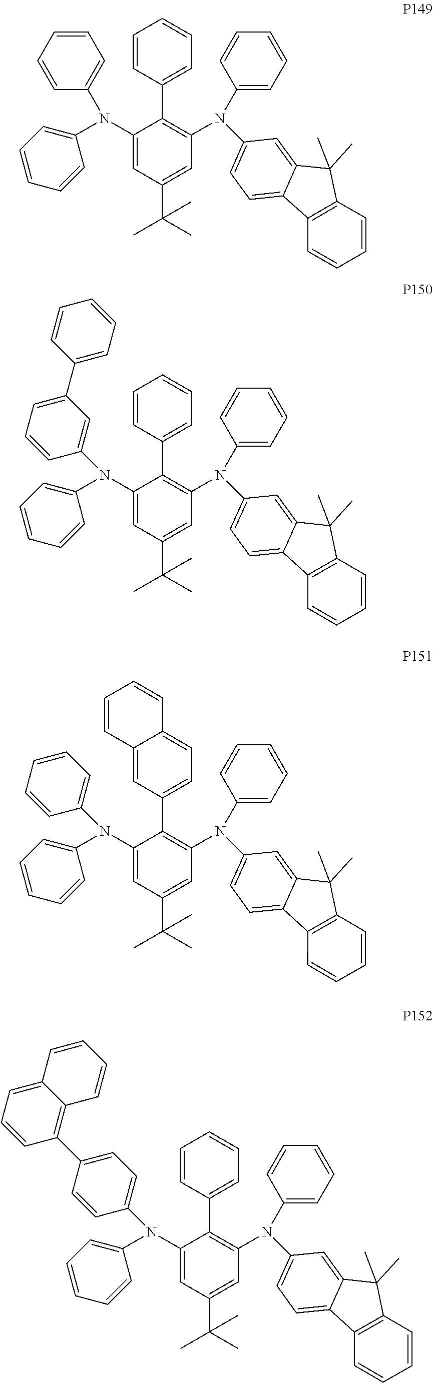 [SMILES: CC(C)(C)c1cc(N(c2ccccc2)c2ccccc2)c(-c2ccccc2)c(N(c2ccccc2)c2ccc3c(c2)C(C)(C)c2ccccc2-3)c1 ; CC(C)(C)c1cc(N(c2ccccc2)c2cccc(-c3ccccc3)c2)c(-c2ccccc2)c(N(c2ccccc2)c2ccc3c(c2)C(C)(C)c2ccccc2-3)c1 ; CC(C)(C)c1cc(N(c2ccccc2)c2ccccc2)c(-c2ccc3ccccc3c2)c(N(c2ccccc2)c2ccc3c(c2)C(C)(C)c2ccccc2-3)c1 ; CC(C)(C)c1cc(N(c2ccccc2)c2ccc(-c3cccc4ccccc34)cc2)c(-c2ccccc2)c(N(c2ccccc2)c2ccc3c(c2)C(C)(C)c2ccccc2-3)c1]

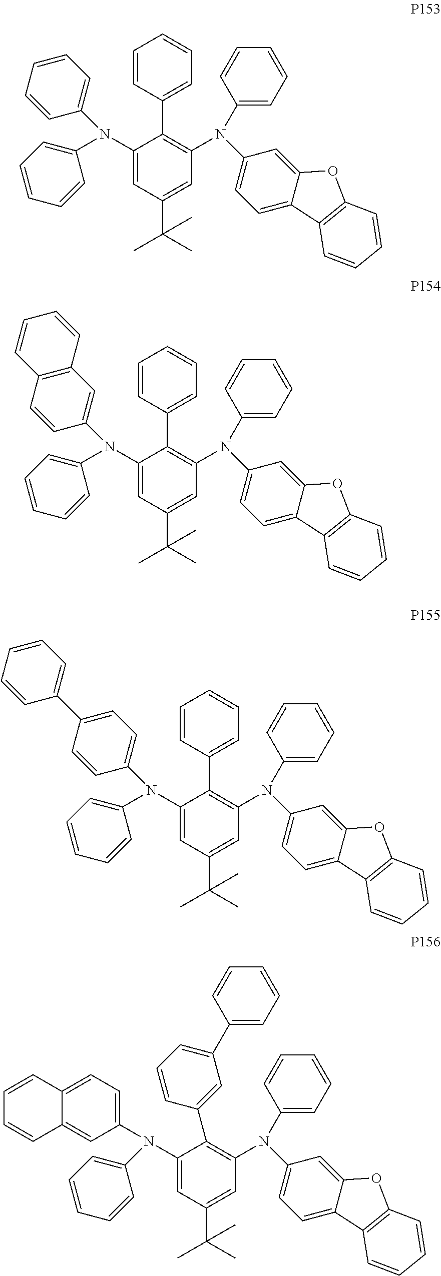 [SMILES: CC(C)(C)c1cc(N(c2ccccc2)c2ccccc2)c(-c2ccccc2)c(N(c2ccccc2)c2ccc3c(c2)oc2ccccc23)c1 ; CC(C)(C)c1cc(N(c2ccccc2)c2ccc3ccccc3c2)c(-c2ccccc2)c(N(c2ccccc2)c2ccc3c(c2)oc2ccccc23)c1 ; CC(C)(C)c1cc(N(c2ccccc2)c2ccc(-c3ccccc3)cc2)c(-c2ccccc2)c(N(c2ccccc2)c2ccc3c(c2)oc2ccccc23)c1 ; CC(C)(C)c1cc(N(c2ccccc2)c2ccc3ccccc3c2)c(-c2cccc(-c3ccccc3)c2)c(N(c2ccccc2)c2ccc3c(c2)oc2ccccc23)c1]

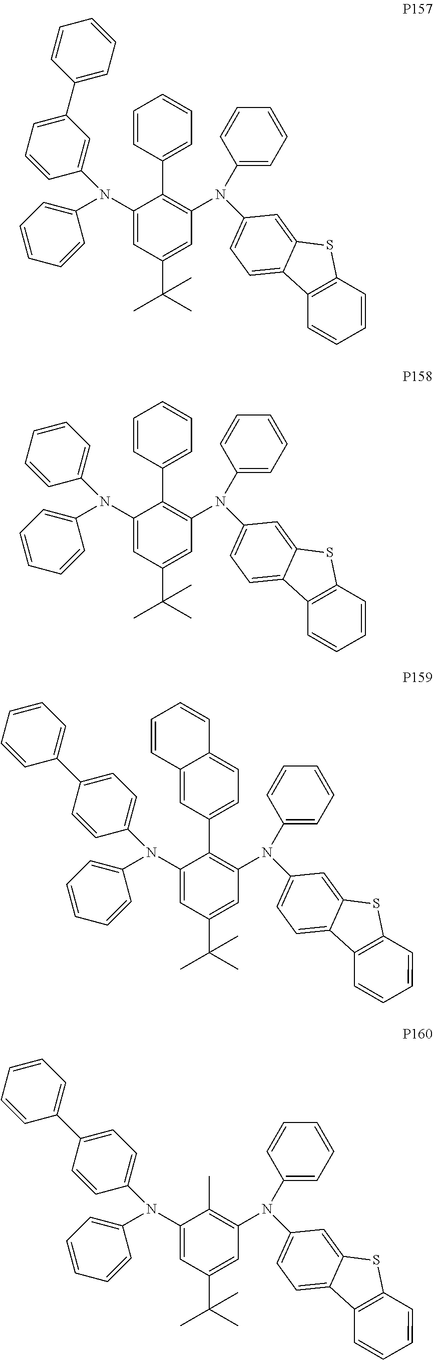 [SMILES: CC(C)(C)c1cc(N(c2ccccc2)c2cccc(-c3ccccc3)c2)c(-c2ccccc2)c(N(c2ccccc2)c2ccc3c(c2)sc2ccccc23)c1 ; CC(C)(C)c1cc(N(c2ccccc2)c2ccccc2)c(-c2ccccc2)c(N(c2ccccc2)c2ccc3c(c2)sc2ccccc23)c1 ; CC(C)(C)c1cc(N(c2ccccc2)c2ccc(-c3ccccc3)cc2)c(-c2ccc3ccccc3c2)c(N(c2ccccc2)c2ccc3c(c2)sc2ccccc23)c1 ; Cc1c(N(c2ccccc2)c2ccc(-c3ccccc3)cc2)cc(C(C)(C)C)cc1N(c1ccccc1)c1ccc2c(c1)sc1ccccc12]

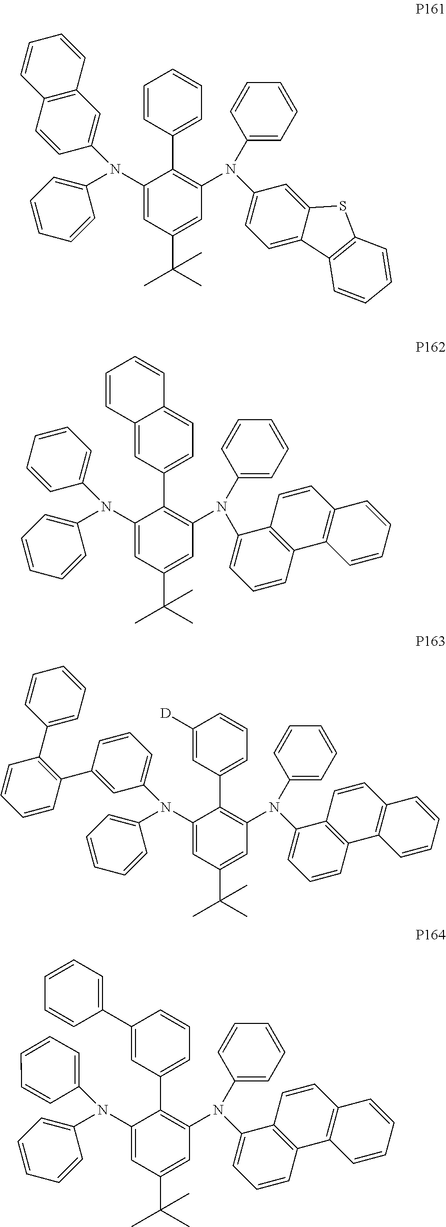 [SMILES: CC(C)(C)c1cc(N(c2ccccc2)c2ccc3ccccc3c2)c(-c2ccccc2)c(N(c2ccccc2)c2ccc3c(c2)sc2ccccc23)c1 ; CC(C)(C)c1cc(N(c2ccccc2)c2ccccc2)c(-c2ccc3ccccc3c2)c(N(c2ccccc2)c2cccc3c2ccc2ccccc23)c1 ; [2H]c1cccc(-c2c(N(c3ccccc3)c3cccc(-c4ccccc4-c4ccccc4)c3)cc(C(C)(C)C)cc2N(c2ccccc2)c2cccc3c2ccc2ccccc23)c1 ; CC(C)(C)c1cc(N(c2ccccc2)c2ccccc2)c(-c2cccc(-c3ccccc3)c2)c(N(c2ccccc2)c2cccc3c2ccc2ccccc23)c1]

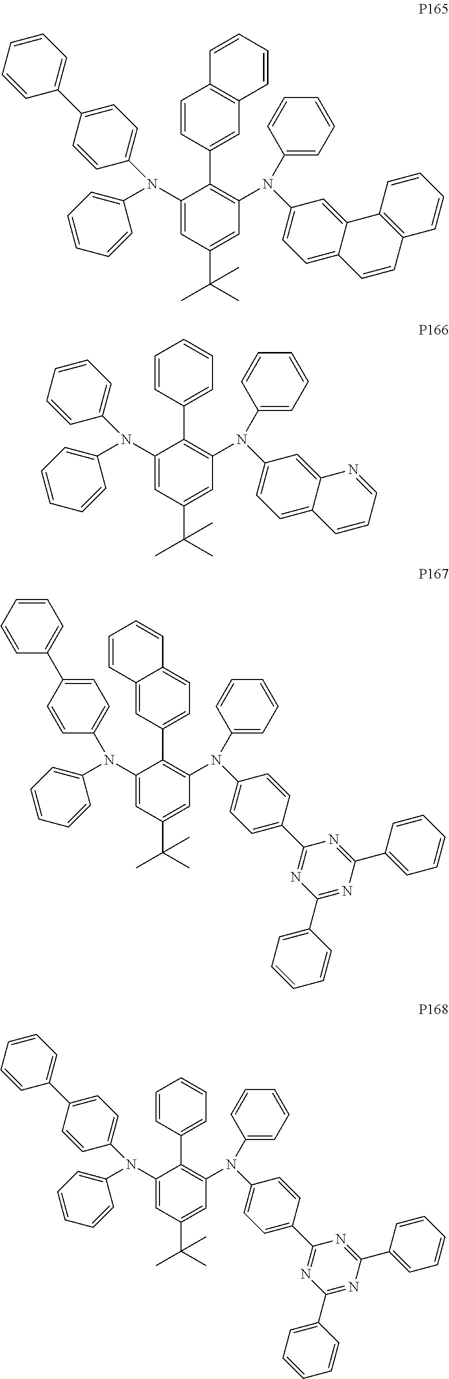

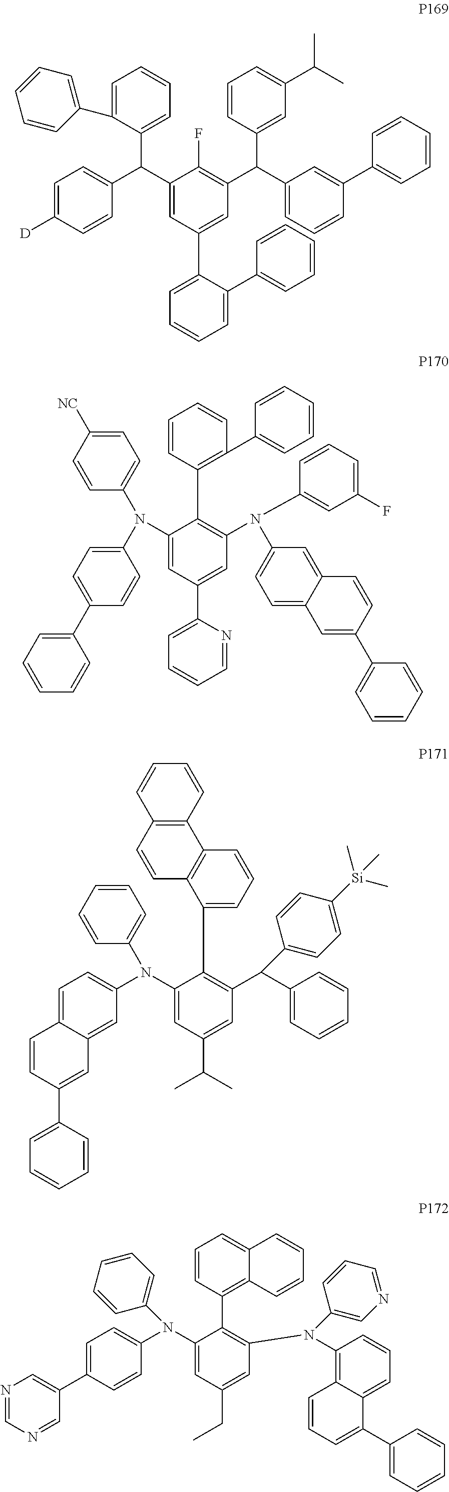 [SMILES: [2H]c1ccc(C(c2ccccc2-c2ccccc2)c2cc(-c3ccccc3-c3ccccc3)cc(C(c3cccc(-c4ccccc4)c3)c3cccc(C(C)C)c3)c2F)cc1 ; N#Cc1ccc(N(c2ccc(-c3ccccc3)cc2)c2cc(-c3ccccn3)cc(N(c3cccc(F)c3)c3ccc4cc(-c5ccccc5)ccc4c3)c2-c2ccccc2-c2ccccc2)cc1 ; CC(C)c1cc(C(c2ccccc2)c2ccc([Si](C)(C)C)cc2)c(-c2cccc3c2ccc2ccccc23)c(N(c2ccccc2)c2ccc3ccc(-c4ccccc4)cc3c2)c1 ; CCc1cc(N(c2ccccc2)c2ccc(-c3cncnc3)cc2)c(-c2cccc3ccccc23)c(N(c2cccnc2)c2cccc3c(-c4ccccc4)cccc23)c1]

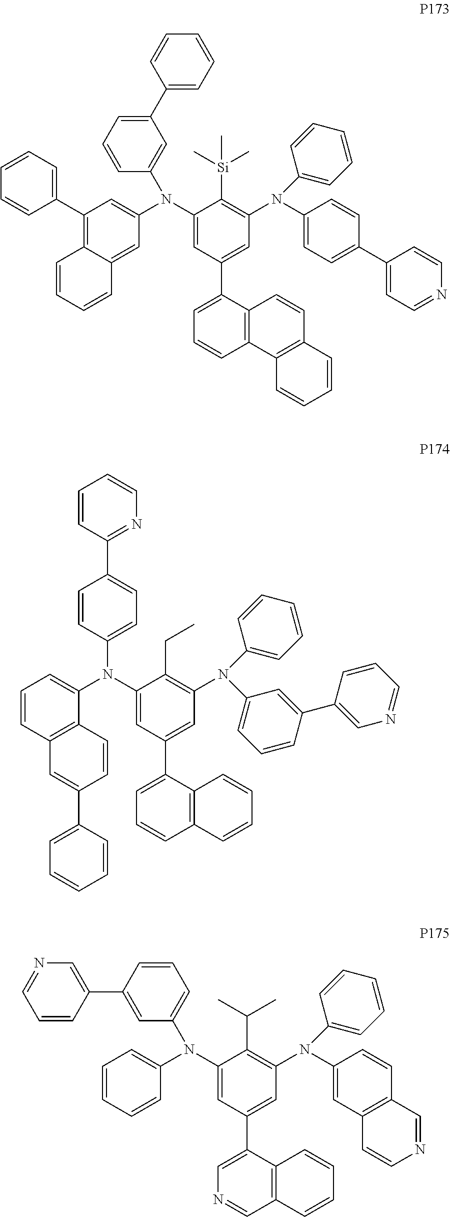 [SMILES: C[Si](C)(C)c1c(N(c2ccccc2)c2ccc(-c3ccncc3)cc2)cc(-c2cccc3c2ccc2ccccc23)cc1N(c1cccc(-c2ccccc2)c1)c1cc(-c2ccccc2)c2ccccc2c1 ; CCc1c(N(c2ccccc2)c2cccc(-c3cccnc3)c2)cc(-c2cccc3ccccc23)cc1N(c1ccc(-c2ccccn2)cc1)c1cccc2cc(-c3ccccc3)ccc12 ; CC(C)c1c(N(c2ccccc2)c2cccc(-c3cccnc3)c2)cc(-c2cncc3ccccc23)cc1N(c1ccccc1)c1ccc2cnccc2c1]

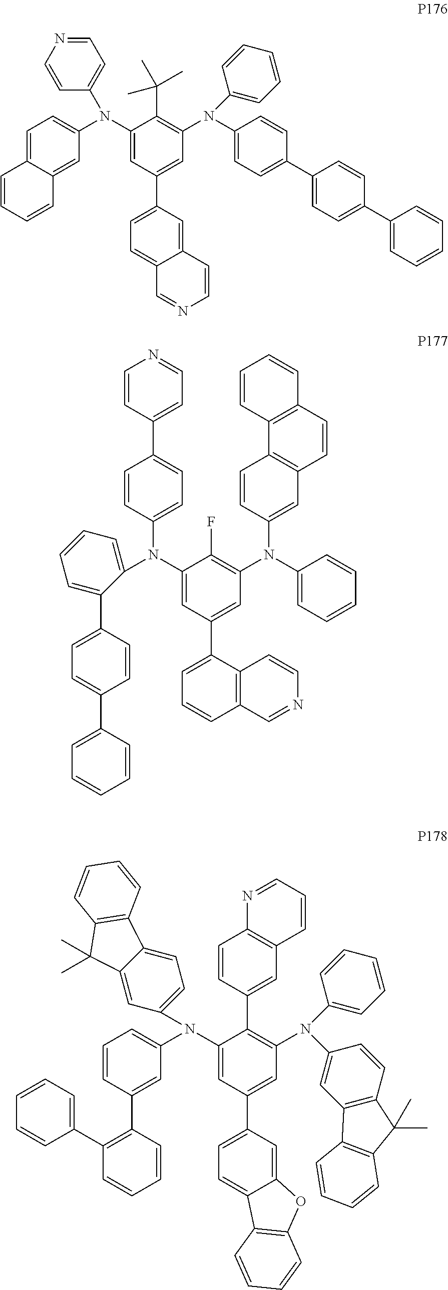 [SMILES: CC(C)(C)c1c(N(c2ccccc2)c2ccc(-c3ccc(-c4ccccc4)cc3)cc2)cc(-c2ccc3cnccc3c2)cc1N(c1ccncc1)c1ccc2ccccc2c1 ; Fc1c(N(c2ccccc2)c2ccc3c(ccc4ccccc43)c2)cc(-c2cccc3cnccc23)cc1N(c1ccc(-c2ccncc2)cc1)c1ccccc1-c1ccc(-c2ccccc2)cc1 ; CC1(C)c2ccccc2-c2cc(N(c3ccccc3)c3cc(-c4ccc5c(c4)oc4ccccc45)cc(N(c4cccc(-c5ccccc5-c5ccccc5)c4)c4ccc5c(c4)C(C)(C)c4ccccc4-5)c3-c3ccc4ncccc4c3)ccc21]

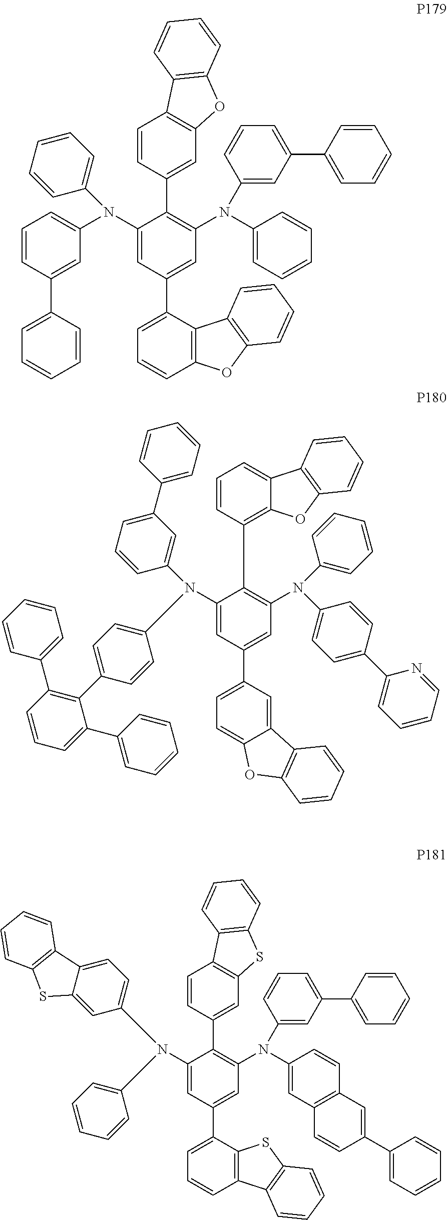 [SMILES: c1ccc(-c2cccc(N(c3ccccc3)c3cc(-c4cccc5oc6ccccc6c45)cc(N(c4ccccc4)c4cccc(-c5ccccc5)c4)c3-c3ccc4c(c3)oc3ccccc34)c2)cc1 ; c1ccc(-c2cccc(N(c3ccc(-c4c(-c5ccccc5)cccc4-c4ccccc4)cc3)c3cc(-c4ccc5oc6ccccc6c5c4)cc(N(c4ccccc4)c4ccc(-c5ccccn5)cc4)c3-c3cccc4c3oc3ccccc34)c2)cc1 ; c1ccc(-c2cccc(N(c3ccc4cc(-c5ccccc5)ccc4c3)c3cc(-c4cccc5c4sc4ccccc45)cc(N(c4ccccc4)c4ccc5c(c4)sc4ccccc45)c3-c3ccc4c(c3)sc3ccccc34)c2)cc1]

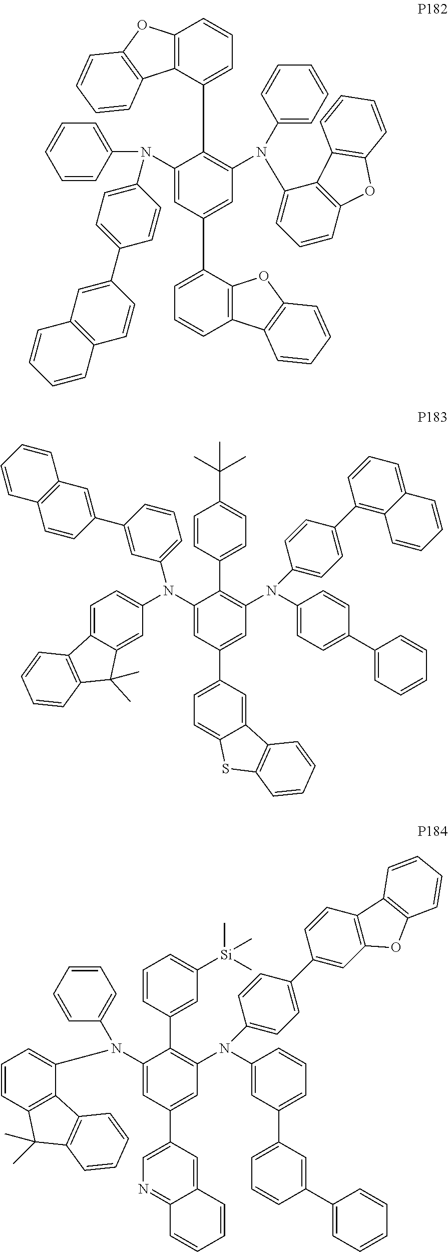 [SMILES: c1ccc(N(c2ccc(-c3ccc4ccccc4c3)cc2)c2cc(-c3cccc4c3oc3ccccc34)cc(N(c3ccccc3)c3cccc4oc5ccccc5c34)c2-c2cccc3oc4ccccc4c23)cc1 ; CC(C)(C)c1ccc(-c2c(N(c3ccc(-c4ccccc4)cc3)c3ccc(-c4cccc5ccccc45)cc3)cc(-c3ccc4sc5ccccc5c4c3)cc2N(c2cccc(-c3ccc4ccccc4c3)c2)c2ccc3c(c2)C(C)(C)c2ccccc2-3)cc1 ; CC1(C)c2ccccc2-c2c(N(c3ccccc3)c3cc(-c4cnc5ccccc5c4)cc(N(c4ccc(-c5ccc6c(c5)oc5ccccc56)cc4)c4cccc(-c5cccc(-c6ccccc6)c5)c4)c3-c3cccc([Si](C)(C)C)c3)cccc21]

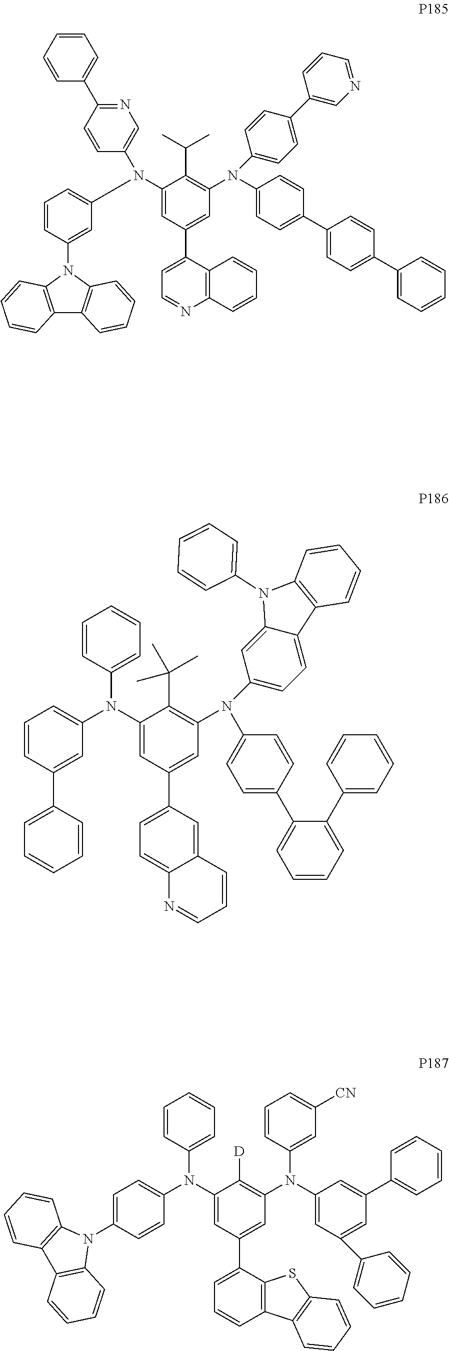 [SMILES: CC(C)c1c(N(c2ccc(-c3ccc(-c4ccccc4)cc3)cc2)c2ccc(-c3cccnc3)cc2)cc(-c2ccnc3ccccc23)cc1N(c1ccc(-c2ccccc2)nc1)c1cccc(-n2c3ccccc3c3ccccc32)c1 ; CC(C)(C)c1c(N(c2ccccc2)c2cccc(-c3ccccc3)c2)cc(-c2ccc3ncccc3c2)cc1N(c1ccc(-c2ccccc2-c2ccccc2)cc1)c1ccc2c3ccccc3n(-c3ccccc3)c2c1 ; [2H]c1c(N(c2ccccc2)c2ccc(-n3c4ccccc4c4ccccc43)cc2)cc(-c2cccc3c2sc2ccccc23)cc1N(c1cccc(C#N)c1)c1cc(-c2ccccc2)cc(-c2ccccc2)c1]

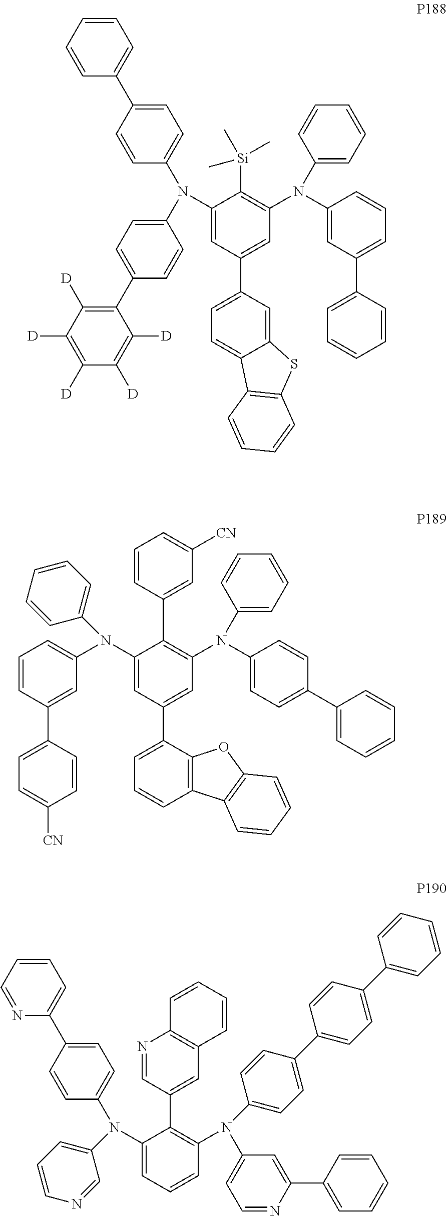 [SMILES: [2H]c1c([2H])c([2H])c(-c2ccc(N(c3ccc(-c4ccccc4)cc3)c3cc(-c4ccc5c(c4)sc4ccccc45)cc(N(c4ccccc4)c4cccc(-c5ccccc5)c4)c3[Si](C)(C)C)cc2)c([2H])c1[2H] ; N#Cc1ccc(-c2cccc(N(c3ccccc3)c3cc(-c4cccc5c4oc4ccccc45)cc(N(c4ccccc4)c4ccc(-c5ccccc5)cc4)c3-c3cccc(C#N)c3)c2)cc1 ; c1ccc(-c2ccc(-c3ccc(N(c4ccnc(-c5ccccc5)c4)c4cccc(N(c5ccc(-c6ccccn6)cc5)c5cccnc5)c4-c4cnc5ccccc5c4)cc3)cc2)cc1]

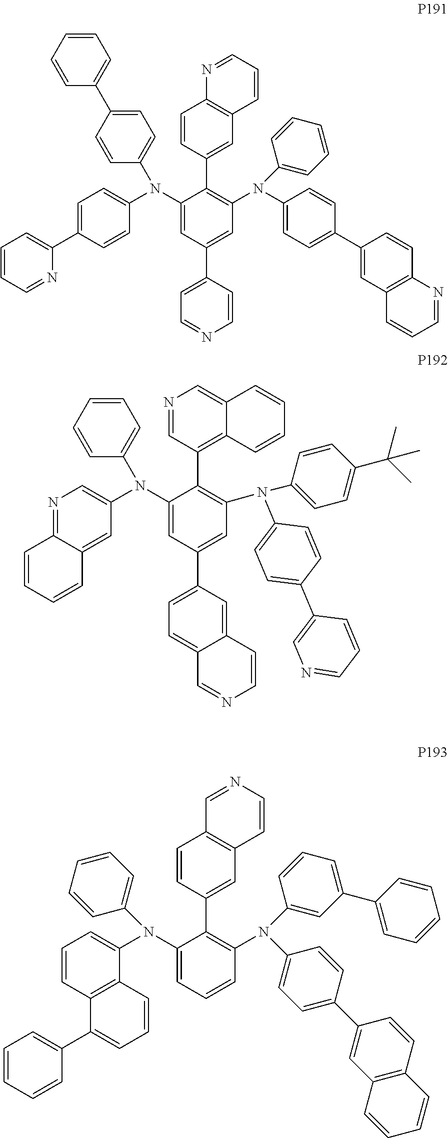 [SMILES: c1ccc(-c2ccc(N(c3ccc(-c4ccccn4)cc3)c3cc(-c4ccncc4)cc(N(c4ccccc4)c4ccc(-c5ccc6ncccc6c5)cc4)c3-c3ccc4ncccc4c3)cc2)cc1 ; CC(C)(C)c1ccc(N(c2ccc(-c3cccnc3)cc2)c2cc(-c3ccc4cnccc4c3)cc(N(c3ccccc3)c3cnc4ccccc4c3)c2-c2cncc3ccccc23)cc1 ; c1ccc(-c2cccc(N(c3ccc(-c4ccc5ccccc5c4)cc3)c3cccc(N(c4ccccc4)c4cccc5c(-c6ccccc6)cccc45)c3-c3ccc4cnccc4c3)c2)cc1]

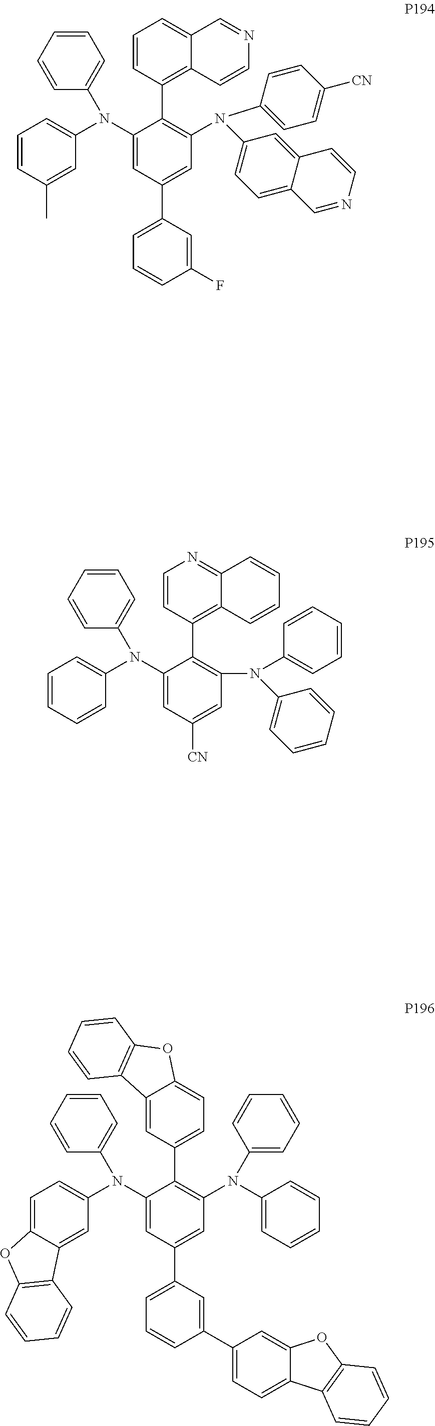 [SMILES: Cc1cccc(N(c2ccccc2)c2cc(-c3cccc(F)c3)cc(N(c3ccc(C#N)cc3)c3ccc4cnccc4c3)c2-c2cccc3cnccc23)c1 ; N#Cc1cc(N(c2ccccc2)c2ccccc2)c(-c2ccnc3ccccc23)c(N(c2ccccc2)c2ccccc2)c1 ; c1ccc(N(c2ccccc2)c2cc(-c3cccc(-c4ccc5c(c4)oc4ccccc45)c3)cc(N(c3ccccc3)c3ccc4oc5ccccc5c4c3)c2-c2ccc3oc4ccccc4c3c2)cc1]

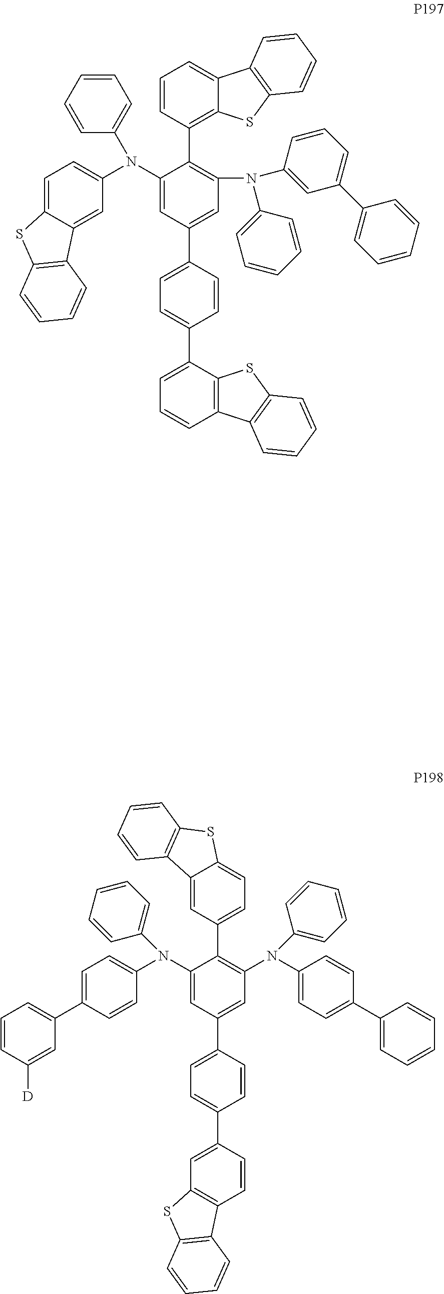 [SMILES: c1ccc(-c2cccc(N(c3ccccc3)c3cc(-c4ccc(-c5cccc6c5sc5ccccc56)cc4)cc(N(c4ccccc4)c4ccc5sc6ccccc6c5c4)c3-c3cccc4c3sc3ccccc34)c2)cc1 ; [2H]c1cccc(-c2ccc(N(c3ccccc3)c3cc(-c4ccc(-c5ccc6c(c5)sc5ccccc56)cc4)cc(N(c4ccccc4)c4ccc(-c5ccccc5)cc4)c3-c3ccc4sc5ccccc5c4c3)cc2)c1]

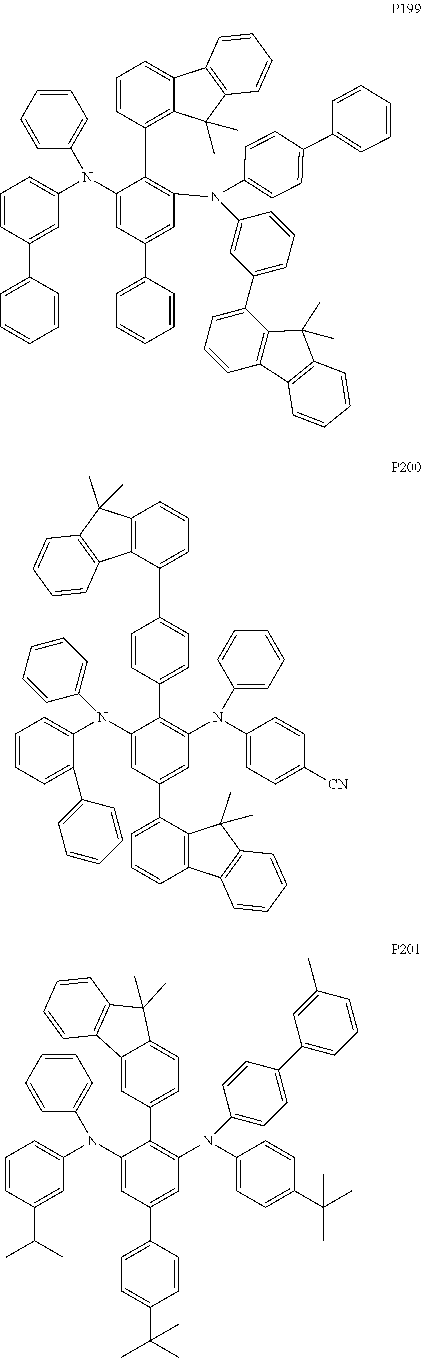 [SMILES: CC1(C)c2ccccc2-c2cccc(-c3cccc(N(c4ccc(-c5ccccc5)cc4)c4cc(-c5ccccc5)cc(N(c5ccccc5)c5cccc(-c6ccccc6)c5)c4-c4cccc5c4C(C)(C)c4ccccc4-5)c3)c21 ; CC1(C)c2ccccc2-c2c(-c3ccc(-c4c(N(c5ccccc5)c5ccc(C#N)cc5)cc(-c5cccc6c5C(C)(C)c5ccccc5-6)cc4N(c4ccccc4)c4ccccc4-c4ccccc4)cc3)cccc21 ; Cc1cccc(-c2ccc(N(c3ccc(C(C)(C)C)cc3)c3cc(-c4ccc(C(C)(C)C)cc4)cc(N(c4ccccc4)c4cccc(C(C)C)c4)c3-c3ccc4c(c3)-c3ccccc3C4(C)C)cc2)c1]

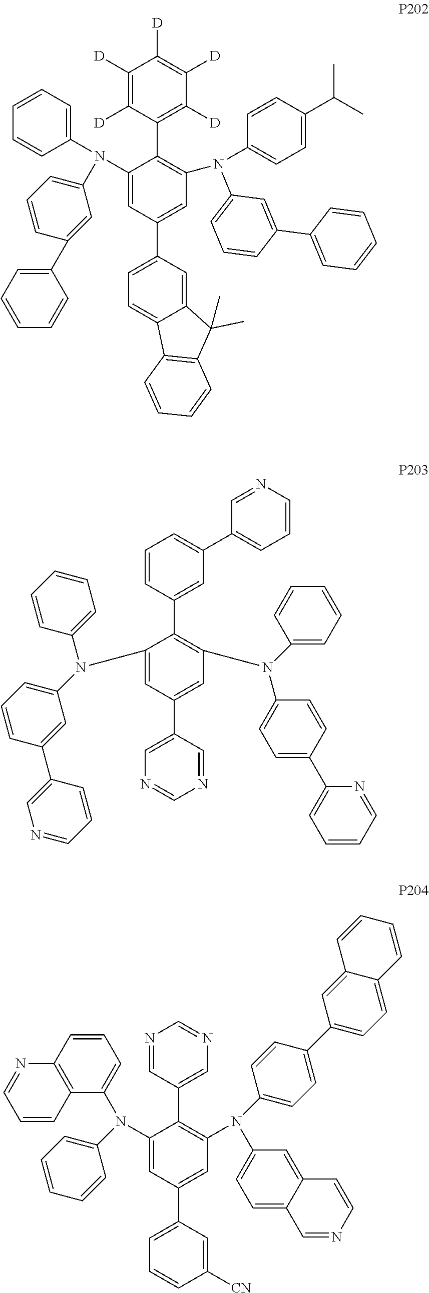 [SMILES: [2H]c1c([2H])c([2H])c(-c2c(N(c3ccccc3)c3cccc(-c4ccccc4)c3)cc(-c3ccc4c(c3)C(C)(C)c3ccccc3-4)cc2N(c2ccc(C(C)C)cc2)c2cccc(-c3ccccc3)c2)c([2H])c1[2H] ; c1ccc(N(c2ccc(-c3ccccn3)cc2)c2cc(-c3cncnc3)cc(N(c3ccccc3)c3cccc(-c4cccnc4)c3)c2-c2cccc(-c3cccnc3)c2)cc1 ; N#Cc1cccc(-c2cc(N(c3ccc(-c4ccc5ccccc5c4)cc3)c3ccc4cnccc4c3)c(-c3cncnc3)c(N(c3ccccc3)c3cccc4ncccc34)c2)c1]

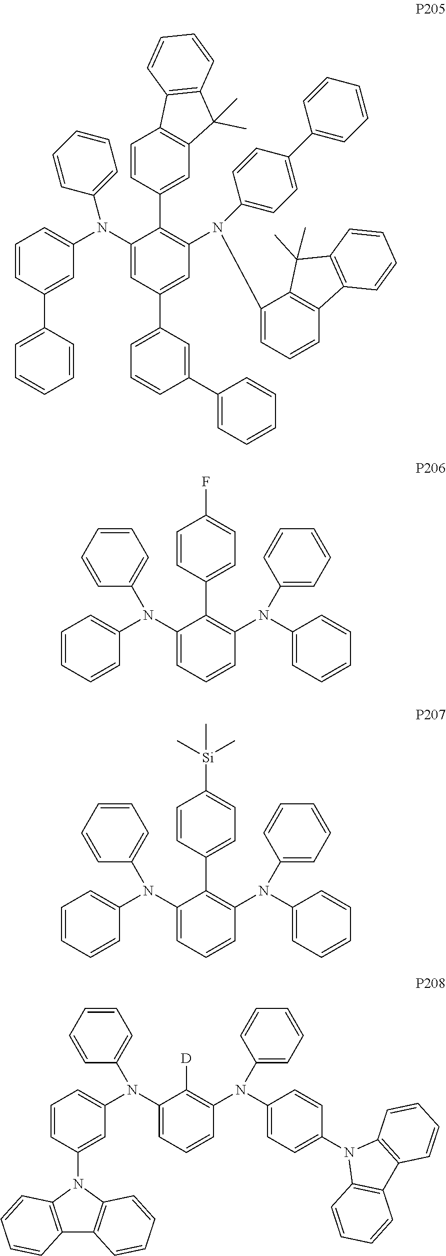

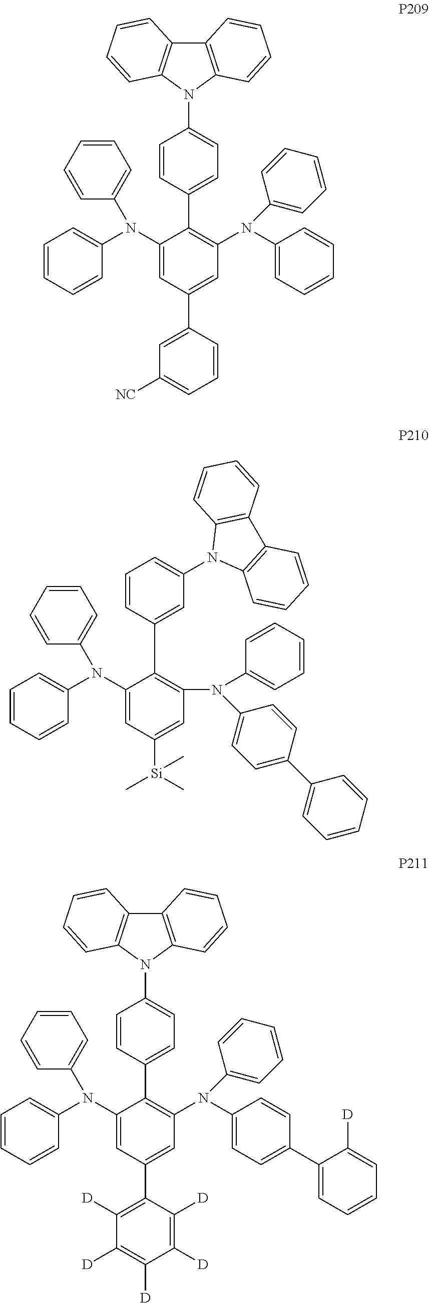 [SMILES: N#Cc1cccc(-c2cc(N(c3ccccc3)c3ccccc3)c(-c3ccc(-n4c5ccccc5c5ccccc54)cc3)c(N(c3ccccc3)c3ccccc3)c2)c1 ; C[Si](C)(C)c1cc(N(c2ccccc2)c2ccccc2)c(-c2cccc(-n3c4ccccc4c4ccccc43)c2)c(N(c2ccccc2)c2ccc(-c3ccccc3)cc2)c1 ; [2H]c1ccccc1-c1ccc(N(c2ccccc2)c2cc(-c3c([2H])c([2H])c([2H])c([2H])c3[2H])cc(N(c3ccccc3)c3ccccc3)c2-c2ccc(-n3c4ccccc4c4ccccc43)cc2)cc1]

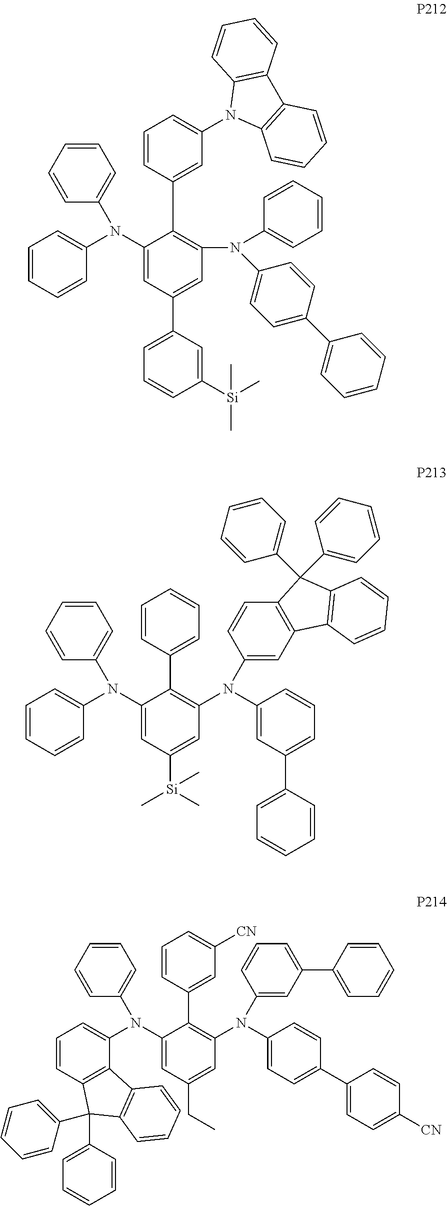 [SMILES: C[Si](C)(C)c1cccc(-c2cc(N(c3ccccc3)c3ccccc3)c(-c3cccc(-n4c5ccccc5c5ccccc54)c3)c(N(c3ccccc3)c3ccc(-c4ccccc4)cc3)c2)c1 ; C[Si](C)(C)c1cc(N(c2ccccc2)c2ccccc2)c(-c2ccccc2)c(N(c2cccc(-c3ccccc3)c2)c2ccc3c(c2)-c2ccccc2C3(c2ccccc2)c2ccccc2)c1 ; CCc1cc(N(c2ccc(-c3ccc(C#N)cc3)cc2)c2cccc(-c3ccccc3)c2)c(-c2cccc(C#N)c2)c(N(c2ccccc2)c2cccc3c2-c2ccccc2C3(c2ccccc2)c2ccccc2)c1]

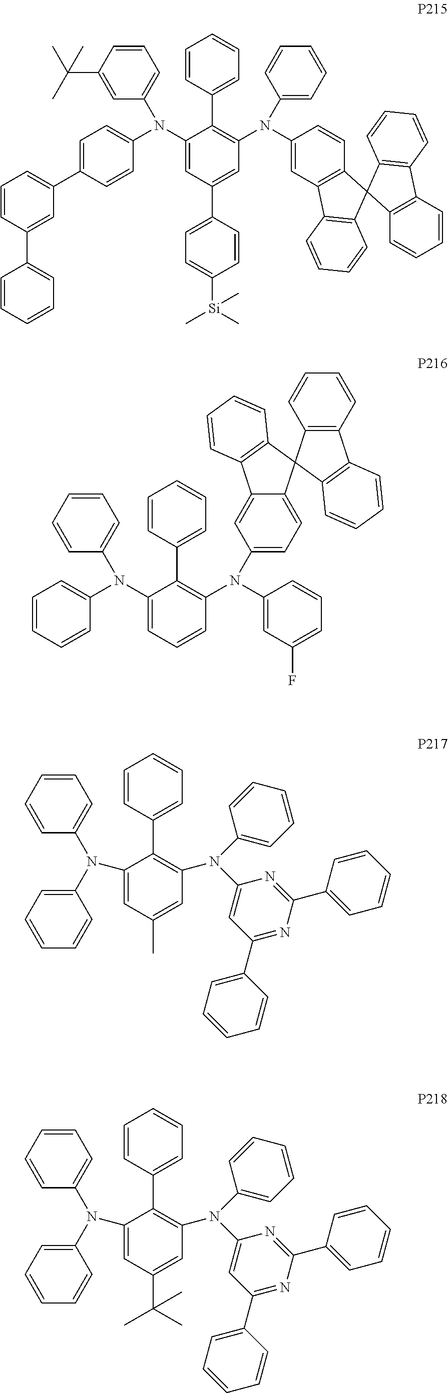 [SMILES: CC(C)(C)c1cccc(N(c2ccc(-c3cccc(-c4ccccc4)c3)cc2)c2cc(-c3ccc([Si](C)(C)C)cc3)cc(N(c3ccccc3)c3ccc4c(c3)-c3ccccc3C43c4ccccc4-c4ccccc43)c2-c2ccccc2)c1 ; Fc1cccc(N(c2ccc3c(c2)-c2ccccc2C32c3ccccc3-c3ccccc32)c2cccc(N(c3ccccc3)c3ccccc3)c2-c2ccccc2)c1 ; Cc1cc(N(c2ccccc2)c2ccccc2)c(-c2ccccc2)c(N(c2ccccc2)c2cc(-c3ccccc3)nc(-c3ccccc3)n2)c1 ; CC(C)(C)c1cc(N(c2ccccc2)c2ccccc2)c(-c2ccccc2)c(N(c2ccccc2)c2cc(-c3ccccc3)nc(-c3ccccc3)n2)c1]

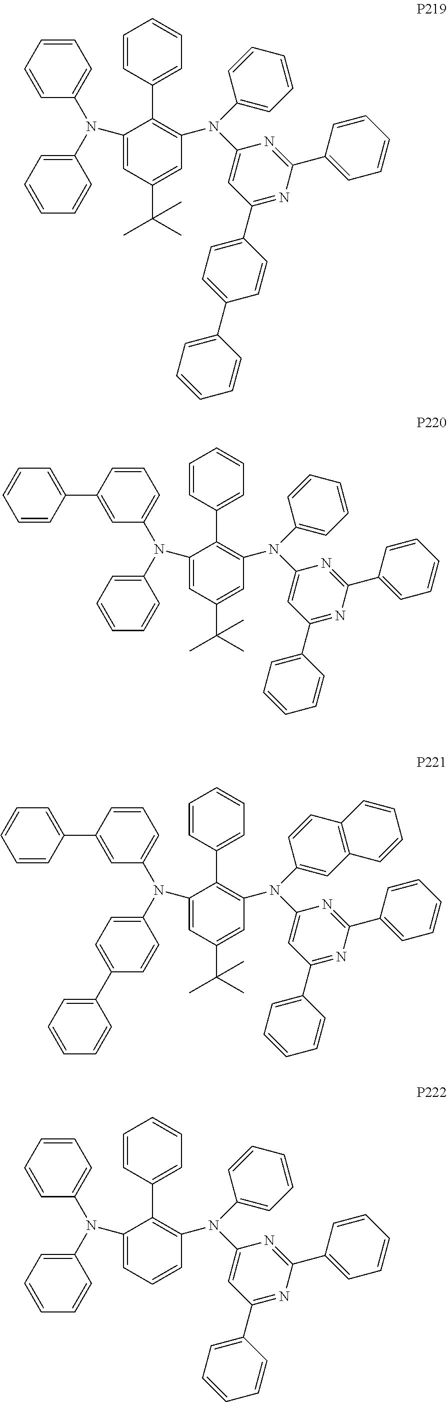 [SMILES: CC(C)(C)c1cc(N(c2ccccc2)c2ccccc2)c(-c2ccccc2)c(N(c2ccccc2)c2cc(-c3ccc(-c4ccccc4)cc3)nc(-c3ccccc3)n2)c1 ; CC(C)(C)c1cc(N(c2ccccc2)c2cccc(-c3ccccc3)c2)c(-c2ccccc2)c(N(c2ccccc2)c2cc(-c3ccccc3)nc(-c3ccccc3)n2)c1 ; CC(C)(C)c1cc(N(c2ccc(-c3ccccc3)cc2)c2cccc(-c3ccccc3)c2)c(-c2ccccc2)c(N(c2ccc3ccccc3c2)c2cc(-c3ccccc3)nc(-c3ccccc3)n2)c1 ; c1ccc(-c2cc(N(c3ccccc3)c3cccc(N(c4ccccc4)c4ccccc4)c3-c3ccccc3)nc(-c3ccccc3)n2)cc1]

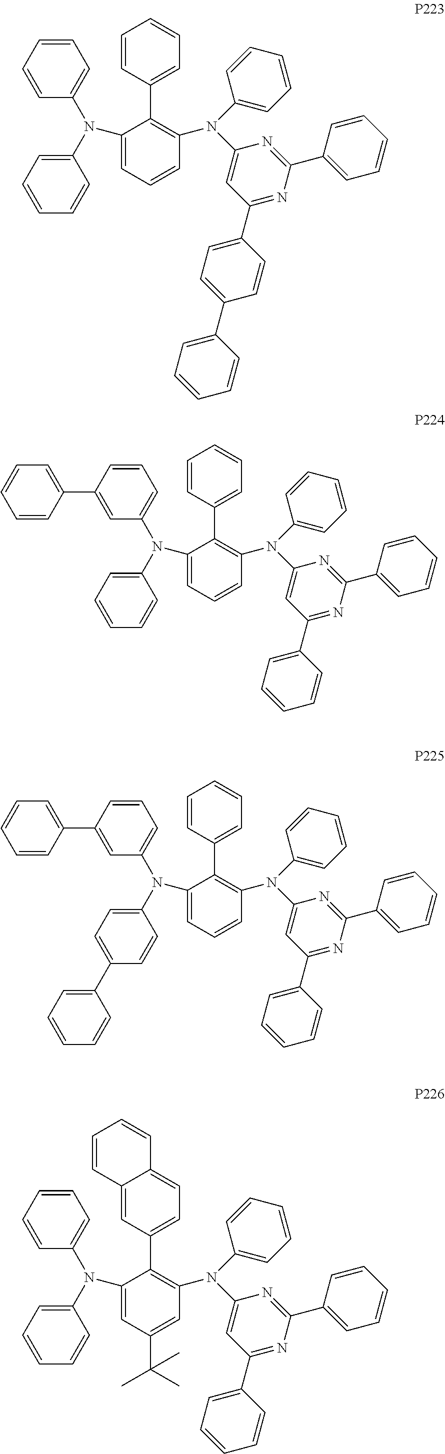 [SMILES: c1ccc(-c2ccc(-c3cc(N(c4ccccc4)c4cccc(N(c5ccccc5)c5ccccc5)c4-c4ccccc4)nc(-c4ccccc4)n3)cc2)cc1 ; c1ccc(-c2cccc(N(c3ccccc3)c3cccc(N(c4ccccc4)c4cc(-c5ccccc5)nc(-c5ccccc5)n4)c3-c3ccccc3)c2)cc1 ; c1ccc(-c2ccc(N(c3cccc(-c4ccccc4)c3)c3cccc(N(c4ccccc4)c4cc(-c5ccccc5)nc(-c5ccccc5)n4)c3-c3ccccc3)cc2)cc1 ; CC(C)(C)c1cc(N(c2ccccc2)c2ccccc2)c(-c2ccc3ccccc3c2)c(N(c2ccccc2)c2cc(-c3ccccc3)nc(-c3ccccc3)n2)c1]

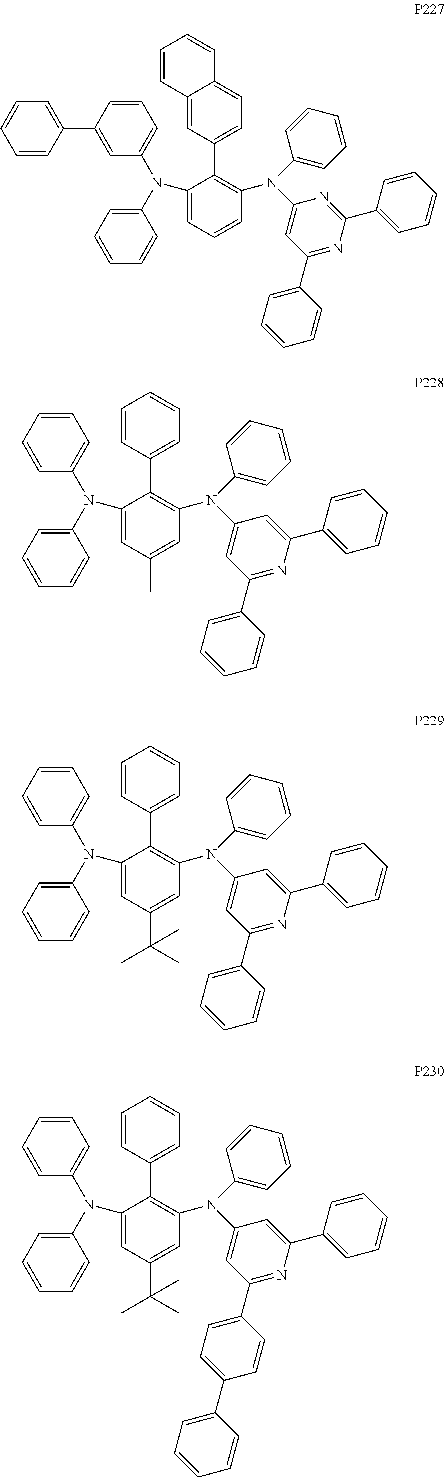 [SMILES: c1ccc(-c2cccc(N(c3ccccc3)c3cccc(N(c4ccccc4)c4cc(-c5ccccc5)nc(-c5ccccc5)n4)c3-c3ccc4ccccc4c3)c2)cc1 ; Cc1cc(N(c2ccccc2)c2ccccc2)c(-c2ccccc2)c(N(c2ccccc2)c2cc(-c3ccccc3)nc(-c3ccccc3)c2)c1 ; CC(C)(C)c1cc(N(c2ccccc2)c2ccccc2)c(-c2ccccc2)c(N(c2ccccc2)c2cc(-c3ccccc3)nc(-c3ccccc3)c2)c1 ; CC(C)(C)c1cc(N(c2ccccc2)c2ccccc2)c(-c2ccccc2)c(N(c2ccccc2)c2cc(-c3ccccc3)nc(-c3ccc(-c4ccccc4)cc3)c2)c1]

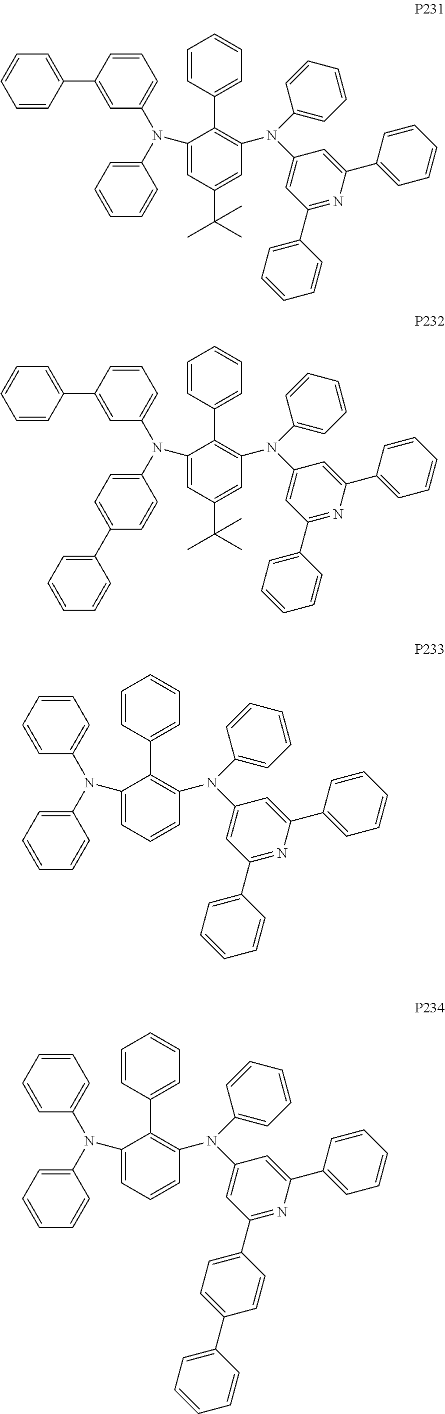 [SMILES: CC(C)(C)c1cc(N(c2ccccc2)c2cccc(-c3ccccc3)c2)c(-c2ccccc2)c(N(c2ccccc2)c2cc(-c3ccccc3)nc(-c3ccccc3)c2)c1 ; CC(C)(C)c1cc(N(c2ccc(-c3ccccc3)cc2)c2cccc(-c3ccccc3)c2)c(-c2ccccc2)c(N(c2ccccc2)c2cc(-c3ccccc3)nc(-c3ccccc3)c2)c1 ; c1ccc(-c2cc(N(c3ccccc3)c3cccc(N(c4ccccc4)c4ccccc4)c3-c3ccccc3)cc(-c3ccccc3)n2)cc1 ; c1ccc(-c2ccc(-c3cc(N(c4ccccc4)c4cccc(N(c5ccccc5)c5ccccc5)c4-c4ccccc4)cc(-c4ccccc4)n3)cc2)cc1]

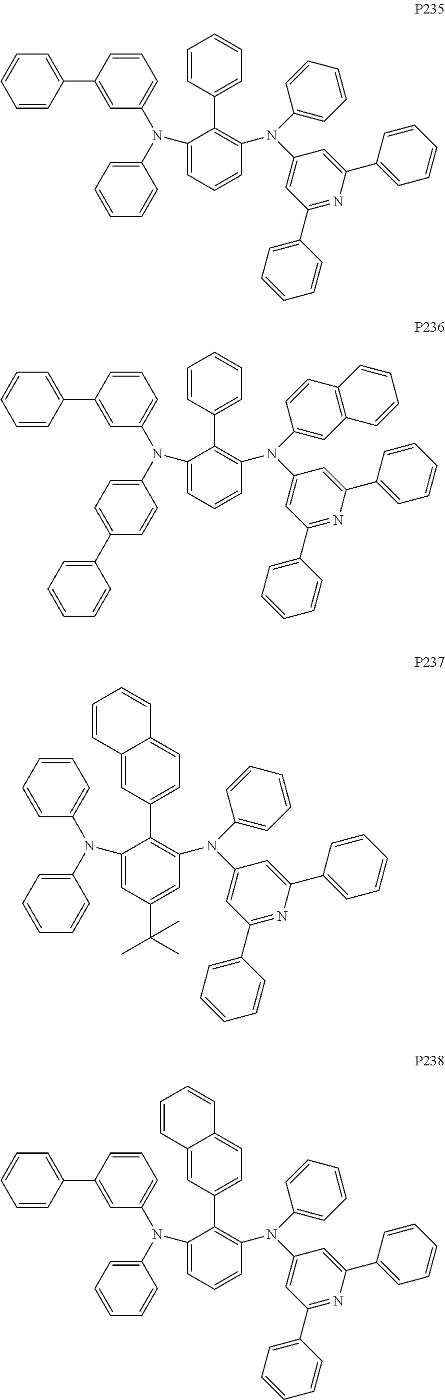 [SMILES: c1ccc(-c2cccc(N(c3ccccc3)c3cccc(N(c4ccccc4)c4cc(-c5ccccc5)nc(-c5ccccc5)c4)c3-c3ccccc3)c2)cc1 ; c1ccc(-c2ccc(N(c3cccc(-c4ccccc4)c3)c3cccc(N(c4cc(-c5ccccc5)nc(-c5ccccc5)c4)c4ccc5ccccc5c4)c3-c3ccccc3)cc2)cc1 ; CC(C)(C)c1cc(N(c2ccccc2)c2ccccc2)c(-c2ccc3ccccc3c2)c(N(c2ccccc2)c2cc(-c3ccccc3)nc(-c3ccccc3)c2)c1 ; c1ccc(-c2cccc(N(c3ccccc3)c3cccc(N(c4ccccc4)c4cc(-c5ccccc5)nc(-c5ccccc5)c4)c3-c3ccc4ccccc4c3)c2)cc1]

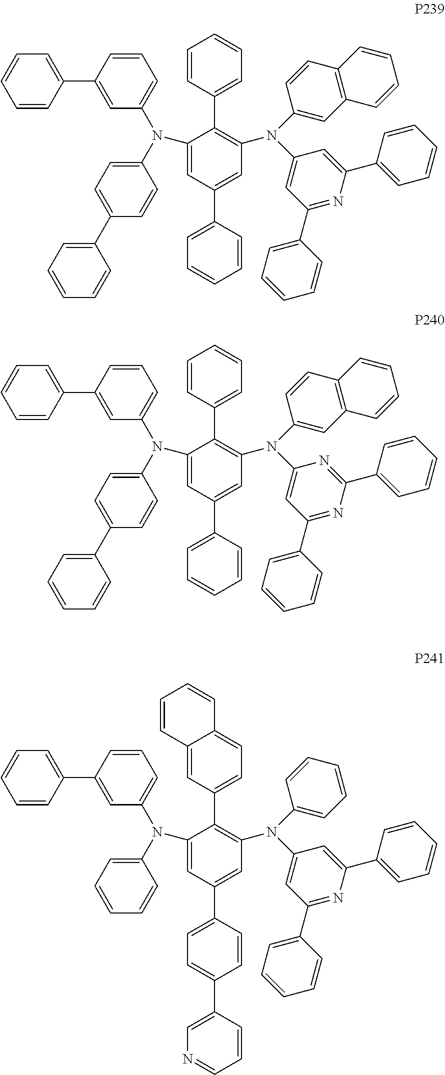 [SMILES: c1ccc(-c2ccc(N(c3cccc(-c4ccccc4)c3)c3cc(-c4ccccc4)cc(N(c4cc(-c5ccccc5)nc(-c5ccccc5)c4)c4ccc5ccccc5c4)c3-c3ccccc3)cc2)cc1 ; c1ccc(-c2ccc(N(c3cccc(-c4ccccc4)c3)c3cc(-c4ccccc4)cc(N(c4ccc5ccccc5c4)c4cc(-c5ccccc5)nc(-c5ccccc5)n4)c3-c3ccccc3)cc2)cc1 ; c1ccc(-c2cccc(N(c3ccccc3)c3cc(-c4ccc(-c5cccnc5)cc4)cc(N(c4ccccc4)c4cc(-c5ccccc5)nc(-c5ccccc5)c4)c3-c3ccc4ccccc4c3)c2)cc1]

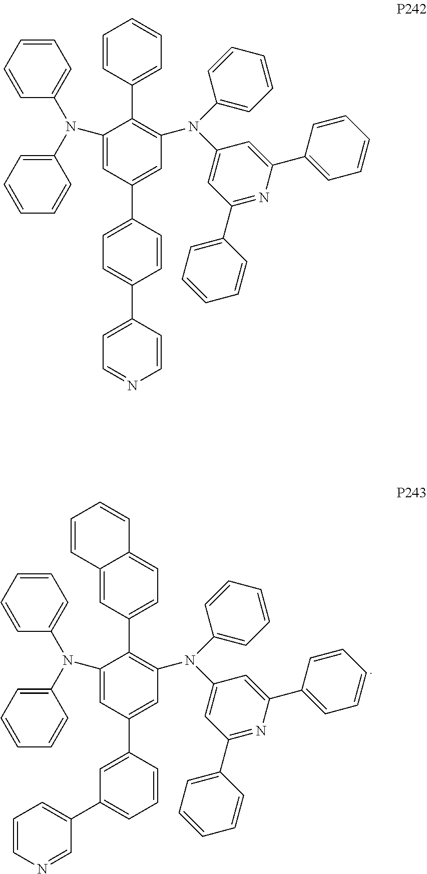 [SMILES: c1ccc(-c2cc(N(c3ccccc3)c3cc(-c4ccc(-c5ccncc5)cc4)cc(N(c4ccccc4)c4ccccc4)c3-c3ccccc3)cc(-c3ccccc3)n2)cc1 ; c1ccc(-c2cc(N(c3ccccc3)c3cc(-c4cccc(-c5cccnc5)c4)cc(N(c4ccccc4)c4ccccc4)c3-c3ccc4ccccc4c3)cc(-c3ccccc3)n2)cc1]

A second aspect of the present disclosure provides a method for preparing the arylamine compound of the first aspect of the present disclosure, and the method includes: under the coupling reaction conditions, in the presence of fifth palladium catalyst and base catalyst, allowing compound shown in Formula (2) to be in contact with compound shown in Formula (3) for a Suzuki reaction to obtain compound shown in the Formula (1):

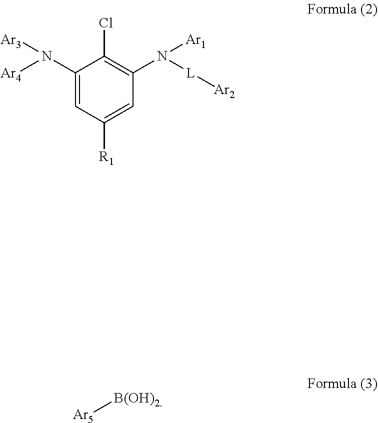

According to the method provided by the present disclosure, the usage amount of the compound shown in Formula (2) and the usage amount of the compound shown in Formula (3) may change in a large range, and optionally, a molar ratio of the compound shown in Formula (2) to the compound shown in Formula (3) may be 1: (1 to 1.05). The coupling reaction conditions can be conventional Suzuki coupling reaction conditions in the field, and in one embodiment, the conditions for the Suzuki reaction may be as follows: the reaction temperature is 65° C. to 70° C., the reaction time is 8 h to 12 h, and the reaction solvent is a mixture of toluene, ethanol and water; the fifth palladium catalyst is palladium acetate and/or palladium chloride, and the base catalyst is at least one of potassium carbonate, sodium carbonate, and sodium bicarbonate.

Optionally, a preparation method of the compound shown in Formula (2) can include: under fourth amination reaction condition, in the presence of fourth palladium catalyst and fourth phosphine ligand catalyst, allowing compound shown in Formula (4) to be in contact with aryl haloalkane (or heteroaryl haloalkane) Ar₄—X′₄ for an Ullmann reaction;

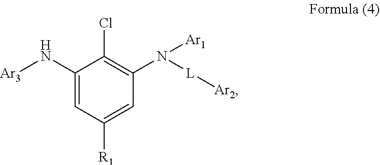

wherein X′₄ can be Br or I.

Optionally, a molar ratio of the compound shown in the Formula (4) to Ar₄—X′₄ can be 1: (1 to 1.1), the reaction temperature can be 100° C. to 110° C., the reaction time is 3 h to 5 h, the reaction solvent can be toluene, the fourth palladium catalyst can be palladium acetate and/or palladium chloride, and the fourth phosphine ligand catalyst can be tricyclohexylphosphine and/or 2-dicyclohexylphosphine-2,4,6-triisopropylbiphenyl.

Optionally, a preparation method of the compound shown in the Formula (4) includes: in the presence of second palladium catalyst and second phosphine ligand catalyst, allowing compound shown in Formula (5) to be in contact with aryl haloalkane (or heteroaryl haloalkane) Ar₂-L-X′₂ for an Ullmann reaction,

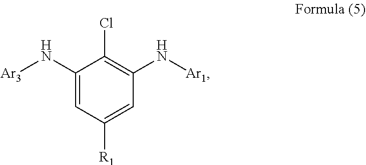

wherein X′₂ is Br or I.

Optionally, a molar ratio of the compound shown in the Formula (5) to Ar₂-L-X′₂ can be 1: (1 to 1.1), the reaction temperature can be 100° C. to 110° C., the reaction time is 3 h to 5 h, the reaction solvent can be toluene, the second palladium catalyst can be palladium acetate and/or palladium chloride, and the second phosphine ligand catalyst can be tricyclohexylphosphine and/or 2-dicyclohexylphosphine-2,4,6-triisopropylbiphenyl.

Optionally, in an embodiment in which Ar₁ and Ar₃ are different, the compound shown in Formula (5) may be prepared by a two-step amination reaction, respectively, and in this embodiment, a preparation method of the compound shown in Formula (5) may include allowing compound shown in Formula (6) to be in contact with Ar₃—NH₂ in the presence of third palladium catalyst and third phosphine ligand catalyst:

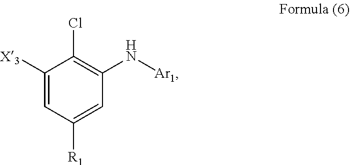

wherein X′₃ is I or Br.

Optionally, a molar ratio of the compound shown in the Formula (6) to the arylamine (or aromatic heteroarylamine) Ar₃—NH₂ can be 1: (1 to 1.1); the reaction temperature can be 100° C. to 110° C., the reaction time can be 3 h to 5 h, and the reaction solvent can be toluene; the third palladium catalyst can be palladium acetate and/or palladium chloride, and the third phosphine ligand catalyst can be tricyclohexylphosphine and/or 2-dicyclohexylphosphine-2,4,6-triisopropylbiphenyl.

Further, a preparation method of the compound shown in Formula (6) can include allowing compound shown in Formula (8) to be in contact with Ar₁—NH₂ in the presence of first palladium catalyst and first phosphine ligand catalyst:

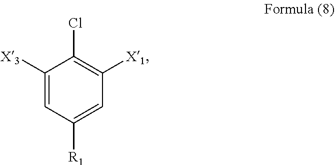

wherein X′₁ is I or Br.

Optionally, a molar ratio of the compound shown in Formula (8) to Ar₁—NH₂ can be 1: (1 to 1.1); the reaction temperature can be 100° C. to 110° C., the reaction time can be 3 h to 5 h, and the reaction solvent can be toluene; the first palladium catalyst can be palladium acetate and/or palladium chloride, and the first phosphine ligand catalyst can be tricyclohexylphosphine and/or 2-dicyclohexylphosphine-2,4,6-triisopropylbiphenyl.

In an embodiment in which Ar₁ and Ar₃ are the same, the compound shown in the Formula (5) may be prepared by a one-step amination reaction, and in this embodiment, a preparation method of the compound shown in Formula (5) may include allowing compound shown in Formula (7) to be in contact with Ar₃—NH₂ in the presence of third palladium catalyst and third phosphine ligand catalyst:

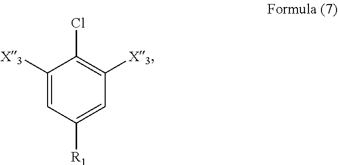

in the Formula (7), X″₃ is I or Br.

Optionally, a molar ratio of the compound shown in Formula (7) to Ar₃—NH₂ can be 1: (2.1 to 2.3); the reaction temperature can be 100° C. to 110° C., the reaction time can be 3 h to 5 h, and the reaction solvent can be toluene; the third palladium catalyst can be palladium acetate and/or palladium chloride, and the third phosphine ligand catalyst can be tricyclohexylphosphine and/or 2-dicyclohexylphosphine-2,4,6-triisopropylbiphenyl.

A third aspect of the present disclosure provides an use of the arylamine compound of the first aspect of the present disclosure in preparation of an organic electroluminescent device.

According to the present disclosure, the arylamine compound has good electron transport performance and suitable energy level, and can be used as one, two or three of an organic light-emitting layer material, an electron transport layer material and a hole transport layer material of the organic electroluminescent device. When being used as the organic light-emitting layer material, the compound of the present disclosure is particularly used as a host material of an organic electroluminescent layer.

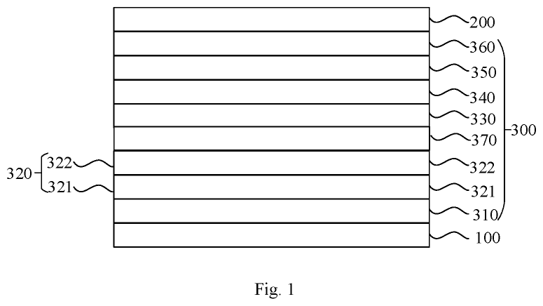

As shown in FIG. 1, a fourth aspect of the present disclosure provides an organic electroluminescent device, including an anode 100, a cathode 200 and at least one functional layer 300 between the anode and the cathode, and the functional layer 300 includes a hole injection layer 310, a hole transport layer 320, an organic electroluminescent layer 330, an electron transport layer 350 and an electron injection layer 360. The hole injection layer 310, the hole transport layer 320, the organic electroluminescent layer 330, the electron transport layer 350 and the electron injection layer 360 can be sequentially formed on the anode 100. In one embodiment, the organic electroluminescent layer 330 may contain the arylamine compound described in the first aspect of the present disclosure, preferably at least one of compounds P1 to P243. In another embodiment, the electron transport layer 350 may contain the arylamine compound described in the first aspect of the present disclosure, preferably at least one of compounds P1 to P243. In another embodiment, the hole transport layer 320 may contain the arylamine compound described in the first aspect of the present disclosure, preferably at least one of compounds P1 to P243; further, the hole transport layer 320 can include a first hole transport layer 321 and a second hole transport layer 322, the first hole transport layer 321 is closer to the anode than the second hole transport layer 322, and the second hole transport layer 322 contains the arylamine compound in the first aspect of the present disclosure.

In a further embodiment, the functional layer 300 of the organic electroluminescent device can further include a hole-blocking layer 340 and an electron-blocking layer 370, the hole-blocking layer 340 can be arranged between the organic electroluminescent layer 330 and the electron transport layer 350, and the electron-blocking layer 370 can be arranged between the hole transport layer 320 and the organic electroluminescent layer 330.

According to the organic electroluminescent device, based on the excellent performance of the compound provided by the present disclosure the device obtained by adopting the compound as an organic electroluminescent layer material can reduce the driving voltage of the organic electroluminescent device, improve the luminous efficiency and prolong the lifetime of the device; the compound is used as a hole transport material, which has better luminous efficiency, better electrical stability and better hole transport performance, and can significantly improve the performance of the organic electroluminescent device when being used in the hole transport layer of the organic electroluminescent device.

The compounds in the synthesis method which are not mentioned in the present disclosure are all commercially available raw material products.

An ICP-7700 mass spectrometer and an M5000 elemental analyzer are used for analysis and detection of intermediates and compounds in the present disclosure.

The synthesis method of the arylamine compound disclosed by the present disclosure is described in detail below in combination with Synthesis Examples 1 to 10.

### Synthesis Example 1 (Compound P4)

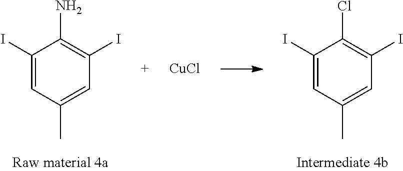

(1) 15 mL of concentrated sulfuric acid was added into a three-mouthed reaction bottle provided with a mechanical stirrer, a thermometer and a constant-pressure dropping funnel, stirring was started, 5 g of sodium nitrite was added in batches, and the temperature of a system was controlled to be 70° C., where the system was light green after feeding. Raw material 4a (50 mmol, 17.95 g) was dissolved in 40 mL of glacial acetic acid, the obtained solution was slowly dropwise added into the concentrated sulfuric acid solution of sodium nitrite while keeping the temperature at 40° C. to 50° C., where heat was slightly released in the system, and the temperature was kept for 2 h after dropwise adding to obtain a A4 solution. Cuprous chloride (100 mmol, 9.90 g) was dissolved in 30 mL of concentrated hydrochloric acid, the obtained solution was cooled to −10° C., the A4 solution was started to be slowly added dropwise, where the temperature was controlled to be −10° C., and dropwise adding was performed in batches at an interval of 30 min for about 4 h, the resulting solution was naturally heated, the system was kept unblocked, and stirring was conducted overnight. After 12 h, the system was heated to 70° C. to 80° C., and a sample was tested every 2 h, where the reaction was complete in 14 h to 20 h. The reaction solution was slowly added into 100 mL of water while stirring, 100 mL of dichloromethane was added, stirring was conducted for 10 min, the stirred material was allowed to stand for 5 min, liquid separation was conducted, water phase was extracted once by using 100 mL of dichloromethane, liquid separation was conducted, organic phase was combined, and water was added for washing to be neutral. 8 g of anhydrous sodium sulfate was added into the obtained organic phase, stirring and drying were performed for 5 min, filtering was performed, a filter cake was drip washed with 13 mL of dichloromethane, and the organic phase was concentrated (−0.06 MPa to 0.075 MPa and 35° C. to 40° C.) until no solvent flowed out to obtain 45 mmol of Intermediate 4b with a yield of 90%.

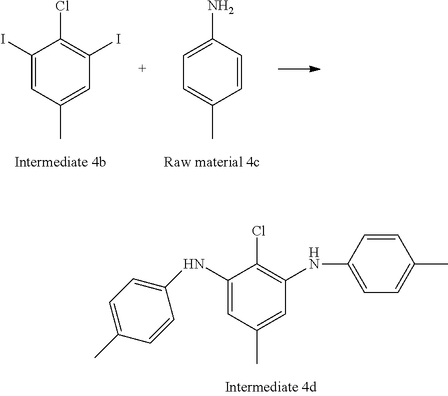

(2) Nitrogen was introduced into a three-mouthed reaction bottle provided with a mechanical stirrer, a thermometer and a constant-pressure dropping funnel for 10 min (2000 mL/min), Intermediate 4b (40 mmol, 15.13 g), p-methylaniline (84 mmol, 9.00 g) and 130 mL of toluene was sequentially added, heating, refluxing and water separation were performed for 0.5 h, cooling was performed to 70° C. to 80° C., 120 mmol of sodium tert-butoxide was slowly added, heating was performed to 100° C. to 110° C. for refluxing, water was separated for 0.5 h while refluxing, cooling was performed to 50° C. to 60° C., and palladium acetate (0.4 mmol) and tricyclohexylphosphine (0.8 mmol) were slowly added in batches, where attention was paid to observe the temperature of the system, and a violent reaction will generate. After the system was stable, heating was performed for refluxing (T=100° C. to 110° C.), a reaction was carried out for 3 h while keeping the temperature, a sample was submitted for inspection, and the reaction was stopped when the reaction was complete. Cooling was performed to 25° C. to 30° C., 130 mL of water and 130 mL of toluene were added, stirring and liquid separation were performed, water phase was extracted once with 130 mL of toluene, liquid was separated, organic phase was combined, 0.7 g of anhydrous sodium sulfate was added into the organic phase, stirring, drying and filtering were performed, the organic phase was concentrated (−0.06 MPa to 0.075 MPa and 55° C. to 60° C.) until no solvent flowed out to obtain a crude product, 20 mL of ethanol was added, stirring was conducted at 25° C. to separate out a large amount of solids, and filtering was performed. A filter cake was drip washed with ethanol to obtain 36 mmol of Intermediate 4d with a yield of 90%.

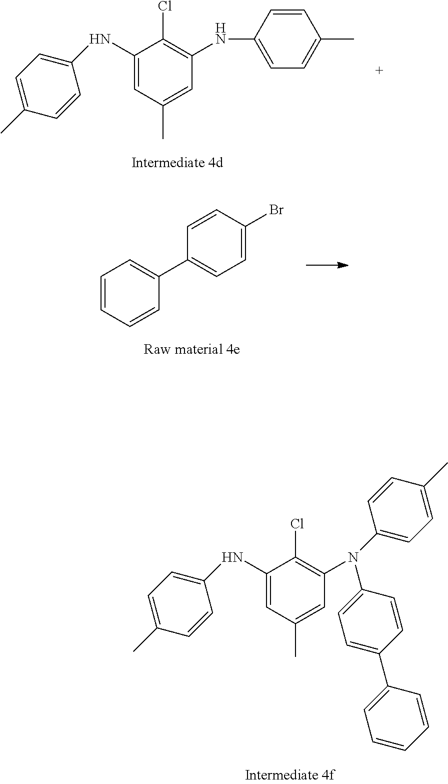

(3) Nitrogen was introduced into a three-mouthed reaction bottle provided with a mechanical stirrer, a thermometer and a constant-pressure dropping funnel for 10 min (2000 mL/min), Intermediate 4d (35 mmol, 11.79 g), Raw material 4e (38 mmol, 8.80 g) and 130 mL of toluene were sequentially added, heating, refluxing and water separation were performed for 0.5 h, cooling was performed to 70° C. to 80° C., 100 mmol of sodium tert-butoxide was slowly added, heating was performed to 100° C. to 110° C. for refluxing, water was separated for 0.5 h while refluxing, cooling was performed to 50° C. to 60° C., and palladium acetate (0.4 mmol) and tricyclohexylphosphine (0.8 mmol) were slowly added in batches, where attention was paid to observe the temperature of the system, and a violent reaction will generate. After the system was stable, heating was performed for refluxing (T=100° C. to 110° C.), a reaction was carried out for 3 h while keeping the temperature, a sample was submitted for inspection, and the reaction was stopped when the reaction was complete. Cooling was performed to 25° C. to 30° C., 130 mL of water and 130 mL of toluene were added, stirring and liquid separation were performed, water phase was extracted once with 130 mL of toluene, liquid was separated, organic phase was combined, 0.7 g of anhydrous sodium sulfate was added into the organic phase, stirring, drying and filtering were performed, the organic phase was concentrated (−0.06 MPa to 0.075 MPa and 55° C. to 60° C.) until no solvent flowed out to obtain a crude product, 20 mL of ethanol was added, stirring was conducted at 25° C. to separate out a large amount of solids, and filtering was performed. A filter cake was drip washed with ethanol to obtain 20 mmol of Intermediate 4f with a yield of 57%.

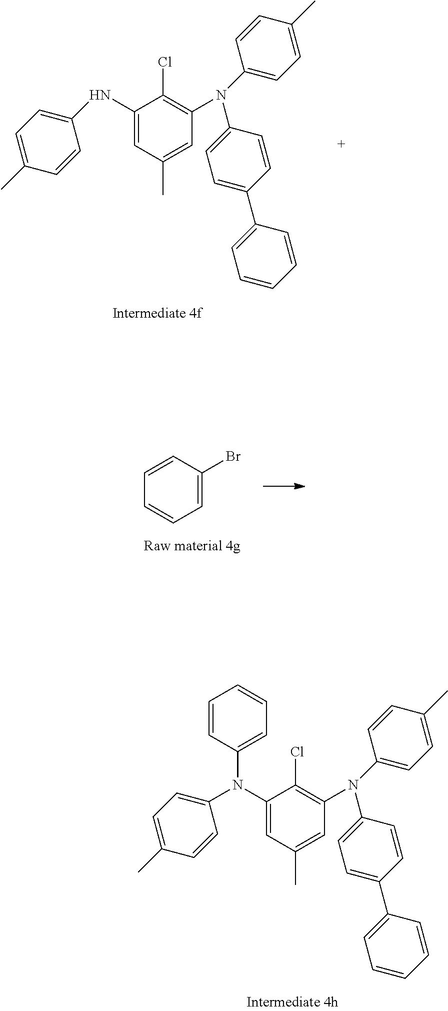

(4) Nitrogen was introduced into a three-mouthed reaction bottle provided with a mechanical stirrer, a thermometer and a constant-pressure dropping funnel for 10 min (2000 mL/min), Intermediate 4f (30 mmol, 14.67 g), Raw material 4g (33 mmol, 5.18 g) and 130 mL of toluene were sequentially added, heating, refluxing and water separation were performed for 0.5 h, cooling was performed to 70° C. to 80° C., 100 mmol of sodium tert-butoxide was slowly added, heating was performed to 100° C. to 110° C. for refluxing, water was separated for 0.5 h while refluxing, cooling was performed to 50° C. to 60° C., and palladium acetate (0.4 mmol) and tricyclohexylphosphine (0.8 mmol) were slowly added in batches, where attention was paid to observe the temperature of the system, and a violent reaction will generate. After the system was stable, heating was performed for refluxing (T=100° C. to 110° C.), a reaction was carried out for 3 h while keeping the temperature, a sample was submitted for inspection, and the reaction was stopped when the reaction was complete. Cooling was performed to 25° C. to 30° C., 130 mL of water and 130 mL of toluene were added, stirring and liquid separation were performed, water phase was extracted once with 130 mL of toluene, liquid was separated, organic phase was combined, 0.7 g of anhydrous sodium sulfate was added into the organic phase, stirring, drying and filtering were performed, the organic phase was concentrated (−0.06 MPa to 0.075 MPa and 55° C. to 60° C.) until no solvent flowed out to obtain a crude product, 20 mL of ethanol was added, stirring was performed at 25° C. to separate out a large amount of solids, and filtering was performed. A filter cake was drip washed with ethanol to obtain 27 mmol of Intermediate 4h with a yield of 90%.

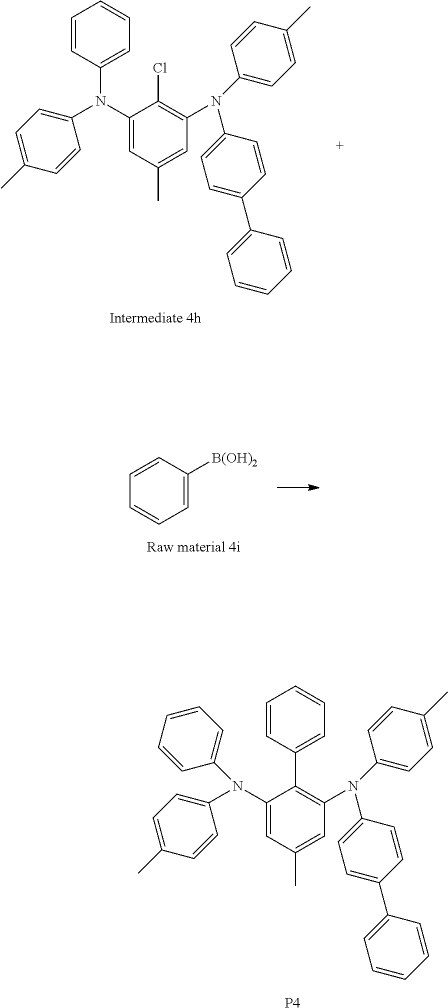

(5) 72 mL of toluene, 54 mL of ethanol and 20 mL of water were added into a three-necked flask provided with a mechanical stirrer, a thermometer and a Y-shaped tube, stirring was started, Intermediate 4h (15 mmol, 8.48 g), Raw material 4i (15.75 mmol, 1.92 g), potassium carbonate (30 mmol, 4.15 g) and tetrabutylammonium bromide (1.5 mmol, 0.48 g) were added, heating was performed to 45° C. to 50° C., palladium acetate (0.075 mmol, 0.017 g) was quickly added, heating was continuously conducted to 65° C. to 70° C., and a reaction was carried out for 8 h while heat preservation. The reaction solution was cooled to 15° C. to 20° C., filtering and draining were conducted, a filter cake was drip washed with ethanol, and draining was performed. Liquid separation was performed on the obtained filtrate, water phase was extracted with toluene, and an organic phase was washed with water for three times. An organic layer was dried by using anhydrous magnesium sulfate, filtering was conducted, the obtained filtrate was allowed to pass through a short silica gel column after filtering, solvent was removed under reduced pressure, and a crude product was recrystallized by using a dichloromethane system for purifying to obtain 6.8 g of the Compound P4 with a yield of 75%. m/z=607.80 [M+H]⁺.

¹H-NMR (CDCl₃, 300 MHz): δ(ppm)=8.12-8.08 (d, 2H), 8.04-7.98 (m, 5H), 7.93-7.88 (m, 4H), 7.86-7.82 (m, 3H), 7.77-7.73 (m, 2H), 7.64-7.60 (m, 6H), 7.58-7.53 (m, 4H), 7.47-7.43 (s, 2H), 7.26-7.22 (m, 1H), 3.26-3.23 (s, 3H), 3.18-3.15 (s, 3H), 2.96-2.92 (s, 3H).

### Synthesis Example 2 (Compound P10)

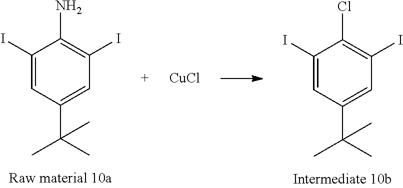

(1) 15 mL of concentrated sulfuric acid was added into a three-mouthed reaction bottle provided with a mechanical stirrer, a thermometer and a constant-pressure dropping funnel, stirring was started, 5 g of sodium nitrite was added in batches, and the temperature of a system was controlled to be 60° C. to 80° C., where the system was light green after feeding. Raw material 10a (50 mmol, 20.00 g) was dissolved in 50 mL of glacial acetic acid, the obtained solution was slowly dropwise added into the concentrated sulfuric acid solution of sodium nitrite while keeping the temperature at 40° C. to 50° C., where heat was slightly released in the system, and the temperature was kept for 2 h after dropwise adding to obtain a A10 solution. Cuprous chloride (100 mmol, 9.79 g) was dissolved in 30 mL of concentrated hydrochloric acid, the obtained solution was cooled to −10° C., the A10 solution was started to be slowly added dropwise, where the temperature was controlled to be −10° C., and dropwise adding was performed in batches at an interval of 30 min for about 4 h, the resulting solution was naturally heated, the system was kept unblocked, and stirring was conducted overnight. After 12 h, the system was heated to 70° C. to 80° C., a sample was tested every other 2 h, where the reaction was complete in 14 h to 20 h. The reaction solution was slowly added into 100 mL of water while stirring, 100 mL of dichloromethane was added, stirring was conducted for 10 min, the stirred material was allowed to stand for 5 min, liquid separation was conducted, water phase was extracted once by using 100 mL of dichloromethane, liquid separation was conducted, organic phase was combined, and water was added for washing to be neutral. 8 g of anhydrous sodium sulfate was added into the obtained organic phase, stirring and drying were performed for 5 min, filtering was performed, a filter cake was drip washed with 13 mL of dichloromethane, and the organic phase was concentrated (−0.06 MPa to 0.075 MPa and 35° C. to 40° C.) until no solvent flowed out to obtain 42 mmol of Intermediate 10b with a yield of 84%.

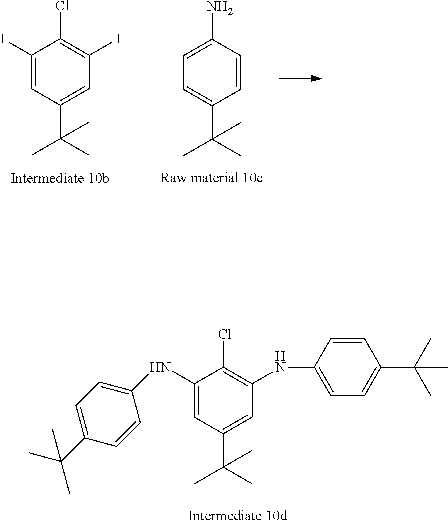 [SMILES: CC(C)(C)c1cc(I)c(Cl)c(I)c1 ; CC(C)(C)c1ccc(N)cc1 ; CC(C)(C)c1ccc(Nc2cc(C(C)(C)C)cc(Nc3ccc(C(C)(C)C)cc3)c2Cl)cc1]

(2) Nitrogen was introduced into a three-mouthed reaction bottle provided with a mechanical stirrer, a thermometer and a constant-pressure dropping funnel for 10 min (2000 mL/min), Intermediate 10b (40 mmol, 16.82 g), p-tert-butylaniline (84 mmol, 12.53 g) and 130 mL of toluene were sequentially added, heating, refluxing and water separation were performed for 0.5 h, cooling was performed to 70° C. to 80° C., 120 mmol of sodium tert-butoxide was slowly added, heating was performed to 100° C. to 110° C. for refluxing, water was separated for 0.5 h while refluxing, cooling was performed to 50° C. to 60° C., and palladium acetate (0.4 mmol) and tricyclohexylphosphine (0.8 mmol) were slowly added in batches, where attention was paid to observe the temperature of the system, and a violent reaction will generate. After the system was stable, heating was performed for refluxing (T=100° C. to 110° C.), a reaction was carried out for 3 h while keeping the temperature, a sample was submitted for inspection, and the reaction was stopped when the reaction was complete. Cooling was performed to 25° C. to 30° C., 130 mL of water and 130 mL of toluene were added, stirring and liquid separation were performed, water phase was extracted once with 130 mL of toluene, liquid was separated, organic phase was combined, 0.7 g of anhydrous sodium sulfate was added into the organic phase, stirring, drying and filtering were performed, the organic phase was concentrated (−0.06 MPa to 0.075 MPa and 55° C. to 60° C.) until no solvent flowed out to obtain a crude product, 20 mL of ethanol was added, stirring was performed at 25° C. to separate out a large amount of solids, and filtering was performed. A filter cake was drip washed with ethanol to obtain 21 mmol of Intermediate 10d with a yield 52.5%.

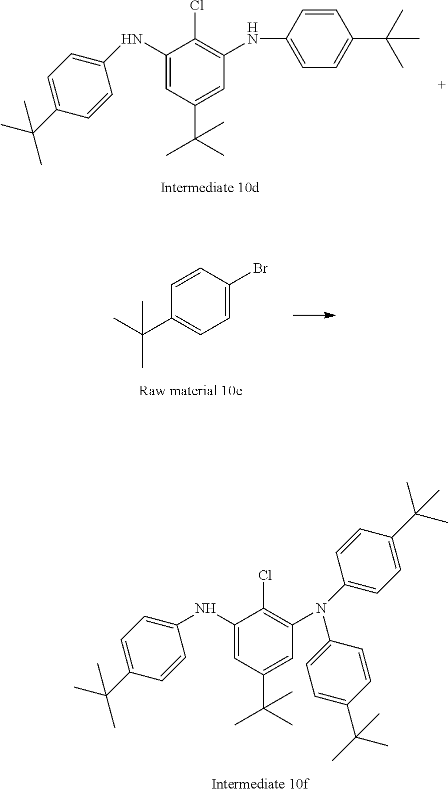

(3) Nitrogen was introduced into a three-mouthed reaction bottle provided with a mechanical stirrer, a thermometer and a constant-pressure dropping funnel for 10 min (2000 mL/min), Intermediate 10d (30 mmol, 13.44 g), Raw material 10e (33 mmol, 7.03 g) and 130 mL of toluene were sequentially added, heating, refluxing and water separation were performed for 0.5 h, cooling was performed to 70° C. to 80° C., 100 mmol of sodium tert-butoxide was slowly added, heating was performed to 100° C. to 110° C. for refluxing, water was separated for 0.5 h while refluxing, cooling was performed to 50° C. to 60° C., and palladium acetate (0.4 mmol) and tricyclohexylphosphine (0.8 mmol) were slowly added in batches, where attention was paid to observe the temperature of the system, and a violent reaction will generate. After the system was stable, heating was performed for refluxing (T=100° C. to 110° C.), a reaction was carried out for 3 h while keeping the temperature, a sample was submitted for inspection, and the reaction was stopped when the reaction was complete. Cooling was performed to 25° C. to 30° C., 130 mL of water and 130 mL of toluene were added, stirring and liquid separation were performed, water phase was extracted once with 130 mL of toluene, liquid was separated, organic phase was combined, 0.7 g of anhydrous sodium sulfate was added into the organic phase, stirring, drying and filtering were performed, the organic phase was concentrated (−0.06 MPa to 0.075 MPa and 55° C. to 60° C.) until no solvent flowed out to obtain a crude product, 20 mL of ethanol was added, stirring was conducted at 25° C. to separate out a large amount of solids, and filtering was performed. A filter cake was drip washed with ethanol to obtain 26.8 mmol of Compound 10f with a yield of 89.3%.

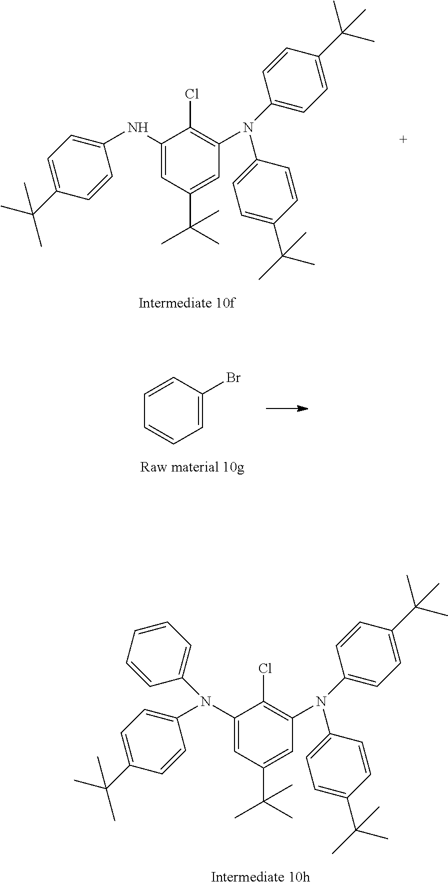

(4) Nitrogen was introduced into a three-mouthed reaction bottle provided with a mechanical stirrer, a thermometer and a constant-pressure dropping funnel for 10 min (2000 mL/min), Intermediate 10f (22 mmol, 13.10 g), Raw material 10g (26.4 mmol, 4.15 g) and 130 mL of toluene were sequentially added, heating, refluxing and water separation were performed for 0.5 h, cooling was performed to 70° C. to 80° C., 85 mmol of sodium tert-butoxide was slowly added, heating was performed to 100° C. to 110° C. for refluxing, water was separated for 0.5 h while refluxing, cooling was performed to 50° C. to 60° C., and palladium acetate (0.38 mmol) and tricyclohexylphosphine (0.65 mmol) were slowly added in batches, where attention was paid to observe the temperature of the system, and a violent reaction will generate. After the system was stable, heating was performed for refluxing (T=100° C. to 110° C.), a reaction was carried out for 3 h while keeping the temperature, a sample was submitted for inspection, and the reaction was stopped when the reaction was complete. Cooling was performed to 25° C. to 30° C., 130 mL of water and 130 mL of toluene were added, stirring and liquid separation were performed, water phase was extracted once with 130 mL of toluene, liquid was separated, organic phase was combined, 0.7 g of anhydrous sodium sulfate was added into the organic phase, stirring, drying and filtering were performed, the organic phase was concentrated (−0.06 MPa to 0.075 MPa and 55° C. to 60° C.) until no solvent flowed out to obtain a crude product, 30 mL of ethanol was added, stirring was conducted at 25° C. to separate out a large amount of solids, and filtering was performed. A filter cake was drip washed with ethanol to obtain 20.2 mmol of Intermediate 10h with a yield of 91.8%.

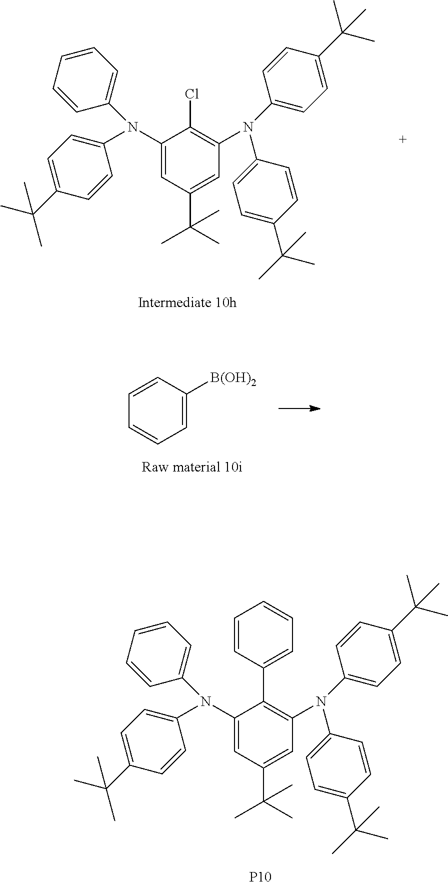

(5) 78 mL of toluene, 58 mL of ethanol and 23 mL of water were added into a three-necked flask provided with a mechanical stirrer, a thermometer and a Y-shaped tube, stirring was started, Intermediate 10h (15 mmol, 10.07 g), Raw material 10i (15.30 mmol, 1.87 g), potassium carbonate (30 mmol, 4.15 g) and tetrabutylammonium bromide (1.5 mmol, 0.48 g) were added, heating was performed to 45° C. to 50° C., palladium acetate (0.075 mmol, 0.017 g) was quickly added, heating was continuously conducted to 65° C. to 70° C., and a reaction was carried out for 8 h while heat preservation. The reaction solution was cooled to 15° C. to 20° C., filtering and draining were conducted, a filter cake was drip washed with ethanol, and draining was performed. Liquid separation was performed on the obtained filtrate, water phase was extracted with toluene, and an organic phase was washed with water for three times. An organic layer was dried by using anhydrous magnesium sulfate, filtering was conducted, the obtained filtrate was allowed to pass through a short silica gel column after filtering, solvent was removed under reduced pressure, and a crude product was recrystallized by using a dichloromethane system for purifying to obtain 6.7 g of Compound P10 with a yield of 63%. m/z=713.08 [M+H]⁺.

¹H-NMR (CDCl₃, 300 MHz): δ(ppm)=8.10-8.06 (d, 2H), 8.02-7.98 (m, 3H), 7.74-7.69 (m, 4H), 7.62-7.58 (m, 8H), 7.52-7.47 (m, 6H), 7.27-7.24 (m, 1H), 3.19-3.15 (s, 18H), 3.01-2.98 (s, 9H), 2.81-2.77 (s, 9H).

### Synthesis Example 3 (Compound P22)

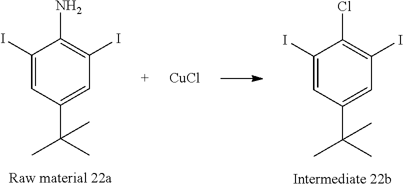

(1) 15 mL of concentrated sulfuric acid was added into a three-mouthed reaction bottle provided with a mechanical stirrer, a thermometer and a constant-pressure dropping funnel, stirring was started, 5 g of sodium nitrite was added in batches, and the temperature of a system was controlled to be 60° C. to 80° C., where the system was light green after feeding. Raw material 22a (50 mmol, 20.05 g) was dissolved in 40 mL of glacial acetic acid, the obtained solution was slowly dropwise added into the concentrated sulfuric acid solution of sodium nitrite while keeping the temperature at 40° C. to 50° C., where heat was slightly released in the system, and the temperature was kept for 2 h after dropwise adding to obtain a A22 solution. Cuprous chloride (100 mmol, 9.79 g) was dissolved in 30 mL of concentrated hydrochloric acid, the obtained solution was cooled to −10° C., the A22 solution was started to be slowly added dropwise, where the temperature was controlled to be −10° C., and dropwise adding was performed in batches at an interval of 30 min for about 4 h, the resulting solution was naturally heated, the system was kept unblocked, and stirring was conducted overnight. After 12 h, the system was heated to 70° C. to 80° C., a sample was tested every other 2 h, where the reaction was complete in 14 h to 20 h. The reaction solution was slowly added into 100 mL of water while stirring, 100 mL of dichloromethane was added, stirring was conducted for 10 min, the stirred material was allowed to stand for 5 min, liquid separation was conducted, water phase was extracted once by using 100 mL of dichloromethane, liquid separation was conducted, organic phase was combined, and water was added for washing to be neutral. 8 g of anhydrous sodium sulfate was added into the obtained organic phase, stirring and drying were performed for 5 min, filtering was performed, a filter cake was drip washed with 13 mL of dichloromethane, and the organic phase was concentrated (−0.06 MPa to 0.075 MPa and 35° C. to 40° C.) until no solvent flowed out to obtain 42 mmol of Intermediate 22b with a yield of 84%.

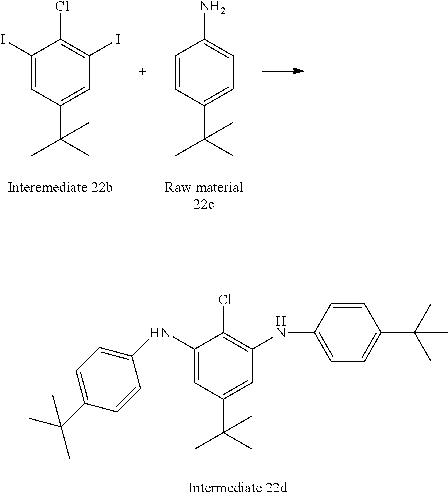 [SMILES: CC(C)(C)c1cc(I)c(Cl)c(I)c1 ; CC(C)(C)c1ccc(N)cc1 ; CC(C)(C)c1ccc(Nc2cc(C(C)(C)C)cc(Nc3ccc(C(C)(C)C)cc3)c2Cl)cc1]

(2) Nitrogen was introduced into a three-mouthed reaction bottle provided with a mechanical stirrer, a thermometer and a constant-pressure dropping funnel for 10 min (2000 mL/min), Intermediate 22b (40 mmol, 16.82 g), Raw material 22c (84 mmol, 16.73 g) and 130 mL of toluene was sequentially added, heating, refluxing and water separation were performed for 0.5 h, cooling was performed to 70° C. to 80° C., 120 mmol of sodium tert-butoxide was slowly added, heating was performed to 100° C. to 110° C. for refluxing, water was separated for 0.5 h while refluxing, cooling was performed to 50° C. to 60° C., and palladium acetate (0.4 mmol) and tricyclohexylphosphine (0.8 mmol) were slowly added in batches, where attention was paid to observe the temperature of the system, and a violent reaction will generate. After the system was stable, heating was performed for refluxing (T=100° C. to 110° C.), a reaction was carried out for 3 h while keeping the temperature, a sample was submitted for inspection, and the reaction was stopped when the reaction was complete. Cooling was performed to 25° C. to 30° C., 130 mL of water and 130 mL of toluene were added, stirring and liquid separation were performed, water phase was extracted once with 130 mL of toluene, liquid was separated, organic phase was combined, 0.7 g of anhydrous sodium sulfate was added into the organic phase, stirring, drying and filtering were performed, the organic phase was concentrated (−0.06 MPa to 0.075 MPa and 55° C. to 60° C.) until no solvent flowed out to obtain a crude product, 20 mL of ethanol was added, stirring was performed at 25° C. to separate out a large amount of solids, and filtering was performed. A filter cake was drip washed with ethanol to obtain 20 mmol of Intermediate 22d with a yield of 50%.

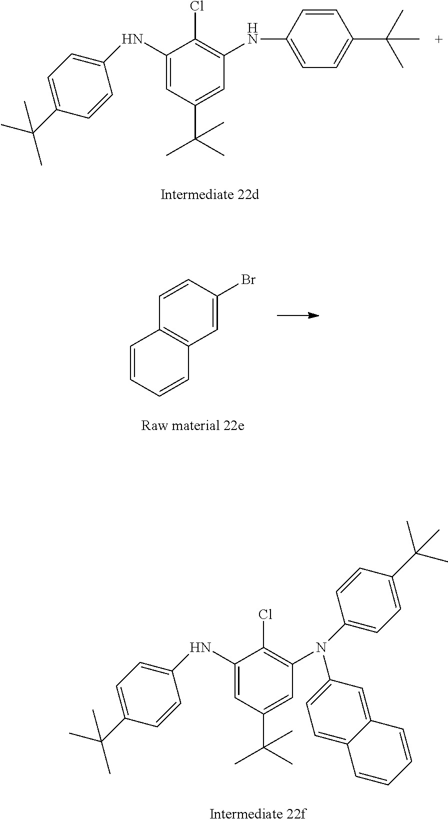

(3) Nitrogen was introduced into a three-mouthed reaction bottle provided with a mechanical stirrer, a thermometer and a constant-pressure dropping funnel for 10 min (2000 mL/min), Intermediate 22d (30 mmol, 13.89 g), Raw material 22e (33 mmol, 7.03 g) and 130 mL of toluene were sequentially added, heating, refluxing and water separation were performed for 0.5 h, cooling was performed to 70° C. to 80° C., 100 mmol of sodium tert-butoxide was slowly added, heating was performed to 100° C. to 110° C. for refluxing, water was separated for 0.5 h while refluxing, cooling was performed to 50° C. to 60° C., and palladium acetate (0.4 mmol) and tricyclohexylphosphine (0.8 mmol) were slowly added in batches, where attention was paid to observe the temperature of the system, and a violent reaction will generate. After the system was stable, heating was performed for refluxing (T=100° C. to 110° C.), a reaction was carried out for 3 h while keeping the temperature, a sample was submitted for inspection, and the reaction was stopped when the reaction was complete. Cooling was performed to 25° C. to 30° C., 130 mL of water and 130 mL of toluene were added, stirring and liquid separation were performed, water phase was extracted once with 130 mL of toluene, liquid was separated, organic phase was combined, 0.7 g of anhydrous sodium sulfate was added into the organic phase, stirring, drying and filtering were performed, the organic phase was concentrated (−0.06 MPa to 0.075 MPa and 55° C. to 60° C.) until no solvent flowed out to obtain a crude product, 20 mL of ethanol was added, stirring was performed at 25° C. to separate out a large amount of solids, and filtering was performed. A filter cake was drip washed with ethanol to obtain 25 mmol of Intermediate 22f with a yield of 83.3%.

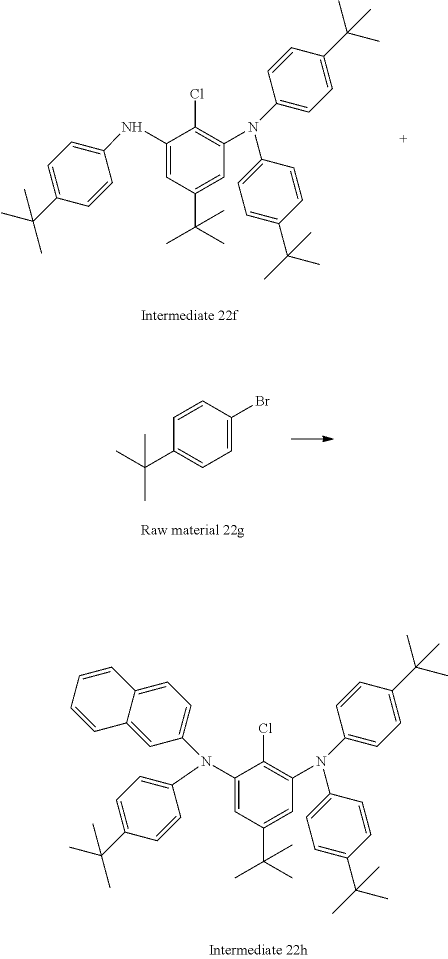

(4) Nitrogen was introduced into a three-mouthed reaction bottle provided with a mechanical stirrer, a thermometer and a constant-pressure dropping funnel for 10 min (2000 mL/min), Intermediate 22f (22 mmol, 13.10 g), Raw material 22g (26.4 mmol, 5.46 g) and 130 mL of toluene were sequentially added, heating, refluxing and water separation were performed for 0.5 h, cooling was performed to 70° C. to 80° C., 85 mmol of sodium tert-butoxide was slowly added, heating was performed to 100° C. to 110° C. for refluxing, water was separated for 0.5 h while refluxing, cooling was performed to 50° C. to 60° C., and palladium acetate (0.38 mmol) and tricyclohexylphosphine (0.65 mmol) were slowly added in batches, where attention was paid to observe the temperature of the system, and a violent reaction will generate. After the system was stable, heating was performed for refluxing (T=100° C. to 110° C.), a reaction was carried out for 3 h while keeping the temperature, a sample was submitted for inspection, and the reaction was stopped when the reaction was complete. Cooling was performed to 25° C. to 30° C., 130 mL of water and 130 mL of toluene were added, stirring and liquid separation were performed, water phase was extracted once with 130 mL of toluene, liquid was separated, organic phase was combined, 0.7 g of anhydrous sodium sulfate was added into the organic phase, stirring, drying and filtering were performed, the organic phase was concentrated (−0.06 MPa to 0.075 MPa and 55° C. to 60° C.) until no solvent flowed out to obtain a crude product, 30 mL of ethanol was added, stirring was conducted at 25° C. to separate out a large amount of solids, and filtering was performed. A filter cake was drip washed with ethanol to obtain 19.8 mmol of Intermediate 22h with a yield of 90%.

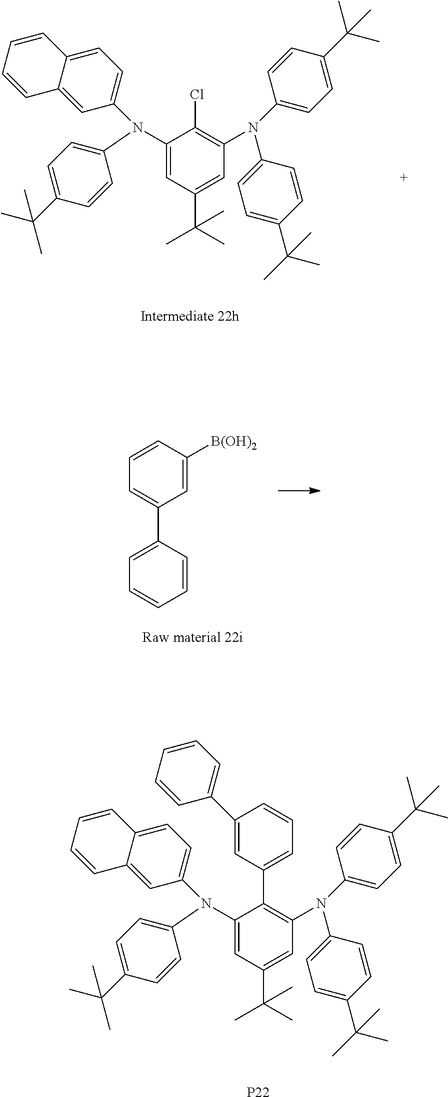

(5) 78 mL of toluene, 58 mL of ethanol and 23 mL of water were added into a three-necked flask provided with a mechanical stirrer, a thermometer and a Y-shaped tube, stirring was started, Intermediate 22h (16 mmol, 11.54 g), Raw material 22i (18 mmol, 3.56 g), potassium carbonate (30 mmol, 4.15 g) and tetrabutylammonium bromide (1.5 mmol, 0.48 g) were added, heating was performed to 45° C. to 50° C., palladium acetate (0.075 mmol, 0.017 g) was quickly added, heating was continuously conducted to 65° C. to 70° C., and a reaction was carried out for 8 h while heat preservation. The reaction solution was cooled to 15° C. to 20° C., filtering and draining were conducted, a filter cake was drip washed with ethanol, and draining was performed. Liquid separation was performed on the obtained filtrate, water phase was extracted with toluene, and an organic phase was washed with water for three times. An organic layer was dried by using anhydrous magnesium sulfate, filtering was conducted, the obtained filtrate was allowed to pass through a short silica gel column after filtering, solvent was removed under reduced pressure, and a crude product was recrystallized by using a dichloromethane system for purifying to obtain 9.1 g of Compound P22 with a yield of 68%. m/z=840.25 [M+H]⁺.

### Synthesis Example 4 (Compound P34)

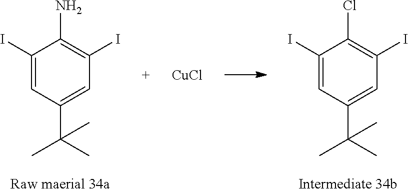

(1) 15 mL of concentrated sulfuric acid was added into a three-mouthed reaction bottle provided with a mechanical stirrer, a thermometer and a constant-pressure dropping funnel, stirring was started, 5 g of sodium nitrite was added in batches, and the temperature of a system was controlled to be 60° C. to 80° C., where the system was light green after feeding. Raw material 34a (50 mmol, 15.05 g) was dissolved in 40 mL of glacial acetic acid, the obtained solution was slowly dropwise added into the concentrated sulfuric acid solution of sodium nitrite while keeping the temperature at 40° C. to 50° C., where heat was slightly released in the system, and the temperature was kept for 2 h after dropwise adding to obtain a A34 solution. Cuprous chloride (100 mmol, 9.79 g) was dissolved in 30 mL of concentrated hydrochloric acid, the obtained solution was cooled to −10° C., the A34 solution was started to be slowly added dropwise, where the temperature was controlled to be −10° C., and dropwise adding was performed in batches at an interval of 30 min for about 4 h, the resulting solution was naturally heated, the system was kept unblocked, and stirring was conducted overnight. After 12 h, the system was heated to 70° C. to 80° C., a sample was tested every other 2 h, where the reaction was complete in 14 h to 20 h, the reaction solution was slowly added into 100 mL of water while stirring, 100 mL of dichloromethane was added, stirring was conducted for 10 min, the stirred material was allowed to stand for 5 min, liquid separation was conducted, water phase was extracted once by using 100 mL of dichloromethane, liquid separation was conducted, organic phase was combined, and water was added for washing to be neutral. 8 g of anhydrous sodium sulfate was added into the obtained organic phase, stirring and drying were performed for 5 min, filtering was performed, a filter cake was drip washed with 13 mL of dichloromethane, and the organic phase was concentrated (−0.06 MPa to 0.075 MPa and 35° C. to 40° C.) until no solvent flowed out to obtain 42 mmol of Intermediate 34b with a yield of 84%.

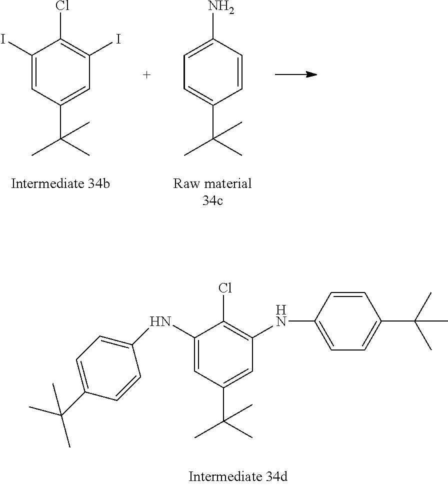 [SMILES: CC(C)(C)c1cc(I)c(Cl)c(I)c1 ; CC(C)(C)c1ccc(N)cc1 ; CC(C)(C)c1ccc(Nc2cc(C(C)(C)C)cc(Nc3ccc(C(C)(C)C)cc3)c2Cl)cc1]

(2) Nitrogen was introduced into a three-mouthed reaction bottle provided with a mechanical stirrer, a thermometer and a constant-pressure dropping funnel for 10 min (2000 mL/min), Intermediate 34b (40 mmol, 16.82 g), p-tert-butylaniline (84 mmol, 12.53 g) and 130 mL of toluene was sequentially added, heating, refluxing and water separation were performed for 0.5 h, cooling was performed to 70° C. to 80° C., 120 mmol of sodium tert-butoxide was slowly added, heating was performed to 100° C. to 110° C. for refluxing, water was separated for 0.5 h while refluxing, cooling was performed to 50° C. to 60° C., and palladium acetate (0.4 mmol) and tricyclohexylphosphine (0.8 mmol) were slowly added in batches, where attention was paid to observe the temperature of the system, and a violent reaction will generate. After the system was stable, heating was performed for refluxing (T=100° C. to 110° C.), a reaction was carried out for 3 h while keeping the temperature, a sample was submitted for inspection, and the reaction was stopped when the reaction was complete. Cooling was performed to 25° C. to 30° C., 130 mL of water and 130 mL of toluene were added, stirring and liquid separation were performed, water phase was extracted once with 130 mL of toluene, liquid was separated, organic phase was combined, 0.7 g of anhydrous sodium sulfate was added into the organic phase, stirring, drying and filtering were performed, the organic phase was concentrated (−0.06 MPa to 0.075 MPa and 55° C. to 60° C.) until no solvent flowed out to obtain a crude product, 20 mL of ethanol was added, stirring was performed at 25° C. to separate out a large amount of solids, and filtering was performed. A filter cake was drip washed with ethanol to obtain 33 mmol of Intermediate 34d with a yield of 82.5%.

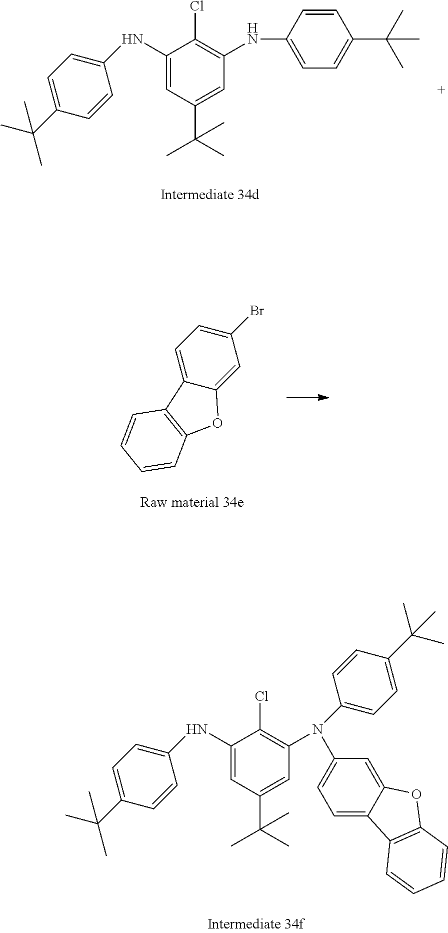

(3) Nitrogen was introduced into a three-mouthed reaction bottle provided with a mechanical stirrer, a thermometer and a constant-pressure dropping funnel for 10 min (2000 mL/min), Intermediate 34d (25 mmol, 11.53 g), Raw material 34e (27.5 mmol, 6.76 g) and 130 mL of toluene were sequentially added, heating, refluxing and water separation were performed for 0.5 h, cooling was performed to 70° C. to 80° C., 100 mmol of sodium tert-butoxide was slowly added, heating was performed to 100° C. to 110° C. for refluxing, water was separated for 0.5 h while refluxing, cooling was performed to 50° C. to 60° C., and palladium acetate (0.3 mmol) and tricyclohexylphosphine (0.6 mmol) were slowly added in batches, where attention was paid to observe the temperature of the system, and a violent reaction will generate. After the system was stable, heating was performed for refluxing (T=100° C. to 110° C.), a reaction was carried out for 3 h while keeping the temperature, a sample was submitted for inspection, and the reaction was stopped when the reaction was complete. Cooling was performed to 25° C. to 30° C., 130 mL of water and 130 mL of toluene were added, stirring and liquid separation were performed, water phase was extracted once with 130 mL of toluene, liquid was separated, organic phase was combined, 0.7 g of anhydrous sodium sulfate was added into the organic phase, stirring, drying and filtering were performed, the organic phase was concentrated (−0.06 MPa to 0.075 MPa and 55° C. to 60° C.) until no solvent flowed out to obtain a crude product, 20 mL of ethanol was added, stirring was performed at 25° C. to separate out a large amount of solids, and filtering was performed. A filter cake was drip washed with ethanol to obtain 15 mmol of Intermediate 34f with a yield of 60%.

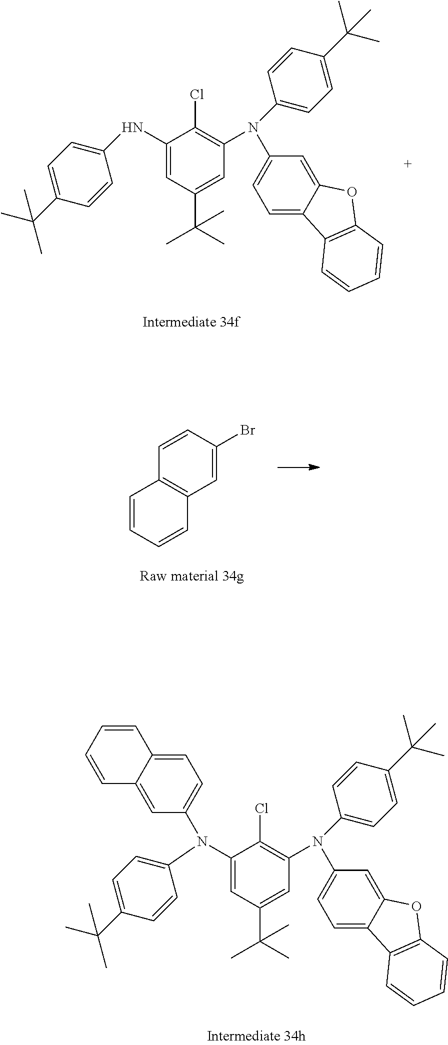 [SMILES: CC(C)(C)c1ccc(Nc2cc(C(C)(C)C)cc(N(c3ccc(C(C)(C)C)cc3)c3ccc4c(c3)oc3ccccc34)c2Cl)cc1 ; Brc1ccc2ccccc2c1 ; CC(C)(C)c1ccc(N(c2ccc3ccccc3c2)c2cc(C(C)(C)C)cc(N(c3ccc(C(C)(C)C)cc3)c3ccc4c(c3)oc3ccccc34)c2Cl)cc1]

(4) Nitrogen was introduced into a three-mouthed reaction bottle provided with a mechanical stirrer, a thermometer and a constant-pressure dropping funnel for 10 min (2000 mL/min), Intermediate 34f (22 mmol, 13.84 g), Raw material 34g (26.4 mmol, 5.46 g) and 130 mL of toluene were sequentially added, heating, refluxing and water separation were performed for 0.5 h, cooling was performed to 70° C. to 80° C., 85 mmol of sodium tert-butoxide was slowly added, heating was performed to 100° C. to 110° C. for refluxing, water was separated for 0.5 h while refluxing, cooling was performed to 50° C. to 60° C., and palladium acetate (0.38 mmol) and tricyclohexylphosphine (0.65 mmol) were slowly added in batches, where attention was paid to observe the temperature of the system, and a violent reaction will generate. After the system was stable, heating was performed for refluxing (T=100° C. to 110° C.), a reaction was carried out for 3 h while keeping the temperature, a sample was submitted for inspection, and the reaction was stopped when the reaction was complete. Cooling was performed to 25° C. to −30° C., 130 mL of water and 130 mL of toluene were added, stirring and liquid separation were performed, water phase was extracted once with 130 mL of toluene, liquid was separated, organic phase was combined, 0.7 g of anhydrous sodium sulfate was added into the organic phase, stirring, drying and filtering were performed, the organic phase was concentrated (−0.06 MPa to 0.075 MPa and 55° C. to 60° C.) until no solvent flowed out to obtain a crude product, 30 mL of ethanol was added, stirring was conducted at 25° C. to separate out a large amount of solids, and filtering was performed. A filter cake was drip washed with ethanol to obtain 19.2 mmol of Intermediate 34h with a yield of 87.3%.

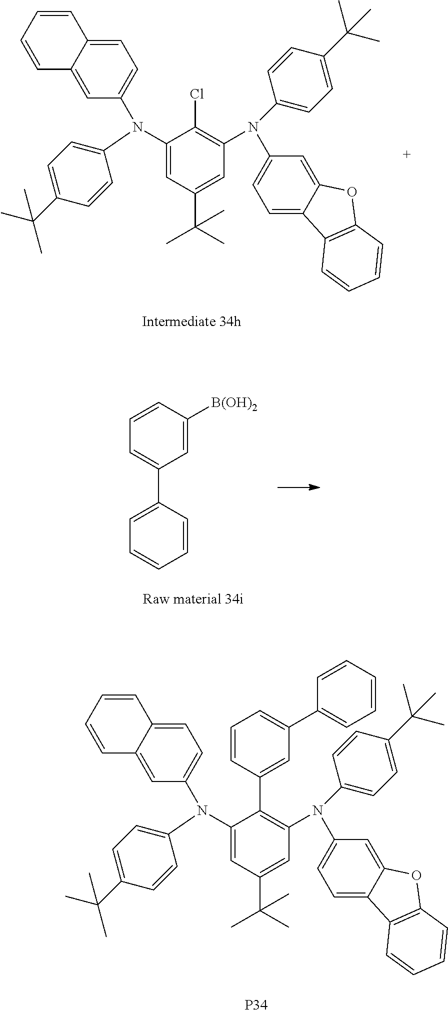

(5) 78 mL of toluene, 58 mL of ethanol and 23 mL of water were added into a three-necked flask provided with a mechanical stirrer, a thermometer and a Y-shaped tube, stirring was started, Intermediate 34h (16 mmol, 12.08 g), Raw material 34i (18 mmol, 3.56 g), potassium carbonate (30 mmol, 4.15 g) and tetrabutylammonium bromide (1.5 mmol, 0.48 g) were added, heating was performed to 45° C. to 50° C., palladium acetate (0.075 mmol, 0.017 g) was quickly added, heating was continuously conducted to 65° C. to 70° C., and a reaction was carried out for 8 h while heat preservation. The reaction solution was cooled to 15° C. to 20° C., filtering and draining were conducted, a filter cake was drip washed with ethanol, and draining was performed. Liquid separation was performed on the obtained filtrate, water phase was extracted with toluene, and an organic phase was washed with water for three times. An organic layer was dried by using anhydrous magnesium sulfate, filtering was conducted, the obtained filtrate was allowed to pass through a short silica gel column after filtering, solvent was removed under reduced pressure, and a crude product was recrystallized by using a dichloromethane system for purifying to obtain 8.2 g of Compound P34 with a yield of 59%. m/z=873.58 [M+H]⁺.

### Synthesis of Intermediate 128a

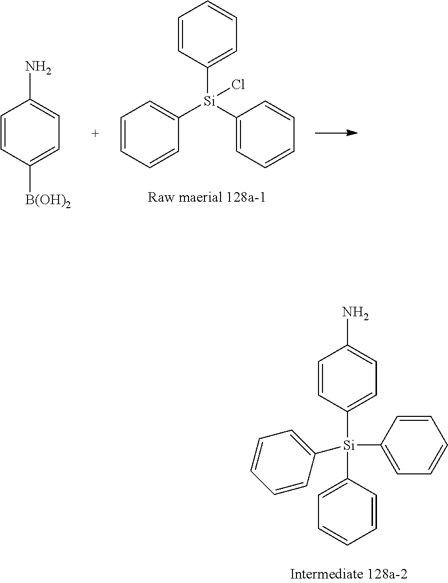 [SMILES: Nc1ccc(B(O)O)cc1 ; Cl[Si](c1ccccc1)(c1ccccc1)c1ccccc1 ; Nc1ccc([Si](c2ccccc2)(c2ccccc2)c2ccccc2)cc1]

100 mL of toluene, 20 mL of ethanol and 20 mL of water were added into a three-necked flask provided with a mechanical stirrer, a thermometer and a Y-shaped tube, stirring was started, Raw material 128a-1 (50 mmol), p-aminophenylboronic acid (55 mmol), potassium carbonate (100 mmol) and tetrabutylammonium bromide (5 mmol) were added, heating was performed to 45° C. to 50° C., palladium acetate (0.5 mmol) was quickly added, heating was continuously conducted to 65° C. to 70° C., and a reaction was carried out for 5 h while heat preservation. 20 mL of water was added, the solution was allowed to stand, liquid was separated, a water phase was extracted by using 50 mL of toluene, and an organic phase was washed with water for three times. An organic layer was dried with anhydrous magnesium sulfate, filtering was conducted, the obtained filtrate was allowed to pass through a short silica gel column after filtering, the solvent was removed under reduced pressure, and a crude product was recrystallized with ethanol of which the amount was five times that of the crude product for purifying to obtain 45 mmol of Intermediate 128a-2 with a yield of 90%.

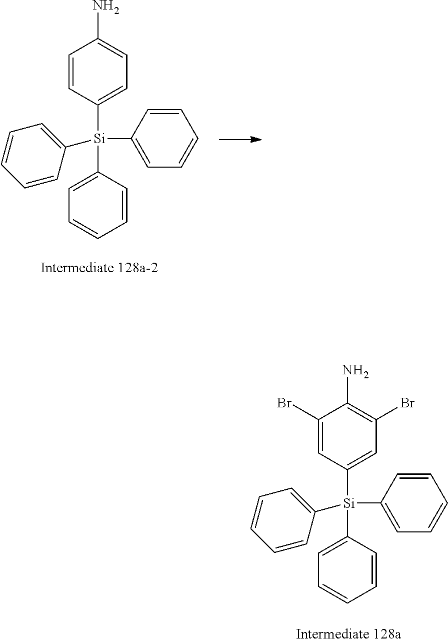 [SMILES: Nc1ccc([Si](c2ccccc2)(c2ccccc2)c2ccccc2)cc1 ; Nc1c(Br)cc([Si](c2ccccc2)(c2ccccc2)c2ccccc2)cc1Br]

(2) 60 mL of dichloroethane was added into a three-necked flask provided with a mechanical stirrer and a thermometer, stirring was started, Intermediate 128a-2 (40 mmol) was added, cooling was performed to −5° C. to 0° C., and NBS (42 mmol) was added in batches. After adding, heat preserving was performed for 1 h, and the reaction solution was washed with water for three times. An organic layer was dried with anhydrous magnesium sulfate, filtering was performed, solvent was removed under reduced pressure, petroleum ether of which the amount was three times that of the obtained residue was added, and filtering was performed to obtain 38 mmol of the product 128a with a yield of 95%.

With reference to the synthesis method for the 128a, raw materials in the following table are used for replacing the Raw material 128a-1 to synthesize related intermediates.

| Synthetic Raw | | | |
|---|---|---|---|
| materials No. | Raw material a-1 | Raw material a | Yield |
| 181 | 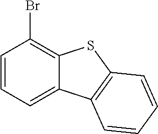 [SMILES: Brc1cccc2c1sc1ccccc12] | 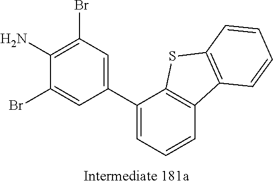 [SMILES: Nc1c(Br)cc(-c2cccc3c2sc2ccccc23)cc1Br] | 82% |
| 183 | 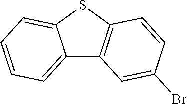 [SMILES: Brc1ccc2sc3ccccc3c2c1] | 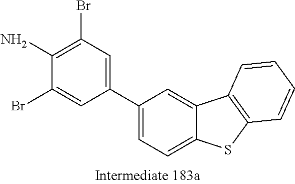 [SMILES: Nc1c(Br)cc(-c2ccc3sc4ccccc4c3c2)cc1Br] | 85% |
| 189 | 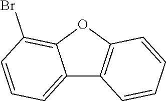 [SMILES: Brc1cccc2c1oc1ccccc12] | 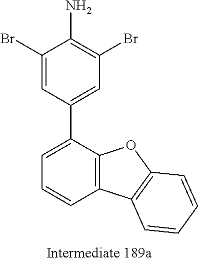 [SMILES: Nc1c(Br)cc(-c2cccc3c2oc2ccccc23)cc1Br] | 86% |
| 191 | 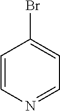 | 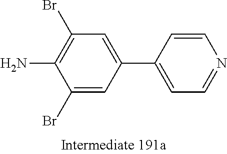 [SMILES: Nc1c(Br)cc(-c2ccncc2)cc1Br] | 79% |
| 205 | 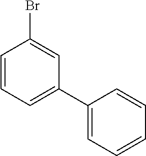 [SMILES: Brc1cccc(-c2ccccc2)c1] | 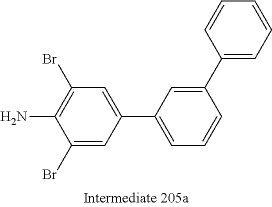 [SMILES: Nc1c(Br)cc(-c2cccc(-c3ccccc3)c2)cc1Br] | 81% |
| 212 | 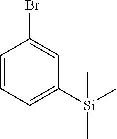 | 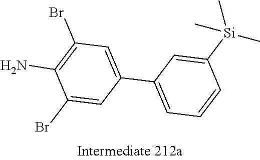 [SMILES: C[Si](C)(C)c1cccc(-c2cc(Br)c(N)c(Br)c2)c1] | 82% |

### Synthesis Examples 5 to 13

The following compounds were synthesized according to the method of Synthesis Example 1, the difference was that Raw material 4a, Raw material 4c, Raw material 4e, Raw material 4g and Raw material 4i in Example 1 are replaced with corresponding raw materials, and the adopted raw materials, and the correspondingly prepared compounds and mass spectrum data are specifically shown in Table 1-1, Table 1-2, Table 1-3, Table 1-4, Table 1-5, Table 1-6 and Table 1-7.

TABLE 1-1

| Synthesis | | | |
|---|---|---|---|
| Example No. | Synthesis Example 5 | Synthesis Example 6 | Synthesis Example 7 |
| Raw material a | 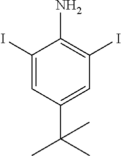 | 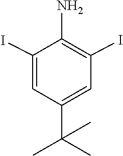 | 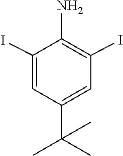 |
| Raw material c | 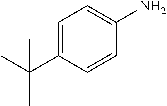 | 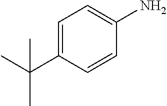 | 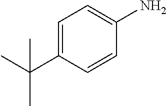 |
| Raw material e | 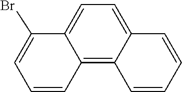 [SMILES: Brc1cccc2c1ccc1ccccc12] | 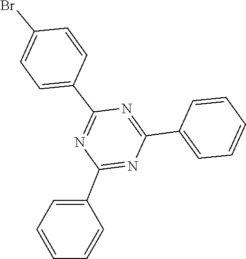 [SMILES: Brc1ccc(-c2nc(-c3ccccc3)nc(-c3ccccc3)n2)cc1] | 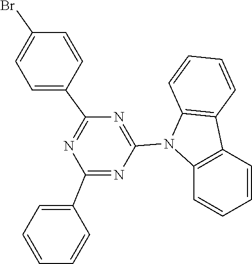 [SMILES: Brc1ccc(-c2nc(-c3ccccc3)nc(-n3c4ccccc4c4ccccc43)n2)cc1] |
| Raw material g | 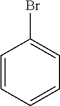 | 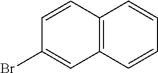 [SMILES: Brc1ccc2ccccc2c1] | 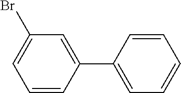 [SMILES: Brc1cccc(-c2ccccc2)c1] |
| Raw material i | 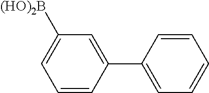 [SMILES: OB(O)c1cccc(-c2ccccc2)c1] | 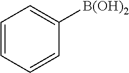 | 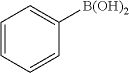 |
| Compound | 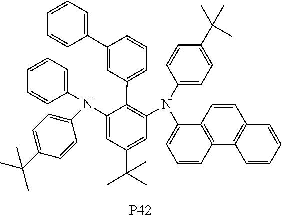 [SMILES: "P42" ; CC(C)(C)c1ccc(N(c2ccccc2)c2cc(C(C)(C)C)cc(N(c3ccc(C(C)(C)C)cc3)c3cccc4c3ccc3ccccc34)c2-c2cccc(-c3ccccc3)c2)cc1] | 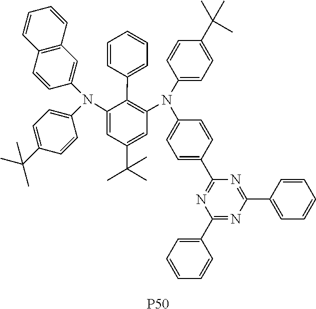 | 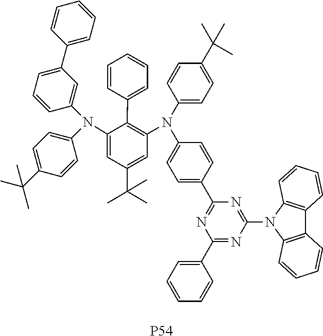 |
| Yield | 25% | 23% | 29% |
| Mass | 833.3 | 938.1 | 1054.5 |
| spectrum | | | |
| data | | | |

TABLE 1-2

| Synthesis | | | |
|---|---|---|---|
| Example | | | |
| No. | Synthesis Example 8 | Synthesis Example 9 | Synthesis Example 10 |
| Raw material a | 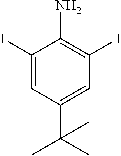 | 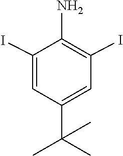 | 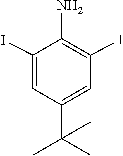 |
| Raw material c | 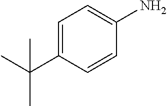 | 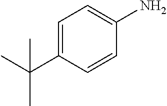 | 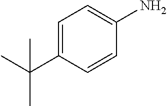 |
| Raw material e | 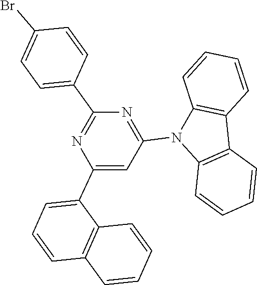 [SMILES: Brc1ccc(-c2nc(-c3cccc4ccccc34)cc(-n3c4ccccc4c4ccccc43)n2)cc1] | 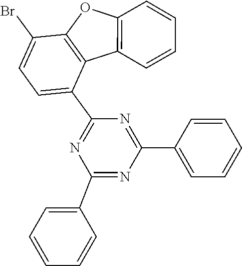 [SMILES: Brc1ccc(-c2nc(-c3ccccc3)nc(-c3ccccc3)n2)c2c1oc1ccccc12] | 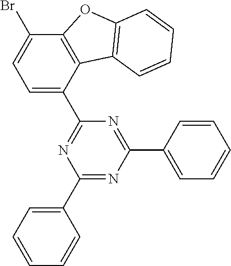 [SMILES: Brc1ccc(-c2nc(-c3ccccc3)nc(-c3ccccc3)n2)c2c1oc1ccccc12] |
| Raw material g | 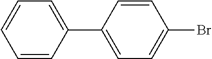 [SMILES: Brc1ccc(-c2ccccc2)cc1] | 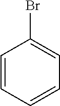 | 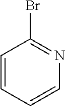 |
| Raw material i | 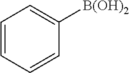 | 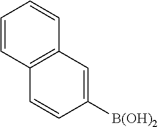 [SMILES: OB(O)c1ccc2ccccc2c1] | 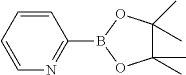 |
| Compound | 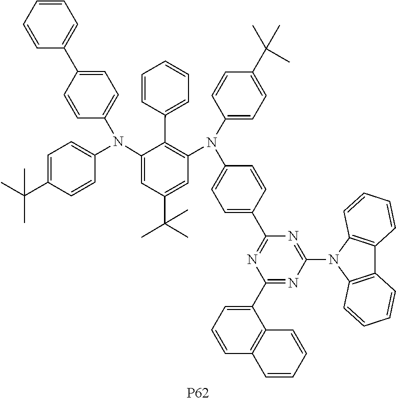 | 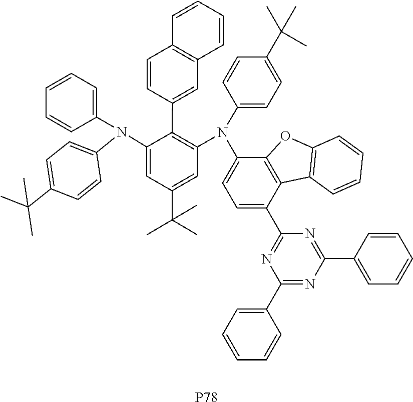 [SMILES: "P78" ; CC(C)(C)c1ccc(N(c2ccccc2)c2cc(C(C)(C)C)cc(N(c3ccc(C(C)(C)C)cc3)c3ccc(-c4nc(-c5ccccc5)nc(-c5ccccc5)n4)c4c3oc3ccccc34)c2-c2ccc3ccccc3c2)cc1] | 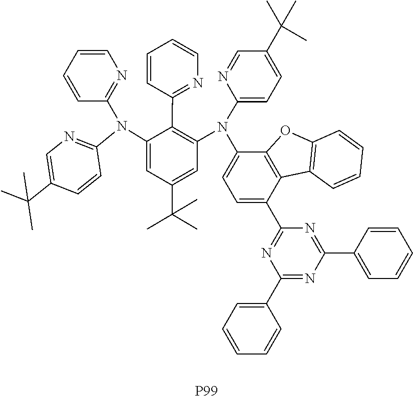 [SMILES: "P99" ; CC(C)(C)c1ccc(N(c2ccccn2)c2cc(C(C)(C)C)cc(N(c3ccc(C(C)(C)C)cn3)c3ccc(-c4nc(-c5ccccc5)nc(-c5ccccc5)n4)c4c3oc3ccccc34)c2-c2ccccn2)nc1] |
| Yield | 22% | 21% | 27% |
| Mass | 1102.8 | 1028 | 982.4 |
| spectrum | | | |
| data | | | |

TABLE 1-3

| Synthesis | | | |
|---|---|---|---|
| Example | | | |
| No. | Synthesis Example 11 | Synthesis Example 12 | Synthesis Example 13 |
| Raw material a | 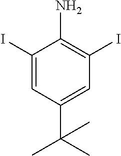 | 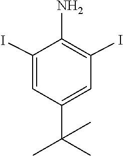 | 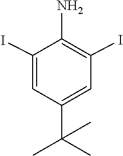 |
| Raw material c | 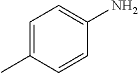 | 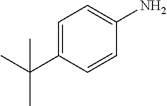 | 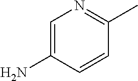 |
| Raw material e | 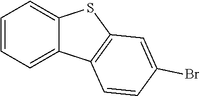 [SMILES: Brc1ccc2c(c1)sc1ccccc12] | 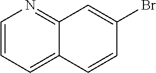 [SMILES: Brc1ccc2cccnc2c1] | 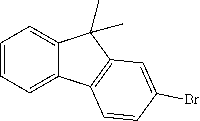 [SMILES: CC1(C)c2ccccc2-c2ccc(Br)cc21] |
| Raw material g | 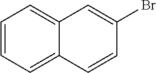 [SMILES: Brc1ccc2ccccc2c1] | 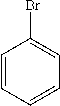 | 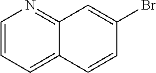 [SMILES: Brc1ccc2cccnc2c1] |
| Raw material i | 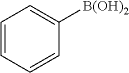 | 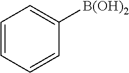 | 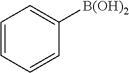 |
| Compound structure | 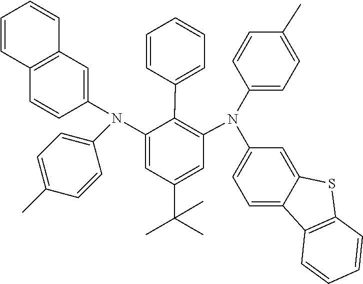 [SMILES: Cc1ccc(N(c2ccc3ccccc3c2)c2cc(C(C)(C)C)cc(N(c3ccc(C)cc3)c3ccc4c(c3)sc3ccccc34)c2-c2ccccc2)cc1] | 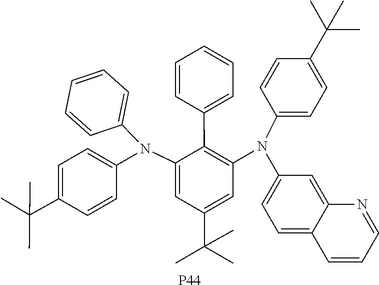 | 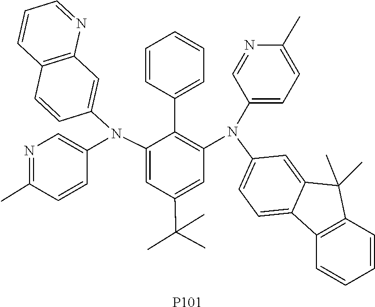 [SMILES: "P101" ; Cc1ccc(N(c2ccc3c(c2)C(C)(C)c2ccccc2-3)c2cc(C(C)(C)C)cc(N(c3ccc(C)nc3)c3ccc4cccnc4c3)c2-c2ccccc2)cn1] |
| Yield | 26% | 22% | 29% |
| Mass | 731.1 | 707.4 | 741.8 |
| spectrum | | | |
| data | | | |

TABLE 1-4

| Synthesis | | | |
|---|---|---|---|
| Example No. | Synthesis Example 14 | Synthesis Example 15 | Synthesis Example 16 |
| Raw material a | 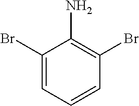 | 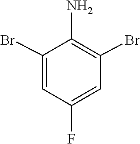 | 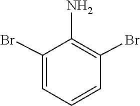 |
| Raw material c | 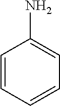 | 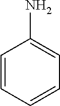 | 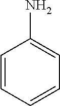 |
| Raw material e | 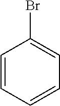 | 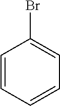 | 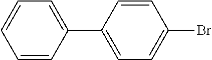 [SMILES: Brc1ccc(-c2ccccc2)cc1] |
| Raw material g | 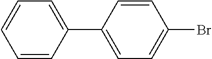 [SMILES: Brc1ccc(-c2ccccc2)cc1] | 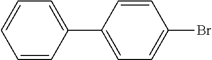 [SMILES: Brc1ccc(-c2ccccc2)cc1] | 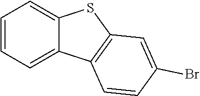 [SMILES: Brc1ccc2c(c1)sc1ccccc12] |
| Raw material i | 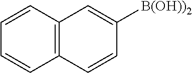 | 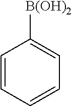 | 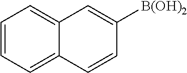 [SMILES: OB(O)c1ccc2ccccc2c1] |
| Compound structure | 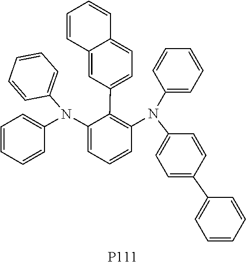 [SMILES: "P111" ; c1ccc(-c2ccc(N(c3ccccc3)c3cccc(N(c4ccccc4)c4ccccc4)c3-c3ccc4ccccc4c3)cc2)cc1] | 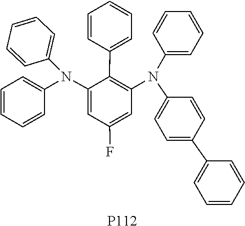 [SMILES: "P112" ; Fc1cc(N(c2ccccc2)c2ccccc2)c(-c2ccccc2)c(N(c2ccccc2)c2ccc(-c3ccccc3)cc2)c1] | 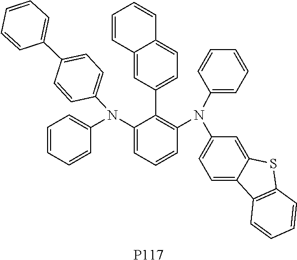 [SMILES: "P117" ; c1ccc(-c2ccc(N(c3ccccc3)c3cccc(N(c4ccccc4)c4ccc5c(c4)sc4ccccc45)c3-c3ccc4ccccc4c3)cc2)cc1] |
| Yield | 23% | 21% | 25% |
| Mass spectrum | 614.8 | 583.1 | 721.1 |
| data | | | |

TABLE 1-5

| Syn- | | | |
|---|---|---|---|
| thesis | | | |
| Ex- | | | |
| ample | | | |
| No. | Synthesis Example 17 | Synthesis Example 18 | Synthesis Example 19 |
| Raw material a | 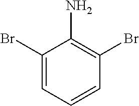 | 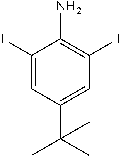 | 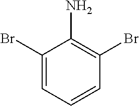 |
| Raw material c | 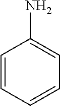 | 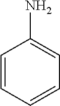 | 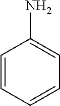 |
| Raw material e | 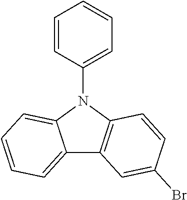 [SMILES: Brc1ccc2c(c1)c1ccccc1n2-c1ccccc1] | 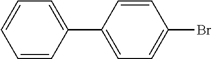 [SMILES: Brc1ccc(-c2ccccc2)cc1] | 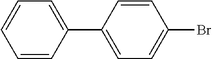 [SMILES: Brc1ccc(-c2ccccc2)cc1] |
| Raw material g | 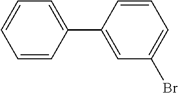 [SMILES: Brc1cccc(-c2ccccc2)c1] | 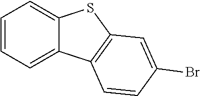 [SMILES: Brc1ccc2c(c1)sc1ccccc12] | 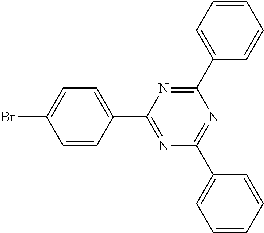 [SMILES: Brc1ccc(-c2nc(-c3ccccc3)nc(-c3ccccc3)n2)cc1] |
| Raw material i | 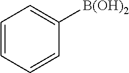 | 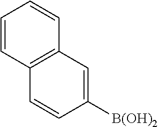 [SMILES: OB(O)c1ccc2ccccc2c1] | 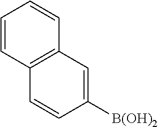 [SMILES: OB(O)c1ccc2ccccc2c1] |
| Com- pound structure | 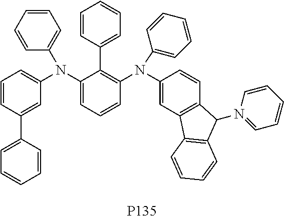 | 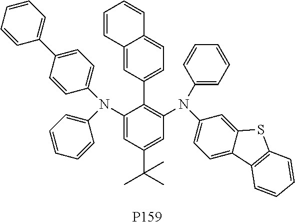 [SMILES: "P159" ; CC(C)(C)c1cc(N(c2ccccc2)c2ccc(-c3ccccc3)cc2)c(-c2ccc3ccccc3c2)c(N(c2ccccc2)c2ccc3c(c2)sc2ccccc23)c1] | 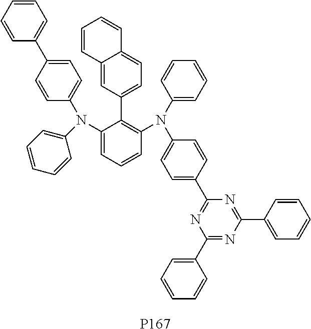 [SMILES: "P167" ; c1ccc(-c2ccc(N(c3ccccc3)c3cccc(N(c4ccccc4)c4ccc(-c5nc(-c6ccccc6)nc(-c6ccccc6)n5)cc4)c3-c3ccc4ccccc4c3)cc2)cc1] |
| Yield | 27% | 21% | 24% |
| Mass | 729.9 | 777.1 | 846.3 |
| spec- | | | |
| trum | | | |
| data | | | |

TABLE 1-6

| Synthesis | | | |
|---|---|---|---|
| Example No. | Synthesis Example 20 | Synthesis Example 21 | Synthesis Example 22 |
| Raw material a | 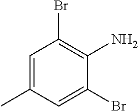 | 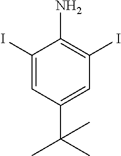 | 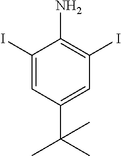 |
| Raw material c | 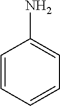 | 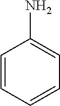 | 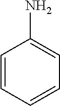 |
| Raw material e | 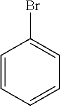 | 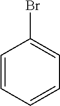 | 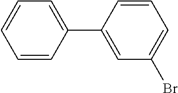 [SMILES: Brc1cccc(-c2ccccc2)c1] |
| Raw material g | 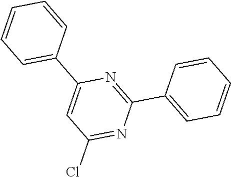 [SMILES: Clc1cc(-c2ccccc2)nc(-c2ccccc2)n1] | 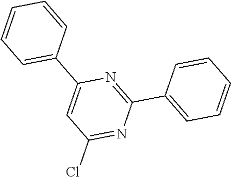 [SMILES: Clc1cc(-c2ccccc2)nc(-c2ccccc2)n1] | 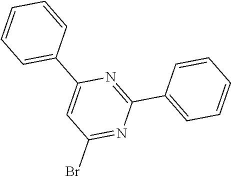 [SMILES: Brc1cc(-c2ccccc2)nc(-c2ccccc2)n1] |
| Raw material i | 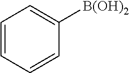 | 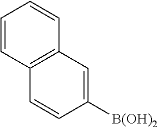 [SMILES: OB(O)c1ccc2ccccc2c1] | 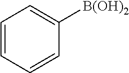 |
| Compound structure | 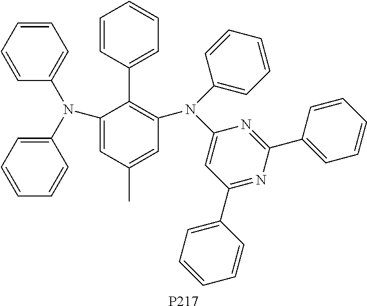 [SMILES: "P217" ; Cc1cc(N(c2ccccc2)c2ccccc2)c(-c2ccccc2)c(N(c2ccccc2)c2cc(-c3ccccc3)nc(-c3ccccc3)n2)c1] | 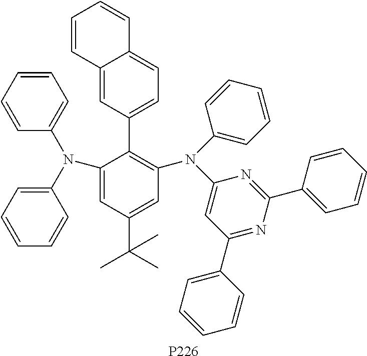 [SMILES: "P226" ; CC(C)(C)c1cc(N(c2ccccc2)c2ccccc2)c(-c2ccc3ccccc3c2)c(N(c2ccccc2)c2cc(-c3ccccc3)nc(-c3ccccc3)n2)c1] | 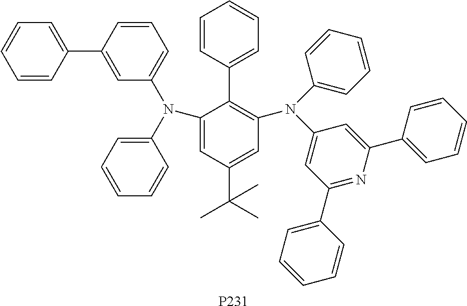 [SMILES: "P231" ; CC(C)(C)c1cc(N(c2ccccc2)c2cccc(-c3ccccc3)c2)c(-c2ccccc2)c(N(c2ccccc2)c2cc(-c3ccccc3)nc(-c3ccccc3)c2)c1] |
| Yield | 23% | 25% | 28% |
| Mass | 657.2 | 749.1 | 774.2 |
| spectrum | | | |
| data | | | |

TABLE 1-7

| Synthesis Example No. | Synthesis Example 23 | Synthesis Example 24 | Synthesis Example 25 |
|---|---|---|---|
| Raw material a | 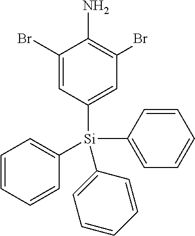 [SMILES: Nc1c(Br)cc([Si](c2ccccc2)(c2ccccc2)c2ccccc2)cc1Br] | 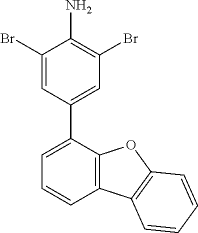 [SMILES: Nc1c(Br)cc(-c2cccc3c2oc2ccccc23)cc1Br] | 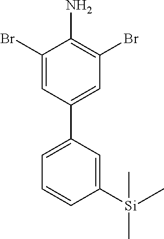 [SMILES: C[Si](C)(C)c1cccc(-c2cc(Br)c(N)c(Br)c2)c1] |
| Raw material c | 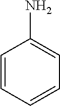 | 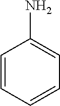 | 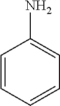 |
| Raw material e | 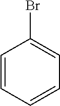 | 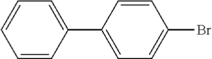 [SMILES: Brc1ccc(-c2ccccc2)cc1] | 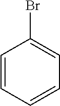 |
| Raw material g | 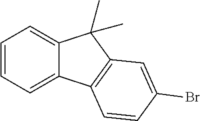 [SMILES: CC1(C)c2ccccc2-c2ccc(Br)cc21] | 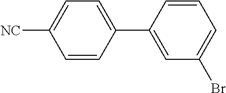 [SMILES: N#Cc1ccc(-c2cccc(Br)c2)cc1] | 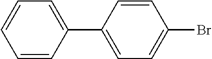 [SMILES: Brc1ccc(-c2ccccc2)cc1] |
| Raw material i | 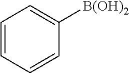 | 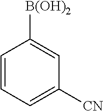 [SMILES: N#Cc1cccc(B(O)O)c1] | 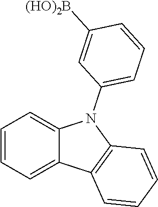 [SMILES: OB(O)c1cccc(-n2c3ccccc3c3ccccc32)c1] |
| Compound structure | 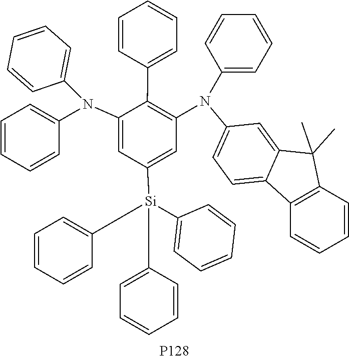 [SMILES: "P128" ; CC1(C)c2ccccc2-c2ccc(N(c3ccccc3)c3cc([Si](c4ccccc4)(c4ccccc4)c4ccccc4)cc(N(c4ccccc4)c4ccccc4)c3-c3ccccc3)cc21] | 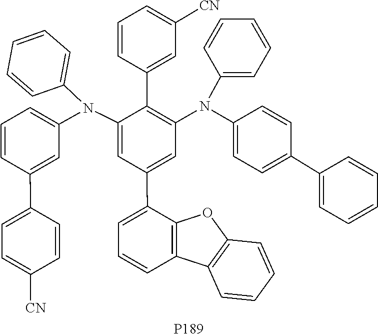 [SMILES: "P189" ; N#Cc1ccc(-c2cccc(N(c3ccccc3)c3cc(-c4cccc5c4oc4ccccc45)cc(N(c4ccccc4)c4ccc(-c5ccccc5)cc4)c3-c3cccc(C#N)c3)c2)cc1] | 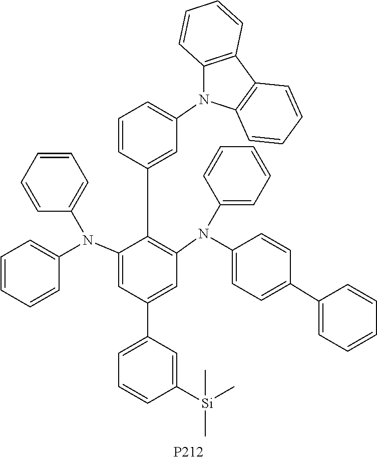 [SMILES: "P212" ; C[Si](C)(C)c1cccc(-c2cc(N(c3ccccc3)c3ccccc3)c(-c3cccc(-n4c5ccccc5c5ccccc54)c3)c(N(c3ccccc3)c3ccc(-c4ccccc4)cc3)c2)c1] |
| Yield | 22% | 25% | 24% |
| Mass spectrum | 864.2 | 858.1 | 879.1 |
| data | | | |

### Synthesis Example 26 (Synthesis of Compound P131)

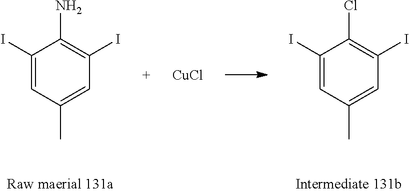

(1) 15 mL of concentrated sulfuric acid was added into a three-mouthed reaction bottle provided with a mechanical stirrer, a thermometer and a constant-pressure dropping funnel, stirring was started, 5 g of sodium nitrite was added in batches, and the temperature of a system was controlled to be 70° C., where the system was light green after feeding. Raw material 131a (50 mmol, 17.95 g) was dissolved in 40 mL of glacial acetic acid, the obtained solution was slowly dropwise added into the concentrated sulfuric acid solution of sodium nitrite while keeping the temperature at 40° C. to 50° C., where heat was slightly released in the system, and the temperature was kept for 2 h after dropwise adding to obtain a A131 solution. Cuprous chloride (100 mmol, 9.90 g) was dissolved in 30 mL of concentrated hydrochloric acid, the obtained solution was cooled to −10° C., the A131 solution was started to be slowly added dropwise, where the temperature was controlled to be −10° C., and dropwise adding was performed in batches at an interval of 30 min for about 4 h, the resulting solution was naturally heated, the system was kept unblocked, and stirring was conducted overnight. After 12 h, the system was heated to 70° C. to 80° C., and a sample was tested every other 2 h, where the reaction was complete in 14 h to 20 h. The reaction solution was slowly added into 100 mL of water while stirring, 100 mL of dichloromethane was added, stirring was conducted for 10 min, the stirred material was allowed to stand for 5 min, liquid separation was conducted, water phase was extracted once by using 100 mL of dichloromethane, liquid separation was conducted, organic phase was combined, and water was added for washing to be neutral. 8 g of anhydrous sodium sulfate was added into the obtained organic phase, stirring and drying were performed for 5 min, filtering was performed, a filter cake was drip washed with 13 mL of dichloromethane, and the organic phase was concentrated (−0.06 MPa to 0.075 MPa and 35° C. to 40° C.) until no solvent flowed out to obtain 45 mmol of Intermediate 131b with a yield of 90%.

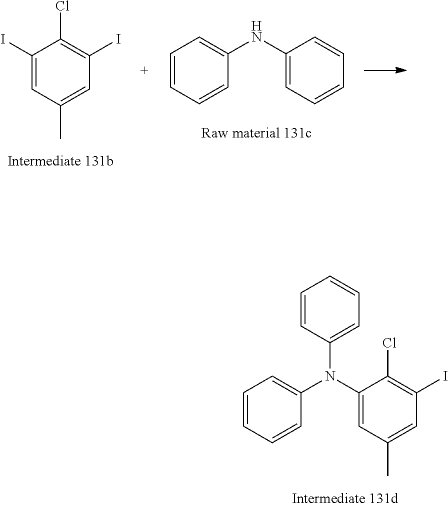

(2) Nitrogen was introduced into a three-mouthed reaction bottle provided with a mechanical stirrer, a thermometer and a condenser for 10 min (2000 mL/min), Intermediate 131b (40 mmol), Raw material 131c (44 mmol) and 100 mL of toluene was sequentially added, heating, refluxing and water separation were performed for 0.5 h, cooling was performed to 70° C. to 80° C., 80 mmol of sodium tert-butoxide was slowly added, heating was performed to 100° C. to 110° C. for refluxing, water was separated for 0.5 h while refluxing, cooling was performed to 50° C. to 60° C., and palladium acetate (0.4 mmol) and tricyclohexylphosphine (0.8 mmol) were slowly added in batches, where attention was paid to observe the temperature of the system, and a violent reaction will generate. After the system was stable, heating was performed for refluxing (T=100° C. to 110° C.), a reaction was carried out for 2 h while keeping the temperature. Cooling was performed to 25° C. to 30° C., 100 mL of water and 100 mL of toluene were added, stirring and liquid separation were performed, water phase was extracted once with 100 mL of toluene, liquid was separated, organic phase was combined, 0.7 g of anhydrous sodium sulfate was added into the organic phase, stirring, drying and filtering were performed, the organic phase was concentrated (−0.06 MPa to 0.075 MPa and 55° C. to 60° C.) until no solvent flowed out to obtain a crude product, 20 mL of xylene was added for recrystallizing, and filtering was performed to obtain 22 mmol of Intermediate 131d with a yield of 55%.

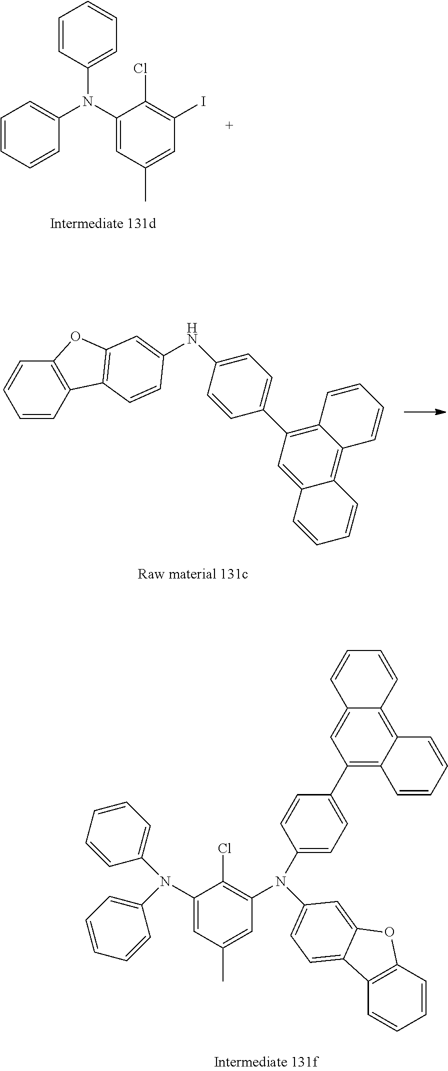 [SMILES: Cc1cc(I)c(Cl)c(N(c2ccccc2)c2ccccc2)c1 ; c1ccc2c(c1)cc(-c1ccc(Nc3ccc4c(c3)oc3ccccc34)cc1)c1ccccc12 ; Cc1cc(N(c2ccccc2)c2ccccc2)c(Cl)c(N(c2ccc(-c3cc4ccccc4c4ccccc34)cc2)c2ccc3c(c2)oc2ccccc23)c1]

(2) Nitrogen was introduced into a three-mouthed reaction bottle provided with a mechanical stirrer, a thermometer and a condenser for 10 min (2000 mL/min), Intermediate 131d (20 mmol), Raw material 131e (21 mmol) and 60 mL of toluene was sequentially added, heating, refluxing and water separation were performed for 0.5 h, cooling was performed to 70° C. to 80° C., 40 mmol of sodium tert-butoxide was slowly added, heating was performed to 100° C. to 110° C. for refluxing, water was separated for 0.5 h while refluxing, cooling was performed to 50° C. to 60° C., and palladium acetate (0.2 mmol) and tricyclohexylphosphine (0.4 mmol) were slowly added in batches, where attention was paid to observe the temperature of the system, and a violent reaction will generate. After the system was stable, heating was performed for refluxing (T=100° C. to 110° C.), a reaction was carried out for 2 h while keeping the temperature. Cooling was performed to 25° C. to 30° C., 50 mL of water was added, and filtering was performed to obtain 18 mmol of Intermediate 131f with a yield of 90%.

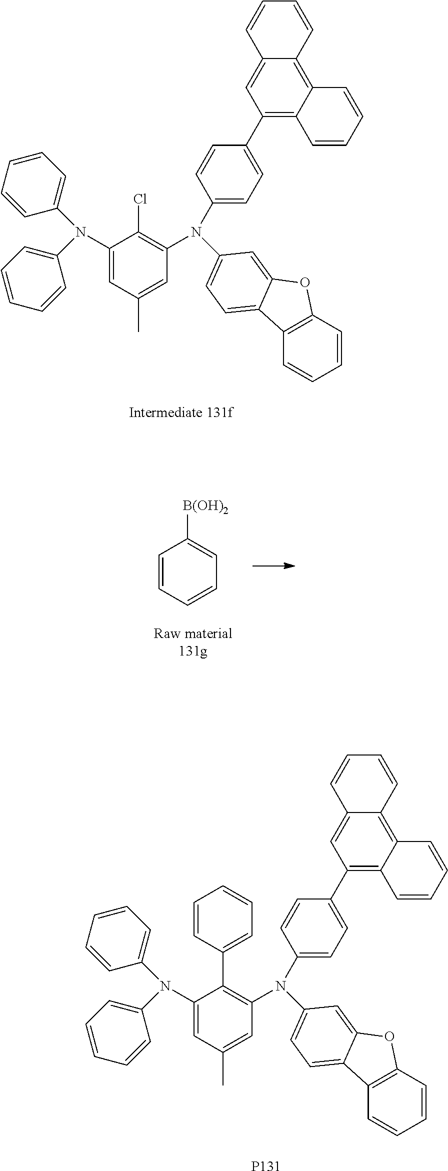 [SMILES: Cc1cc(N(c2ccccc2)c2ccccc2)c(Cl)c(N(c2ccc(-c3cc4ccccc4c4ccccc34)cc2)c2ccc3c(c2)oc2ccccc23)c1 ; OB(O)c1ccccc1 ; "P131" ; Cc1cc(N(c2ccccc2)c2ccccc2)c(-c2ccccc2)c(N(c2ccc(-c3cc4ccccc4c4ccccc34)cc2)c2ccc3c(c2)oc2ccccc23)c1]

(4) 50 mL of toluene, 10 mL of ethanol and 10 mL of water were added into a three-necked flask provided with a mechanical stirrer, a thermometer and a Y-shaped tube, stirring was started, Intermediate 131f (10 mmol), Raw material 131g (11 mmol), potassium carbonate (20 mmol) and tetrabutylammonium bromide (1 mmol) were added, heating was performed to 45° C. to 50° C., palladium acetate (0.01 mmol) was quickly added, heating was continuously conducted to 65° C. to 70° C., and a reaction was carried out for 6 h while heat preservation. The reaction solution was cooled to 15° C. to 20° C., filtering and draining were conducted, a filter cake was drip washed with ethanol, and draining was performed. Liquid separation was performed on the obtained filtrate, a water phase was extracted with toluene, and an organic phase was washed with water for three times. An organic layer was dried by using anhydrous magnesium sulfate, filtering was conducted, the obtained filtrate was allowed to pass through a short silica gel column after filtering, solvent was removed under reduced pressure, and a crude product was recrystallized by using a dichloromethane system for purifying to obtain 4.76 g of Compound P131 with a yield of 62%. m/z=769.8[M+H]⁺.

TABLE 1-8

| Synthesis | | | |
|---|---|---|---|
| Example | | | |
| No. | Synthesis Example 27 | Synthesis Example 28 | Synthesis Example 29 |
| Raw material a | 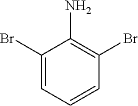 | 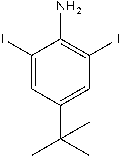 | 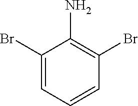 |
| Raw material c | 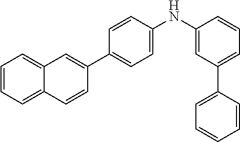 [SMILES: c1ccc(-c2cccc(Nc3ccc(-c4ccc5ccccc5c4)cc3)c2)cc1] | 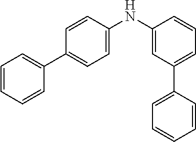 [SMILES: c1ccc(-c2ccc(Nc3cccc(-c4ccccc4)c3)cc2)cc1] | 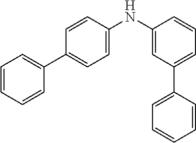 [SMILES: c1ccc(-c2ccc(Nc3cccc(-c4ccccc4)c3)cc2)cc1] |
| Raw material e | 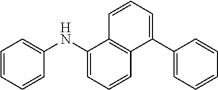 [SMILES: c1ccc(Nc2cccc3c(-c4ccccc4)cccc23)cc1] | 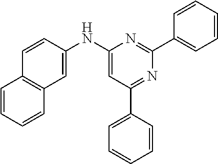 [SMILES: c1ccc(-c2cc(Nc3ccc4ccccc4c3)nc(-c3ccccc3)n2)cc1] | 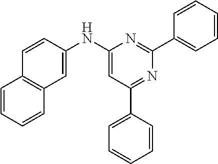 [SMILES: c1ccc(-c2cc(Nc3ccc4ccccc4c3)nc(-c3ccccc3)n2)cc1] |
| Raw material g | 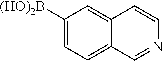 [SMILES: OB(O)c1ccc2cnccc2c1] | 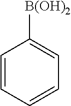 | 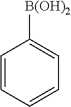 |
| Compound structure | 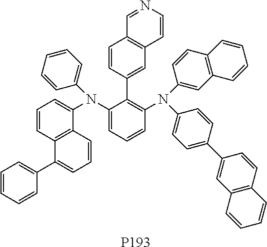 [SMILES: "P193" ; c1ccc(-c2cccc3c(N(c4ccccc4)c4cccc(N(c5ccc(-c6ccc7ccccc7c6)cc5)c5ccc6ccccc6c5)c4-c4ccc5cnccc5c4)cccc23)cc1] | 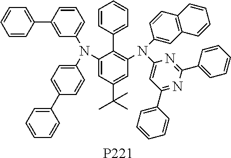 [SMILES: "P221" ; CC(C)(C)c1cc(N(c2ccc(-c3ccccc3)cc2)c2cccc(-c3ccccc3)c2)c(-c2ccccc2)c(N(c2ccc3ccccc3c2)c2cc(-c3ccccc3)nc(-c3ccccc3)n2)c1] | 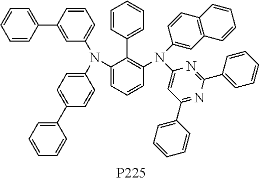 [SMILES: "P225" ; c1ccc(-c2ccc(N(c3cccc(-c4ccccc4)c3)c3cccc(N(c4ccc5ccccc5c4)c4cc(-c5ccccc5)nc(-c5ccccc5)n4)c3-c3ccccc3)cc2)cc1] |
| Yield | 28.2% | 26.4% | 27.5% |
| Mass | 869.0 | 902.1 | 846.1 |
| spectrum | | | |
| data | | | |
| [M+H]⁺ | | | |

TABLE 1-9

| Synthesis Example No. | Synthesis Example 30 | Synthesis Example 31 |
|---|---|---|
| Raw material a | 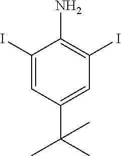 | 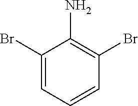 |
| Raw material c | 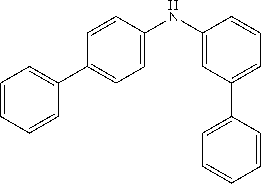 [SMILES: c1ccc(-c2ccc(Nc3cccc(-c4ccccc4)c3)cc2)cc1] | 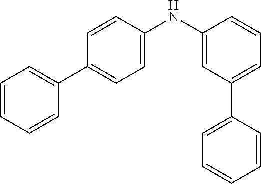 [SMILES: c1ccc(-c2ccc(Nc3cccc(-c4ccccc4)c3)cc2)cc1] |
| Raw material e | 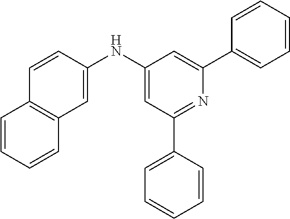 [SMILES: c1ccc(-c2cc(Nc3ccc4ccccc4c3)cc(-c3ccccc3)n2)cc1] | 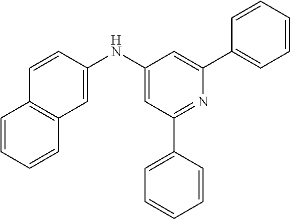 [SMILES: c1ccc(-c2cc(Nc3ccc4ccccc4c3)cc(-c3ccccc3)n2)cc1] |
| Raw material g | 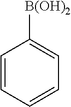 | 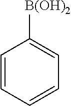 |
| Compound structure | 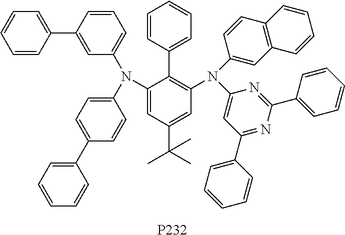 [SMILES: "P232" ; CC(C)(C)c1cc(N(c2ccc(-c3ccccc3)cc2)c2cccc(-c3ccccc3)c2)c(-c2ccccc2)c(N(c2ccc3ccccc3c2)c2cc(-c3ccccc3)nc(-c3ccccc3)n2)c1] | 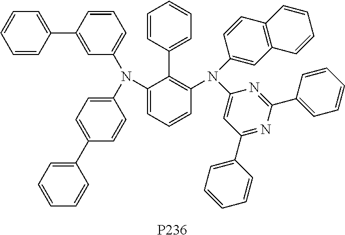 [SMILES: "P236" ; c1ccc(-c2ccc(N(c3cccc(-c4ccccc4)c3)c3cccc(N(c4ccc5ccccc5c4)c4cc(-c5ccccc5)nc(-c5ccccc5)n4)c3-c3ccccc3)cc2)cc1] |
| Yield | 28.5% | 25.6% |
| Mass spectrum data | 901.2 | 845.0 |
| [M+H]⁺ | | |

TABLE 1-10

| Synthesis | | | |
|---|---|---|---|
| No. | | | |
| Example | Synthesis Example 32 | Synthesis Example 33 | Synthesis Example 34 |
| Raw material a | 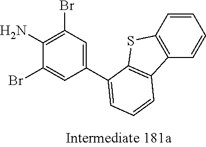 [SMILES: Nc1c(Br)cc(-c2cccc3c2sc2ccccc23)cc1Br] | 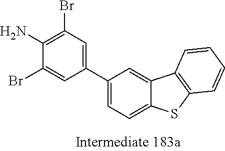 [SMILES: Nc1c(Br)cc(-c2ccc3sc4ccccc4c3c2)cc1Br] | 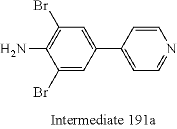 [SMILES: Nc1c(Br)cc(-c2ccncc2)cc1Br] |
| Raw material c | 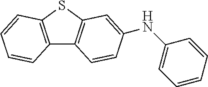 [SMILES: c1ccc(Nc2ccc3c(c2)sc2ccccc23)cc1] | 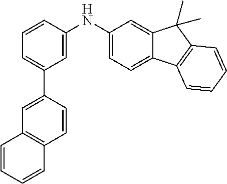 [SMILES: CC1(C)c2ccccc2-c2ccc(Nc3cccc(-c4ccc5ccccc5c4)c3)cc21] | 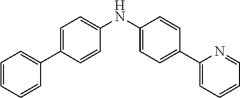 [SMILES: c1ccc(-c2ccc(Nc3ccc(-c4ccccn4)cc3)cc2)cc1] |
| Raw material e | 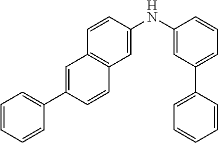 [SMILES: c1ccc(-c2cccc(Nc3ccc4cc(-c5ccccc5)ccc4c3)c2)cc1] | 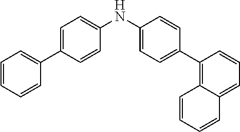 [SMILES: c1ccc(-c2ccc(Nc3ccc(-c4cccc5ccccc45)cc3)cc2)cc1] | 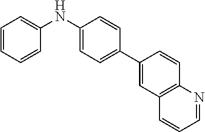 [SMILES: c1ccc(Nc2ccc(-c3ccc4ncccc4c3)cc2)cc1] |
| Raw material g | 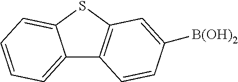 [SMILES: OB(O)c1ccc2c(c1)sc1ccccc12] | 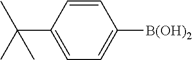 | 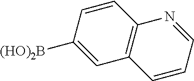 [SMILES: OB(O)c1ccc2ncccc2c1] |
| Compound structure | 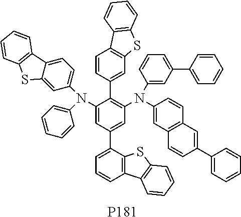 [SMILES: "P181" ; c1ccc(-c2cccc(N(c3ccc4cc(-c5ccccc5)ccc4c3)c3cc(-c4cccc5c4sc4ccccc45)cc(N(c4ccccc4)c4ccc5c(c4)sc4ccccc45)c3-c3ccc4c(c3)sc3ccccc34)c2)cc1] | 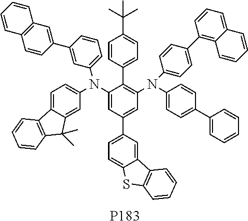 [SMILES: "P183" ; CC(C)(C)c1ccc(-c2c(N(c3ccc(-c4ccccc4)cc3)c3ccc(-c4cccc5ccccc45)cc3)cc(-c3ccc4sc5ccccc5c4c3)cc2N(c2cccc(-c3ccc4ccccc4c3)c2)c2ccc3c(c2)C(C)(C)c2ccccc2-3)cc1] | 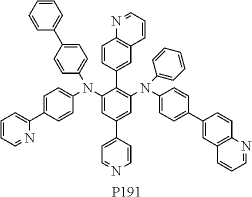 [SMILES: "P191" ; c1ccc(-c2ccc(N(c3ccc(-c4ccccn4)cc3)c3cc(-c4ccncc4)cc(N(c4ccccc4)c4ccc(-c5ccc6ncccc6c5)cc4)c3-c3ccc4ncccc4c3)cc2)cc1] |
| Yield | 30.2% | 29.4% | 27.5% |
| Mass | 1086.3 | 1172.5 | 898.0 |
| spectrum | | | |
| data | | | |
| [M+H] | | | |

TABLE 1-11

| Synthesis | | | |
|---|---|---|---|
| Example No. | Synthesis Example 35 | Synthesis Example 36 | Synthesis Example 37 |
| Raw material a | 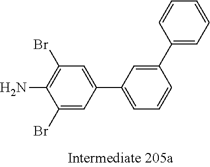 [SMILES: Nc1c(Br)cc(-c2cccc(-c3ccccc3)c2)cc1Br] | 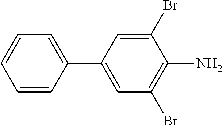 [SMILES: Nc1c(Br)cc(-c2ccccc2)cc1Br] | 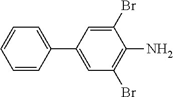 [SMILES: Nc1c(Br)cc(-c2ccccc2)cc1Br] |
| Raw material c | 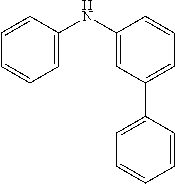 [SMILES: c1ccc(Nc2cccc(-c3ccccc3)c2)cc1] | 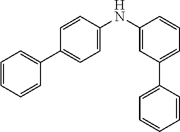 [SMILES: c1ccc(-c2ccc(Nc3cccc(-c4ccccc4)c3)cc2)cc1] | 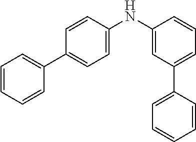 [SMILES: c1ccc(-c2ccc(Nc3cccc(-c4ccccc4)c3)cc2)cc1] |
| Raw material e | 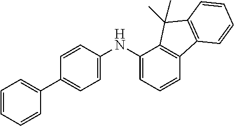 [SMILES: CC1(C)c2ccccc2-c2cccc(Nc3ccc(-c4ccccc4)cc3)c21] | 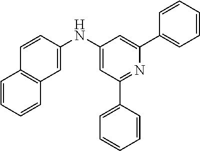 [SMILES: c1ccc(-c2cc(Nc3ccc4ccccc4c3)cc(-c3ccccc3)n2)cc1] | 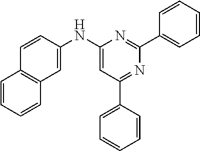 [SMILES: c1ccc(-c2cc(Nc3ccc4ccccc4c3)nc(-c3ccccc3)n2)cc1] |
| Raw material g | 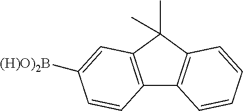 | 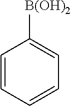 | 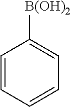 |
| Compound structure | 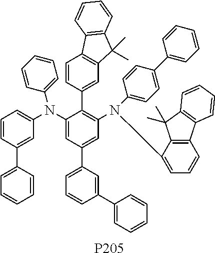 [SMILES: "P205" ; CC1(C)c2ccccc2-c2ccc(-c3c(N(c4ccccc4)c4cccc(-c5ccccc5)c4)cc(-c4cccc(-c5ccccc5)c4)cc3N(c3ccc(-c4ccccc4)cc3)c3cccc4c3C(C)(C)c3ccccc3-4)cc21] | 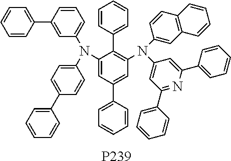 [SMILES: "P239" ; c1ccc(-c2ccc(N(c3cccc(-c4ccccc4)c3)c3cc(-c4ccccc4)cc(N(c4cc(-c5ccccc5)nc(-c5ccccc5)c4)c4ccc5ccccc5c4)c3-c3ccccc3)cc2)cc1] | 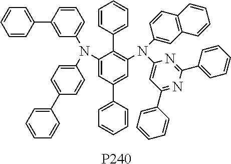 [SMILES: "P240" ; c1ccc(-c2ccc(N(c3cccc(-c4ccccc4)c3)c3cc(-c4ccccc4)cc(N(c4ccc5ccccc5c4)c4cc(-c5ccccc5)nc(-c5ccccc5)n4)c3-c3ccccc3)cc2)cc1] |
| Yield | 26.3% | 29.6% | 27.8% |
| Mass spectrum | 1026.3 | 921.2 | 922.1 |
| data [M+H]⁺ | | | |

Manufacture of Organic electroluminescent device (Second hole transport layer)

### Example 1

An anode is prepared by the following processes: an ITO substrate with an ITO thickness of 1500 Å is cut into a size of 40 mm (length)×40 mm (width)×0.7 mm (thickness) to be prepared into an experimental substrate with cathode, anode and insulating layer patterns by adopting a photoetching process, and surface treatment is performed by utilizing ultraviolet ozone and O₂: N₂ plasma to increase the work function of the anode and remove scum.

TCNQ is vacuum-evaporated on the experiment substrate (the anode) to form a hole injection layer with a thickness of 100 Å, and HT-1 is vacuum-evaporated on the hole injection layer to form a first hole transport layer with a thickness of 800 Å. Compound P4 is vacuum-evaporated on the first hole transport layer to form a hole auxiliary layer (the second hole transport layer) with a thickness of 340 Å.

CBP is evaporated on the hole auxiliary layer to serve as a host, and Ir(piq)₂(acac) is doped according to a film thickness ratio of 100:3, so that a light-emitting layer with a thickness of 350 Å is formed.

DBimiBphen and LiQ are mixed according to a weight ratio of 1:1, and an electron transport layer with a thickness of 300 Å can be formed through an evaporation process. Then, LiQ is evaporated on the electron transport layer to form an electron injection layer with a thickness of 10 Å.

Then, magnesium (Mg) and silver (Ag) are mixed at an evaporation rate of 1:9, and the mixture is vacuum-evaporated on the electron injection layer to form a cathode with a thickness of 105 Å.

In addition, CP-1 with a thickness of 650 Å is evaporated on the cathode to form a capping layer (CPL), thereby completing the manufacturing of the organic electroluminescent device. The chemical structure of main materials used for preparing the device is as follows.

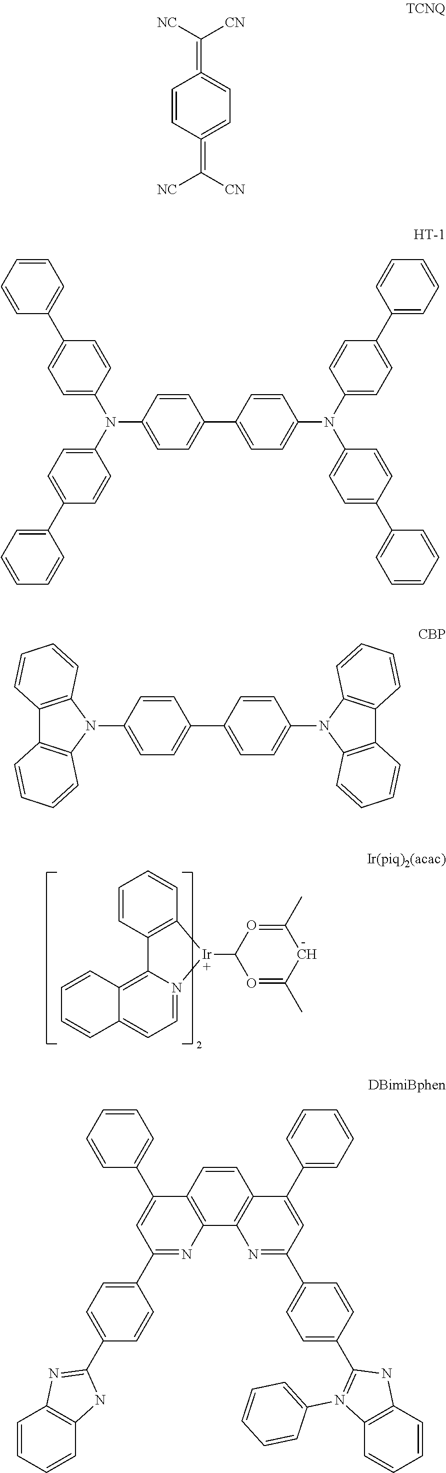

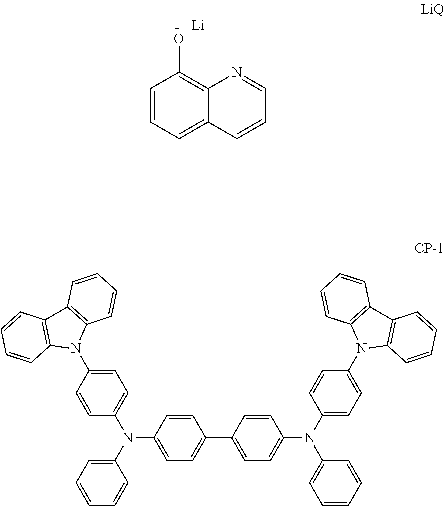 [SMILES: [Li+] ; Oc1cccc2cccnc12 ; c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1]

### Examples 2 to 23

In addition to replacing Compound P4 with the compounds shown in the following table when forming the hole auxiliary layer, the organic electroluminescent device is manufactured by using the same method as Example 1.

### Comparative Examples 1 to 2

In addition to replacing Compound P4 with Compound A and Compound B when forming the hole auxiliary layer, the organic electroluminescent device is manufactured by using the same method as Example 1. The chemical structures of Compound A and Compound B are as follows:

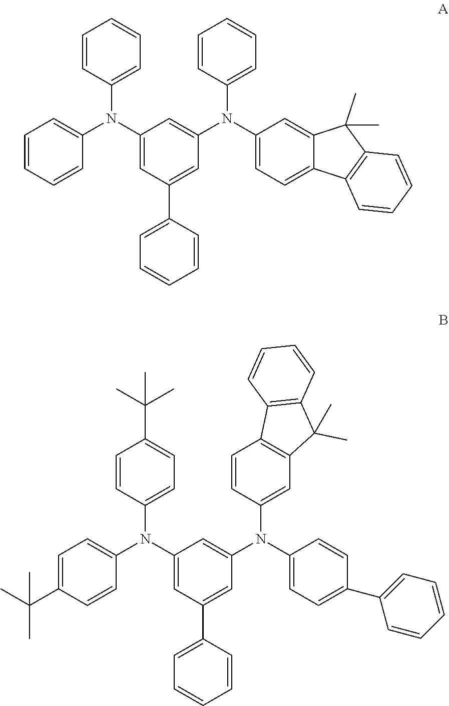 [SMILES: CC1(C)c2ccccc2-c2ccc(N(c3ccccc3)c3cc(-c4ccccc4)cc(N(c4ccccc4)c4ccccc4)c3)cc21 ; CC(C)(C)c1ccc(N(c2ccc(C(C)(C)C)cc2)c2cc(-c3ccccc3)cc(N(c3ccc(-c4ccccc4)cc3)c3ccc4c(c3)C(C)(C)c3ccccc3-4)c2)cc1]

The T95 lifetime of the organic electroluminescent device prepared above is tested under the condition of 15 mA/cm², the driving voltage, the efficiency and the chromaticity coordinate of the organic electroluminescent device prepared above are under a constant current density of 10 mA/cm², and the test results are shown in the following table.

| | | | | | External | |
|---|---|---|---|---|---|---|
| | | Driving | Current | Chromaticity | quantum | T95 |
| | Hole auxiliary | voltage | efficiency | coordinate | efficiency | lifetime |
| No. | layer | (V) | Cd/A | CIEx | EQE % | (h) |
| Example 1 | Compound P4 | 3.94 | 33.45 | 0.679 | 23.65 | 421 |
| Example 2 | Compound P10 | 3.95 | 33.63 | 0.677 | 23.81 | 416 |
| Example 3 | Compound P22 | 3.89 | 33.24 | 0.679 | 23.58 | 458 |
| Example 4 | Compound P34 | 4.01 | 33.17 | 0.681 | 23.51 | 464 |
| Example 5 | Compound P42 | 3.92 | 33.18 | 0.680 | 23.54 | 415 |
| Example 6 | Compound P50 | 3.88 | 33.56 | 0.678 | 23.71 | 426 |
| Example 7 | Compound P54 | 4.01 | 34.08 | 0.679 | 24.05 | 434 |
| Example 8 | Compound P38 | 4.00 | 33.80 | 0.678 | 23.87 | 418 |
| Example 9 | Compound P101 | 3.94 | 33.76 | 0.681 | 23.84 | 428 |
| Example 10 | Compound P111 | 4.00 | 32.99 | 0.680 | 23.48 | 398 |
| Example 11 | Compound P112 | 3.89 | 32.87 | 0.678 | 23.45 | 387 |
| Example 12 | Compound P117 | 3.96 | 33.36 | 0.677 | 23.61 | 356 |
| Example 13 | Compound P135 | 3.97 | 32.86 | 0.682 | 23.41 | 379 |
| Example 14 | Compound P159 | 3.95 | 33.56 | 0.680 | 23.74 | 408 |
| Example 15 | Compound P128 | 4.05 | 32.46 | 0.678 | 23.26 | 416 |
| Example 16 | Compound P189 | 4.09 | 33.58 | 0.679 | 23.85 | 412 |
| Example 17 | Compound p212 | 3.89 | 33.47 | 0.681 | 23.66 | 395 |
| Example 18 | Compound P131 | 4.05 | 32.87 | 0.680 | 23.44 | 396 |
| Example 19 | Compound P193 | 3.89 | 33.48 | 0.682 | 23.70 | 375 |
| Example 20 | Compound P181 | 3.88 | 33.59 | 0.680 | 23.88 | 346 |
| Example 21 | Compound P183 | 3.94 | 32.68 | 0.679 | 23.35 | 354 |
| Example 22 | Compound P191 | 4.05 | 33.56 | 0.680 | 23.72 | 348 |
| Example 23 | Compound P205 | 4.02 | 32.68 | 0.681 | 23.37 | 359 |
| Comparative | Compound A | 3.99 | 29.41 | 0.681 | 19.87 | 287 |
| Example 1 | | | | | | |
| Comparative | Compound B | 4.05 | 30.56 | 0.679 | 21.56 | 305 |
| Example 2 | | | | | | |

In combination with the results shown in the table, it can be known that compared with those in Comparative Example 1 and Comparative Example 2, the current efficiency of the organic electroluminescent devices prepared by Examples 1 to 23 is improved at least by 6.22%, the external quantum efficiency is improved at least by 7.9%, and the T95 lifetime of the organic electroluminescent devices prepared by Examples 1 to 23 is improved at least by 13.4%; in addition, the organic electroluminescent devices prepared by Examples 1 to 23 also have relatively low driving voltage. Therefore, the arylamine compound provided by the present disclosure is used as a second hole transport material, and the lifetime and the photoelectric efficiency of the device can be further improved under the condition of ensuring that the device has relatively low driving voltage.

Manufacture of Organic electroluminescent device (Electron transport layer)

### Example 24

An anode is prepared by the following processes: a substrate (manufactured by Corning) with an ITO thickness of 1200 Å is cut into a size of 40 mm (length)×40 mm (width)×0.7 mm (thickness) to be prepared into an experimental substrate with a cathode lap joint area, an anode and an insulating layer pattern by adopting a photoetching process, and surface treatment is performed by utilizing ultraviolet ozone and O₂: N₂ plasma to increase the work function of the anode (the experiment substrate), and remove scum.

PPDN is vacuum-evaporated on the experiment substrate (the anode) to form a hole injection layer with a thickness of 100 Å, and HT-1 is vacuum-evaporated on the hole injection layer to form a hole transport layer with a thickness of 800 Å. HT-2 is evaporated on the hole transport layer to form an electron-blocking layer with a thickness of 310 Å.

And then, α,β-ADN is taken as a host, and BD-1 is doped at the same time according to a film thickness ratio of 100:3 to form a light-emitting layer with a thickness of 400 Å.

Compound P62 and LiQ are mixed according to a weight ratio of 1:1, and an electron transport layer with a thickness of 330 Å can be formed through an evaporation process. Then, LiQ is evaporated on the electron transport layer to form an electron injection layer with a thickness of 10 Å.

Then, magnesium (Mg) and silver (Ag) are mixed at an evaporation rate of 1:9, and the mixture is vacuum-evaporated on the electron injection layer to form a cathode with a thickness of 120 Å.

In addition, CP-1 with a thickness of 650 Å is evaporated on the cathode to form a capping layer (CPL), thereby completing the manufacturing of the organic electroluminescent device. The chemical structure of main materials used for preparing the device is as follows.

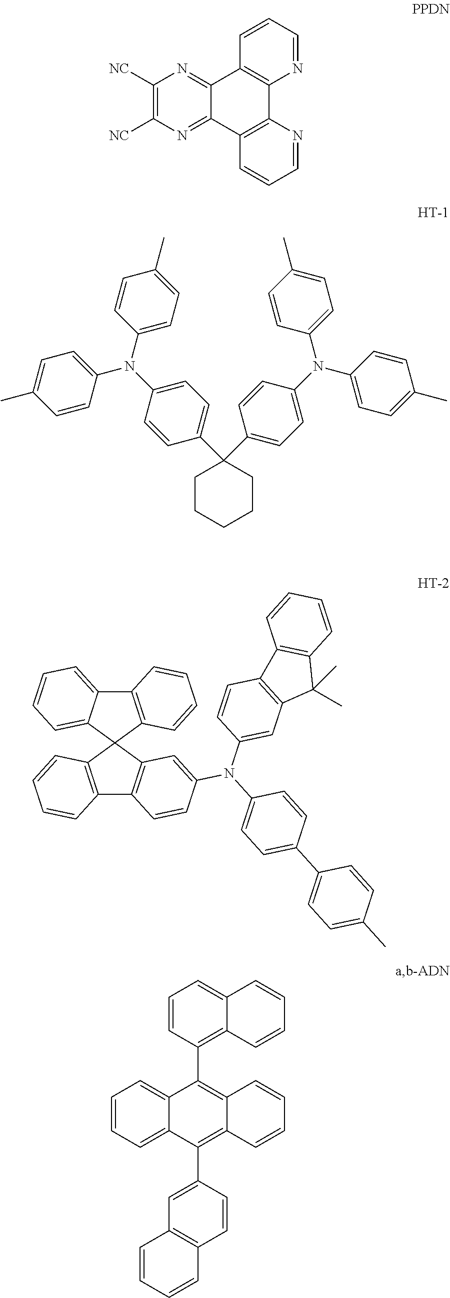

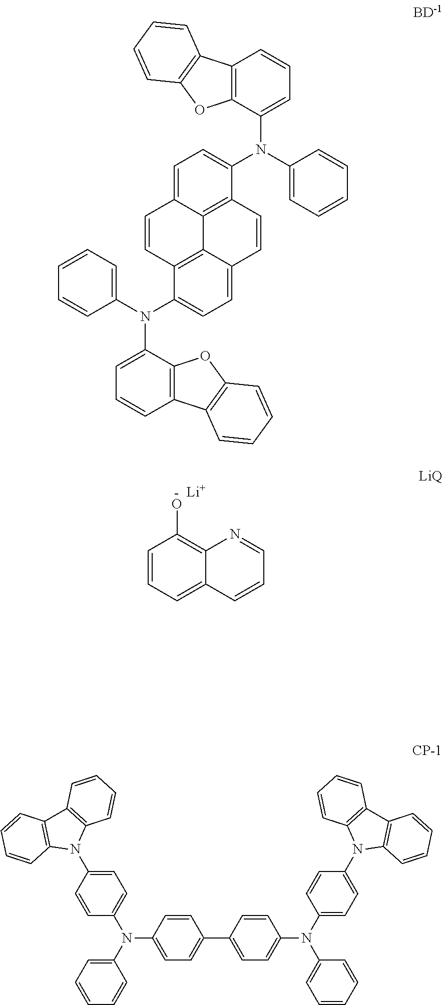

### Examples 25 to 37

In addition to replacing Compound P62 with the compounds shown in the following table when forming the electron transport layer, the organic electroluminescent device is manufactured by using the same method as Example 24.

### Comparative Examples 3 to 4

In addition to replacing Compound P62 with Compound C and Compound D when forming the electron transport layer, the organic electroluminescent device is manufactured by using the same method as Example 24. The chemical structures of Compound C and Compound D are as follows:

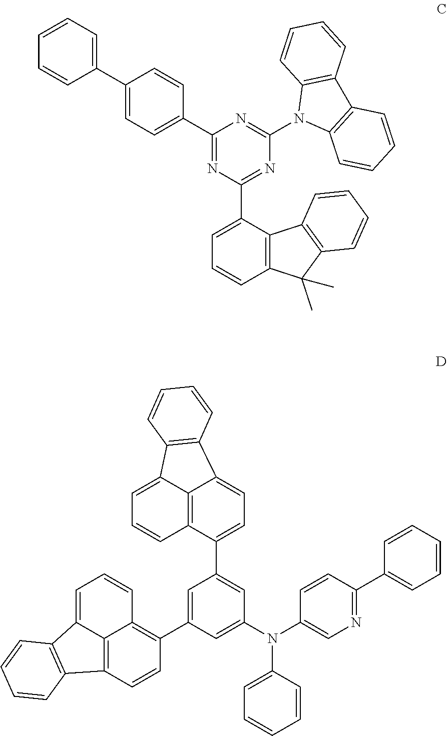 [SMILES: CC1(C)c2ccccc2-c2c(-c3nc(-c4ccc(-c5ccccc5)cc4)nc(-n4c5ccccc5c5ccccc54)n3)cccc21 ; c1ccc(-c2ccc(N(c3ccccc3)c3cc(-c4ccc5c6c(cccc46)-c4ccccc4-5)cc(-c4ccc5c6c(cccc46)-c4ccccc4-5)c3)cn2)cc1]

The T95 lifetime of the organic electroluminescent device prepared above is tested under the condition of 15 mA/cm², the driving voltage, the efficiency and the chromaticity coordinate of the organic electroluminescent device prepared above are under a constant current density of 10 mA/cm², and the test results are shown in the following table.

| | | | | | External | |
|---|---|---|---|---|---|---|
| | | Operating | Current | Chromaticity | quantum | |
| | Electron | voltage | efficiency | coordinate | efficiency | lifetime |
| No. | transport layer | (V) | Cd/A | CIEy | EQE % | T95 (h) |
| Example 24 | Compound P62 | 3.82 | 6.30 | 0.050 | 13.31 | 184 |
| Example 25 | Compound P78 | 3.90 | 6.31 | 0.051 | 13.36 | 194 |
| Example 26 | Compound P99 | 3.87 | 6.28 | 0.049 | 13.26 | 197 |
| Example 27 | Compound P44 | 3.88 | 6.45 | 0.048 | 13.51 | 184 |
| Example 28 | Compound P167 | 3.94 | 6.56 | 0.051 | 13.56 | 188 |
| Example 29 | Compound P217 | 3.91 | 6.26 | 0.053 | 13.14 | 174 |
| Example 30 | Compound P226 | 3.90 | 6.54 | 0.049 | 13.53 | 182 |
| Example 31 | Compound P231 | 3.89 | 6.68 | 0.048 | 13.57 | 197 |
| Example 32 | Compound P221 | 3.87 | 6.42 | 0.050 | 13.48 | 204 |
| Example 33 | Compound P225 | 3.90 | 6.43 | 0.049 | 13.49 | 201 |
| Example 34 | Compound P232 | 3.91 | 6.38 | 0.051 | 13.40 | 199 |
| Example 35 | Compound P236 | 3.87 | 6.26 | 0.051 | 13.15 | 210 |
| Example 36 | Compound 239 | 3.84 | 6.28 | 0.050 | 13.27 | 172 |
| Example 37 | Compound 240 | 3.95 | 6.33 | 0.048 | 13.35 | 183 |
| Comparative | Compound C | 4.09 | 5.81 | 0.050 | 11.68 | 182 |
| Example 3 | | | | | | |
| Comparative | Compound D | 4.13 | 6.05 | 0.048 | 12.15 | 197 |
| Example 4 | | | | | | |

In combination with the results shown in the table, it can be known that compared with those of Comparative Examples 3 to 4, the driving voltage of the organic electroluminescent devices prepared in Examples 24 to 37 is reduced at least by 0.14V, the current efficiency is improved at least by 3.5%, and the external quantum efficiency is improved at least by 8.2%; therefore, compared with Comparative Examples 3 to 4, the organic electroluminescent devices prepared in the Examples 24 to 37 have lower driving voltage and higher luminous efficiency.

According to the above table, it can be known that compared with Comparative Examples 3 and 4, the blue light organic electroluminescent devices prepared in Examples 24 to 37 have higher current efficiency, external quantum efficiency and lower driving voltage.

The preferable embodiments of the present disclosure are described in detail above, however, the present application is not limited to the specific details in the above embodiments, in the technical concept range of the present application, the technical solution of the present disclosure can be subjected to various simple variations, and these simple variations all belong to the protection range of the present application.

In addition, it should be noted that the specific technical features described in the above embodiments can be combined in any suitable mode without contradiction. In order to avoid unnecessary repetition, various possible combination modes of the present application are not illustrated separately.

In addition, various different embodiments of the present application can also be combined arbitrarily, and as long as the embodiments do not violate the idea of the present application, the embodiments also should be regarded as the content disclosed by the present application.

1. An arylamine compound having a structure represented by Formula (1): 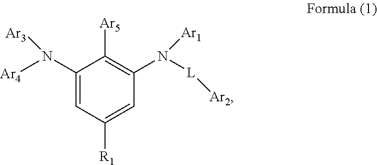
wherein R₁ is selected from deuterium, a cyano, a fluorine, a trimethylsilyl, a triphenylsilyl, an alkyl with 1 to 10 carbon atoms, a substituted or unsubstituted phenyl, a substituted or unsubstituted naphthyl, a substituted or unsubstituted biphenyl, a substituted or unsubstituted pyridyl, a substituted or unsubstituted pyrimidinyl, a substituted or unsubstituted dibenzofuranyl, a substituted or unsubstituted dibenzothienyl, or a substituted or unsubstituted fluorenyl;
Ar₅ is selected from deuterium, a cyano, a fluorine, a trimethylsilyl, a deuterated methyl, a methyl, an ethyl, an isopropyl, a tert-butyl, a substituted or unsubstituted phenyl, a substituted or unsubstituted pyridyl, a substituted or unsubstituted pyrimidyl, a substituted or unsubstituted naphthyl, a substituted or unsubstituted biphenyl, a substituted or unsubstituted quinolinyl, a substituted or unsubstituted isoquinoly, a substituted or unsubstituted phenanthryl, a substituted or unsubstituted dibenzofuranyl, a substituted or unsubstituted dibenzothienyl, or a substituted or unsubstituted fluorenyl;
the substituents of Ar₅ are the same or different, and are respectively independently selected from deuterium, a fluorine, a cyano, a methyl, an ethyl, an isopropyl, a tert-butyl, a trimethylsilyl, a phenyl, or a carbazolyl;
Ar₁, Ar₂, Ar₃ and Ar₄ are the same or different, and are respectively independently selected from a substituted or unsubstituted phenyl, a substituted or unsubstituted pyridyl, a substituted or unsubstituted pyrimidyl, a substituted or unsubstituted naphthyl, a substituted or unsubstituted biphenyl, a substituted or unsubstituted quinolinyl, a substituted or unsubstituted isoquinolinyl, a substituted or unsubstituted anthracenyl, a substituted or unsubstituted phenanthryl, a substituted or unsubstituted fluorenyl, a substituted or unsubstituted dibenzofuranyl, a substituted or unsubstituted dibenzothienyl, a substituted or unsubstituted carbazolyl, a substituted or unsubstituted triazinyl, a substituted or unsubstituted terphenyl, or a substituted or unsubstituted 9,9′-spirobifluorenyl;
the substituents of Ar₁, Ar₂, Ar₃, and Ar₄ are the same or different, and are respectively independently selected from deuterium, a fluorine, a cyano, a trimethylsilyl, a phenyl, a naphthyl, a phenanthryl, a pyridyl, a pyrimidyl, a methyl, an ethyl, an isopropyl, a tert-butyl, a carbazolyl, a dibenzofuranyl, or a dibenzothienyl;
L is selected from a single bond, a substituted or unsubstituted arylene with 6 to 12 carbon atoms, or a substituted or unsubstituted heteroarylene with 4 to 12 carbon atoms;
the substituents of R₁ and L are the same or different, and are respectively independently selected from deuterium, a fluorine, a cyano, a methyl, an ethyl, an isopropyl, a tert-butyl, a phenyl, or a trimethylsilyl.

2. The arylamine compound according to claim 1, wherein the arylamine compound has a structure represented by Formula (1): 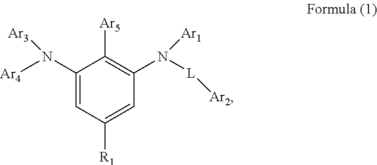
wherein R₁ is an alkyl with 1 to 10 carbon atoms;
Ar₁, Ar₂, Ar₃, and Ar₄ are the same or different, and are each independently selected from a substituted or unsubstituted phenyl, a substituted or unsubstituted pyridyl, a substituted or unsubstituted pyrimidyl, a substituted or unsubstituted naphthyl, a substituted or unsubstituted biphenyl, a substituted or unsubstituted quinolinyl, a substituted or unsubstituted isoquinolinyl, a substituted or unsubstituted anthracenyl, a substituted or unsubstituted phenanthryl, a substituted or unsubstituted fluorenyl, a substituted or unsubstituted dibenzofuranyl, a substituted or unsubstituted dibenzothienyl, a substituted or unsubstituted carbazolyl, a substituted or unsubstituted triazinyl, a substituted or unsubstituted terphenyl, or a substituted or unsubstituted 9,9′-spirobifluorenyl;
L is selected from a single bond, a substituted or unsubstituted arylene with 6 to 12 carbon atoms, or a substituted or unsubstituted heteroarylene with 4 to 12 carbon atoms;
the substituents of Ar₁, Ar₂, Ar₃, Ar₄, and L are the same or different, and are respectively independently selected from deuterium, a fluorine, a cyano, a methyl, an ethyl, an isopropyl, a tert-butyl, or a trimethylsilyl.

3. The arylamine compound according to claim 1, wherein the L is selected from a single bond, a substituted or unsubstituted phenylene, a substituted or unsubstituted naphthylene, a substituted or unsubstituted biphenylene, or a substituted or unsubstituted dibenzofuranylene.

4. The arylamine compound according to claim 1, wherein
the L is selected from a substituted or unsubstituted pyridylene, a substituted or unsubstituted dibenzothienylene, or a substituted or unsubstituted pyrimidinylene.

5. The arylamine compound according to claim 1, wherein the L is selected from a single bond or a group consisting of groups represented by the following general formulas: 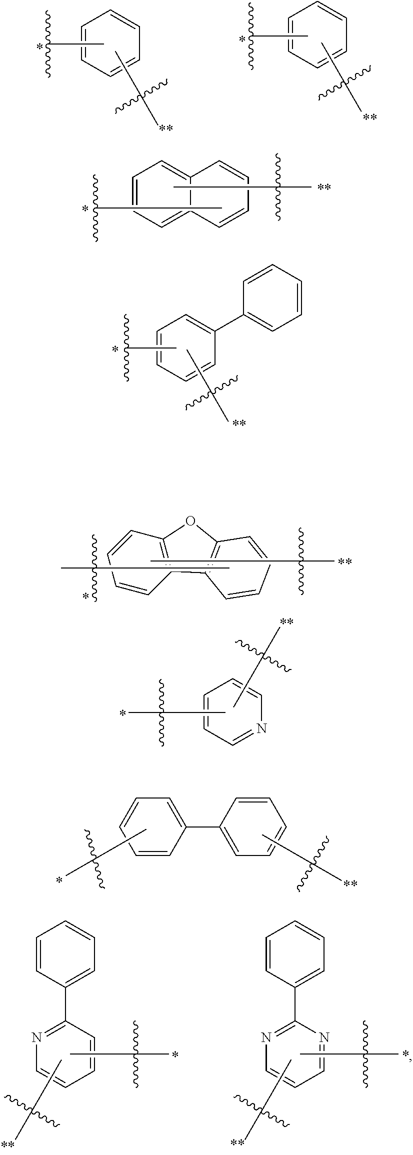
wherein * is a linking point of L connected to 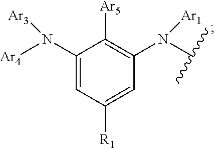
and ** is a linking point of L connected to

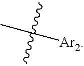

6. (canceled)

7. The arylamine compound according to claim 1, wherein the Ar₁, Ar₃ and Ar₄ are the same or different, and are respectively independently selected from a group consisting of the following groups:

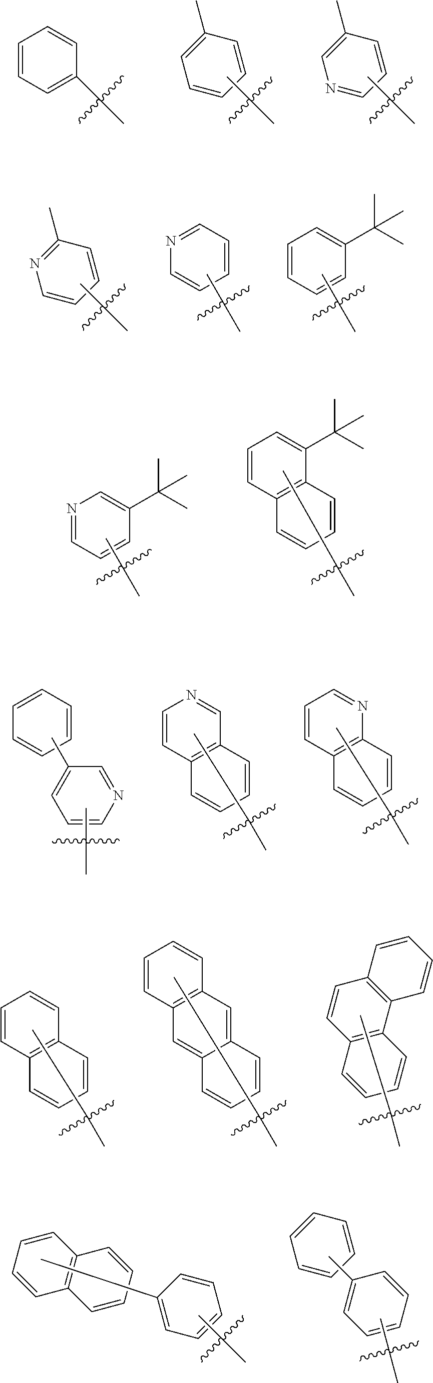

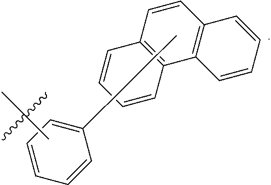

8. (canceled)

9. The arylamine compound according to claim 1, wherein the Ar₁, Ar₂, Ar₃ and Ar₄ are the same or different, and are respectively independently selected from a substituted or unsubstituted group T, and the unsubstituted group T is selected from a group consisting of the following groups: 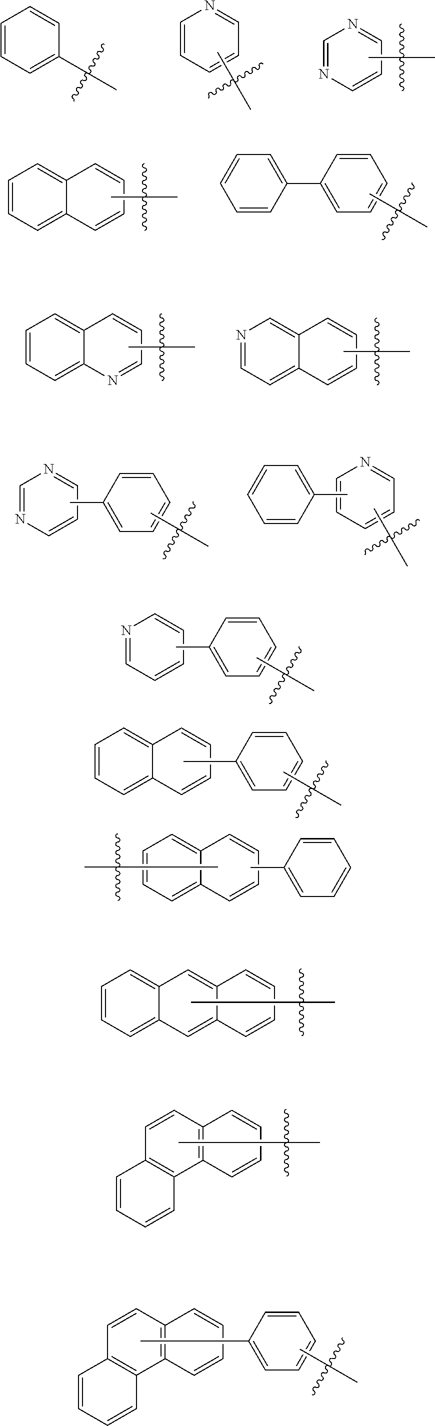 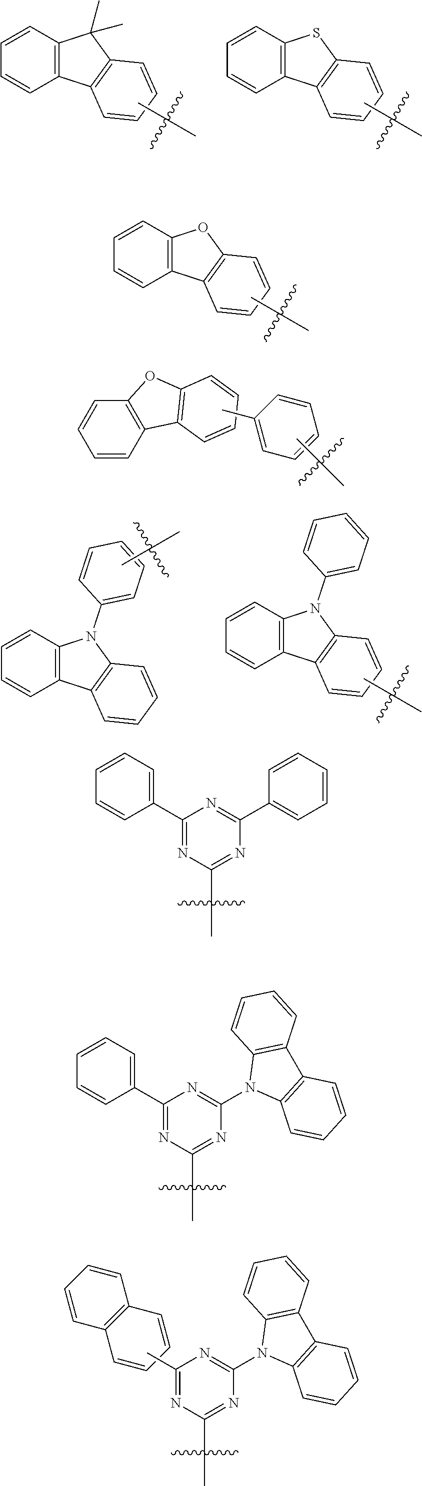 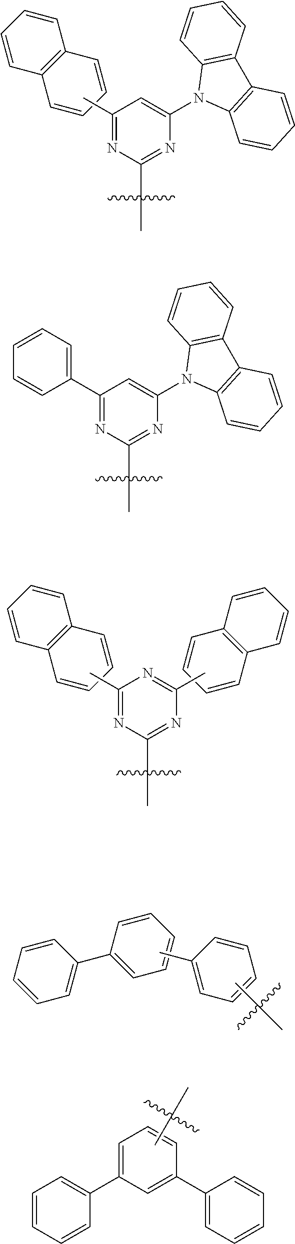 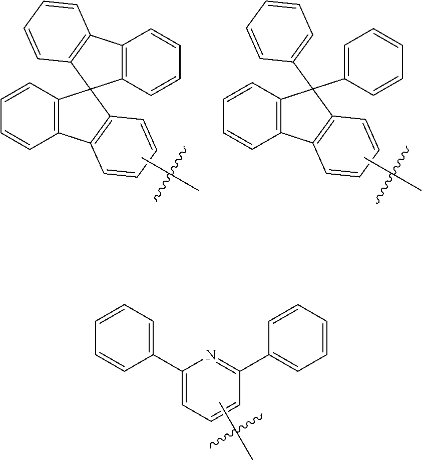
the substituted group T has one or more substituents, and the substituents of T are independently selected from deuterium, a fluorine, a cyano, a trimethylsilyl, a methyl, an ethyl, an isopropyl, a tert-butyl, a phenyl, a pyridyl, a pyrimidyl, a phenanthryl, a naphthyl, a carbazolyl, a dibenzofuranyl, or a dibenzothienyl.

10. (canceled)

11. The arylamine compound according to claim 1, wherein the Ar₅ is selected from a group consisting of the following groups:

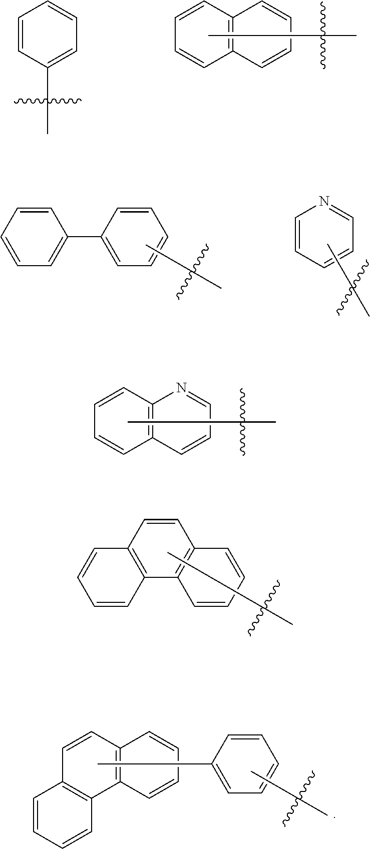

12. (canceled)

13. (canceled)

14. The arylamine compound according to claim 1, wherein the Ar₅ is selected from deuterium, a cyano, a fluorine, a trimethylsilyl, a deuterated methyl, a methyl, an ethyl, an isopropyl, a tert-butyl or a group consisting of the following groups:

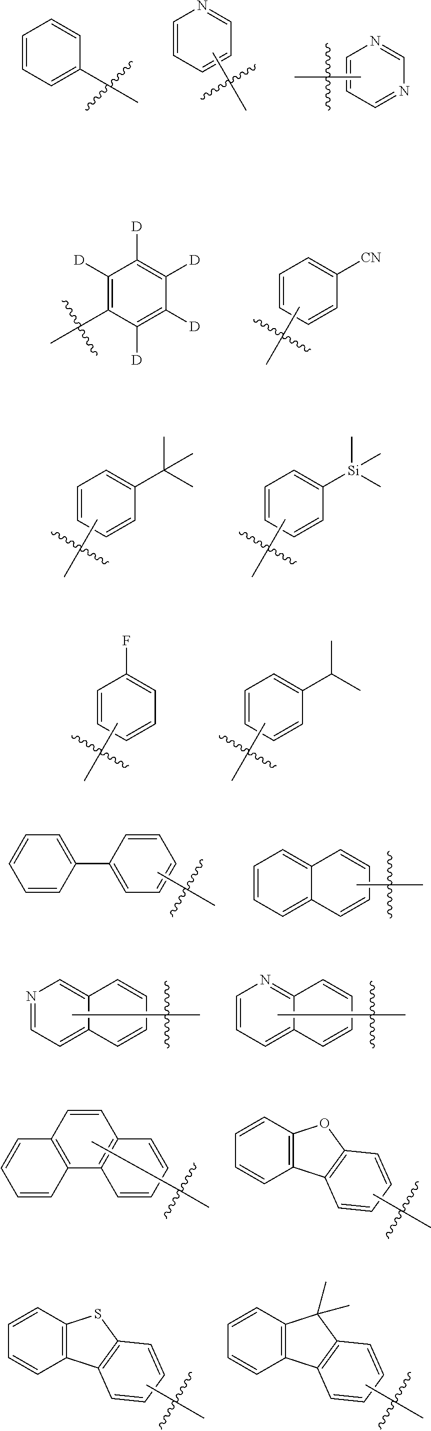

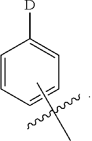

15. (canceled)

16. (canceled)

17. The arylamine compound according to claim 1, wherein the R₁ is selected from hydrogen, deuterium, a cyano, a fluorine, a trimethylsilyl, a triphenylsilyl, an alkyl with 1 to 10 carbon atoms or a group consisting of the following groups:

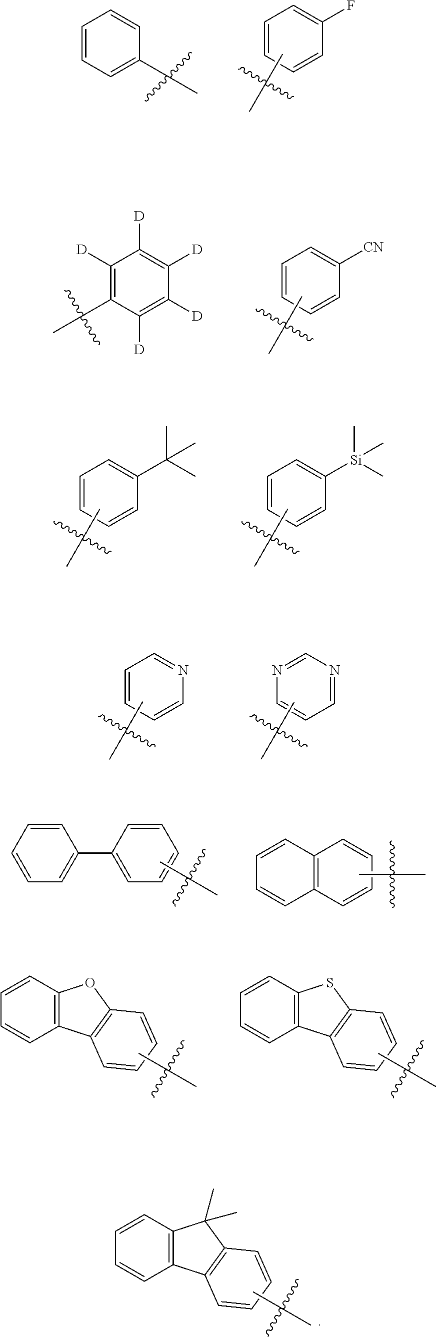

18. The arylamine compound according to claim 1, wherein the Ar₂ is selected from a group consisting of the following groups:

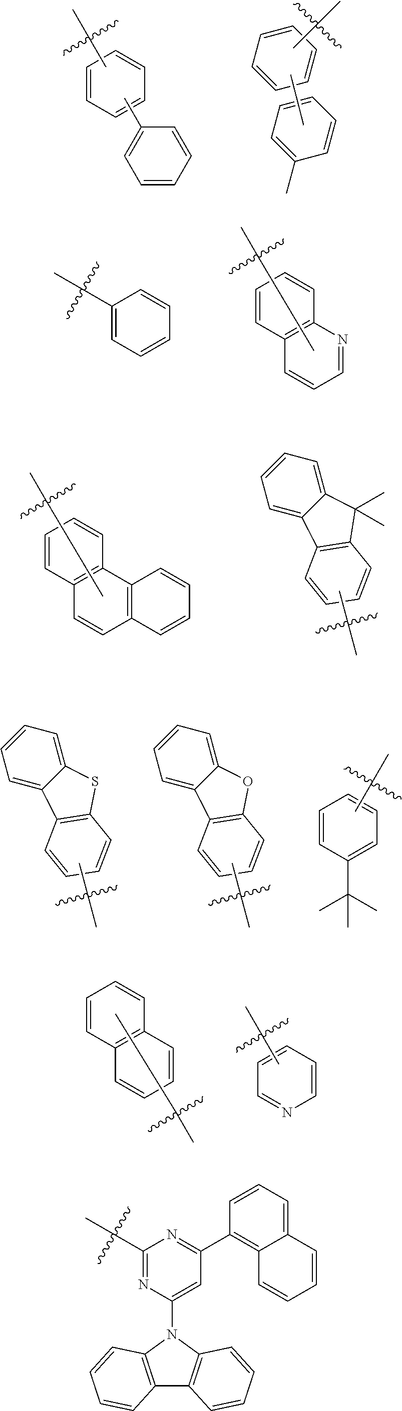

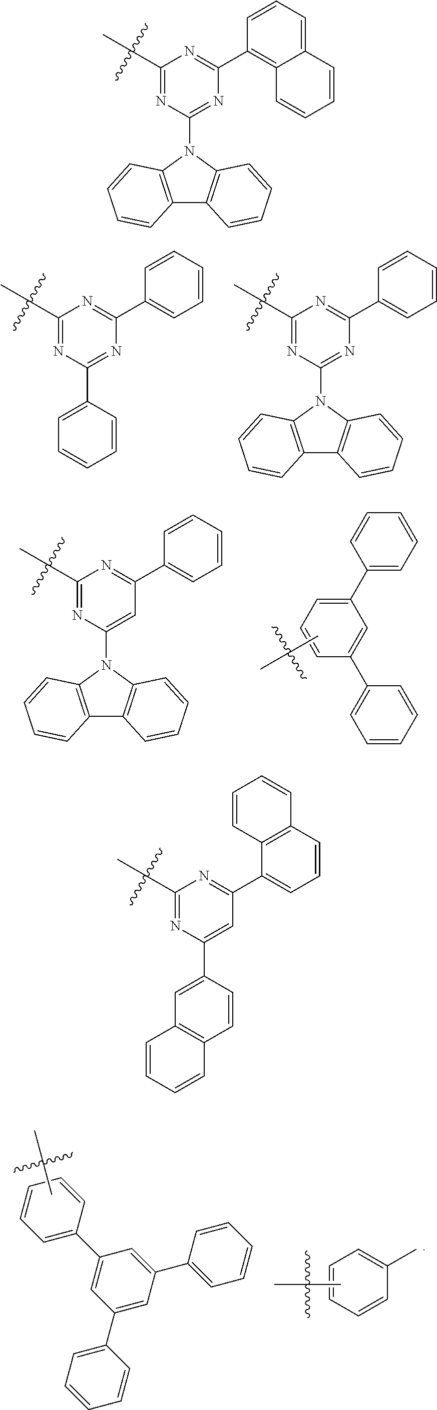

19. The arylamine compound according to claim 1, wherein the arylamine compound is selected from one or more of the following compounds:

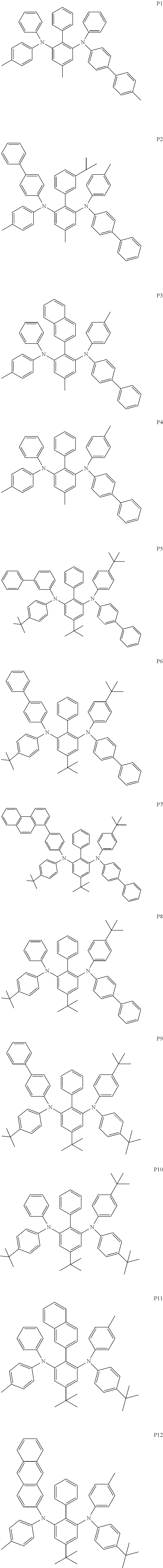

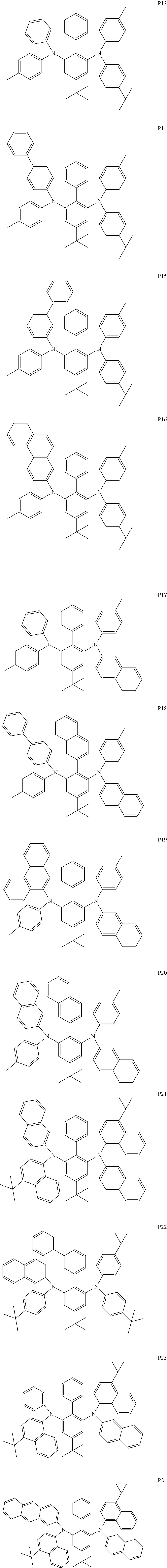

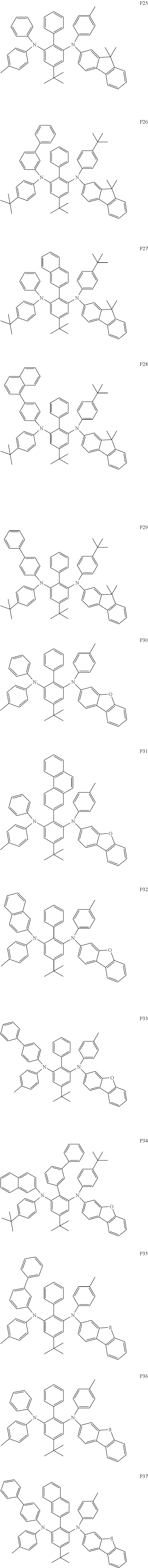

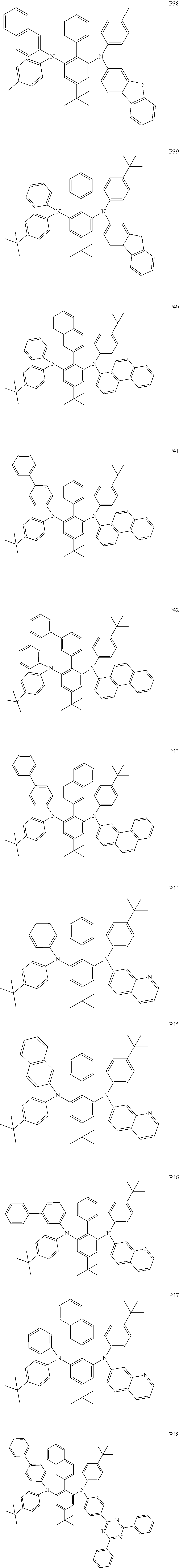

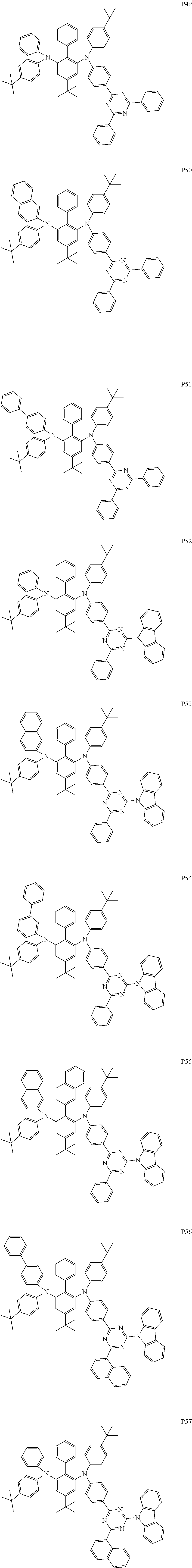

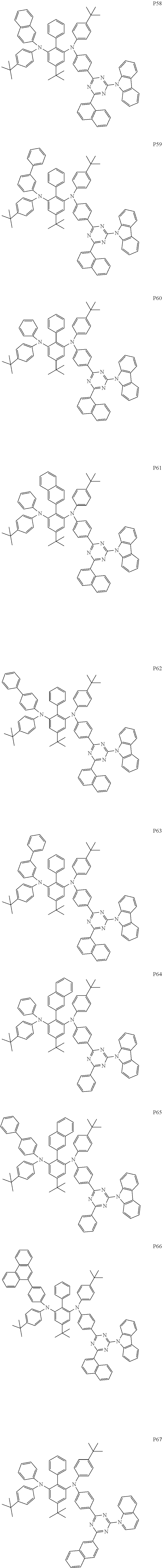

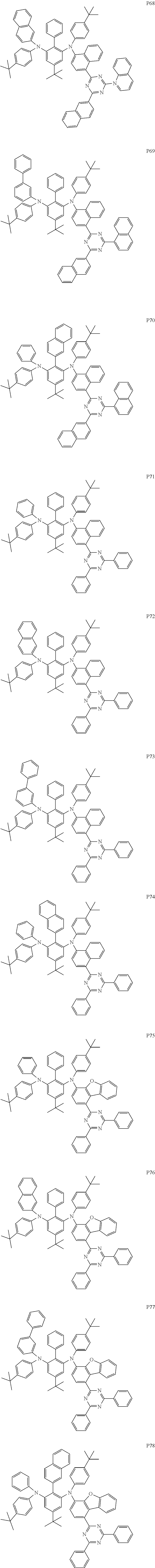

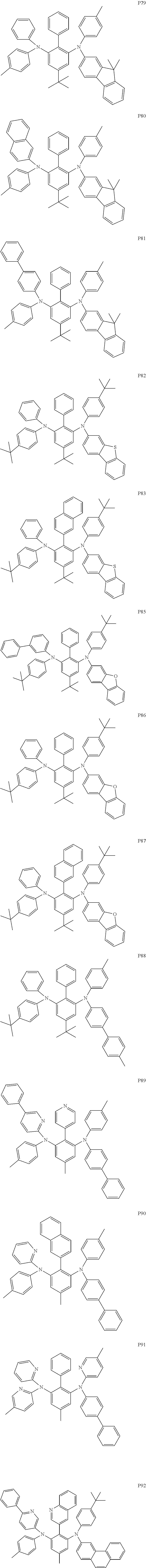

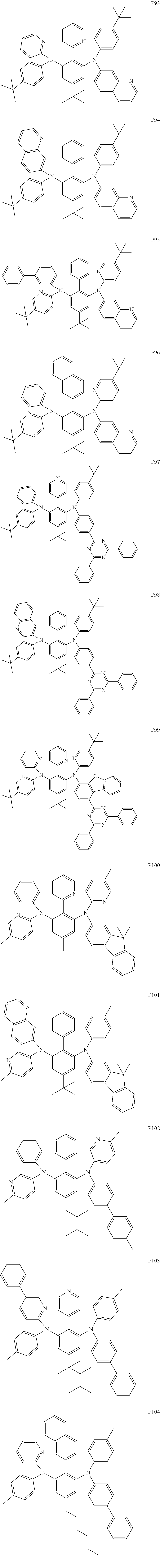

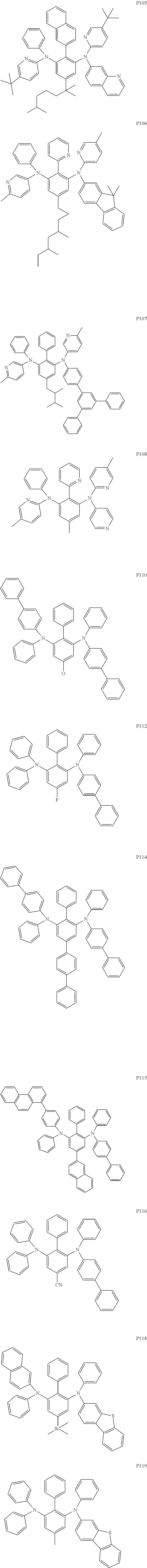

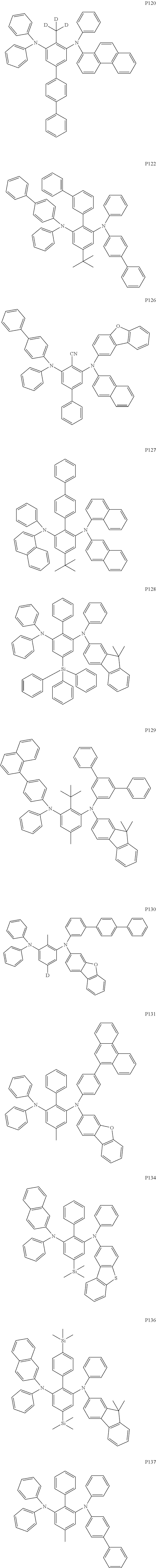

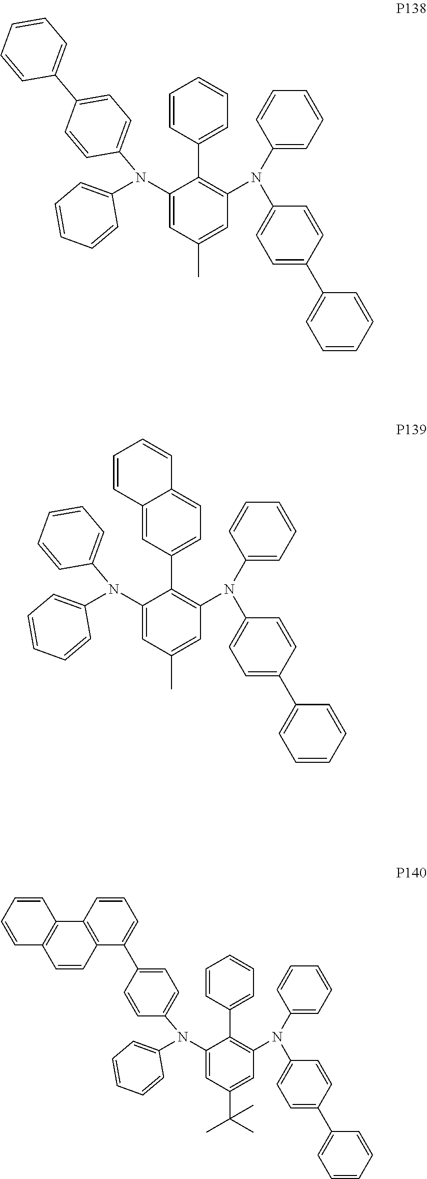 [SMILES: Cc1cc(N(c2ccccc2)c2ccc(-c3ccccc3)cc2)c(-c2ccccc2)c(N(c2ccccc2)c2ccc(-c3ccccc3)cc2)c1 ; Cc1cc(N(c2ccccc2)c2ccccc2)c(-c2ccc3ccccc3c2)c(N(c2ccccc2)c2ccc(-c3ccccc3)cc2)c1 ; CC(C)(C)c1cc(N(c2ccccc2)c2ccc(-c3ccccc3)cc2)c(-c2ccccc2)c(N(c2ccccc2)c2ccc(-c3cccc4c3ccc3ccccc34)cc2)c1]

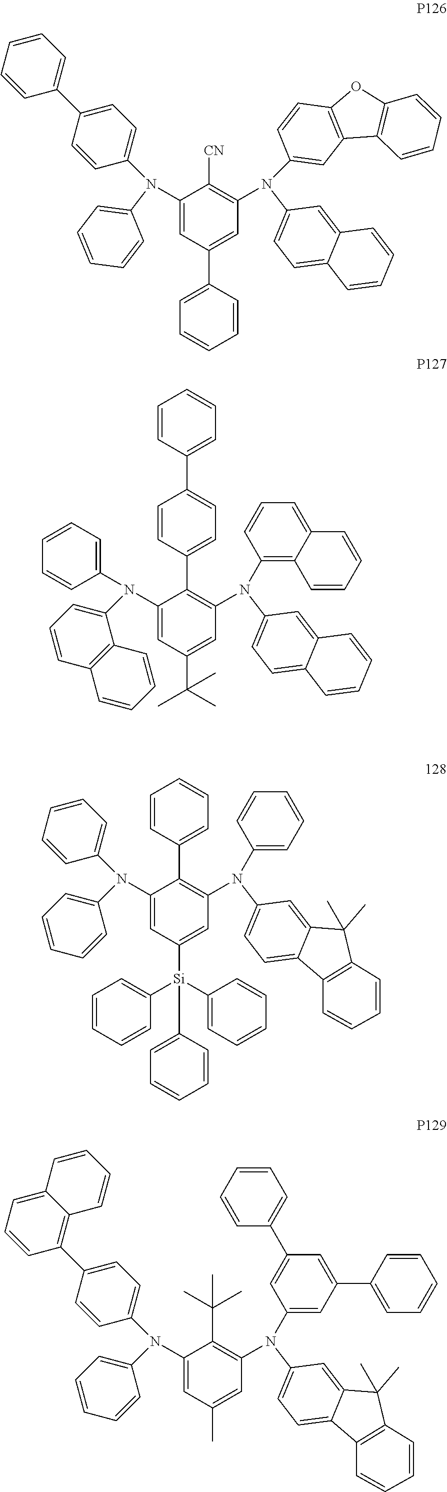

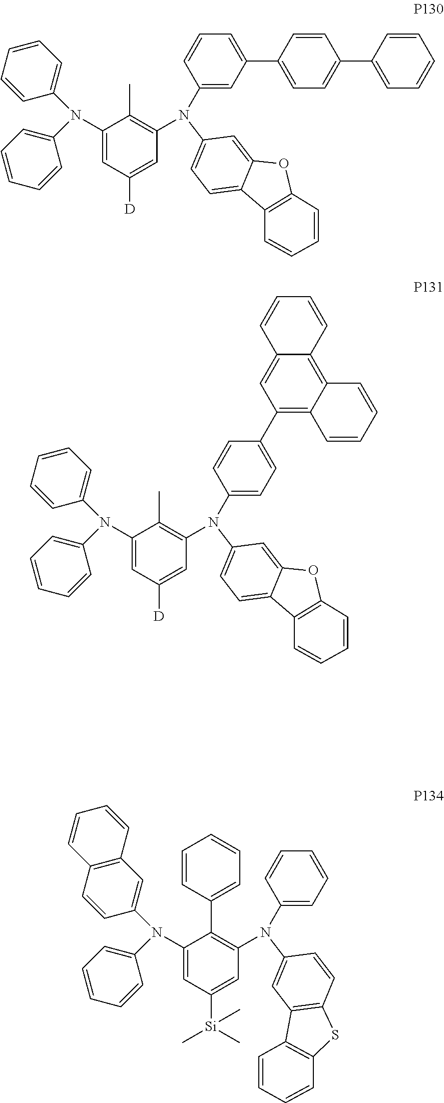

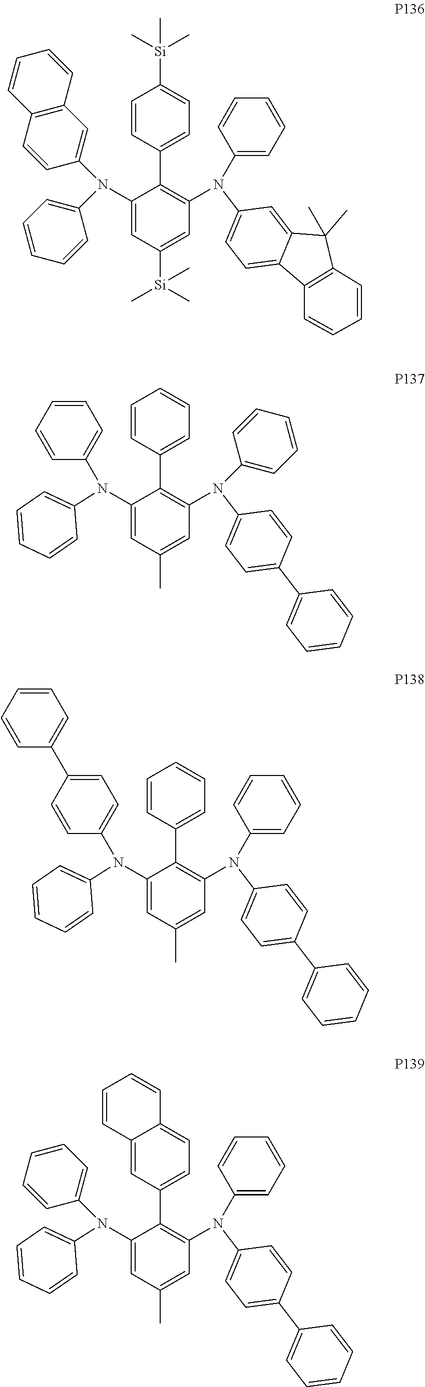 [SMILES: CC1(C)c2ccccc2-c2ccc(N(c3ccccc3)c3cc([Si](C)(C)C)cc(N(c4ccccc4)c4ccc5ccccc5c4)c3-c3ccc([Si](C)(C)C)cc3)cc21 ; Cc1cc(N(c2ccccc2)c2ccccc2)c(-c2ccccc2)c(N(c2ccccc2)c2ccc(-c3ccccc3)cc2)c1 ; Cc1cc(N(c2ccccc2)c2ccc(-c3ccccc3)cc2)c(-c2ccccc2)c(N(c2ccccc2)c2ccc(-c3ccccc3)cc2)c1 ; Cc1cc(N(c2ccccc2)c2ccccc2)c(-c2ccc3ccccc3c2)c(N(c2ccccc2)c2ccc(-c3ccccc3)cc2)c1]

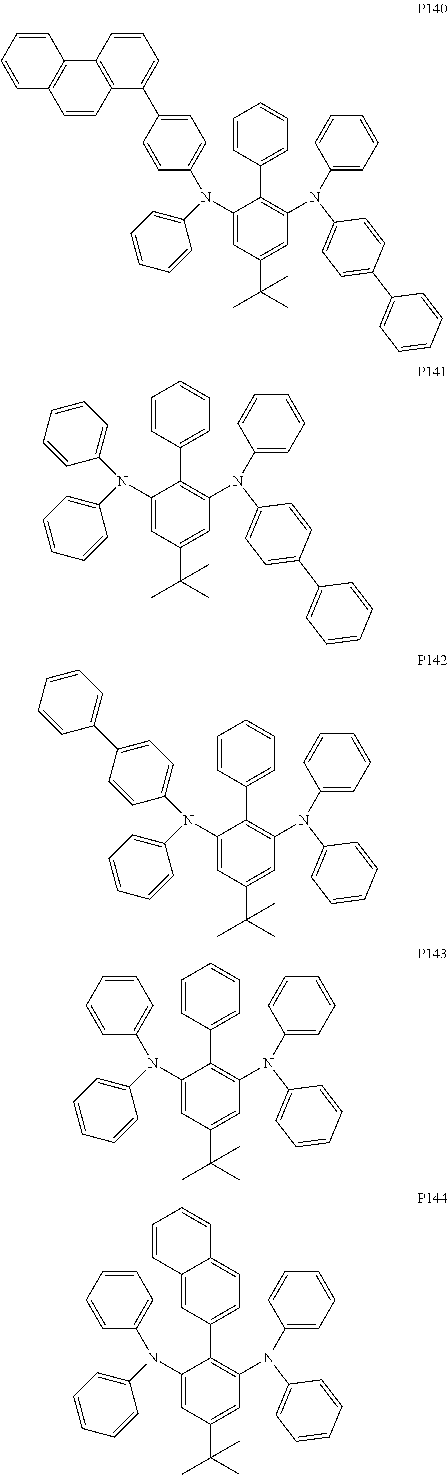

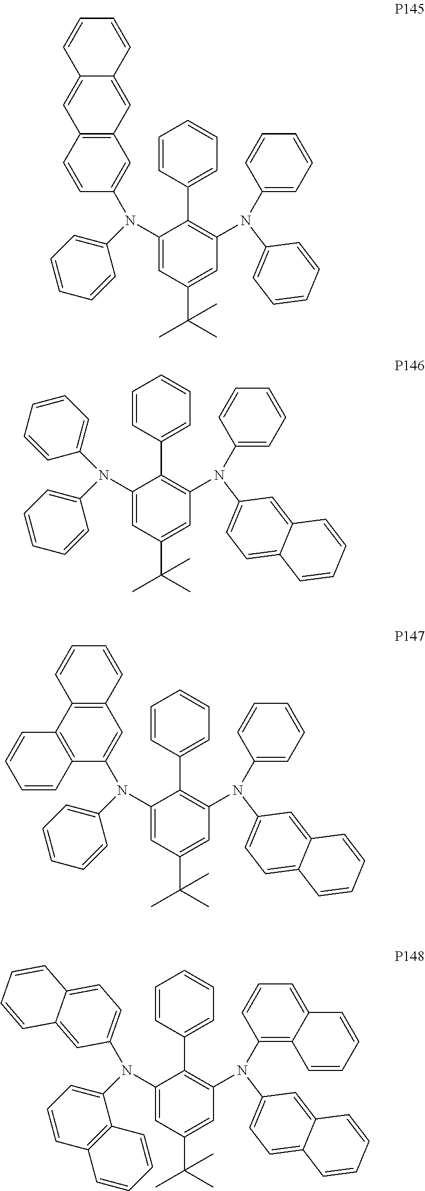

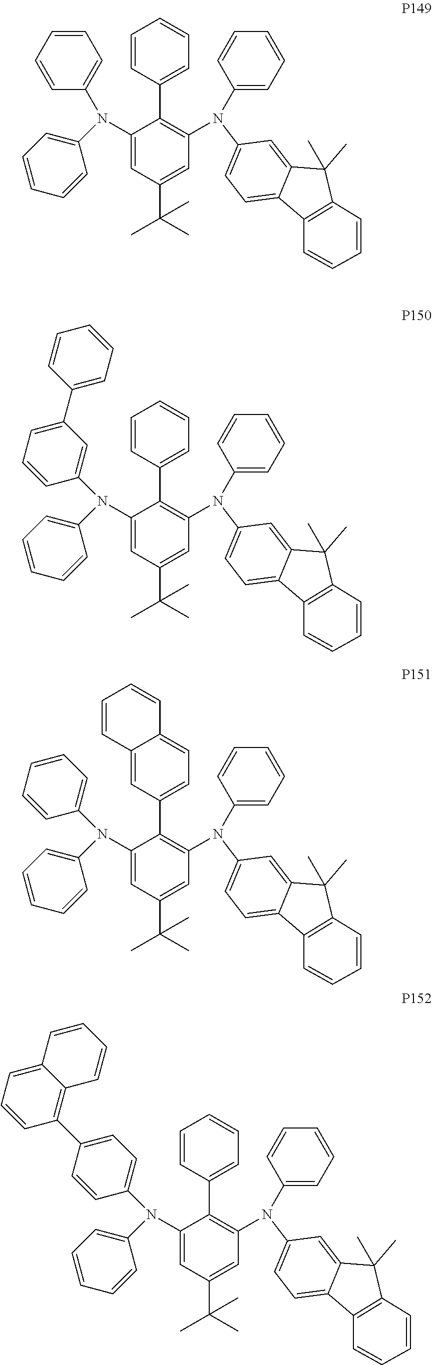 [SMILES: CC(C)(C)c1cc(N(c2ccccc2)c2ccccc2)c(-c2ccccc2)c(N(c2ccccc2)c2ccc3c(c2)C(C)(C)c2ccccc2-3)c1 ; CC(C)(C)c1cc(N(c2ccccc2)c2cccc(-c3ccccc3)c2)c(-c2ccccc2)c(N(c2ccccc2)c2ccc3c(c2)C(C)(C)c2ccccc2-3)c1 ; CC(C)(C)c1cc(N(c2ccccc2)c2ccccc2)c(-c2ccc3ccccc3c2)c(N(c2ccccc2)c2ccc3c(c2)C(C)(C)c2ccccc2-3)c1 ; CC(C)(C)c1cc(N(c2ccccc2)c2ccc(-c3cccc4ccccc34)cc2)c(-c2ccccc2)c(N(c2ccccc2)c2ccc3c(c2)C(C)(C)c2ccccc2-3)c1]

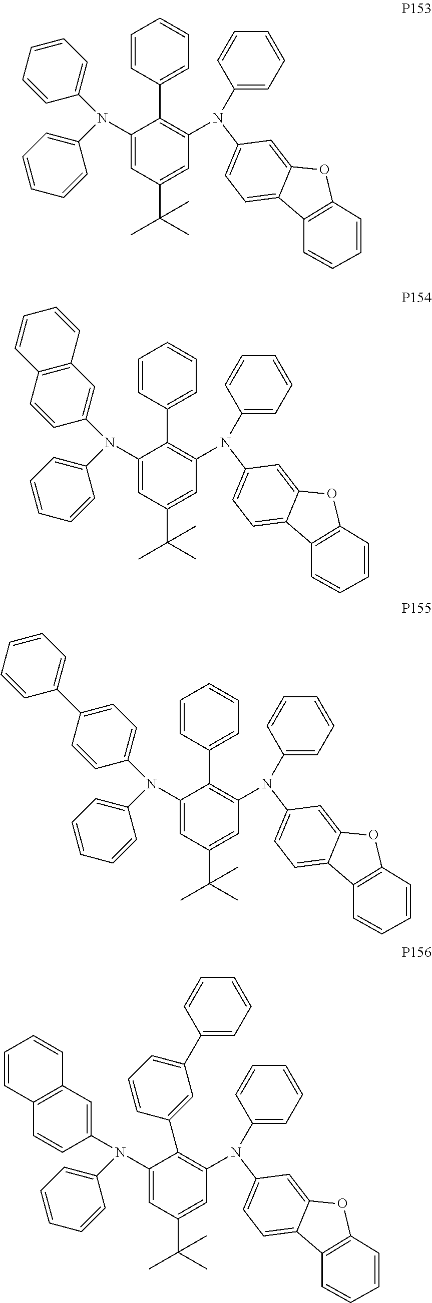 [SMILES: CC(C)(C)c1cc(N(c2ccccc2)c2ccccc2)c(-c2ccccc2)c(N(c2ccccc2)c2ccc3c(c2)oc2ccccc23)c1 ; CC(C)(C)c1cc(N(c2ccccc2)c2ccc3ccccc3c2)c(-c2ccccc2)c(N(c2ccccc2)c2ccc3c(c2)oc2ccccc23)c1 ; CC(C)(C)c1cc(N(c2ccccc2)c2ccc(-c3ccccc3)cc2)c(-c2ccccc2)c(N(c2ccccc2)c2ccc3c(c2)oc2ccccc23)c1 ; CC(C)(C)c1cc(N(c2ccccc2)c2ccc3ccccc3c2)c(-c2cccc(-c3ccccc3)c2)c(N(c2ccccc2)c2ccc3c(c2)oc2ccccc23)c1]

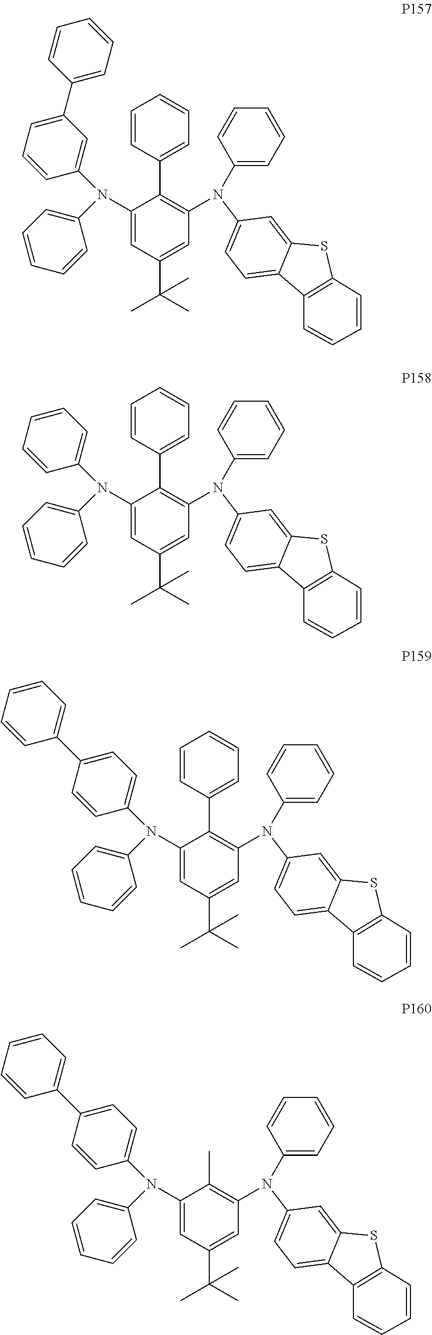 [SMILES: CC(C)(C)c1cc(N(c2ccccc2)c2cccc(-c3ccccc3)c2)c(-c2ccccc2)c(N(c2ccccc2)c2ccc3c(c2)sc2ccccc23)c1 ; CC(C)(C)c1cc(N(c2ccccc2)c2ccccc2)c(-c2ccccc2)c(N(c2ccccc2)c2ccc3c(c2)sc2ccccc23)c1 ; CC(C)(C)c1cc(N(c2ccccc2)c2ccc(-c3ccccc3)cc2)c(-c2ccccc2)c(N(c2ccccc2)c2ccc3c(c2)sc2ccccc23)c1 ; Cc1c(N(c2ccccc2)c2ccc(-c3ccccc3)cc2)cc(C(C)(C)C)cc1N(c1ccccc1)c1ccc2c(c1)sc1ccccc12]

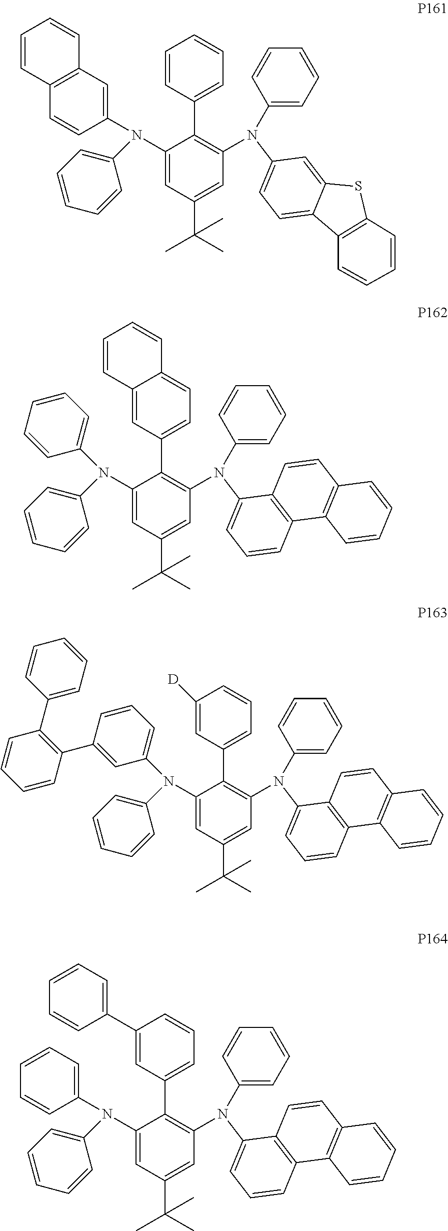 [SMILES: CC(C)(C)c1cc(N(c2ccccc2)c2ccc3ccccc3c2)c(-c2ccccc2)c(N(c2ccccc2)c2ccc3c(c2)sc2ccccc23)c1 ; CC(C)(C)c1cc(N(c2ccccc2)c2ccccc2)c(-c2ccc3ccccc3c2)c(N(c2ccccc2)c2cccc3c2ccc2ccccc23)c1 ; [2H]c1cccc(-c2c(N(c3ccccc3)c3cccc(-c4ccccc4-c4ccccc4)c3)cc(C(C)(C)C)cc2N(c2ccccc2)c2cccc3c2ccc2ccccc23)c1 ; CC(C)(C)c1cc(N(c2ccccc2)c2ccccc2)c(-c2cccc(-c3ccccc3)c2)c(N(c2ccccc2)c2cccc3c2ccc2ccccc23)c1]

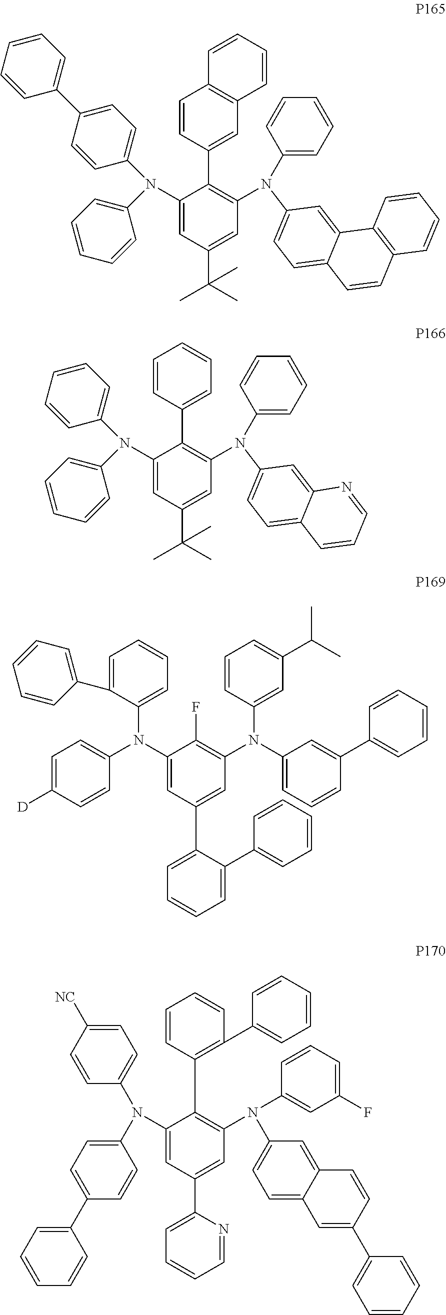

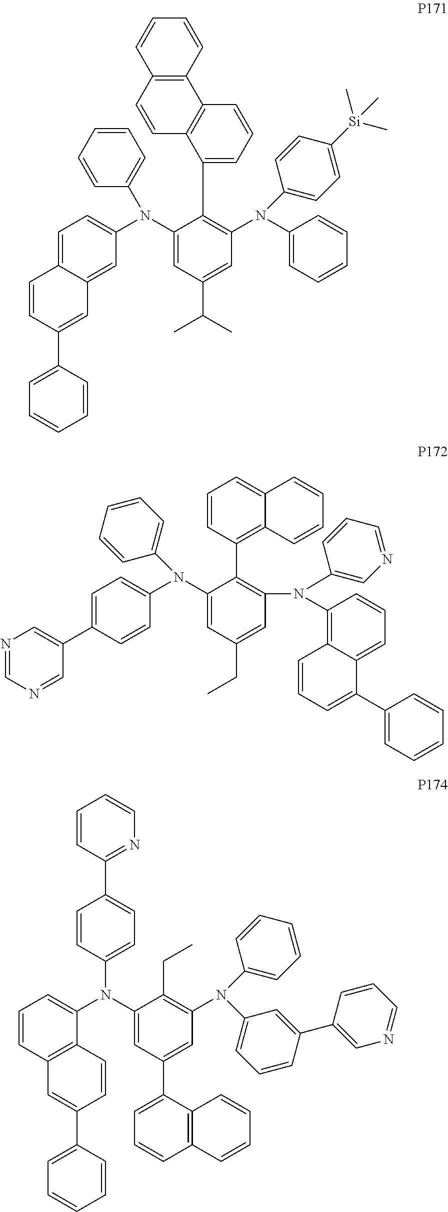 [SMILES: CC(C)c1cc(N(c2ccccc2)c2ccc([Si](C)(C)C)cc2)c(-c2cccc3c2ccc2ccccc23)c(N(c2ccccc2)c2ccc3ccc(-c4ccccc4)cc3c2)c1 ; CCc1cc(N(c2ccccc2)c2ccc(-c3cncnc3)cc2)c(-c2cccc3ccccc23)c(N(c2cccnc2)c2cccc3c(-c4ccccc4)cccc23)c1 ; CCc1c(N(c2ccccc2)c2cccc(-c3cccnc3)c2)cc(-c2cccc3ccccc23)cc1N(c1ccc(-c2ccccn2)cc1)c1cccc2cc(-c3ccccc3)ccc12]

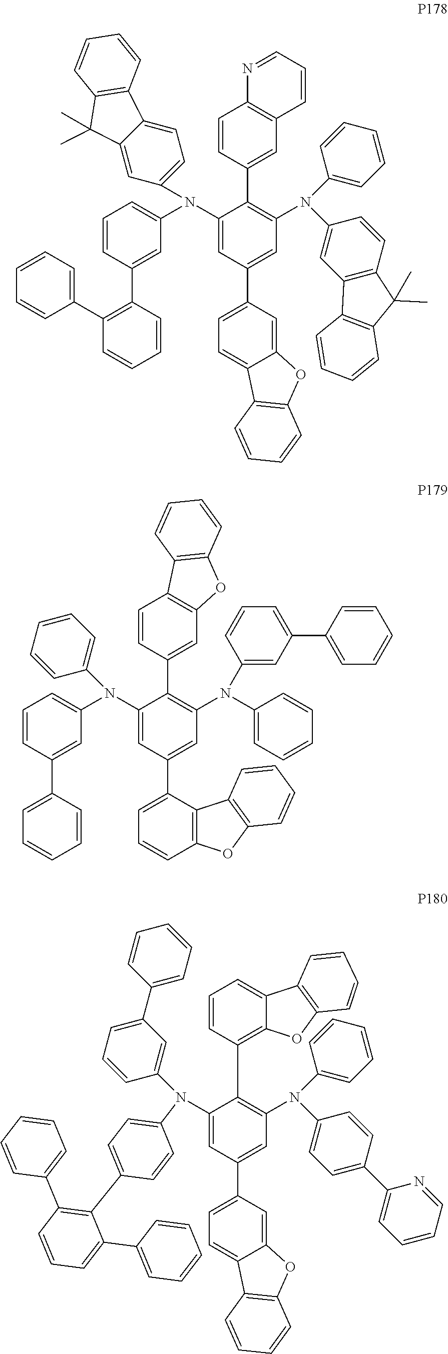 [SMILES: CC1(C)c2ccccc2-c2cc(N(c3ccccc3)c3cc(-c4ccc5c(c4)oc4ccccc45)cc(N(c4cccc(-c5ccccc5-c5ccccc5)c4)c4ccc5c(c4)C(C)(C)c4ccccc4-5)c3-c3ccc4ncccc4c3)ccc21 ; c1ccc(-c2cccc(N(c3ccccc3)c3cc(-c4cccc5oc6ccccc6c45)cc(N(c4ccccc4)c4cccc(-c5ccccc5)c4)c3-c3ccc4c(c3)oc3ccccc34)c2)cc1 ; c1ccc(-c2cccc(N(c3ccc(-c4c(-c5ccccc5)cccc4-c4ccccc4)cc3)c3cc(-c4ccc5c(c4)oc4ccccc45)cc(N(c4ccccc4)c4ccc(-c5ccccn5)cc4)c3-c3cccc4c3oc3ccccc34)c2)cc1]

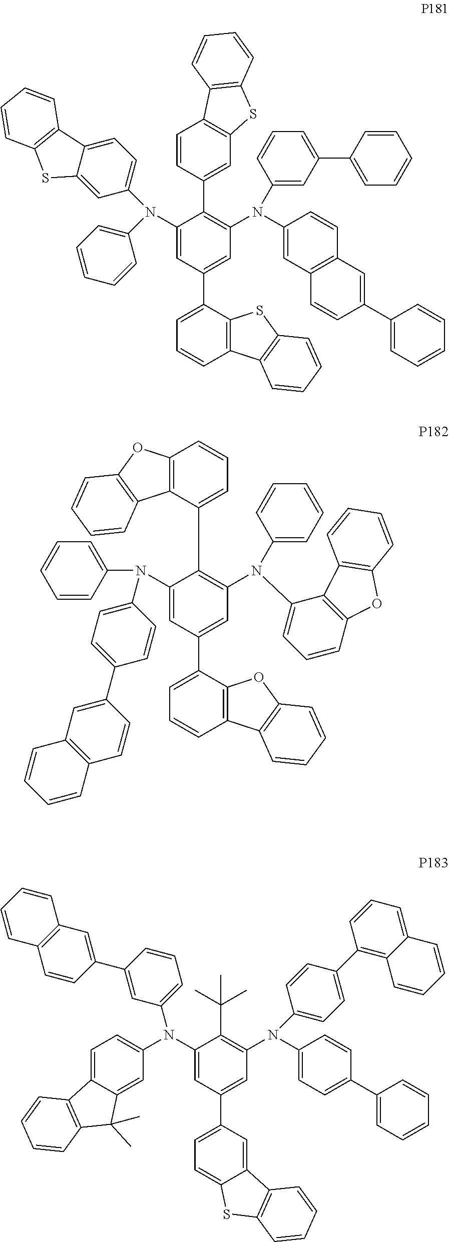 [SMILES: c1ccc(-c2cccc(N(c3ccc4cc(-c5ccccc5)ccc4c3)c3cc(-c4cccc5c4sc4ccccc45)cc(N(c4ccccc4)c4ccc5c(c4)sc4ccccc45)c3-c3ccc4c(c3)sc3ccccc34)c2)cc1 ; c1ccc(N(c2ccc(-c3ccc4ccccc4c3)cc2)c2cc(-c3cccc4c3oc3ccccc34)cc(N(c3ccccc3)c3cccc4oc5ccccc5c34)c2-c2cccc3oc4ccccc4c23)cc1 ; CC(C)(C)c1c(N(c2ccc(-c3ccccc3)cc2)c2ccc(-c3cccc4ccccc34)cc2)cc(-c2ccc3sc4ccccc4c3c2)cc1N(c1cccc(-c2ccc3ccccc3c2)c1)c1ccc2c(c1)C(C)(C)c1ccccc1-2]

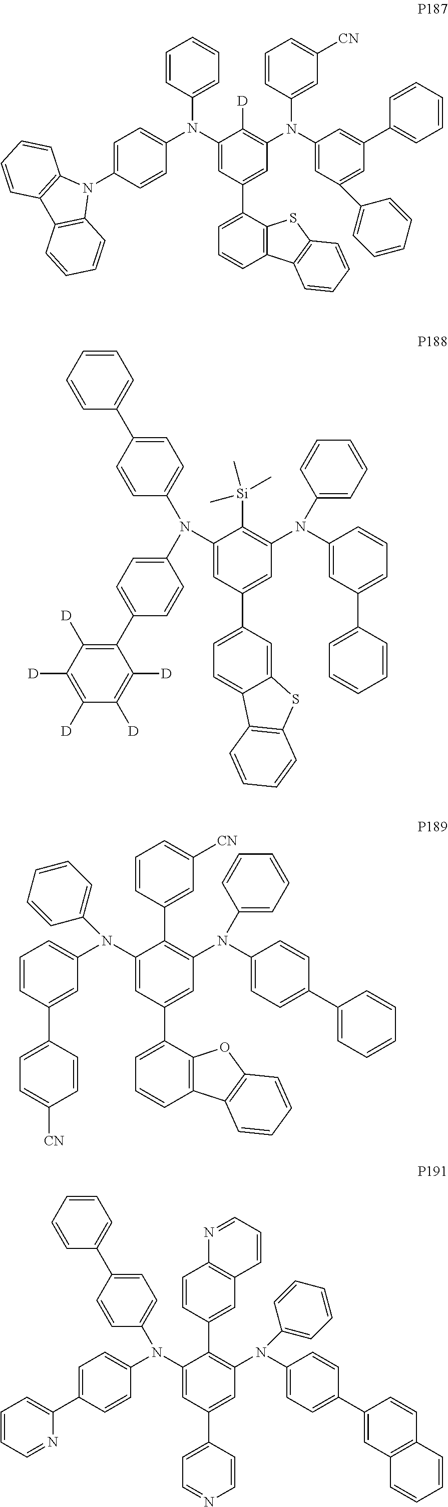

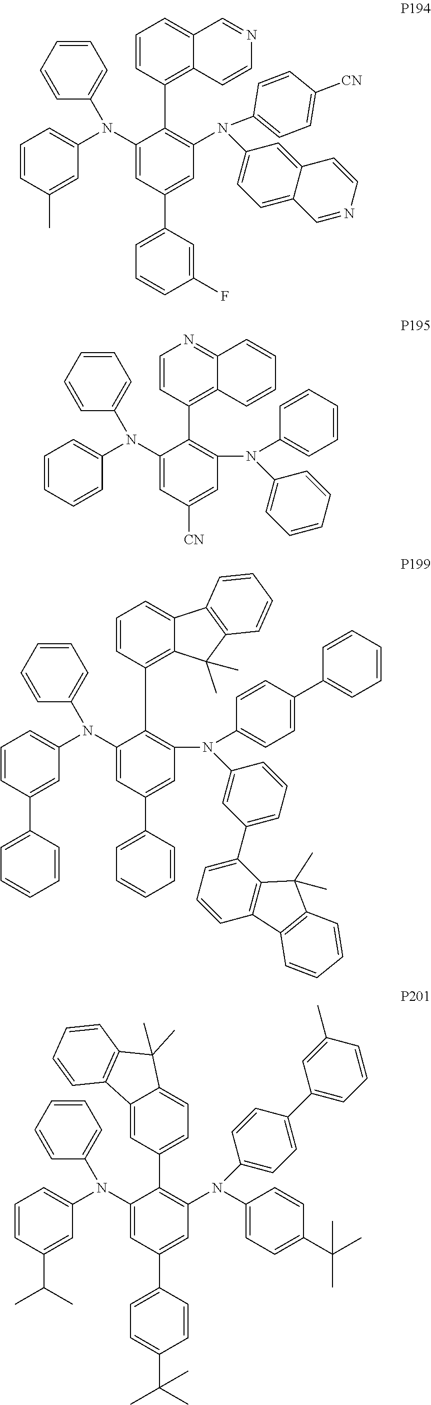

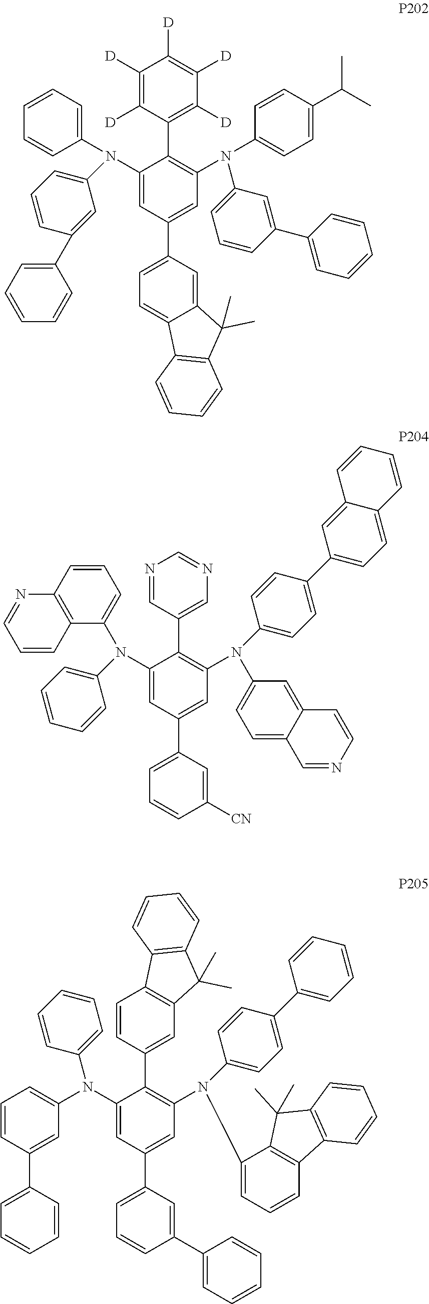 [SMILES: [2H]c1c([2H])c([2H])c(-c2c(N(c3ccccc3)c3cccc(-c4ccccc4)c3)cc(-c3ccc4c(c3)C(C)(C)c3ccccc3-4)cc2N(c2ccc(C(C)C)cc2)c2cccc(-c3ccccc3)c2)c([2H])c1[2H] ; N#Cc1cccc(-c2cc(N(c3ccc(-c4ccc5ccccc5c4)cc3)c3ccc4cnccc4c3)c(-c3cncnc3)c(N(c3ccccc3)c3cccc4ncccc34)c2)c1 ; CC1(C)c2ccccc2-c2ccc(-c3c(N(c4ccccc4)c4cccc(-c5ccccc5)c4)cc(-c4cccc(-c5ccccc5)c4)cc3N(c3ccc(-c4ccccc4)cc3)c3cccc4c3C(C)(C)c3ccccc3-4)cc21]

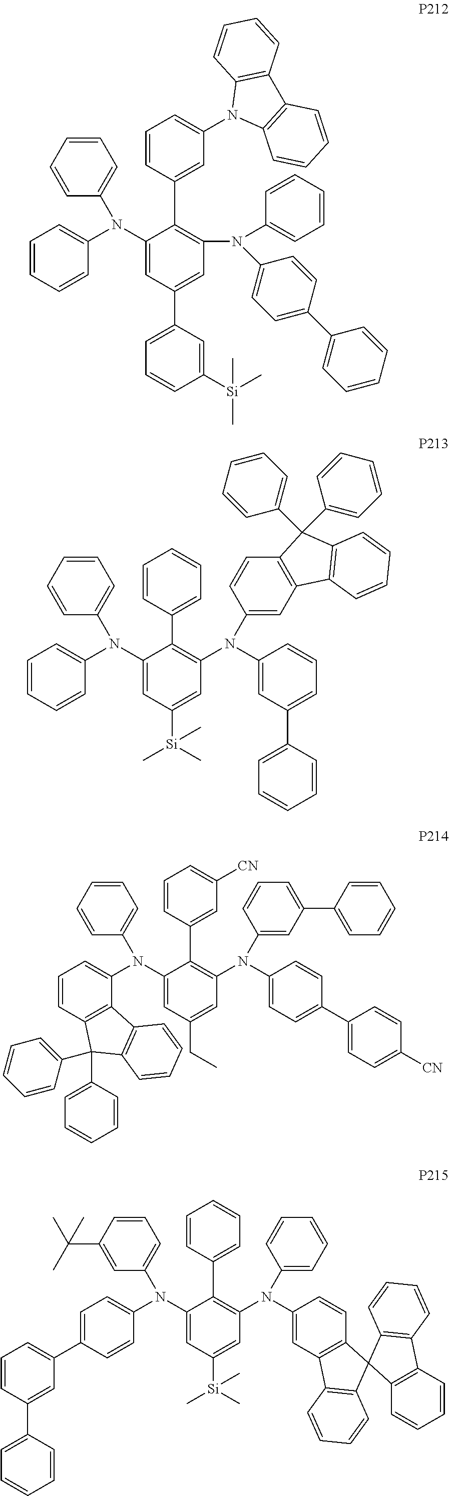

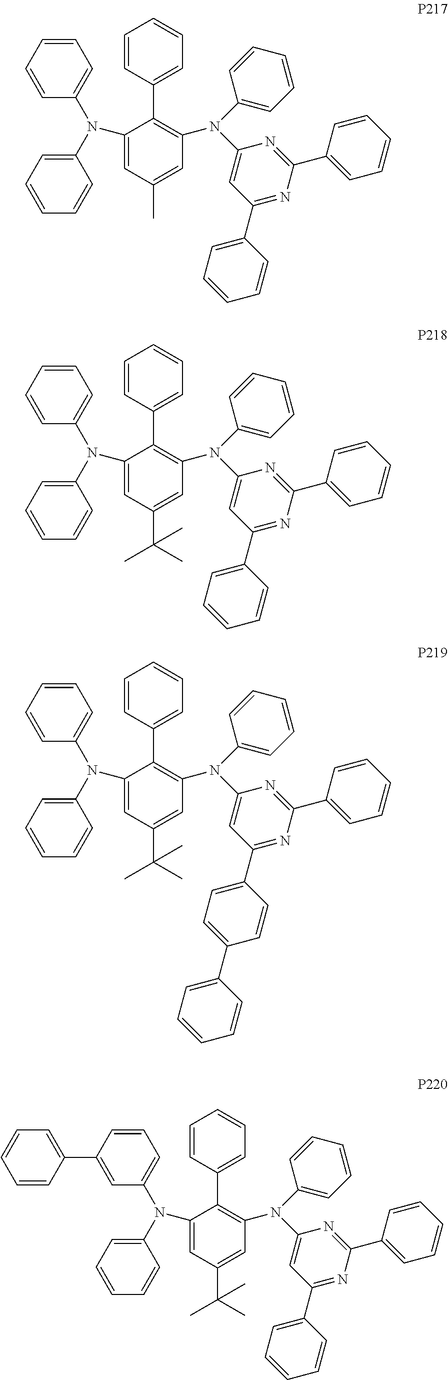 [SMILES: Cc1cc(N(c2ccccc2)c2ccccc2)c(-c2ccccc2)c(N(c2ccccc2)c2cc(-c3ccccc3)nc(-c3ccccc3)n2)c1 ; CC(C)(C)c1cc(N(c2ccccc2)c2ccccc2)c(-c2ccccc2)c(N(c2ccccc2)c2cc(-c3ccccc3)nc(-c3ccccc3)n2)c1 ; CC(C)(C)c1cc(N(c2ccccc2)c2ccccc2)c(-c2ccccc2)c(N(c2ccccc2)c2cc(-c3ccc(-c4ccccc4)cc3)nc(-c3ccccc3)n2)c1 ; CC(C)(C)c1cc(N(c2ccccc2)c2cccc(-c3ccccc3)c2)c(-c2ccccc2)c(N(c2ccccc2)c2cc(-c3ccccc3)nc(-c3ccccc3)n2)c1]

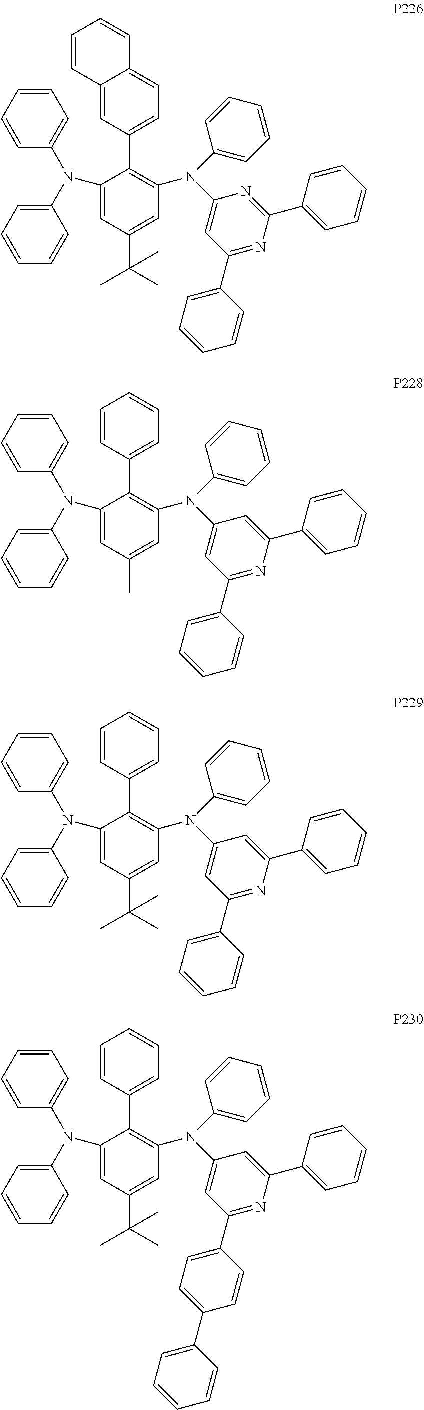 [SMILES: CC(C)(C)c1cc(N(c2ccccc2)c2ccccc2)c(-c2ccc3ccccc3c2)c(N(c2ccccc2)c2cc(-c3ccccc3)nc(-c3ccccc3)n2)c1 ; Cc1cc(N(c2ccccc2)c2ccccc2)c(-c2ccccc2)c(N(c2ccccc2)c2cc(-c3ccccc3)nc(-c3ccccc3)c2)c1 ; CC(C)(C)c1cc(N(c2ccccc2)c2ccccc2)c(-c2ccccc2)c(N(c2ccccc2)c2cc(-c3ccccc3)nc(-c3ccccc3)c2)c1 ; CC(C)(C)c1cc(N(c2ccccc2)c2ccccc2)c(-c2ccccc2)c(N(c2ccccc2)c2cc(-c3ccccc3)nc(-c3ccc(-c4ccccc4)cc3)c2)c1]

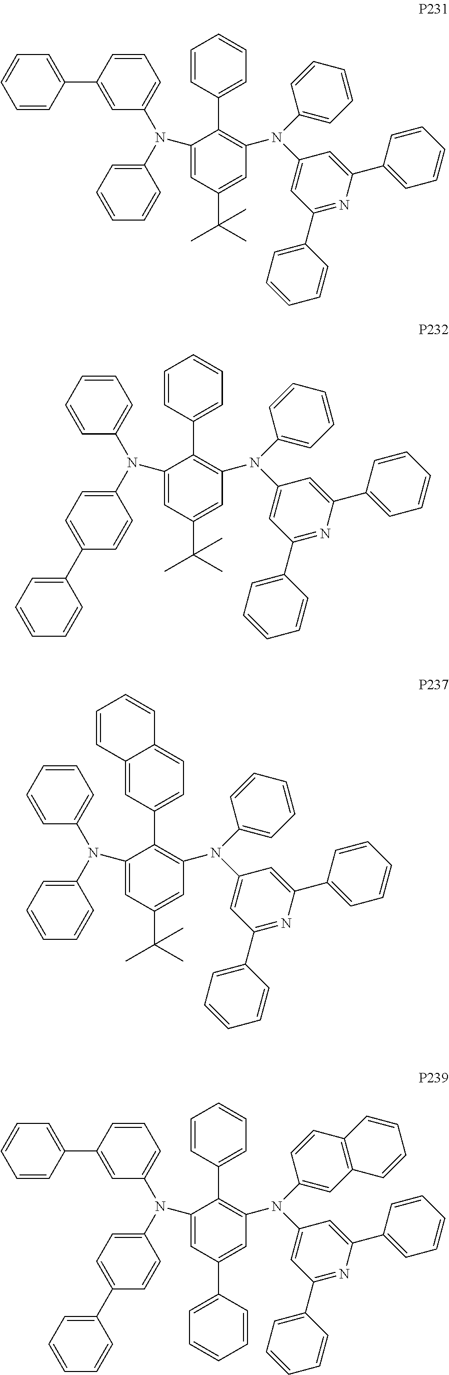 [SMILES: CC(C)(C)c1cc(N(c2ccccc2)c2cccc(-c3ccccc3)c2)c(-c2ccccc2)c(N(c2ccccc2)c2cc(-c3ccccc3)nc(-c3ccccc3)c2)c1 ; CC(C)(C)c1cc(N(c2ccccc2)c2ccc(-c3ccccc3)cc2)c(-c2ccccc2)c(N(c2ccccc2)c2cc(-c3ccccc3)nc(-c3ccccc3)c2)c1 ; CC(C)(C)c1cc(N(c2ccccc2)c2ccccc2)c(-c2ccc3ccccc3c2)c(N(c2ccccc2)c2cc(-c3ccccc3)nc(-c3ccccc3)c2)c1 ; c1ccc(-c2ccc(N(c3cccc(-c4ccccc4)c3)c3cc(-c4ccccc4)cc(N(c4cc(-c5ccccc5)nc(-c5ccccc5)c4)c4ccc5ccccc5c4)c3-c3ccccc3)cc2)cc1]

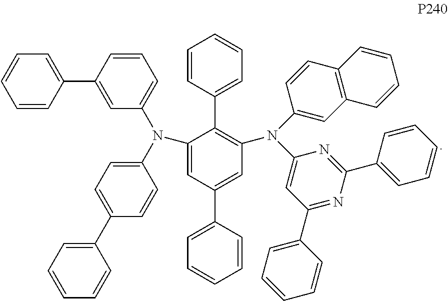 [SMILES: c1ccc(-c2ccc(N(c3cccc(-c4ccccc4)c3)c3cc(-c4ccccc4)cc(N(c4ccc5ccccc5c4)c4cc(-c5ccccc5)nc(-c5ccccc5)n4)c3-c3ccccc3)cc2)cc1]

20. An organic electroluminescent device, comprising an anode, a cathode and at least one functional layer between the anode and the cathode, wherein the functional layer contains a hole injection layer, a hole transport layer, an organic electroluminescent layer, an electron transport layer and an electron injection layer, and at least one of the hole transport layer and the electron transport layer contains the arylamine compound according to claim 1.
